(19) United States
(12) Patent Application Publication
(10) Pub. No.: US 2014/0378524 A1
(43) Pub. Date: Dec. 25, 2014

(54) METHODS AND COMPOSITIONS COMPRISING AKT INHIBITORS AND/OR PHOSPHOLIPASE D INHIBITORS

(71) Applicant: Vanderbilt University, Nashville, TN (US)

(72) Inventors: H. Alex Brown, Franklin, TN (US); Craig W. Lindsley, Brentwood, TN (US)

(21) Appl. No.: 14/103,819

(22) Filed: Dec. 11, 2013

Related U.S. Application Data
(60) Provisional application No. 61/736,003, filed on Dec. 11, 2012.

(51) Int. Cl. A61K 31/713; A61K 31/454; A61K 31/436; A61K 31/435

(52) U.S. Cl. CPC A61K 31/713; A61K 31/435; A61K 31/454; A61K 31/436
USPC 514/44 A; 514/278; 514/291

(57) ABSTRACT

Disclosed are methods of treating viral infections or disorders of uncontrolled proliferation comprising, in one aspect, administering compounds that are phospholipase D inhibitors and/or Akt therapeutic agents. This abstract is intended as a scanning tool for purposes of searching in the particular art and is not intended to be limiting of the present invention.

### CROSS-REFERENCE TO RELATED APPLICATIONS

This Application claims the benefit of U.S. Provisional Application No. 61/736,003, filed on Dec. 11, 2012, which is incorporated herein by reference in its entirety.

### STATEMENT REGARDING FEDERALLY SPONSORED RESEARCH

This invention was made with government support under grant numbers U54 MH084659, P01 ESO013125, NIAID HHSN2722008000058C awarded by the National Institutes of Health (NIH). The United States government has certain rights in the invention.

### BACKGROUND

Among the most frequently deregulated pathways in caner are components of the phosphoinositide 3-kinase (PI3K)/Akt pathway (Cheng, C. K., et al. (2009) Brain Pathol. 19, 112-120). Activation of PI3K either by cell-surface receptor stimulation or constitutively activating mutations results in phosphatidylinositol-3,4,5-trisphosphate (PIP₃) production and subsequently initiates signaling cascades by recruiting a variety of molecules containing lipid-binding domains to membranes (Cantley, L. C. (2002) Science 296, 1655-1657). The serine/threonine kinase Akt was identified as the eukaryotic homolog of the retroviral oncogene v-Aid, which becomes activated following PI3K generation of PIP₃ (Bellacosa, A., et al. (1991) Science 254, 274-277; Franke, T. F., et al. (1995) Cell 81, 727-736). Aid mediates a variety of intracellular functions critical to oncogenic processes, including cell growth, proliferation, metabolism, and survival (Manning, B. D., and Cantley, L. C. (2007) Cell 129, 1261-1274). Mutations that result in PI3K activation, such as constitutive growth factor receptor activation (Libermann, T. A., et al. (1985) Nature 313, 144-147) or inactivation of phosphatase and tensin homologue (“PTEN”; Haas-Kogan, D., et al. (1998) Curr. Biol. 8, 1195-1198), the lipid phosphatase that hydrolyzes PIP₃, are common in GBM. While small-molecule inhibitors of the PI3K/Akt pathway hold promise in clinical trials for cancer, e.g. glioblastoma (Furnari, F. B., et al. (2007) Gene. Dev. 21, 2683-2710), global inhibition of the Akt isoenzymes results in side effects that limit their clinical potential (Yap, T. A., et al. W. (2011) J. Clin. Oncol. 29, 4688-4695).

Despite extensive research focused on potential therapeutic agents that directly modulate the PI3K/Akt pathway, e.g. inhibition of the Akt isoenzymes, the side effect of global inhibition has limited their clinical utility. A preferred approach that can offer more selective and exquisite therapeutic modulation of the PI3K/Akt pathway is modulation of another protein that inhibits Akt activity in a particular context, but does not adversely modulate the PI3K/Akt pathway globally. Therefore, there remains a need for methods and compositions that overcome these deficiencies and that effectively provide therapeutic modulation of the PI3K/Akt pathway without the currently observed side effects associated with global inhibition of the pathway.

### SUMMARY

In accordance with the purpose(s) of the invention, as embodied and broadly described herein, the invention, in one aspect, relates to pharmaceutical compositions, kits, methods of treatment, medicaments, and uses comprising inhibitors of phospholipase D, Akt, and/or mTor inhibitors.

Disclosed are methods for treating a subject diagnosed with an infectious disease, the method comprising the step of administering to the subject an effective amount of an Akt therapeutic agent, thereby treating the subject for the infectious disease. These methods can further comprise co-administration of an effective amount of a PLD inhibitor.

Also disclosed are PLD inhibitors useful in the pharmaceutical compositions, kits, methods of treatment, medicaments, and uses of the present invention, wherein the PLD inhibitor is a compound having a structure represented by a formula:

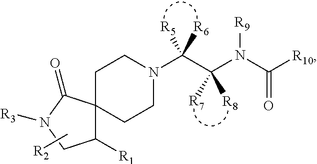

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

Also disclosed are PLD inhibitors useful in the pharmaceutical compositions, kits, methods of treatment, medicaments, and uses of the present invention, wherein the PLD inhibitor is a compound having a structure represented by a formula:

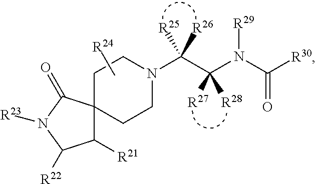

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

Also disclosed are PLD inhibitors useful in the pharmaceutical compositions, kits, methods of treatment, medicaments, and uses of the present invention, wherein the PLD inhibitor is a compound having a structure represented by a formula:

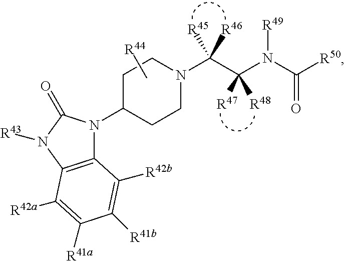

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

Also disclosed are PLD inhibitors useful in the pharmaceutical compositions, kits, methods of treatment, medicaments, and uses of the present invention, wherein the PLD inhibitor is a compound selected from: (a) trans-diethylstilbestrol; (b) resveratrol; (c) honokiol; (d) SCH420789; (e) presqualene diphosphate; (f) raloxifene; (g) 4-hydroxytamoxifen; (h) 5-fluoro-2-indoyl des-chlorohalopemide; and (i) halopemide.

Also disclosed are methods for treating a subject for a viral infection comprising the step of co-administering an effective amount of: a) a phospholipase D inhibitor, or a pharmaceutically acceptable prodrug, salt, solvate, or polymorph thereof; and b) a mTor inhibitor, or a pharmaceutically acceptable prodrug, salt, solvate, or polymorph thereof.

Also disclosed are methods modulating autophagy in at least one cell, comprising the step of contacting the cell with an effective amount of a phospholipase D inhibitor, thereby modulating autophagy in the cell.

Also disclosed are methods for treating a disorder in a subject, comprising the step of co-administering to the subject an Akt therapeutic agent and a phospholipase D inhibitor, thereby treating the disorder in the subject.

Also disclosed are pharmaceutical compositions comprising an effective amount of an Akt therapeutic agent, or a pharmaceutically acceptable prodrug, salt, solvate, or polymorph thereof; an effective amount of an antiviral therapeutic agent; and a pharmaceutically acceptable carrier.

Also disclosed are pharmaceutical compositions comprising an effective amount of an Akt therapeutic agent, or a pharmaceutically acceptable prodrug, salt, solvate, or polymorph thereof; an effective amount of at least one antibacterial therapeutic agent; and a pharmaceutically acceptable carrier.

Also disclosed are pharmaceutical compositions comprising: (a) a first therapeutic agent comprising an effective amount of a phospholipase D inhibitor, or a pharmaceutically acceptable prodrug, salt, solvate, or polymorph thereof; and (b) a second therapeutic agent comprising an effective amount of a mTor inhibitor, or a pharmaceutically acceptable prodrug, salt, solvate, or polymorph thereof; and a pharmaceutically acceptable carrier.

Also disclosed are kits comprising an Akt therapeutic agent, or a pharmaceutically acceptable prodrug, salt, solvate, or polymorph thereof, and one or more of: a) at least one therapeutic agent known to treat an HIV infection; b) at least one therapeutic agent known to treat an opportunistic infection associated with an HIV infection; c) instructions for treating an HIV infection; d) instructions for treating an opportunistic infection associated with an HIV infection; e) instructions for administering the Akt therapeutic agent in connection with treating an HIV infection; or f) instructions for administering the Akt therapeutic agent in connection with reducing the risk of HIV infection.

Also disclosed are kits comprising an Akt therapeutic agent, or pharmaceutically acceptable salt, solvate, or polymorph thereof, and one or more of: a) at least one therapeutic agent known to decrease the severity of symptoms associated with an influenza infection; b) at least one therapeutic agent known to treat an influenza infection; c) instructions for treating an influenza infection; d) instructions for administering the Akt therapeutic agent in connection with treating an influenza infection; or f) instructions for administering the Akt therapeutic agent in connection with reducing the risk of influenza infection.

Also disclosed are kits comprising an effective amount of at least one phospholipase D inhibitor, or a pharmaceutically acceptable prodrug, salt, solvate, or polymorph thereof; an effective amount of at least one mTor inhibitor, or a pharmaceutically acceptable prodrug, salt, solvate, or polymorph thereof; and one or more of: a) an effective amount of at least one agent known to treat an HIV infection; b) an effective amount of at least one agent known to treat an opportunistic infection associated with an HIV infection; c) instructions for treating an HIV infection; d) instructions for treating an opportunistic infection associated with an HIV infection; e) instructions for administering the phospholipase D inhibitor in connection with treating an HIV infection; or f) instructions for administering the phospholipase D inhibitor in connection with reducing the risk of HIV infection.

Also disclosed are kits comprising a phospholipase D inhibitor, or a pharmaceutically acceptable prodrug, salt, solvate, or polymorph thereof; a mTor inhibitor, or a pharmaceutically acceptable prodrug, salt, solvate, or polymorph thereof; and one or more of: a) at least one agent known to decrease the severity of symptoms associated with an influenza infection; b) at least one agent known to treat an influenza infection; c) instructions for treating an influenza infection; d) instructions for administering the Akt inhibitor in connection with treating an influenza infection; or e) instructions for administering the Akt inhibitor in connection with reducing the risk of influenza infection.

Also disclosed are kits comprising an effective amount of a phospholipase D inhibitor, or a pharmaceutically acceptable prodrug, salt, solvate, or polymorph thereof; an effective amount of a mTor inhibitor, or a pharmaceutically acceptable prodrug, salt, solvate, or polymorph thereof; and one or more of: a) an effective amount of at least one agent known to treat a disorder of uncontrolled cellular proliferation; b) an effective amount of an Akt therapeutic agent; c) at least one agent known to increase Akt activity; or d) instructions for treating a disorder of uncontrolled cellular proliferation.

Also disclosed are kits comprising an effective amount of at least one phospholipase D inhibitor, or a pharmaceutically acceptable prodrug, salt, solvate, or polymorph thereof; instructions for administering the phospholipase D inhibitor to a subject identified with a mutation associated with activation of Akt; and one or more of: a) at least one anticancer therapeutic agent; b) an effective amount of an Akt therapeutic agent; c) at least one agent known to increase Akt activity; d) instructions for treating a disorder of uncontrolled cellular proliferation; or f) instructions for administering the phospholipase D inhibitor with the anticancer therapeutic agent and/or Akt therapeutic agent.

Also disclosed are kits comprising an effective amount of a phospholipase D inhibitor, or a pharmaceutically acceptable prodrug, salt, solvate, or polymorph thereof; an effective amount of an autophagy inducer, or a pharmaceutically acceptable prodrug, salt, solvate, or polymorph thereof; and one or more of: a) at least one agent known to decrease the severity of symptoms associated with an infectious disease; b) at least one agent known to treat an infectious disease; c) instructions for treating an infectious disease; d) instructions for administering the phospholipase D inhibitor and autophagy inducer in connection with treating an infectious disease; or e) instructions for administering the phospholipase D inhibitor and autophagy inducer in connection with reducing the risk of an infectious disease.

Also disclosed are kits comprising an effective amount of a phospholipase D inhibitor, or a pharmaceutically acceptable prodrug, salt, solvate, or polymorph thereof; and one or more of: a) at least one agent known to increase Akt activity; b) at least one agent known to decrease Akt activity; c) instructions for treating an infectious disease; or d) instructions for administering the phospholipase D inhibitor in connection with treating a disorder associated with an increase in Akt activity.

Also disclosed are kits comprising an effective amount of a phospholipase D inhibitor, or a pharmaceutically acceptable prodrug, salt, solvate, or polymorph thereof; an effective amount of an autophagy inducer, or a pharmaceutically acceptable prodrug, salt, solvate, or polymorph thereof; and one or more of: a) at least one agent known to decrease the severity of symptoms associated with an neurodegenerative disease; b) at least one agent known to treat to a neurodegenerative disorder; c) instructions for administering the phospholipase D inhibitor and autophagy inducer in connection with treating an neurodegenerative disorder; or e) instructions for administering the phospholipase D inhibitor and autophagy inducer in connection with reducing the severity of symptoms associated with a neurodegenerative disorder.

Also disclosed are methods for manufacturing a medicament comprising an effective amount of an Akt therapeutic agent, or a pharmaceutically acceptable prodrug, salt, solvate, or polymorph thereof; an effective amount of an antiviral therapeutic agent; and a pharmaceutically acceptable carrier, wherein the medicament is used to treat a viral infection, a bacterial infection, or a disorder of uncontrolled cellular proliferation.

Also disclosed are methods for manufacturing a medicament comprising an effective amount of an Akt therapeutic agent, or a pharmaceutically acceptable prodrug, salt, solvate, or polymorph thereof; an effective amount of at least one antibacterial therapeutic agent; and a pharmaceutically acceptable carrier, wherein the medicament is used to treat a viral infection, a bacterial infection, or a disorder of uncontrolled cellular proliferation.

Also disclosed are methods for manufacturing a medicament comprising: (a) a first therapeutic agent comprising an effective amount of a phospholipase D inhibitor, or a pharmaceutically acceptable prodrug, salt, solvate, or polymorph thereof; and (b) a second therapeutic agent comprising an effective amount of a mTor inhibitor, or a pharmaceutically acceptable prodrug, salt, solvate, or polymorph thereof; and a pharmaceutically acceptable carrier, wherein the medicament is used to treat a viral infection, a bacterial infection, or a disorder of uncontrolled cellular proliferation.

Also disclosed are uses of a disclosed compound, an Akt inhibitor, and/or an mTor inhibitor for use in the treatment of a viral infection, a bacterial infection, a neurodegenerative disorder, or a disorder of uncontrolled cellular proliferation.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows representative data pertaining to PLD activity following serum-withdrawal.

FIG. 2 shows representative data demonstrating that PLD activity is required for cell viability in GBM cells.

FIG. 3 shows representative data demonstrating that PLD activity is required for cell viability and anchorage independent growth in GBM cells.

FIG. 4 shows representative data demonstrating that Akt activation requires PLD activity in U87MG GBM cells.

FIG. 5 shows representative data demonstrating that Akt activation requires PLD activity in U118MG GBM cells.

FIG. 6 shows representative data demonstrating that Akt activation requires PLD activity in HEK293 GBM cells.

FIG. 7 shows representative data indicating that Akt activation requires PLD activity in GBM cells.

FIG. 8 shows representative data pertaining to Akt and PLD2 interaction.

FIG. 9 shows representative data indicating that Akt and PLD2 form a direct protein complex.

FIG. 10 shows representative data indicating that Akt recruitment to membranes is enhanced by binding to PtdOH.

FIG. 11 shows representative data pertaining to Akt activity.

FIG. 12 shows additional representative data pertaining to Akt activity.

FIG. 13 shows representative data indicating that phosphatidic acid enhances Akt binding to PIP₃.

FIG. 14 shows representative data indicating that PLD inhibitors induce autophagy dependent cell death in GBM.

FIG. 15A and FIG. 15B show representative data pertaining to the quantification of LC3-II and p62 from FIG. 14C. ANOVA with Dunnett's post-hoc test was used to compare inhibitor treatment to vehicle control within the PtdOH treatment conditions (* p<0.05, ** p<0.01).

FIG. 16 shows representative data pertaining to autophagy dependent cell-death in glioma.

FIG. 17 shows representative data indicating that glioblastoma cell death resulting from PLD inhibition is predominantly through an autophagy-dependent mechanism.

FIG. 18 shows that PLD inhibition decreases autophagic flux.

FIG. 19 shows representative images of U87MG stable cells expressing a GFP/RFP-LC3 tandem-fluorescent tag.

FIG. 20 shows representative data pertaining to autophagy in gliobastoma cells following PLD inhibition.

FIG. 21 shows representative data pertaining to cell viability following restoration of Akt function.

FIG. 22 shows representative data demonstrating that PLD and Akt promote autophagic flux by dissociating Rubicon from Beclin 1.

FIG. 23 shows representative data demonstrating that restoration of Akt function rescues cell viability following PLD inhibitor treatment.

FIG. 24 shows representative data pertaining to restoration of Akt function.

FIG. 25 shows representative data indicating that PLD activity is required for full Akt activation in GBM cells and that when inhibited, cells undergo autophagic death.

FIG. 26 shows representative data pertaining to PLD signaling.

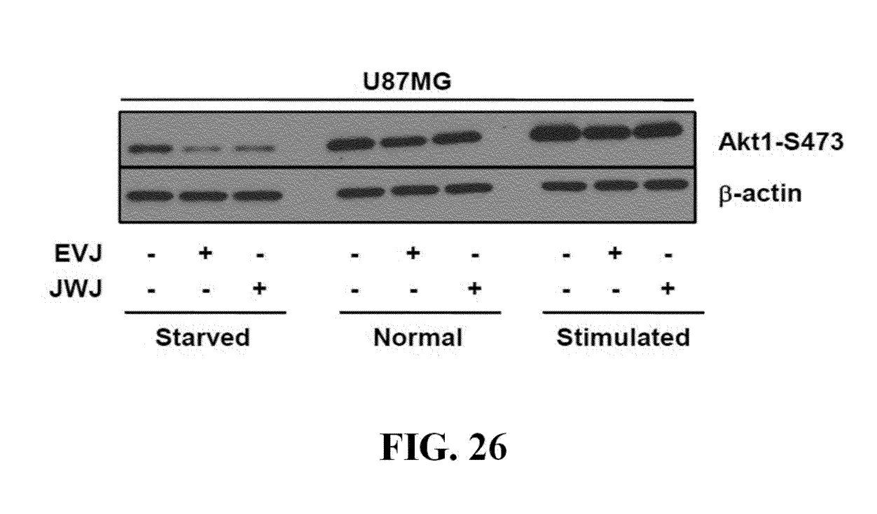

FIG. 27 shows the mechanism of Akt and autophagy regulation by PLD. In the absence of inhibitors, PLD generates PtdOH and recruits Akt to the membrane allowing for phosphorylation of Beclin1 by Akt at serine 295 and disruption of the Beclin1/Rubicon complex and promotion of autophagic flux (27A). PLD inhibitors reduce PtdOH production and subsequent Akt membrane recruitment. The inactivation of Akt results in reduced phosphorylation of Beclin1 at serine 295 and formation of the Beclin1/Rubicon complex (27B).

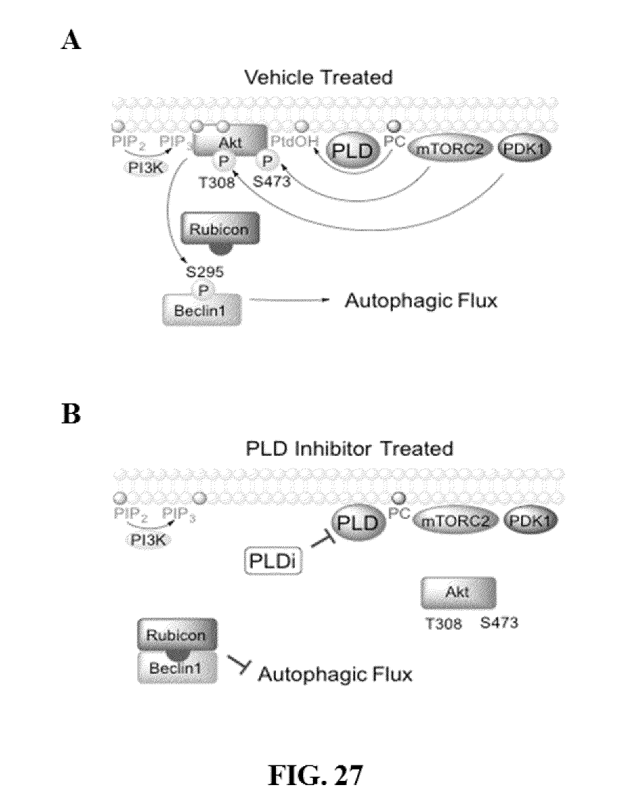

FIG. 28A shows data indicating constitutive PLD activity in U87MG glioblastoma cells. FIG. 28B shows that the PLD1 selective inhibitor VU0155069 blocks PtdBuOH production.

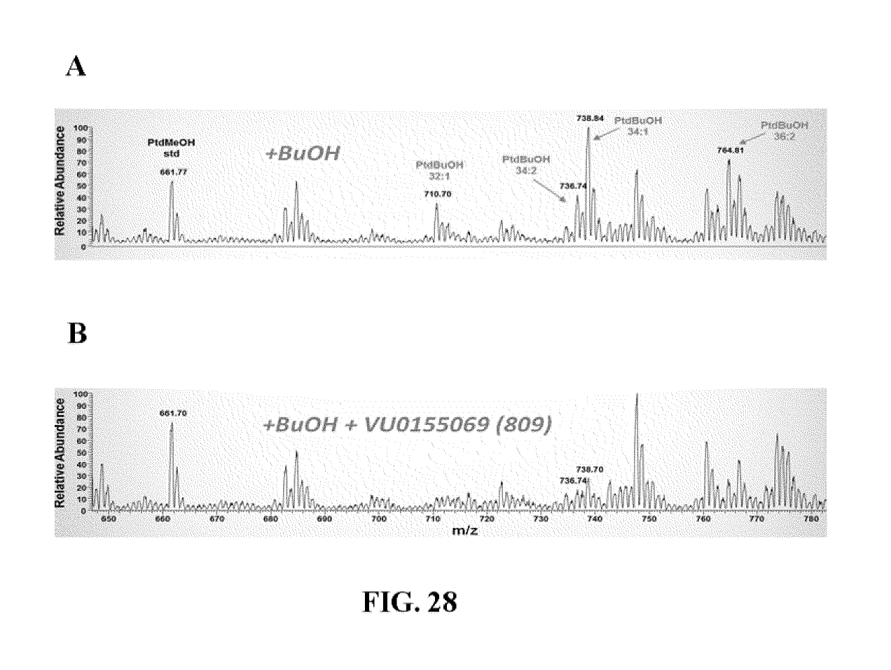

FIG. 29A shows a concentration-effect curve for the PLD1 selective inhibitor EVJ. FIG. 29B shows a concentration-effect curve for the PLD1 selective inhibitor JWJ.

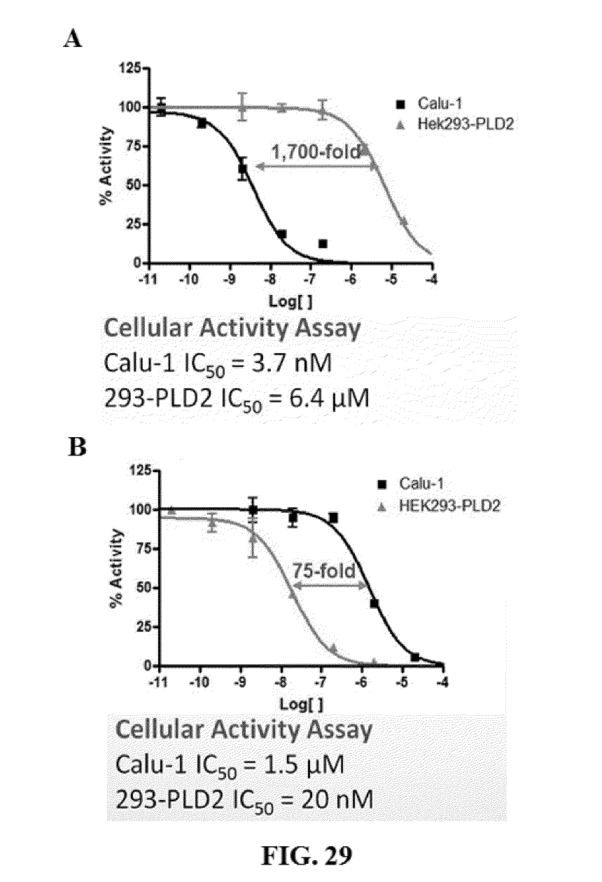

FIG. 30 shows the proposed mechanism of modulators of PLD function, as noncompetitive and allosteric modulators.

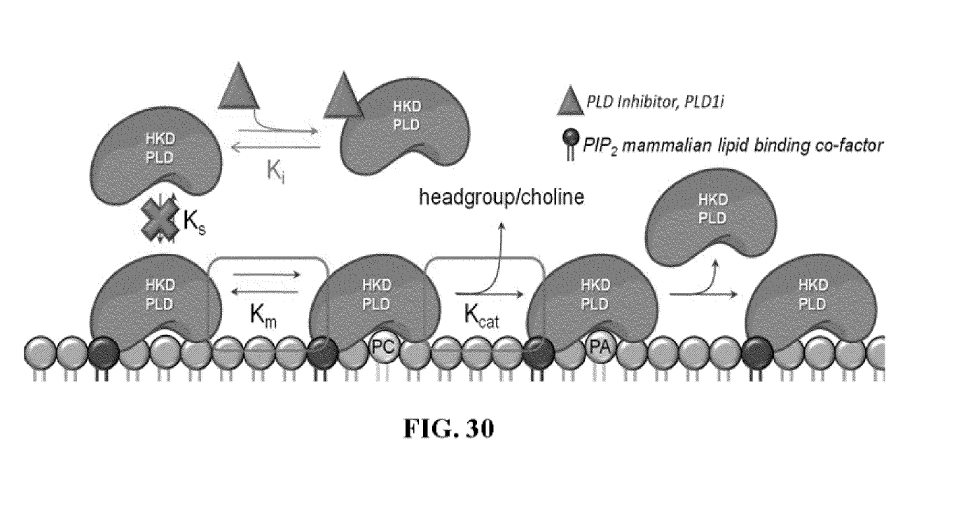

FIG. 31 shows that PLD or Akt inhibitors block anchorage independent growth in CD133+ stem cells from primary malignant glioblastomas.

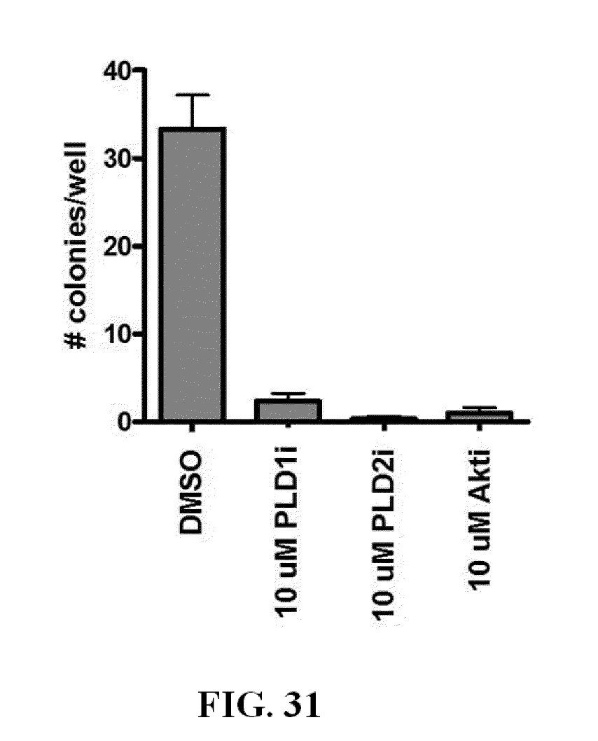

FIG. 32A shows that inhibition of PLD activity leads to decreased S473 and T308 phosphorylation of Akt. FIG. 32B shows that Akt has a PA binding site that modulates PIP₃ affinity, demonstrating that PLD directly modulates Akt.

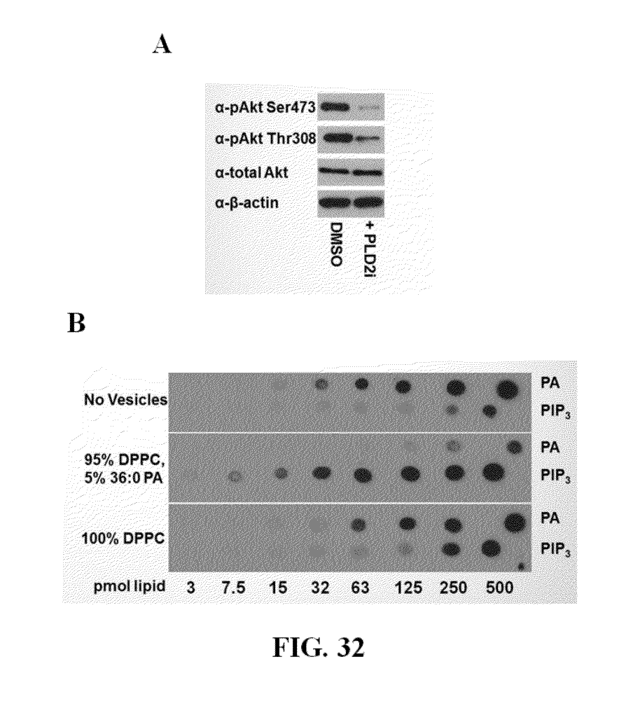

FIG. 33 shows the role of phospholipase D and phosphatidic acid in viral infections.

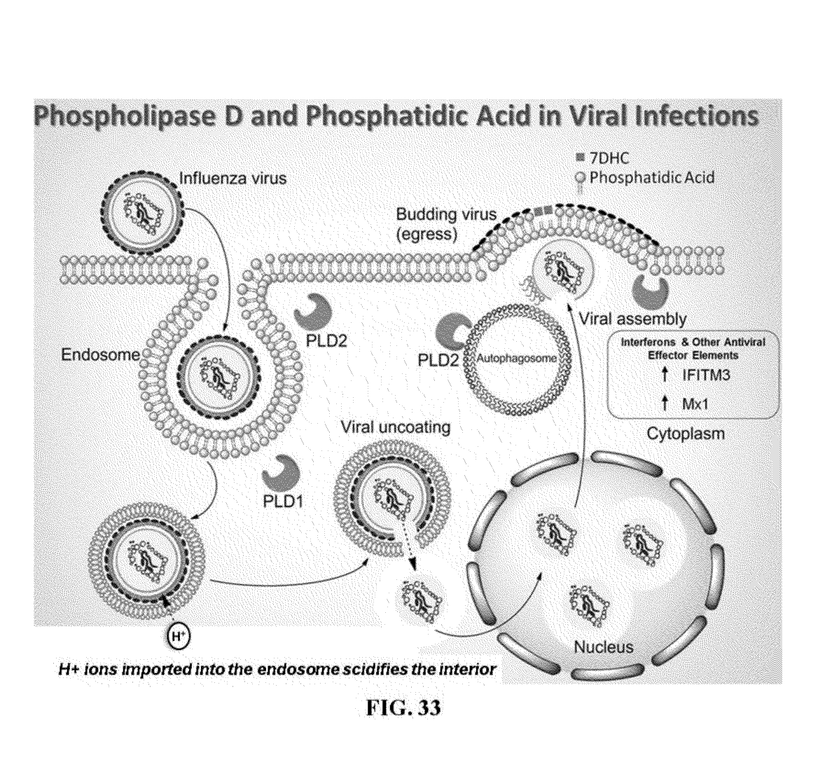

FIG. 34 shows representative data demonstrating that influenza infection increases PtdBuOH formation, which in turn, is decreased by PLD2i.

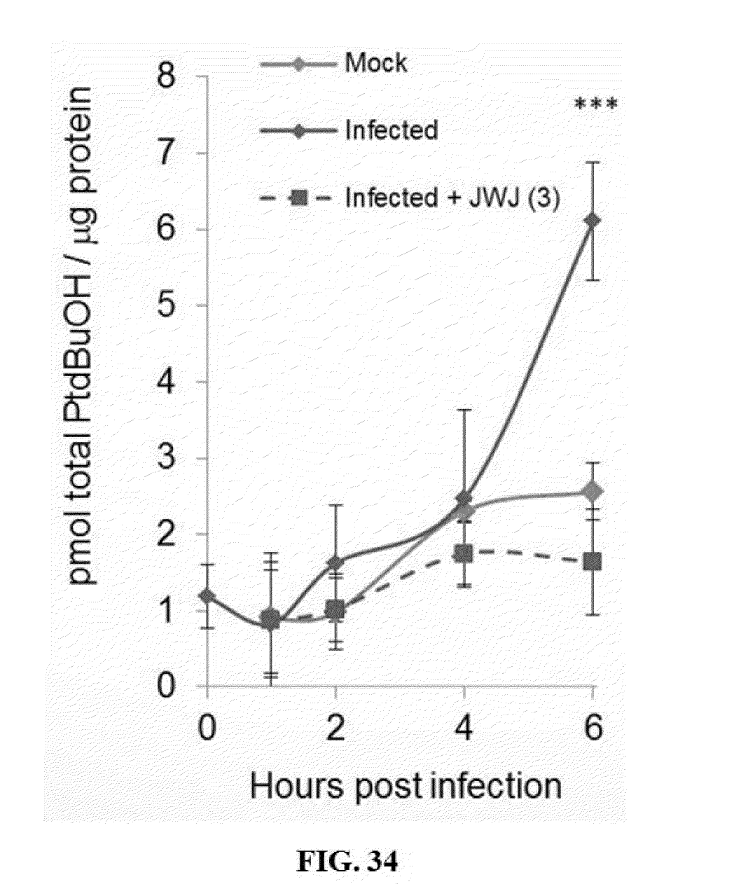

FIG. 35 shows that PLD inhibitors block influenza replication in human airway epithelial cells.

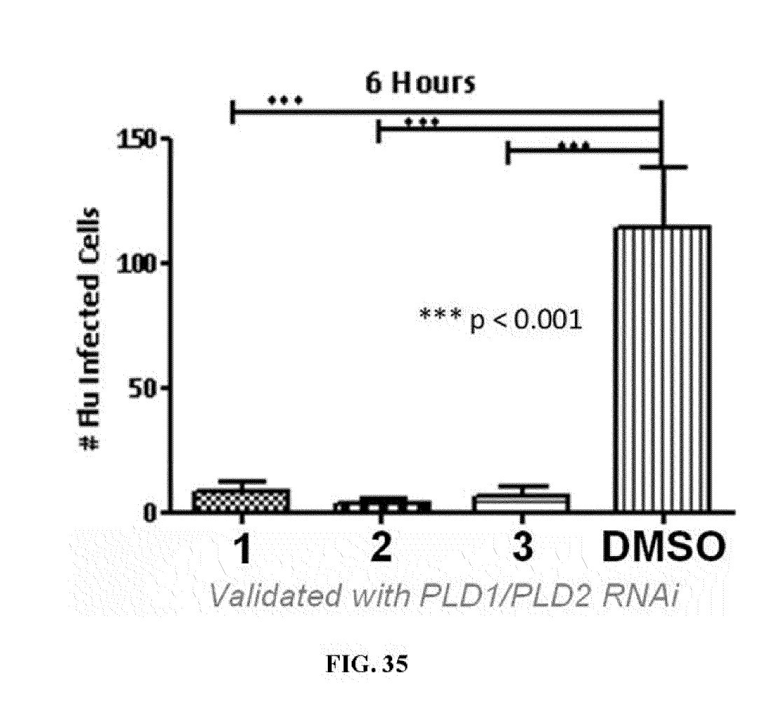

FIG. 36A shows representative data pertaining to the survival rate of mice infected with influenza. FIG. 36B shows representative data pertaining to the viral titer following influenza infection.

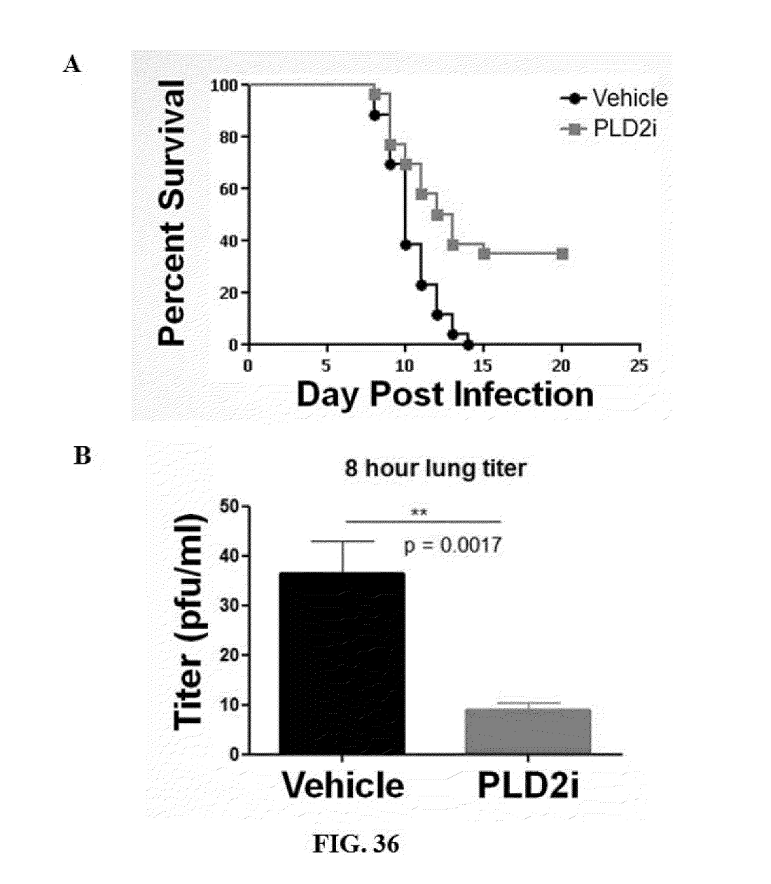

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about,” “approximate,” and “at or about” mean that the amount or value in question can be the exact value designated or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

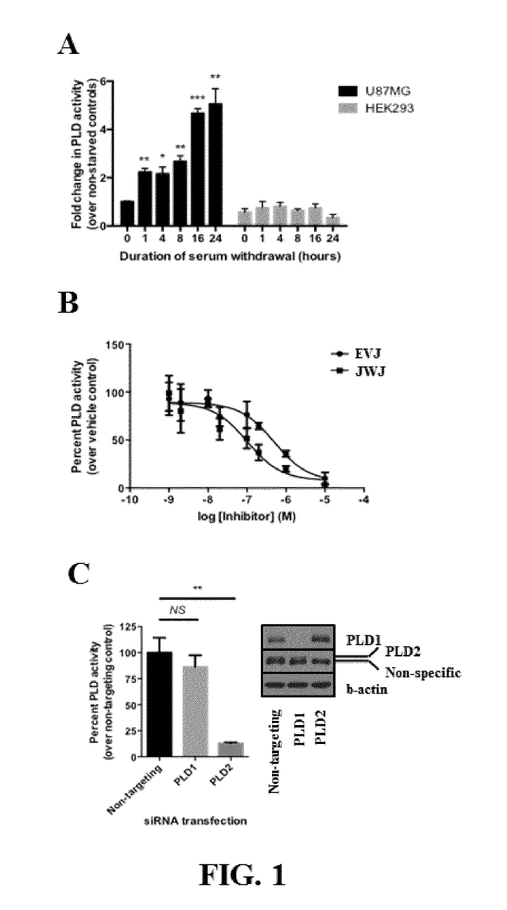

As used herein, the terms “phospholipase D” and “PLD” can be used interchangeably, and refer to a protein family comprising at least the following members: PLD1 and PLD2. Activation of PLDs occurs as a consequence of agonist stimulation of both tyrosine kinase and G protein-coupled receptors. PC-specific PLDs have been proposed to function in regulated secretion, cytoskeletal reorganization, transcriptional regulation, and cell cycle control. PLDs may also be involved in the regulation of perinuclear intravesicular membrane traffic. Several domains are described for the protein, with the overall primary domain structure for PLD1 as given in FIG. 1. PLD2 lacks the “loop” domain, but otherwise has the same domains located at about the same relative positions in the protein.

The PLD protein family catalyzes a variety of reaction. The most well-characterized reaction is the hydrolysis of phosphatidylcholine to produce phosphatidic acid and choline, as follows:

a phosphatidylcholine+H₂O→choline+a phosphatidate

Although the foregoing is the most well-characterized reaction catalyzed by PLD, there are additional reactions which are also catalyzed by PLD. These reactions include:

a lysophosphatidylcholine+H₂O→choline+a lysophosphatidate

a lysophosphotidylcholine→choline+a cyclic lysophosphotidate

a phosphotidylcholine+ROH→choline+a phosphotidylalcohol

The reactions catalyzed by PLD can involve headgroups other than choline. For example, hydrolysis of the headgroup can be generalized as follows:

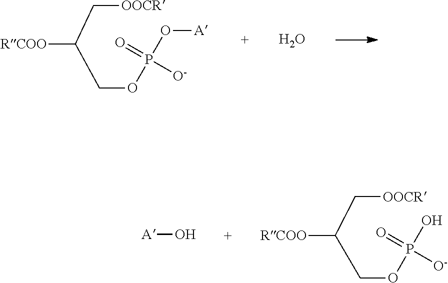

In the foregoing reaction scheme, the R′COO and R″COO moieties derive from fatty acids, e.g. C₁₆-C₂₂ saturated and unsaturated fatty acids (including polyenoic acids). It should be understood that A′ represents an amine containing moiety, e.g. choline.

Alternatively, PLD can also catalyze a transphosphatidylation reaction as follows:

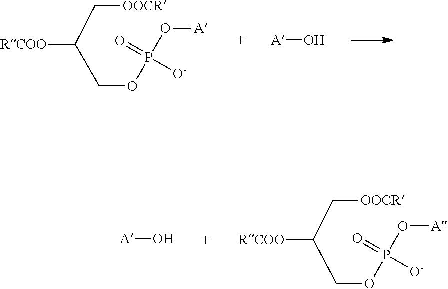

In the foregoing reaction scheme, the R′COO, R″COO, and A′ moieties have the same meaning as in the previous reaction. In addition, the A″-OH moiety represents is a primary alcohol.

As used herein, the terms “phospholipase D1” and “PLD1” refer to the phospholipase D1 protein encoded by a gene designated in human as the PLD1 gene, which has a human gene map locus described by Entrez Gene cytogenetic band: 3q26; Ensembl cytogenetic band: 3q26.31; and, HGNC cytogenetic band: 3q26. The term PLD1 refers to a human protein that has about 1074 amino acids and has a molecular weight of about 124,184 Da. The term is inclusive of splice isoforms or mRNA transcript variants, e.g. the alternative mRNA splicing products that code for the isoforms designated as PLD1A, PLD1B, PLD1C, and PLD1D. The term is also inclusive of that protein referred to by such alternative designations as: “PLD1,” “phospholipase D1, phosphatidylcholine-specific,” “choline phosphatase 1,” “phosphatidylcholine-hydrolyzing phospholipase D1,” “PLD1,” “PLD 1,” “EC 3.1.4.4,” “phospholipase D1,” and “phospholipase D1, phophatidylcholine-specific,” as used by those skilled in the art to refer to that protein encoded by human gene PLD 1 or to the gene itself. The term is also inclusive of the non-human orthologs or homologs thereof, as well as splice variants and alternative transcripts of the PLD1 gene.

As used herein, the terms “phospholipase D2” and “PLD2” refer to the phospholipase D2 protein encoded by a gene designated in human as the PLD2 gene, which has a human gene map locus described by Entrez Gene cytogenetic band: 17p13.1; Ensembl cytogenetic band: 17p13.2; and, HGNC cytogenetic band: 17p13.3. The term PLD2 refers to a human protein that has about 933 amino acids and has a molecular weight of about 105,987 Da. The term is inclusive of splice isoforms or mRNA transcript variants, e.g. the alternative mRNA splicing products that code for the isoforms designated as PLD2A, PLD2B, and PLD2C. The term is also inclusive of that protein referred to by such alternative designations as: “PLD2,” “phospholipase D2,” “Choline phosphatase 2,” “Phosphatidylcholine-hydrolyzing phospholipase D2,” “PLD1C,” “hPLD2,” “PLD 2,” and “EC 3.1.4.4,” as used by those skilled in the art to refer to that protein encoded by human gene PLD2 or to the gene itself. The term is also inclusive of the non-human orthologs or homologs thereof, as well as splice variants and alternative transcripts of the PLD2 gene.

As used herein, the term “PLD inhibitor” refers to any exogenously administered compound or agent that directly inhibits the activity of a PLD gene product. In this context, an inhibitor is understood to directly decrease the activity of the target PLD gene product compared to the activity of the gene product in the absence of the exogenously administered compound or agent. Examples of directly acting compounds or agents are allosteric inhibitors, competitive inhibitors, noncompetitive inhibitors, irreversible inhibitors, and uncompetitive inhibitors.

As used herein, the term “PLD1 inhibitor” refers to any exogenously administered compound or agent that directly inhibits the activity of a PLD1 gene product. In this context, an inhibitor is understood to directly decrease the activity of the target PLD1 gene product compared to the activity of the gene product in the absence of the exogenously administered compound or agent. Examples of directly acting compounds or agents are allosteric inhibitors, competitive inhibitors, noncompetitive inhibitors, irreversible inhibitors, and uncompetitive inhibitors.

As used herein, the term “PLD2 inhibitor” refers to any exogenously administered compound or agent that directly inhibits the activity of a PLD2 gene product. In this context, an inhibitor is understood to directly decrease the activity of the target PLD2 gene product compared to the activity of the gene product in the absence of the exogenously administered compound or agent. Examples of directly acting compounds or agents are allosteric inhibitors, competitive inhibitors, noncompetitive inhibitors, irreversible inhibitors, and uncompetitive inhibitors.

As used herein, “inhibition of enzyme activity” refers to both direct and indirect inhibition of a particular enzymatic activity or function. In particular instances, e.g. “inhibition of PLD activity” or “inhibition of PLD,” which can be used interchangeably, refer to, and include, both direct and indirect inhibition of PLD enzymatic activity. Likewise, “inhibition of Akt activity” or “inhibition of Akt,” which can be used interchangeably, refer to, and include, both direct and indirect inhibition of Akt enzymatic activity. For example, an agent inhibiting an enzyme activity, e.g. PLD or Akt enzymatic activity, can bind to discrete sites on the target enzyme with the overall effect of decreasing enzyme catalytic activity or modulating an essential protein-protein interaction, thus a diversity of structures can achieve the desired function. It is understood that binding that directly affects catalytic activity can occur at an orthosteric or allosteric site. In addition, inhibition of an enzyme activity, this can also be accomplished via interaction of a compound with a protein other than the target enzyme, e.g. interaction with a protein that modulates the activity or expression of the target enzyme, thus a diversity of structures can achieve the desired function indirectly as well.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein.

As used herein, “gene product” refers to transcription or translation products that are derived from a specific gene locus or gene. The “gene locus” or “gene” includes coding sequences as well as regulatory, flanking and intron sequences.

The term “viral infection” refers to the introduction of a virus into cells or tissues, e.g., an influenza virus. In general, the introduction of a virus is also associated with replication. Viral infection may be determined by measuring virus antibody titer in samples of a biological fluid, such as blood, using, e.g., enzyme immunoassay. Other suitable diagnostic methods include molecular based techniques, such as RT-PCR, direct hybrid capture assay, nucleic acid sequence based amplification, and the like. A virus may infect a particular organ, e.g., lung, and cause disease, e.g., localized effects such as respiratory impairment and edema, and systemic effects.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder, e.g. an infection with an influenza virus. The term “patient” includes human and veterinary subjects. In some aspects of the disclosed methods, the subject has been diagnosed with a need for treatment of one or more viral infections prior to the administering step. In some aspects of the disclosed method, the subject has been diagnosed with a need for inhibition of PLD1, PLD2, or both PLD1 and PLD2 activity prior to the administering step. In some aspects of the disclosed method, the subject has been diagnosed with a viral infection, e.g. an influenza virus such as H₅N₁. In some aspects of the disclosed method, the subject has been identified with a disorder treatable by inhibition of PLD1, PLD2, or both PLD1 and PLD2 activity prior to the administering step. In one aspect, a subject can be treated prophylactically with a compound or composition disclosed herein, as discussed herein elsewhere. It is understood that a subject can be a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; supportive treatment, that is, treatment employed to supplement another specific therapy directed toward the improvement of the associated disease, pathological condition, or disorder; and prophylactic treatment, that is, treatment directed to preventing a disease or disorder in a subject, preventing the occurrence of symptoms in a subject with a disease or disorder, preventing the recurrence of symptoms in a subject with a disease or disorder, and/or decreasing the severity of frequency of outward symptoms of disease or disorder in a subject. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

As used herein, the term “prophylaxis” refers to the complete prevention of infection, the prevention of occurrence of symptoms in an infected subject, the prevention of recurrence of symptoms in an infected subject, or a decrease in severity or frequency of outward symptoms of viral infection or disease in the subject.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder treatable by selective inhibition of Phospholipase D1” means having been subjected to a physical examination by a person of skill, for example, a physician, and found to have a condition that can be diagnosed or treated by a compound or composition that can inhibit PLD1. As a further example, “diagnosed with a need for selective inhibition of Phospholipase D2” refers to having been subjected to a physical examination by a person of skill, for example, a physician, and found to have a condition characterized by PLD2 activity. Such a diagnosis can be in reference to a disorder, such as a disease of uncontrolled cellular proliferation, and the like, as discussed herein.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to PLD2 activity) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The terms “co-administer(s),” “co-administering,” and “co-administration” all refer to with respect to compounds or compositions, is meant either simultaneous administration or any manner of separate sequential administration of one or more PLD inhibitor compounds, e.g. a PLD1 selective inhibitor, a PLD2 selective inhibitor, or a non-selective inhibitor of PLD1 and PLD2, with one or more pharmaceutically active agents, such as, but not limited to, those agents included in antiviral therapy. Preferably, if the administration is not simultaneous, the compounds are administered in a close time proximity to each other. Furthermore, it does not matter if the compounds are administered in the same dosage form, e.g. one compound may be administered topically and another compound may be administered orally. “Substantially simultaneously” means that the compound, i.e. a PLD inhibitor compound, is typically administered during or within a reasonably short time either before or after the administration of other compounds, such as a pharmaceutically active agent that treats the disease in question. Additionally, “co-administration,” “co-administer(s),” and “co-administering” include administering more than one dose of the pharmaceutically active agent within 24 hours after a dose of a PLD inhibitor compound. In other words, PLD inhibitors need not be administered again before or with every administration of a pharmaceutically active agent, but may be administered intermittently during the course of treatment. “Co-administration,” “co-administer(s),” and “co-administering” also includes administering a pharmaceutically active agent and a PLD inhibitor compound as a part of one or more pharmaceutical compositions, and such one or more pharmaceutical compositions may contain a co-formulation of a PLD inhibitor compound and a pharmaceutically active agent or individual formulations of a pharmaceutically active agent and a PLD inhibitor compound.

It is understood that co-administration a PLD inhibitor compound and an anti-viral agent or other therapeutic agent can be independently co-administered by any appropriate route of administration. The active agents, i.e. a PLD inhibitor compound and an anti-viral agent or other therapeutic agent, can be administered by the same or different routes of administration, as appropriate. For example, one of the active ingredients can be administered orally and the other administered orally or by some other appropriate route of administration. Alternatively, the combination of active ingredients can be concurrently orally administered. In a further example, consistent with this understanding, one of the active ingredients can be administered parenterally, for example, intravenously, intramuscularly, subcutaneously, topically, intravaginally, rectally, intranasally, inhalationally, intrathecally, intraocularly, and one or more of the other active ingredients administrated by a similar or distinct route of administration. Moreover, it is understood, that a PLD inhibitor compound and an anti-viral agent or other therapeutic agent can be co-administered or independently administered by distinct routes of administration such as parenterally, orally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, or intrathecally.

As used herein, “combination therapy” (or “co-therapy”) refers to the administration of a PLD inhibitor compound and an anti-viral agent or other therapeutic agent during the course of therapy or treatment for a viral infection. Such combination therapy may involve the administration of the PLD inhibitor compound before, during, and/or after the administration of the anti-viral agent or other therapeutic agent administered to ameliorate, treat, reverse, or cure the viral infection or symptoms associated with the viral infection. The administration of the PLD inhibitor compound may be separated in time from the administration of anti-viral agent or other therapeutic agent by up to several weeks, and may precede it or follow it, but more commonly the administration of the PLD inhibitor compound will accompany at least one aspect of the administration of the anti-viral agent or other therapeutic agent.

As used herein, “concurrently” means (1) simultaneously in time, or (2) at different times during the course of a common treatment schedule.

The term “contacting” as used herein refers to bringing a disclosed compound and a cell, target histamine receptor, or other biological entity together in such a manner that the compound can affect the activity of the target (e.g., spliceosome, cell, etc.), either directly; i.e., by interacting with the target itself, or indirectly; i.e., by interacting with another molecule, co-factor, factor, or protein on which the activity of the target is dependent.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount or dosage that can effectively prevent a disease or disorder in a subject, prevent the occurrence of symptoms in a subject with a disease or disorder, prevent the recurrence of symptoms in a subject with a disease or disorder, and/or decrease the severity of frequency of outward symptoms of a disease or disorder in a subject.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug,” as used herein means a compound that is metabolized, for example hydrolyzed or oxidized, in the host to form the compound of the present invention without forming fragments with toxicological liabilities. Typical examples of prodrugs include compounds that have biologically labile protecting groups linked to a functional moiety of the active compound. For example, a prodrug can comprise alkylation, acylation or other lipophilic modification of one or more hydroxy group(s) present in a compound of the invention, e.g. a PLD inhibitor compound. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).'

The term “excipient” as used herein refers to a compound that is used to prepare a pharmaceutical composition, and is generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

The term “immune modulator” refers to any substance meant to alter the working of the humoral or cellular immune system of a subject. Such immune modulators include inhibitors of mast cell-mediated inflammation, interferons, interleukins, prostaglandins, steroids, corticosteroids, colony-stimulating factors, chemotactic factors, etc.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the terms “optional” or “optionally” means that the subsequently described event or circumstance may or may not occur, and that the description includes instances where said event or circumstance occurs and instances where it does not.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “aliphatic” refers to a non-aromatic carbon-based moiety. Aliphatic can include both acyclic and cyclic moieties (e.g., alkyl and cycloalkyl) and can include both saturated and unsaturated moieties (e.g., alkyl, alkenyl, and alkynyl).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of from 1 to 24 carbon atoms, for example from 1 to 12 carbons, from 1 to 8 carbons, from 1 to 6 carbons, or from 1 to 4 carbons, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The cycloalkyl group can be substituted or unsubstituted. The cycloalkyl group can be substituted with one or more groups including, but not limited to, alkyl, cycloalkyl, alkoxy, amino, ether, halide, hydroxy, nitro, silyl, sulfo-oxo, or thiol as described herein.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein. A specific example of amino is —NH₂.

The term “carboxylic acid” as used herein is represented by the formula —C(O)OH.

The term “ester” as used herein is represented by the formula —OC(O)A¹ or —C(O)OA¹, where A¹ can be an optionally substituted alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an optionally substituted alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, pyrazolopyrimidinyl, 1,2-oxazol-4-yl, 1,2-oxazol-5-yl, 1,3-oxazolyl, 1,2,4-oxadiazol-5-yl, 1,2,3-triazolyl, 1,3-thiazol-4-yl, pyridinyl, and pyrimidin-5-yl.

The term “heterocycle,” as used herein refers to single and multi-cyclic aromatic or non-aromatic ring systems in which at least one of the ring members is other than carbon. Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “heterocycloalkyl” as used herein is a non-aromatic carbon-based ring composed of at least two carbon atoms and at least one non-carbon heteroatom. For example, the non-carbon heteroatom can include, but is not limited to, oxygen, nitrogen, sulphur, phosphorus and the like. Examples of heterocycloalkyl groups include, aziridine, oxirane, thiirane, azetidine, oxetane, thietane, pyrrolidine, tetrahydrofuran, tetrahydrothiophene, piperidine, tetrahydro-2H-pyran, tetrahydro-2H-thipyran, azepane, oxepane, thiepane, azocane, oxocane, thiocane, pyrazolidine, imidazolidine, diazetidine, hexahydropyridazine, piperazine, diazepane, oxazinane, oxazepane, oxazolidine, oxazetine, and the like. The heterocycloalkyl group can be substituted or unsubstituted. The heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “hydroxyl” as used herein is represented by the formula —OH.

The term “ketone” as used herein is represented by the formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an optionally substituted alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “thiol” as used herein is represented by the formula —SH.

The term “organic residue” defines a carbon containing residue, i.e., a residue comprising at least one carbon atom, and includes but is not limited to the carbon-containing groups, residues, or radicals defined herein above. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure

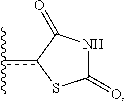

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

In some aspects, a structure of a compound can be represented by a formula:

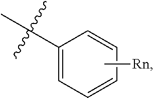

which is understood to be equivalent to a formula:

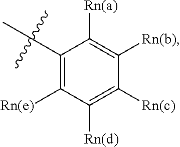

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain instances of the above defined terms may occur more than once in the structural formulae, and upon such occurrence each term shall be defined independently of the other.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “hydrolysable residue” is meant to refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “leaving group” refers to an atom (or a group of atoms) with electron withdrawing ability that can be displaced as a stable species, taking with it the bonding electrons. Examples of suitable leaving groups include sulfonate esters, including triflate, mesylate, tosylate, brosylate, and halides.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (ee). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

### B. PHOSPHOLIPASE D INHIBITORS

In one aspect, the invention relates to compounds, or pharmaceutically acceptable derivatives thereof, useful as isoform selective phospholipase D inhibitors. In general, it is contemplated that each disclosed compound or derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

In one aspect, the compounds of the invention are useful in the treatment of viral infection. In a further aspect, the compounds are useful in the treatment of disease associated with a viral infection. In a still further aspect, the compounds are useful in the treatment of a disorder of uncontrolled cellular proliferation.

1. Structure

In one aspect, the invention relates to phospholipase D inhibitors comprising a compound with a structure represented by a formula:

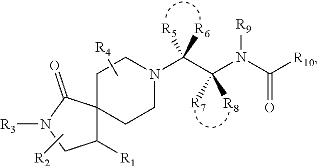

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, trifluoromethyl, carboxamido, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, trifluoromethyl, carboxamido, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof.

In a further aspect, the compound has a structure represented by a formula:

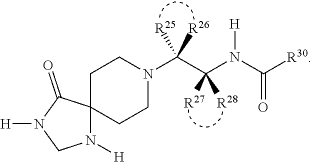

In one aspect, the invention relates to phospholipase D inhibitors comprising a compound with a structure represented by a formula:

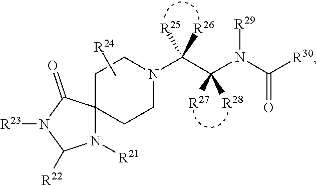

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, trifluoromethyl, carboxamido, alkylsulfonyl, an optionally substituted C1 to C6 alkyl, or an optionally substituted C3 to C6 cycloalkyl or R²⁵ and R²⁶, together with the intermediate carbon, comprise an optionally substituted C3 to C6 cycloalkyl; wherein each of R²⁷ and R²⁸ independently comprises hydrogen, trifluoromethyl, carboxamido, alkylsulfonyl, an optionally substituted C1 to C6 alkyl, or an optionally substituted C3 to C6 cycloalkyl or R²⁷ and R²⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof.

In a further aspect, the compound has a structure represented by a formula:

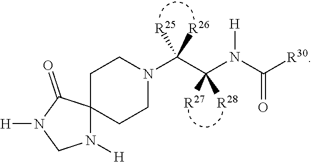

In one aspect, the invention relates to phospholipase D inhibitors comprising a compound with a structure represented by a formula:

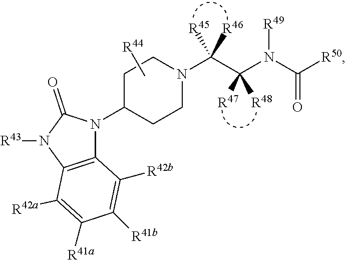

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, trifluoromethyl, carboxamido, alkylsulfonyl, an optionally substituted C1 to C6 alkyl, or an optionally substituted C3 to C6 cycloalkyl or R⁴⁵ and R⁴⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁴⁷ and R⁴⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for viral infection.

In a further aspect, the compound has a structure represented by a formula:

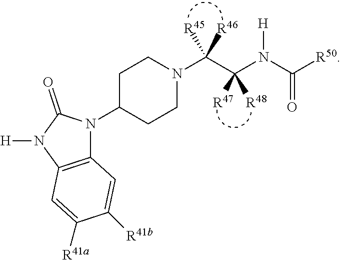

It is understood that the disclosed compounds can be used in connection with the disclosed methods, compositions, kits, and uses.

a. R¹ GROUPS

In one aspect, R¹ is an optionally substituted C3 to C9 organic residue selected from aryl, heteroaryl, cycloalkyl, heterocycloalkyl, cycloalkenyl, and heterocycloalkenyl.

In a further aspect, R¹ is optionally substituted aryl selected from phenyl and naphthyl.

In a further aspect, R¹ is optionally substituted heteroaryl selected from furanyl, pyranyl, imidazolyl, thiophenyl, pyridinyl, pyridazinyl, pyrimidinyl, pyrazinyl, triazinyl, tetrazinyl, benzofuranyl, benzothiophenyl, indolyl, indazolyl, quinolinyl, naphthyridinyl, benzothiazolyl, benzooxazolyl, benzoimidazolyl, and benzotriazolyl.

In a further aspect, R¹ is optionally substituted cycloalkyl selected from cyclopropyl, cyclobutyl, cyclopentyl, cyclohexyl, cycloheptyl, cyclooctyl, cyclononyl, bicyclo[3.1.0]hexyl, bicyclo[4.1.0]heptyl, bicyclo[5.1.0]octyl, bicyclo[6.1.0]nonyl, bicyclo[3.2.0]heptyl, bicyclo[4.2.0]octyl, bicyclo[5.2.0]nonyl, bicyclo[3.3.0]octyl, bicyclo[4.3.0]nonyl, bicyclo[2.2.1]heptyl, bicyclo[3.2.1]octyl, bicyclo[4.2.1]nonyl, bicyclo[2.2.2]octyl, bicyclo[3.2.2]nonyl, and bicyclo[3.3.1]nonyl.

In a further aspect, R¹ is optionally substituted heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R¹ is optionally substituted cycloalkenyl selected from cyclobutenyl, cyclopentenyl, cyclopentadienyl, cyclohexenyl, cyclohexadienyl, cycloheptenyl, cycloheptadienyl, cyclooctenyl, cyclooctadienyl, cyclononenyl, and cyclononadienyl.

In a further aspect, R¹ is optionally substituted heterocycloalkenyl comprising a mono-, di- or tri-unsaturated analog of a heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R¹ is halophenyl, for example 4-fluorophenyl.

b. R² GROUPS

In one aspect, R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue.

In a further aspect, each R² is hydrogen. In a further aspect, each R² is independently selected from halide, hydroxyl, trifluoromethyl, amino, cyano, nitro, azide, carboxamido, alkoxy, thiol, alkylsulfonyl, and an optionally substituted C1 to C6 organic residue. In a further aspect, each R² is independently selected from halide, hydroxyl, trifluoromethyl, amino, cyano, nitro, azide, carboxamido, alkoxy, thiol, and alkylsulfonyl. In a further aspect, at least one R² is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.
c. R³ GROUPS

In one aspect, R³ comprises hydrogen, an optionally substituted C1 to C6 alkyl, an optionally substituted C3 to C6 cycloalkyl, or a hydrolysable residue.

In a further aspect, R³ is hydrogen. In a further aspect, R³ is an optionally substituted C1 to C6 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, and cyclohexyl. In a further aspect, R³ is an optionally substituted C3 to C6 cycloalkyl selected from cyclopropyl, cyclobutyl, cyclopentyl, cyclohexyl, and bicyclo[3.1.0]hexyl. In a further aspect, R³ is a hydrolysable residue.
d. R⁴ GROUPS

In one aspect, R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue.

In a further aspect, each R⁴ is hydrogen. In a further aspect, each R⁴ is independently selected from halide, hydroxyl, trifluoromethyl, amino, cyano, nitro, azide, carboxamido, alkoxy, thiol, alkylsulfonyl, and an optionally substituted C1 to C6 organic residue. In a further aspect, each R⁴ is independently selected from halide, hydroxyl, trifluoromethyl, amino, cyano, nitro, azide, carboxamido, alkoxy, thiol, and alkylsulfonyl. In a further aspect, at least one R⁴ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

e. R⁵ AND R⁶ GROUPS

In one aspect, each of R⁵ and R⁶ independently comprises hydrogen, trifluoromethyl, carboxamido, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl.

In a further aspect, R⁵ is hydrogen. In a further aspect, R⁵ is selected from trifluoromethyl, carboxamido, alkylsulfonyl, and an optionally substituted C1 to C6 organic residue. In a further aspect, R⁵ is selected from trifluoromethyl, carboxamido, and alkylsulfonyl. In a further aspect, R⁵ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁶ is hydrogen. In a further aspect, R⁶ is selected from trifluoromethyl, carboxamido, alkylsulfonyl, and an optionally substituted C1 to C6 organic residue. In a further aspect, R⁶ is selected from trifluoromethyl, carboxamido, and alkylsulfonyl. In a further aspect, R⁶ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁶ is hydrogen and wherein R⁵ is selected from trifluoromethyl, carboxamido, alkylsulfonyl, and an optionally substituted C1 to C6 organic residue. In a further aspect, R⁶ is hydrogen and wherein R⁵ is selected from trifluoromethyl, carboxamido, and alkylsulfonyl. In a further aspect, R⁶ is hydrogen and wherein R⁵ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁵ is hydrogen and wherein R⁶ is selected from trifluoromethyl, carboxamido, alkylsulfonyl, and an optionally substituted C1 to C6 organic residue. In a further aspect, R⁵ is hydrogen and wherein R⁶ is selected from trifluoromethyl, carboxamido, and alkylsulfonyl. In a further aspect, R⁵ is hydrogen and wherein R⁶ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁵ and R⁶, together with the intermediate carbon, comprise an optionally substituted C3 to C6 cycloalkyl. In a further aspect, wherein R⁵ and R⁶, together with the intermediate carbon, comprise cyclopropyl, cyclobutyl, cyclopentyl, or cyclohexyl.
f. R⁷ AND R⁸ GROUPS

In one aspect, each of R⁷ and R⁸ independently comprises hydrogen, trifluoromethyl, carboxamido, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl.

In a further aspect, R⁷ is hydrogen. In a further aspect, R⁷ is selected from trifluoromethyl, carboxamido, alkylsulfonyl, and an optionally substituted C1 to C6 organic residue. In a further aspect, R⁷ is selected from trifluoromethyl, carboxamido, and alkylsulfonyl. In a further aspect, R⁷ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl. In a further aspect, R⁷ is methyl.

In a further aspect, R⁸ is hydrogen. In a further aspect, R⁸ is selected from trifluoromethyl, carboxamido, alkylsulfonyl, and an optionally substituted C1 to C6 organic residue. In a further aspect, R⁸ is selected from trifluoromethyl, carboxamido, and alkylsulfonyl. In a further aspect, R⁸ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl. In a further aspect, R⁸ is methyl.

In a further aspect, R⁸ is hydrogen and wherein R⁷ is selected from trifluoromethyl, carboxamido, alkylsulfonyl, and an optionally substituted C1 to C6 organic residue. In a further aspect, R⁸ is hydrogen and wherein R⁷ is selected from trifluoromethyl, carboxamido, and alkylsulfonyl. In a further aspect, R⁸ is hydrogen and wherein R⁷ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁷ is hydrogen and wherein R⁸ is selected from trifluoromethyl, carboxamido, alkylsulfonyl, and an optionally substituted C1 to C6 organic residue. In a further aspect, R⁷ is hydrogen and wherein R⁸ is selected from trifluoromethyl, carboxamido, and alkylsulfonyl. In a further aspect, R⁷ is hydrogen and wherein R⁸ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁷ and R⁸, together with the intermediate carbon, comprise an optionally substituted C3 to C6 cycloalkyl. In a further aspect, R⁷ and R⁸, together with the intermediate carbon, comprise cyclopropyl, cyclobutyl, cyclopentyl, or cyclohexyl.
g. R⁹ GROUPS

In one aspect, R⁹ comprises hydrogen, an optionally substituted C1 to C6 alkyl, an optionally substituted C3 to C6 cycloalkyl, or a hydrolysable residue.

In a further aspect, R⁹ is hydrogen. In a further aspect, R⁹ is an optionally substituted C1 to C6 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, and cyclohexyl. In a further aspect, R⁹ is an optionally substituted C3 to C6 cycloalkyl selected from cyclopropyl, cyclobutyl, cyclopentyl, and cyclohexyl. In a further aspect, R⁹ is a hydrolysable residue.
h. R¹ GROUPS

In one aspect, R¹⁰ comprises an optionally substituted C1 to C12 organic residue selected from alkyl, aryl, heteroaryl, cycloalkyl, heterocycloalkyl, cycloalkenyl, and heterocycloalkenyl.

In a further aspect, R¹⁰ is an optionally substituted alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, cyclohexyl, heptyl, cycloheptyl, octyl, cyclooctyl, nonyl, cyclononyl, decyl, cyclodecyl, undecyl, cycloundecyl, dodecyl, or cyclododecyl.

In a further aspect, R¹⁰ is an optionally substituted aryl selected from phenyl and naphthyl.

In a further aspect, R¹⁰ is an optionally substituted heteroaryl selected from furanyl, pyranyl, imidazolyl, thiophenyl, pyridinyl, pyridazinyl, pyrimidinyl, pyrazinyl, triazinyl, tetrazinyl, benzofuranyl, benzothiophene, indolyl, indazolyl, quinolinyl, naphthyridinyl, benzothiazolyl, benzooxazolyl, benzoimidazolyl, and benzotriazolyl.

In a further aspect, R¹⁰ is an optionally substituted cycloalkyl selected from cyclopropyl, cyclobutyl, cyclopentyl, cyclohexyl, cycloheptyl, cyclooctyl, cyclononyl, bicyclo[3.1.0]hexyl, bicyclo[4.1.0]heptyl, bicyclo[5.1.0]octyl, bicyclo[6.1.0]nonyl, bicyclo[3.2.0]heptyl, bicyclo[4.2.0]octyl, bicyclo[5.2.0]nonyl, bicyclo[3.3.0]octyl, bicyclo[4.3.0]nonyl, bicyclo[2.2.1]heptyl, bicyclo[3.2.1]octyl, bicyclo[4.2.1]nonyl, bicyclo[2.2.2]octyl, bicyclo[3.2.2]nonyl, and bicyclo[3.3.1]nonyl.

In a further aspect, R¹⁰ is an optionally substituted heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R¹⁰ is optionally substituted cycloalkenyl selected from cyclobutenyl, cyclopentenyl, cyclopentadienyl, cyclohexenyl, cyclohexadienyl, cycloheptenyl, cycloheptadienyl, cyclooctenyl, cyclooctadienyl, cyclononenyl, and cyclononadienyl.

In a further aspect, R¹⁰ is optionally substituted heterocycloalkenyl comprising a mono-, di- or tri-unsaturated analog of a heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R¹⁰ is phenylethynyl, indolyl, quinolinyl, naphthyl, phenylcyclopropyl, or fluorophenyl.

i. R²¹ GROUPS

In one aspect, R²¹ is an optionally substituted C3 to C9 organic residue selected from aryl, heteroaryl, cycloalkyl, heterocycloalkyl, cycloalkenyl, and heterocycloalkenyl.

In a further aspect, R²¹ is optionally substituted aryl selected from phenyl and naphthyl.

In a further aspect, R²¹ is optionally substituted heteroaryl selected from furanyl, pyranyl, imidazolyl, thiophenyl, pyridinyl, pyridazinyl, pyrimidinyl, pyrazinyl, triazinyl, tetrazinyl, benzofuranyl, benzothiophene, indolyl, indazolyl, quinolinyl, naphthyridinyl, benzothiazolyl, benzooxazolyl, benzoimidazolyl, and benzotriazolyl.

In a further aspect, R²¹ is optionally substituted cycloalkyl selected from cyclopropyl, cyclobutyl, cyclopentyl, cyclohexyl, cycloheptyl, cyclooctyl, cyclononyl, bicyclo[3.1.0]hexyl, bicyclo[4.1.0]heptyl, bicyclo[5.1.0]octyl, bicyclo[6.1.0]nonyl, bicyclo[3.2.0]heptyl, bicyclo[4.2.0]octyl, bicyclo[5.2.0]nonyl, bicyclo[3.3.0]octyl, bicyclo[4.3.0]nonyl, bicyclo[2.2.1]heptyl, bicyclo[3.2.1]octyl, bicyclo[4.2.1]nonyl, bicyclo[2.2.2]octyl, bicyclo[3.2.2]nonyl, and bicyclo[3.3.1]nonyl.

In a further aspect, R²¹ is optionally substituted heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R²¹ is optionally substituted cycloalkenyl selected from cyclobutenyl, cyclopentenyl, cyclopentadienyl, cyclohexenyl, cyclohexadienyl, cycloheptenyl, cycloheptadienyl, cyclooctenyl, cyclooctadienyl, cyclononenyl, and cyclononadienyl.

In a further aspect, R²¹ is optionally substituted heterocycloalkenyl comprising a mono-, di- or tri-unsaturated analog of a heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R²¹ is halophenyl, for example 4-fluorophenyl.

j. R²² GROUPS

In one aspect, R²² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue.

In a further aspect, each R²² is hydrogen. In a further aspect, each R²² is independently selected from halide, hydroxyl, trifluoromethyl, amino, cyano, nitro, azide, carboxamido, alkoxy, thiol, alkylsulfonyl, and an optionally substituted C1 to C6 organic residue. In a further aspect, each R²² is independently selected from halide, hydroxyl, trifluoromethyl, amino, cyano, nitro, azide, carboxamido, alkoxy, thiol, and alkylsulfonyl. In a further aspect, at least one R²² is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

k. R²³ GROUPS

In one aspect, R²³ comprises hydrogen, an optionally substituted C1 to C6 alkyl, an optionally substituted C3 to C6 cycloalkyl, or a hydrolysable residue.

In a further aspect, R²³ is hydrogen. In a further aspect, R²³ is an optionally substituted C1 to C6 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, and cyclohexyl. In a further aspect, R²³ is an optionally substituted C3 to C6 cycloalkyl selected from cyclopropyl, cyclobutyl, cyclopentyl, cyclohexyl, and bicyclo[3.1.0]hexyl. In a further aspect, R²³ is a hydrolysable residue.

l. R²⁴ GROUPS

In one aspect, R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue.

In a further aspect, each R²⁴ is hydrogen. In a further aspect, each R²⁴ is independently selected from halide, hydroxyl, trifluoromethyl, amino, cyano, nitro, azide, carboxamido, alkoxy, thiol, alkylsulfonyl, and an optionally substituted C1 to C6 organic residue. In a further aspect, each R²⁴ is independently selected from halide, hydroxyl, trifluoromethyl, amino, cyano, nitro, azide, carboxamido, alkoxy, thiol, and alkylsulfonyl. In a further aspect, at least one R²⁴ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

m. R²⁵ AND R²⁶ GROUPS

In one aspect, each of R²⁵ and R²⁶ independently comprises hydrogen, trifluoromethyl, carboxamido, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl.

In a further aspect, R²⁵ is hydrogen. In a further aspect, R²⁵ is selected from trifluoromethyl, carboxamido, alkylsulfonyl, and an optionally substituted C1 to C6 organic residue. In a further aspect, R²⁵ is selected from trifluoromethyl, carboxamido, and alkylsulfonyl. In a further aspect, R²⁵ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R²⁶ is hydrogen. In a further aspect, R²⁶ is selected from trifluoromethyl, carboxamido, alkylsulfonyl, and an optionally substituted C1 to C6 organic residue. In a further aspect, R²⁶ is selected from trifluoromethyl, carboxamido, and alkylsulfonyl. In a further aspect, R²⁶ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R²⁶ is hydrogen and wherein R²⁵ is selected from trifluoromethyl, carboxamido, alkylsulfonyl, and an optionally substituted C1 to C6 organic residue. In a further aspect, R²⁶ is hydrogen and wherein R²⁵ is selected from trifluoromethyl, carboxamido, and alkylsulfonyl. In a further aspect, R²⁶ is hydrogen and wherein R²⁵ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R²⁵ is hydrogen and wherein R²⁶ is selected from trifluoromethyl, carboxamido, thiol, alkylsulfonyl, and an optionally substituted C1 to C6 organic residue. In a further aspect, R²⁵ is hydrogen and wherein R²⁶ is selected from trifluoromethyl, carboxamido, and alkylsulfonyl. In a further aspect, R²⁵ is hydrogen and wherein R²⁶ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R²⁵ and R²⁶, together with the intermediate carbon, comprise an optionally substituted C3 to C6 cycloalkyl. In a further aspect, wherein R²⁵ and R²⁶, together with the intermediate carbon, comprise cyclopropyl, cyclobutyl, cyclopentyl, or cyclohexyl.

n. R²⁷ AND R²⁸ GROUPS

In one aspect, each of R²⁷ and R²⁸ independently comprises hydrogen, trifluoromethyl, carboxamido, alkylsulfonyl, an optionally substituted C1 to C6 alkyl, or an optionally substituted C3 to C6 cycloalkyl or R²⁷ and R²⁸, together with the intermediate carbon, comprise an optionally substituted C3 to C6 cycloalkyl.

In a further aspect, R²⁷ is hydrogen. In a further aspect, R²⁷ is selected from trifluoromethyl, carboxamido, alkylsulfonyl, and an optionally substituted C1 to C6 organic residue. In a further aspect, R²⁷ is selected from trifluoromethyl, carboxamido, and alkylsulfonyl. In a further aspect, R²⁷ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl. In a further aspect, R²⁷ is methyl.

In a further aspect, R²⁸ is hydrogen. In a further aspect, R²⁸ is selected from trifluoromethyl, carboxamido, alkylsulfonyl, and an optionally substituted C1 to C6 organic residue. In a further aspect, R²⁸ is selected from trifluoromethyl, carboxamido, and alkylsulfonyl. In a further aspect, R²⁸ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl. In a further aspect, R²⁸ is methyl.

In a further aspect, R²⁸ is hydrogen and wherein R²⁷ is selected from trifluoromethyl, carboxamido, alkylsulfonyl, and an optionally substituted C1 to C6 organic residue. In a further aspect, R²⁸ is hydrogen and wherein R²⁷ is selected from trifluoromethyl, carboxamido, and alkylsulfonyl. In a further aspect, R²⁸ is hydrogen and wherein R²⁷ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R²⁷ is hydrogen and wherein R²⁸ is selected from trifluoromethyl, carboxamido, alkylsulfonyl, and an optionally substituted C1 to C6 organic residue. In a further aspect, R²⁷ is hydrogen and wherein R²⁸ is selected from trifluoromethyl, carboxamido, and alkylsulfonyl. In a further aspect, R²⁷ is hydrogen and wherein R²⁸ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R²⁷ and R²⁸, together with the intermediate carbon, comprise an optionally substituted C3 to C6 cycloalkyl. In a further aspect, R²⁷ and R²⁸, together with the intermediate carbon, comprise cyclopropyl, cyclobutyl, cyclopentyl, or cyclohexyl.

o. R²⁹ GROUPS

In one aspect, R²⁹ comprises hydrogen, an optionally substituted C1 to C6 alkyl, an optionally substituted C3 to C6 cycloalkyl, or a hydrolysable residue.

In a further aspect, R²⁹ is hydrogen. In a further aspect, R²⁹ is an optionally substituted C1 to C6 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, and cyclohexyl. In a further aspect, R²⁹ is an optionally substituted C3 to C6 cycloalkyl selected from cyclopropyl, cyclobutyl, cyclopentyl, and cyclohexyl. In a further aspect, R⁹ is a hydrolysable residue.

p. R³⁰ GROUPS

In one aspect, R³⁰ comprises an optionally substituted C1 to C12 organic residue selected from alkyl, aryl, heteroaryl, cycloalkyl, heterocycloalkyl, cycloalkenyl, and heterocycloalkenyl.

In a further aspect, R³⁰ is an optionally substituted alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, cyclohexyl, heptyl, cycloheptyl, octyl, cyclooctyl, nonyl, cyclononyl, decyl, cyclodecyl, undecyl, cycloundecyl, dodecyl, or cyclododecyl.

In a further aspect, R³⁰ is an optionally substituted aryl selected from phenyl and naphthyl.

In a further aspect, R³⁰ is an optionally substituted heteroaryl selected from furanyl, pyranyl, imidazolyl, thiophenyl, pyridinyl, pyridazinyl, pyrimidinyl, pyrazinyl, triazinyl, tetrazinyl, benzofuranyl, benzothiophene, indolyl, indazolyl, quinolinyl, naphthyridinyl, benzothiazolyl, benzooxazolyl, benzoimidazolyl, and benzotriazolyl.

In a further aspect, R³⁰ is an optionally substituted cycloalkyl selected from cyclopropyl, cyclobutyl, cyclopentyl, cyclohexyl, cycloheptyl, cyclooctyl, cyclononyl, bicyclo[3.1.0]hexyl, bicyclo[4.1.0]heptyl, bicyclo[5.1.0]octyl, bicyclo[6.1.0]nonyl, bicyclo[3.2.0]heptyl, bicyclo[4.2.0]octyl, bicyclo[5.2.0]nonyl, bicyclo[3.3.0]octyl, bicyclo[4.3.0]nonyl, bicyclo[2.2.1]heptyl, bicyclo[3.2.1]octyl, bicyclo[4.2.1]nonyl, bicyclo[2.2.2]octyl, bicyclo[3.2.2]nonyl, and bicyclo[3.3.1]nonyl.

In a further aspect, R³⁰ is an optionally substituted heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R³⁰ is optionally substituted cycloalkenyl selected from cyclobutenyl, cyclopentenyl, cyclopentadienyl, cyclohexenyl, cyclohexadienyl, cycloheptenyl, cycloheptadienyl, cyclooctenyl, cyclooctadienyl, cyclononenyl, and cyclononadienyl.

In a further aspect, R³⁰ is optionally substituted heterocycloalkenyl comprising a mono-, di- or tri-unsaturated analog of a heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R³⁰ is phenylethynyl, indolyl, quinolinyl, naphthyl, phenylcyclopropyl, or fluorophenyl.

q. R^{41A} AND R^{41B} GROUPS

In one aspect, each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue.

In a further aspect, each of R^{41a} and R^{41b} is hydrogen. In a further aspect, each of R^{41a} and R^{41b} is independently selected from halide, hydroxyl, trifluoromethyl, amino, cyano, nitro, azide, carboxamido, alkoxy, thiol, alkylsulfonyl, and an optionally substituted C1 to C6 organic residue. In a further aspect, each of R^{41a} and R^{41b} is independently selected from halide, hydroxyl, trifluoromethyl, amino, cyano, nitro, azide, carboxamido, alkoxy, thiol, and alkylsulfonyl. In a further aspect, at least one of R^{41a} and R^{41b} is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

r. R^{42A} AND R^{42B} GROUPS

In one aspect, each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue.

In a further aspect, each of R^{42a} and R^{42b} is hydrogen. In a further aspect, each of R^{42a} and R^{42b} is independently selected from halide, hydroxyl, trifluoromethyl, amino, cyano, nitro, azide, carboxamido, alkoxy, thiol, alkylsulfonyl, and an optionally substituted C1 to C6 organic residue. In a further aspect, each of R^{42a} and R^{42b} is independently selected from halide, hydroxyl, trifluoromethyl, amino, cyano, nitro, azide, carboxamido, alkoxy, thiol, and alkylsulfonyl. In a further aspect, at least one of R^{42a} and R^{42b} is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

s. R⁴³ GROUPS

In one aspect, R⁴³ comprises hydrogen, an optionally substituted C1 to C6 alkyl, an optionally substituted C3 to C6 cycloalkyl, or a hydrolysable residue.

In a further aspect, R⁴³ is hydrogen. In a further aspect, R⁴³ is an optionally substituted C1 to C6 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, and cyclohexyl. In a further aspect, R⁴³ is an optionally substituted C3 to C6 cycloalkyl selected from cyclopropyl, cyclobutyl, cyclopentyl, cyclohexyl, and bicyclo[3.1.0]hexyl. In a further aspect, R⁴³ is a hydrolysable residue.

t. R⁴⁴ GROUPS

In one aspect, R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue.

In a further aspect, each R⁴⁴ is hydrogen. In a further aspect, each R⁴⁴ is independently selected from halide, hydroxyl, trifluoromethyl, amino, cyano, nitro, azide, carboxamido, alkoxy, thiol, alkylsulfonyl, and an optionally substituted C1 to C6 organic residue. In a further aspect, each R⁴⁴ is independently selected from halide, hydroxyl, trifluoromethyl, amino, cyano, nitro, azide, carboxamido, alkoxy, thiol, and alkylsulfonyl. In a further aspect, at least one R⁴⁴ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

u. R⁴⁵ AND R⁴⁶ GROUPS

In one aspect, each of R⁴⁵ and R⁴⁶ independently comprises hydrogen, trifluoromethyl, carboxamido, alkylsulfonyl, an optionally substituted C1 to C6 alkyl, or an optionally substituted C3 to C6 cycloalkyl or R⁴⁵ and R⁴⁶, together with the intermediate carbon, comprise an optionally substituted C3 to C6 cycloalkyl.

In a further aspect, R⁴⁵ is hydrogen. In a further aspect, R⁴⁵ is selected from trifluoromethyl, carboxamido, alkylsulfonyl, and an optionally substituted C1 to C6 organic residue. In a further aspect, R⁴⁵ is selected from trifluoromethyl, carboxamido, and alkylsulfonyl. In a further aspect, R⁴⁵ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁴⁶ is hydrogen. In a further aspect, R⁴⁶ is selected from trifluoromethyl, alkoxy, thiol, alkylsulfonyl, and an optionally substituted C1 to C6 organic residue. In a further aspect, R⁴⁶ is selected from trifluoromethyl, carboxamido, and alkylsulfonyl. In a further aspect, R⁴⁶ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁴⁶ is hydrogen and wherein R⁴⁵ is selected from trifluoromethyl, carboxamido, alkylsulfonyl, and an optionally substituted C1 to C6 organic residue. In a further aspect, R⁴⁶ is hydrogen and wherein R⁴⁵ is selected from trifluoromethyl, carboxamido, and alkylsulfonyl. In a further aspect, R⁴⁶ is hydrogen and wherein R⁴⁵ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁴⁵ is hydrogen and wherein R⁴⁶ is selected from trifluoromethyl, carboxamido, alkylsulfonyl, and an optionally substituted C1 to C6 organic residue. In a further aspect, R⁴⁵ is hydrogen and wherein R⁴⁶ is selected from trifluoromethyl, carboxamido, and alkylsulfonyl. In a further aspect, R⁴⁵ is hydrogen and wherein R⁴⁶ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁴⁵ and R⁴⁶, together with the intermediate carbon, comprise an optionally substituted C3 to C6 cycloalkyl. In a further aspect, wherein R⁴⁵ and R⁴⁶, together with the intermediate carbon, comprise cyclopropyl, cyclobutyl, cyclopentyl, or cyclohexyl.

v. R⁴⁷ AND R⁴⁸ GROUPS

In one aspect, each of R⁴⁷ and R⁴⁸ independently comprises hydrogen, trifluoromethyl, carboxamido, alkylsulfonyl, an optionally substituted C1 to C6 alkyl, or an optionally substituted C3 to C6 cycloalkyl or R⁴⁷ and R⁴⁸, together with the intermediate carbon, comprise an optionally substituted C3 to C6 cycloalkyl.

In a further aspect, R⁴⁷ is hydrogen. In a further aspect, R⁴⁷ is selected from trifluoromethyl, carboxamido, alkylsulfonyl, and an optionally substituted C1 to C6 organic residue. In a further aspect, R⁴⁷ is selected from trifluoromethyl, carboxamido, and alkylsulfonyl. In a further aspect, R⁴⁷ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl. In a further aspect, R⁴⁷ is methyl.

In a further aspect, R⁴⁸ is hydrogen. In a further aspect, R⁴⁸ is selected from trifluoromethyl, carboxamido, alkylsulfonyl, and an optionally substituted C1 to C6 organic residue. In a further aspect, R⁴⁸ is selected from trifluoromethyl, carboxamido, and alkylsulfonyl. In a further aspect, R⁴⁸ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl. In a further aspect, R⁴⁸ is methyl.

In a further aspect, R⁴⁸ is hydrogen and wherein R⁴⁷ is selected from trifluoromethyl, carboxamido, alkoxy, thiol, alkylsulfonyl, and an optionally substituted C1 to C6 organic residue. In a further aspect, R⁴⁸ is hydrogen and wherein R⁴⁷ is selected from trifluoromethyl, carboxamido, and alkylsulfonyl. In a further aspect, R⁴⁸ is hydrogen and wherein R⁴⁷ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁴⁷ is hydrogen and wherein R⁴⁸ is selected from trifluoromethyl, carboxamido, alkylsulfonyl, and an optionally substituted C1 to C6 organic residue. In a further aspect, R⁴⁷ is hydrogen and wherein R⁴⁸ is selected from trifluoromethyl, carboxamido, and alkylsulfonyl. In a further aspect, R⁴⁷ is hydrogen and wherein R⁴⁸ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁴⁷ and R⁴⁸, together with the intermediate carbon, comprise an optionally substituted C3 to C6 cycloalkyl. In a further aspect, R⁴⁷ and R⁴⁸, together with the intermediate carbon, comprise cyclopropyl, cyclobutyl, cyclopentyl, or cyclohexyl.

w. R⁴⁹ GROUPS

In one aspect, R⁴⁹ comprises hydrogen, an optionally substituted C1 to C6 alkyl, an optionally substituted C3 to C6 cycloalkyl, or a hydrolysable residue.

In a further aspect, R⁴⁹ is hydrogen. In a further aspect, R⁴⁹ is an optionally substituted C1 to C6 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, and cyclohexyl. In a further aspect, R⁴⁹ is an optionally substituted C3 to C6 cycloalkyl selected from cyclopropyl, cyclobutyl, cyclopentyl, and cyclohexyl. In a further aspect, R⁹ is a hydrolysable residue.

x. R⁵⁰ GROUPS

In one aspect, R⁵⁰ comprises an optionally substituted C1 to C16 organic residue selected from alkyl, aryl, heteroaryl, cycloalkyl, heterocycloalkyl, cycloalkenyl, and heterocycloalkenyl.

In a further aspect, R⁵⁰ is an optionally substituted alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, cyclohexyl, heptyl, cycloheptyl, octyl, cyclooctyl, nonyl, cyclononyl, decyl, cyclodecyl, undecyl, cycloundecyl, dodecyl, or cyclododecyl.

In a further aspect, R⁵⁰ is an optionally substituted aryl selected from phenyl and naphthyl.

In a further aspect, R⁵⁰ is an optionally substituted heteroaryl selected from furanyl, pyranyl, imidazolyl, thiophenyl, pyridinyl, pyridazinyl, pyrimidinyl, pyrazinyl, triazinyl, tetrazinyl, benzofuranyl, benzothiophene, indolyl, indazolyl, quinolinyl, naphthyridinyl, benzothiazolyl, benzooxazolyl, benzoimidazolyl, and benzotriazolyl.

In a further aspect, R⁵⁰ is an optionally substituted cycloalkyl selected from cyclopropyl, cyclobutyl, cyclopentyl, cyclohexyl, cycloheptyl, cyclooctyl, cyclononyl, bicyclo[3.1.0]hexyl, bicyclo[4.1.0]heptyl, bicyclo[5.1.0]octyl, bicyclo[6.1.0]nonyl, bicyclo[3.2.0]heptyl, bicyclo[4.2.0]octyl, bicyclo[5.2.0]nonyl, bicyclo[3.3.0]octyl, bicyclo[4.3.0]nonyl, bicyclo[2.2.1]heptyl, bicyclo[3.2.1]octyl, bicyclo[4.2.1]nonyl, bicyclo[2.2.2]octyl, bicyclo[3.2.2]nonyl, and bicyclo[3.3.1]nonyl.

In a further aspect, R⁵⁰ is an optionally substituted heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R⁵⁰ is optionally substituted cycloalkenyl selected from cyclobutenyl, cyclopentenyl, cyclopentadienyl, cyclohexenyl, cyclohexadienyl, cycloheptenyl, cycloheptadienyl, cyclooctenyl, cyclooctadienyl, cyclononenyl, and cyclononadienyl.

In a further aspect, R⁵⁰ is optionally substituted heterocycloalkenyl comprising a mono-, di- or tri-unsaturated analog of a heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R⁵⁰ is phenylethynyl, indolyl, quinolinyl, naphthyl, phenylcyclopropyl, or fluorophenyl.

2. Example Compounds

In one aspect, the invention relates to phospholipase D inhibitors comprising one or more compounds selected from:

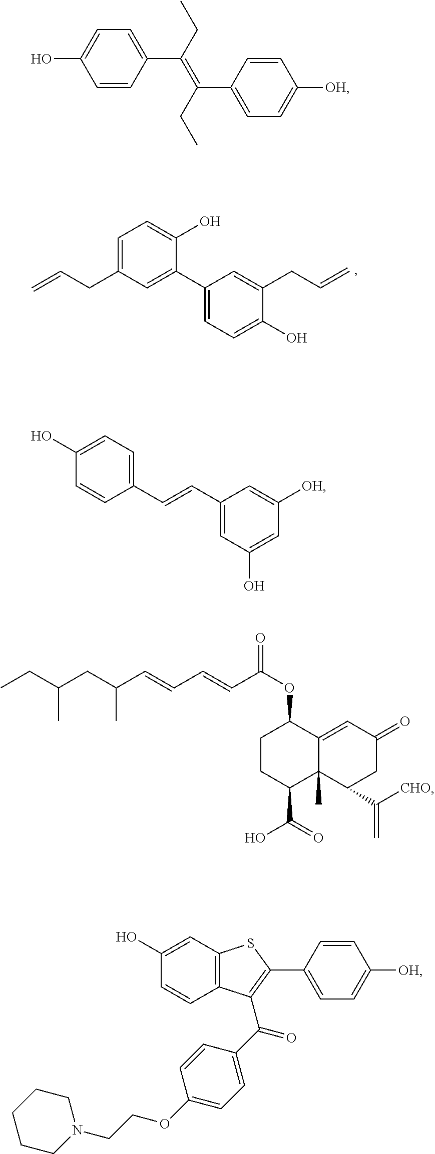

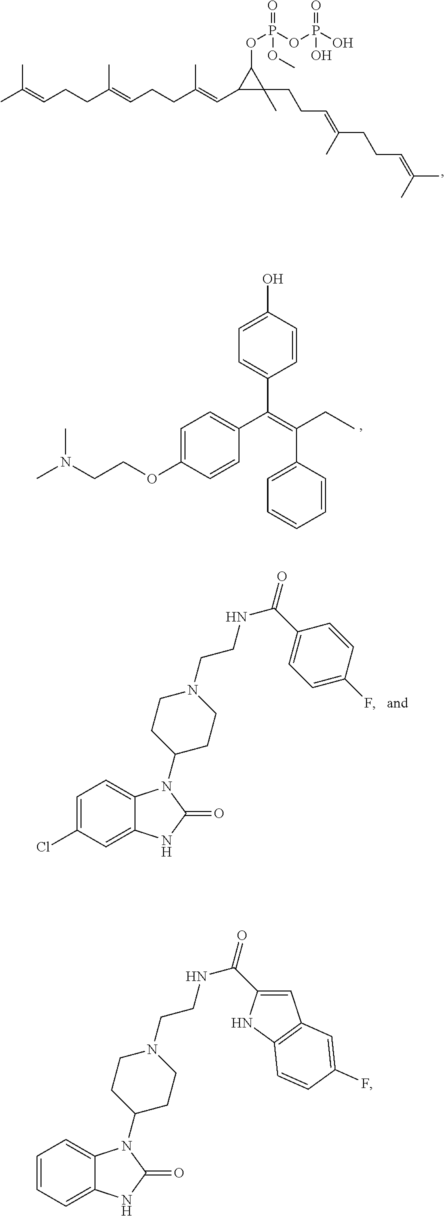

or a subgroup thereof.

In a further aspect, the invention relates to phospholipase D inhibitors comprising a compound selected from trans-diethylstilbestrol ((E)-4,4′-(hex-3-ene-3,4-diyl)diphenol); resveratrol (5-[2-(4-hydroxyphenyl)ethenyl]benzene-1,3-diol); honokiol (3′,5-diallyl-[1,1′-biphenyl]-2,4′-diol); SCH420789 ((1S,4R,8S,8aR)-4-(((2E,4E)-6,8-dimethyldeca-2,4-dienoyl)oxy)-8a-methyl-6-oxo-8-(3-oxoprop-1-en-2-yl)-1,2,3,4,6,7,8,8a-octahydronaphthalene-1-carboxylic acid); presqualene diphosphate ([[2-(4,8-dimethylnona-3,7-dienyl)-2-methyl-3-(2,6,10-trimethylundeca-1,5,9-trienyl)cyclopropyl]methoxy-hydroxy-phosphoryl]oxyphosphonic acid); raloxifene ((6-hydroxy-2-(4-hydroxyphenyl)benzo[b]thiophen-3-yl)(4-(2-(piperidin-1-yl)ethoxy)phenyl)methanone); 4-hydroxytamoxifen (4-[(Z)-1-[4-[2-(dimethylamino)ethoxy]phenyl]-2-phenylbut-1-enyl]phenol); 5-fluoro-2-indoyl des-chlorohalopemide (N-[2-[4-(2,3-dihydro-2-oxo-1H-benzimidazol-1-yl)-1-piperidinyl]ethyl]-5-fluoro-1H-indole-2-carboxamide), and halopemide (N-[2-[4-(5-Chloro-2,3-dihydro-2-oxo-1H-benzimidazol-1-yl)piperidino]ethyl]-4-fluorobenzamide).

In various aspects, a phospholipase D inhibitor compound can be present as:

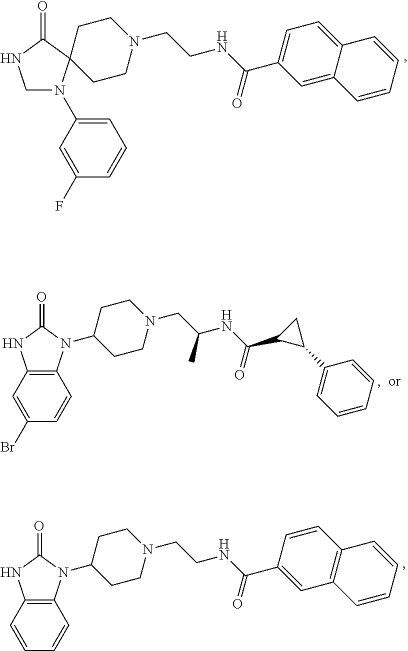

or a subgroup thereof.

In various aspects, a phospholipase D inhibitor compound can be present as:

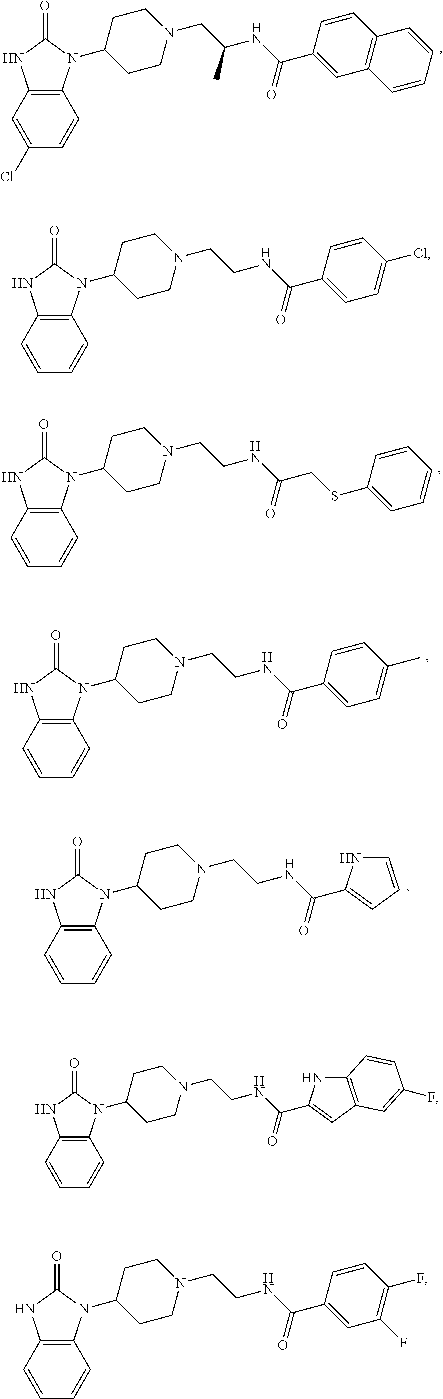

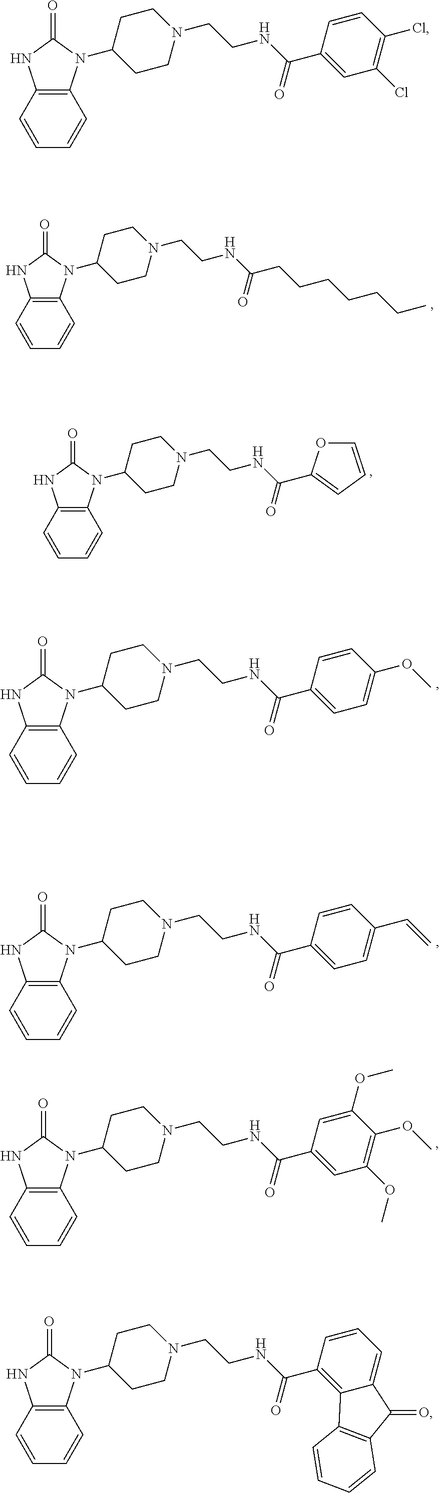

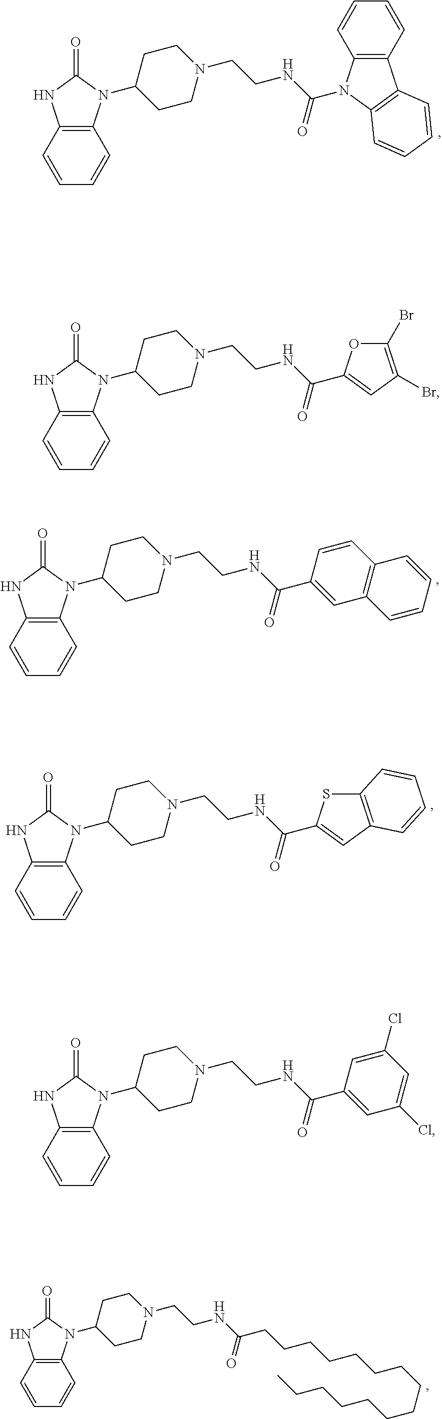 [SMILES: O=C(NCCN1CCC(n2c(=O)[nH]c3ccccc32)CC1)n1c2ccccc2c2ccccc21 ; O=C(NCCN1CCC(n2c(=O)[nH]c3ccccc32)CC1)c1cc(Br)c(Br)o1 ; O=C(NCCN1CCC(n2c(=O)[nH]c3ccccc32)CC1)c1ccc2ccccc2c1 ; O=C(NCCN1CCC(n2c(=O)[nH]c3ccccc32)CC1)c1cc2ccccc2s1 ; O=C(NCCN1CCC(n2c(=O)[nH]c3ccccc32)CC1)c1cc(Cl)cc(Cl)c1 ; CCCCCCCCCCCCCCCC(=O)NCCN1CCC(n2c(=O)[nH]c3ccccc32)CC1]

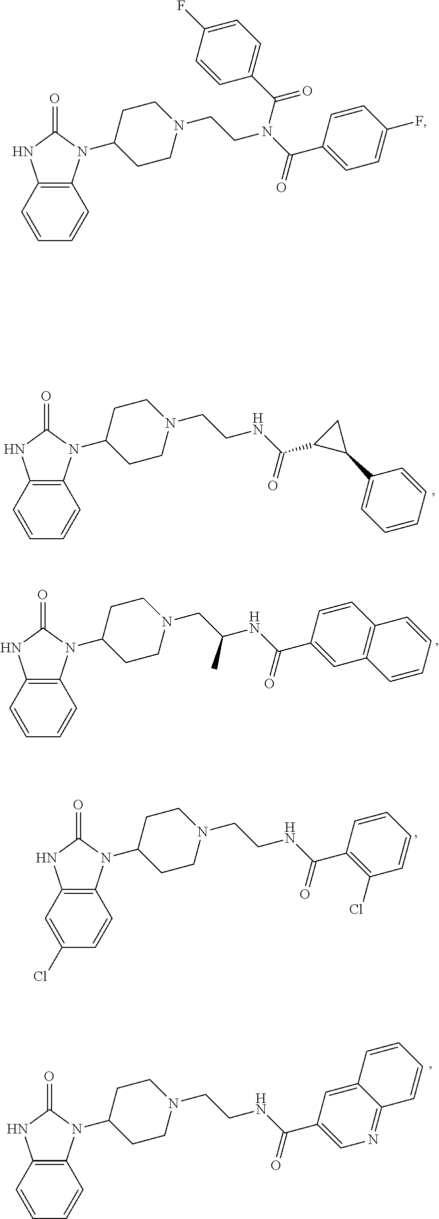

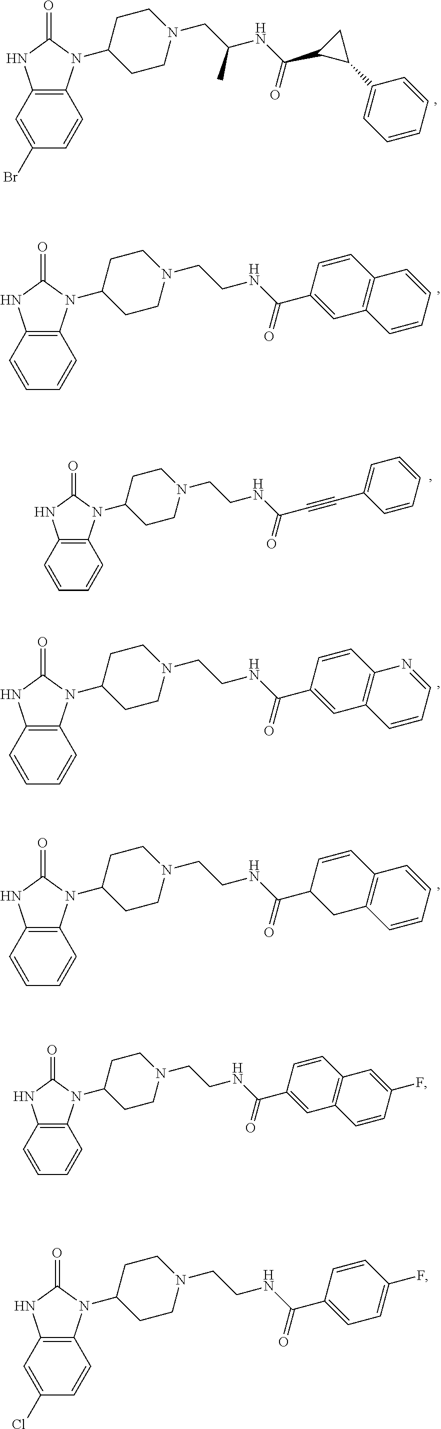

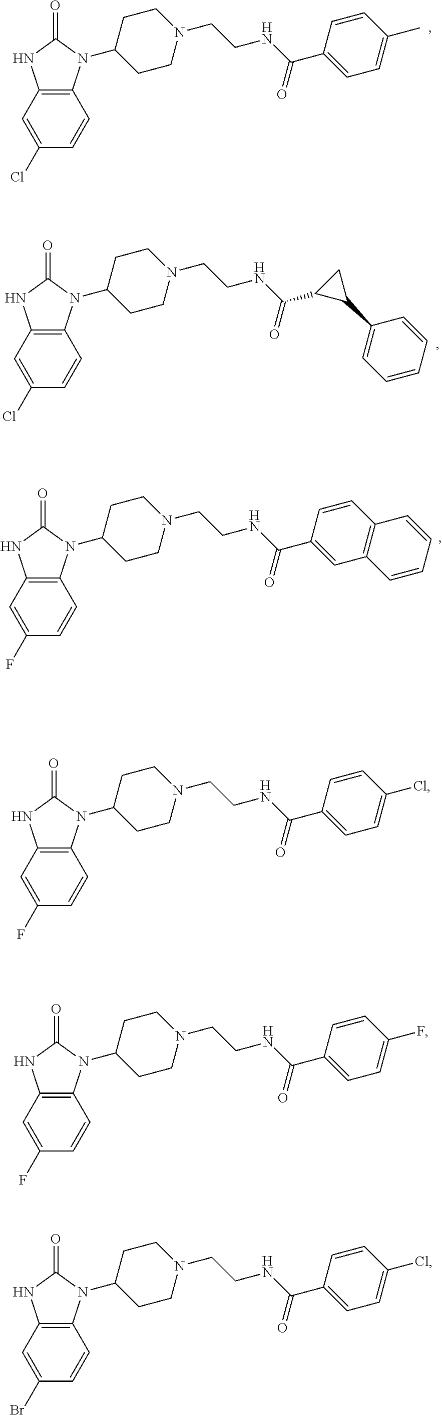

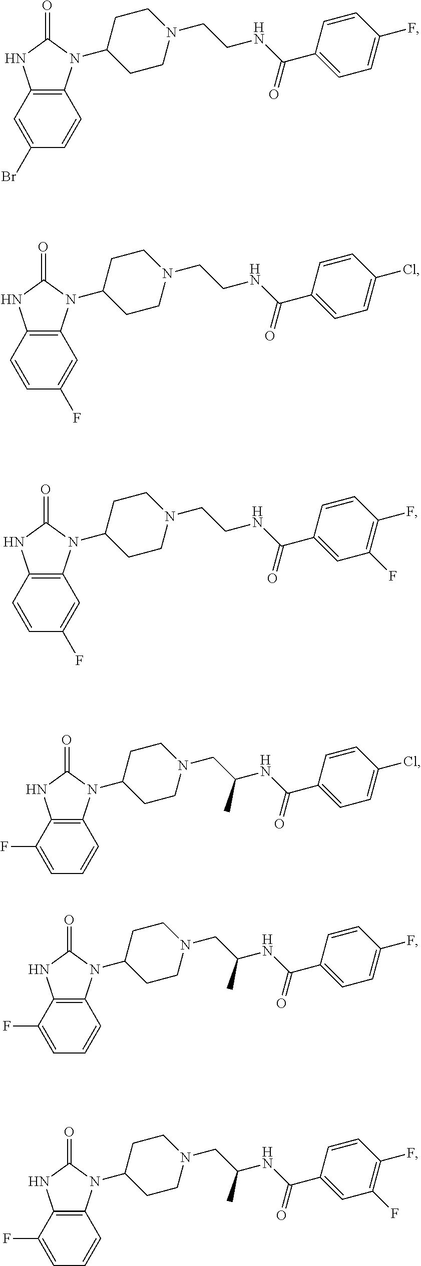 [SMILES: O=C(NCCN1CCC(n2c(=O)[nH]c3cc(Br)ccc32)CC1)c1ccc(F)cc1 ; O=C(NCCN1CCC(n2c(=O)[nH]c3ccc(F)cc32)CC1)c1ccc(Cl)cc1 ; O=C(NCCN1CCC(n2c(=O)[nH]c3ccc(F)cc32)CC1)c1ccc(F)c(F)c1 ; C[C@@H](CN1CCC(n2c(=O)[nH]c3c(F)cccc32)CC1)NC(=O)c1ccc(Cl)cc1 ; C[C@@H](CN1CCC(n2c(=O)[nH]c3c(F)cccc32)CC1)NC(=O)c1ccc(F)cc1 ; C[C@@H](CN1CCC(n2c(=O)[nH]c3c(F)cccc32)CC1)NC(=O)c1ccc(F)c(F)c1]

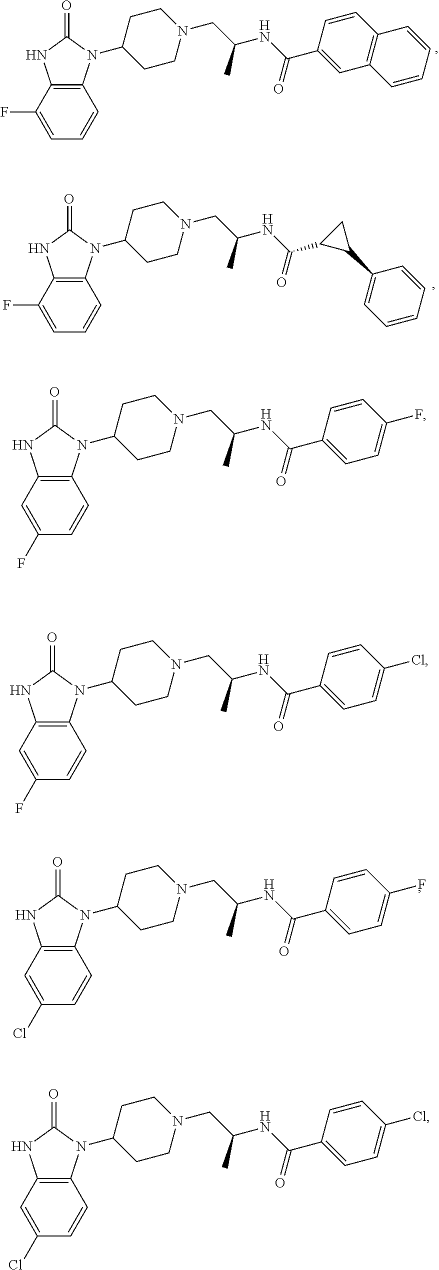

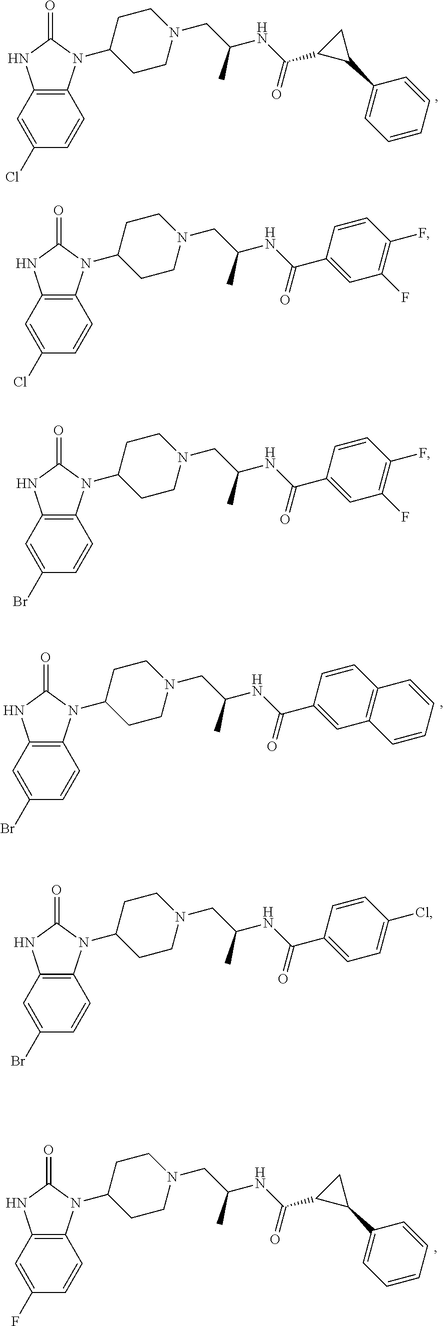

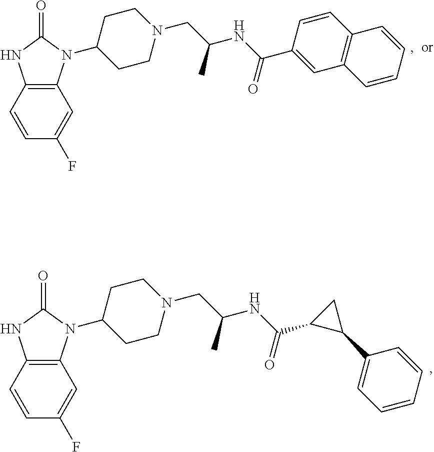 [SMILES: C[C@@H](CN1CCC(n2c(=O)[nH]c3ccc(F)cc32)CC1)NC(=O)c1ccc2ccccc2c1 ; C[C@@H](CN1CCC(n2c(=O)[nH]c3ccc(F)cc32)CC1)NC(=O)[C@@H]1C[C@H]1c1ccccc1]

a subgroup thereof.

In various aspects, a phospholipase D inhibitor compound can be present as:

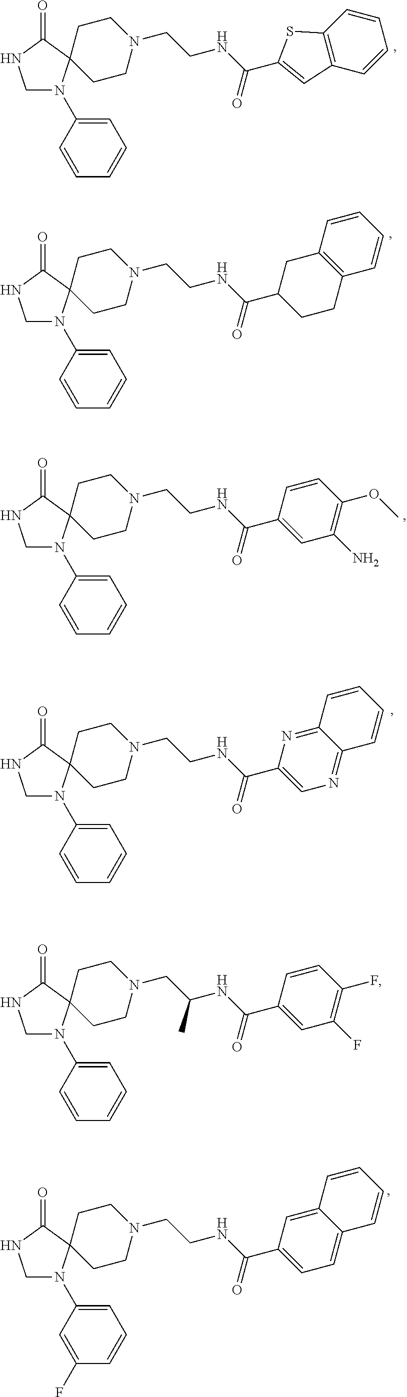 [SMILES: O=C(NCCN1CCC2(CC1)C(=O)NCN2c1ccccc1)c1cc2ccccc2s1 ; O=C(NCCN1CCC2(CC1)C(=O)NCN2c1ccccc1)C1CCc2ccccc2C1 ; COc1ccc(C(=O)NCCN2CCC3(CC2)C(=O)NCN3c2ccccc2)cc1N ; O=C(NCCN1CCC2(CC1)C(=O)NCN2c1ccccc1)c1cnc2ccccc2n1 ; C[C@@H](CN1CCC2(CC1)C(=O)NCN2c1ccccc1)NC(=O)c1ccc(F)c(F)c1 ; O=C(NCCN1CCC2(CC1)C(=O)NCN2c1cccc(F)c1)c1ccc2ccccc2c1]

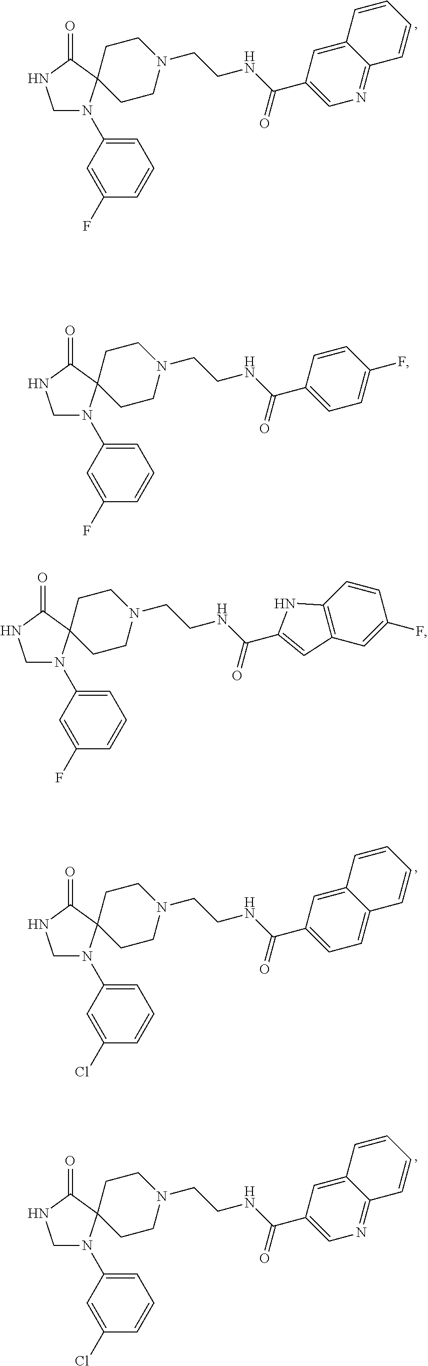 [SMILES: O=C(NCCN1CCC2(CC1)C(=O)NCN2c1cccc(F)c1)c1cnc2ccccc2c1 ; O=C(NCCN1CCC2(CC1)C(=O)NCN2c1cccc(F)c1)c1ccc(F)cc1 ; O=C(NCCN1CCC2(CC1)C(=O)NCN2c1cccc(F)c1)c1cc2cc(F)ccc2[nH]1 ; O=C(NCCN1CCC2(CC1)C(=O)NCN2c1cccc(Cl)c1)c1ccc2ccccc2c1 ; O=C(NCCN1CCC2(CC1)C(=O)NCN2c1cccc(Cl)c1)c1cnc2ccccc2c1]

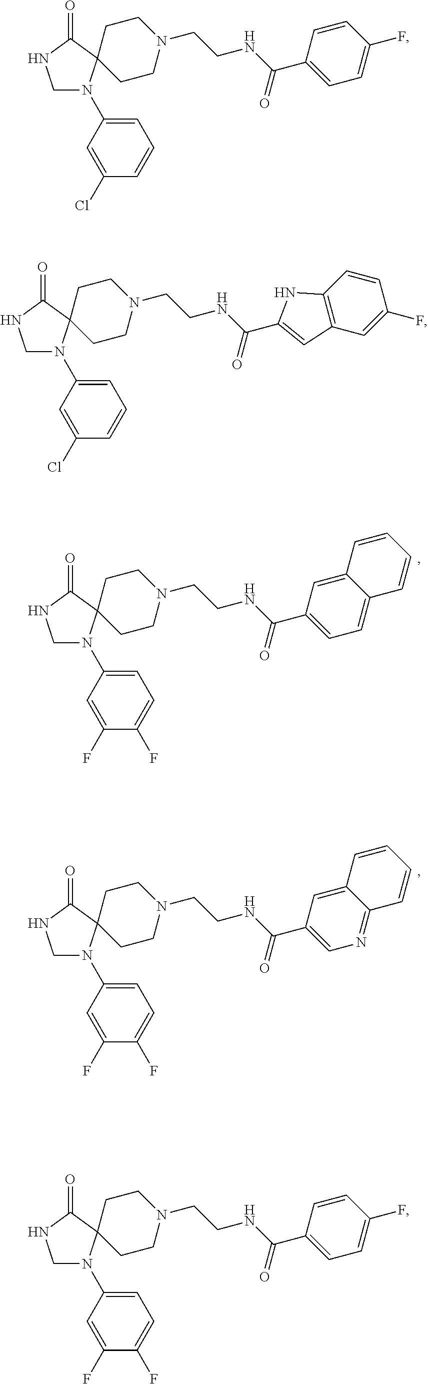 [SMILES: O=C(NCCN1CCC2(CC1)C(=O)NCN2c1cccc(Cl)c1)c1ccc(F)cc1 ; O=C(NCCN1CCC2(CC1)C(=O)NCN2c1cccc(Cl)c1)c1cc2cc(F)ccc2[nH]1 ; O=C(NCCN1CCC2(CC1)C(=O)NCN2c1ccc(F)c(F)c1)c1ccc2ccccc2c1 ; O=C(NCCN1CCC2(CC1)C(=O)NCN2c1ccc(F)c(F)c1)c1cnc2ccccc2c1 ; O=C(NCCN1CCC2(CC1)C(=O)NCN2c1ccc(F)c(F)c1)c1ccc(F)cc1]

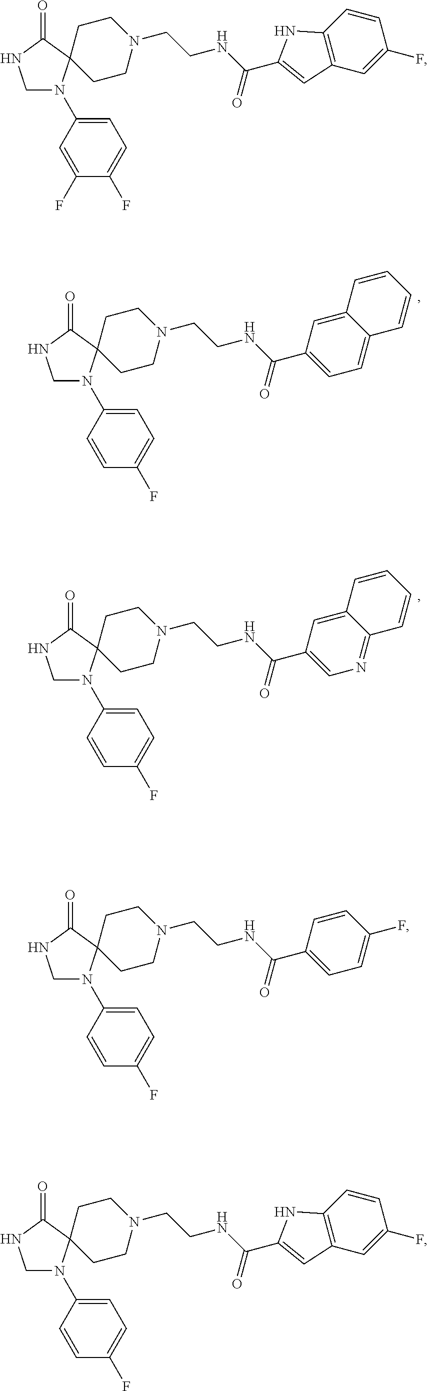 [SMILES: O=C(NCCN1CCC2(CC1)C(=O)NCN2c1ccc(F)c(F)c1)c1cc2cc(F)ccc2[nH]1 ; O=C(NCCN1CCC2(CC1)C(=O)NCN2c1ccc(F)cc1)c1ccc2ccccc2c1 ; O=C(NCCN1CCC2(CC1)C(=O)NCN2c1ccc(F)cc1)c1cnc2ccccc2c1 ; O=C(NCCN1CCC2(CC1)C(=O)NCN2c1ccc(F)cc1)c1ccc(F)cc1 ; O=C(NCCN1CCC2(CC1)C(=O)NCN2c1ccc(F)cc1)c1cc2cc(F)ccc2[nH]1]

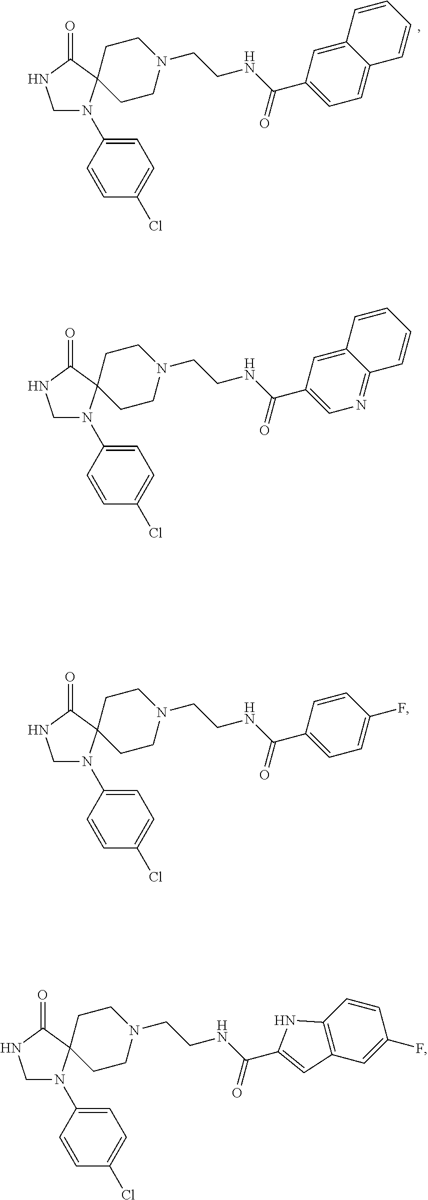 [SMILES: O=C(NCCN1CCC2(CC1)C(=O)NCN2c1ccc(Cl)cc1)c1ccc2ccccc2c1 ; O=C(NCCN1CCC2(CC1)C(=O)NCN2c1ccc(Cl)cc1)c1cnc2ccccc2c1 ; O=C(NCCN1CCC2(CC1)C(=O)NCN2c1ccc(Cl)cc1)c1ccc(F)cc1 ; O=C(NCCN1CCC2(CC1)C(=O)NCN2c1ccc(Cl)cc1)c1cc2cc(F)ccc2[nH]1]

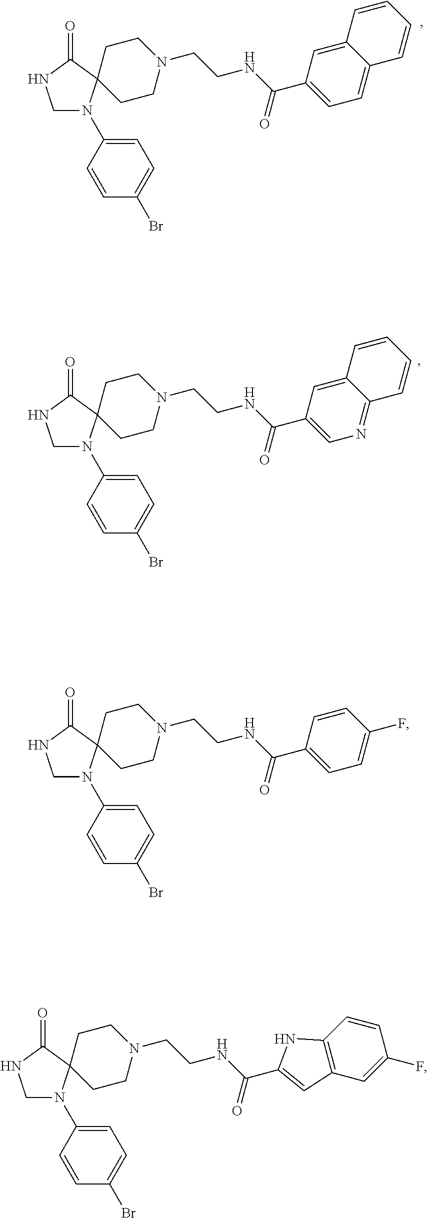 [SMILES: O=C(NCCN1CCC2(CC1)C(=O)NCN2c1ccc(Br)cc1)c1ccc2ccccc2c1 ; O=C(NCCN1CCC2(CC1)C(=O)NCN2c1ccc(Br)cc1)c1cnc2ccccc2c1 ; O=C(NCCN1CCC2(CC1)C(=O)NCN2c1ccc(Br)cc1)c1ccc(F)cc1 ; O=C(NCCN1CCC2(CC1)C(=O)NCN2c1ccc(Br)cc1)c1cc2cc(F)ccc2[nH]1]

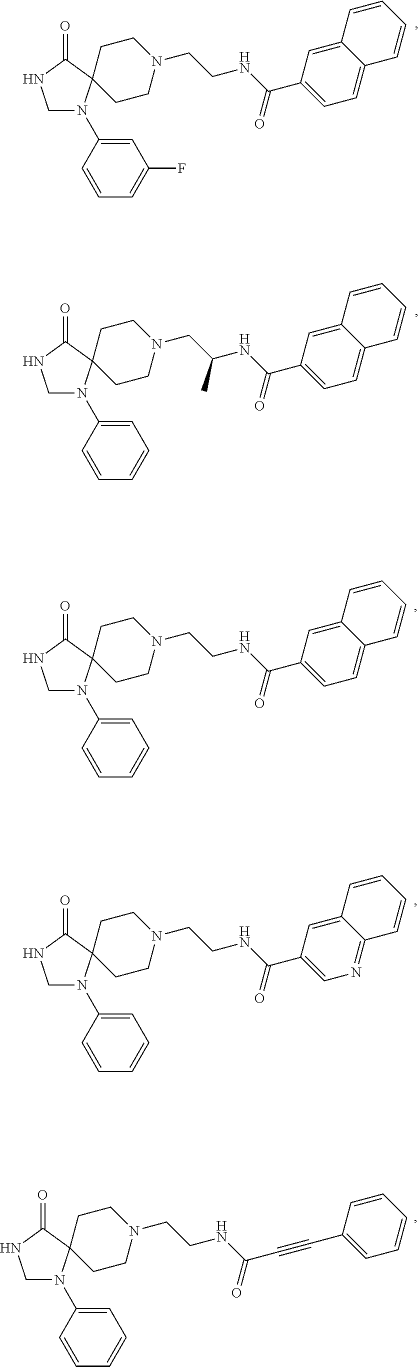 [SMILES: O=C(NCCN1CCC2(CC1)C(=O)NCN2c1cccc(F)c1)c1ccc2ccccc2c1 ; C[C@@H](CN1CCC2(CC1)C(=O)NCN2c1ccccc1)NC(=O)c1ccc2ccccc2c1 ; O=C(NCCN1CCC2(CC1)C(=O)NCN2c1ccccc1)c1ccc2ccccc2c1 ; O=C(NCCN1CCC2(CC1)C(=O)NCN2c1ccccc1)c1cnc2ccccc2c1 ; O=C(C#Cc1ccccc1)NCCN1CCC2(CC1)C(=O)NCN2c1ccccc1]

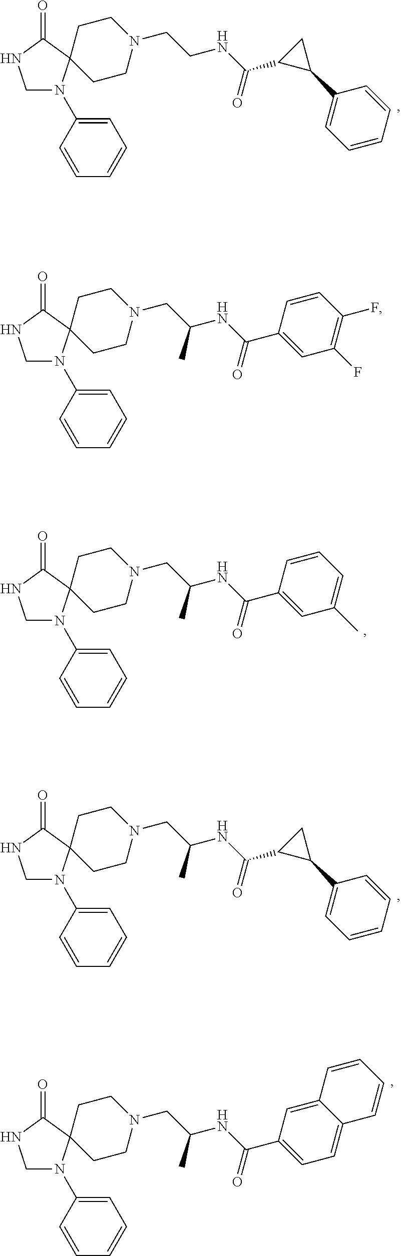

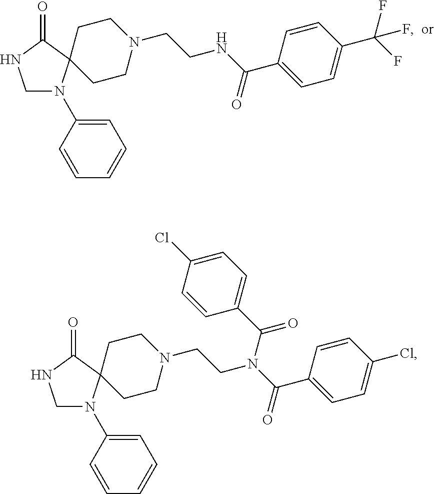

or a subgroup thereof.

In various aspects, a phospholipase D inhibitor compound can be present as:

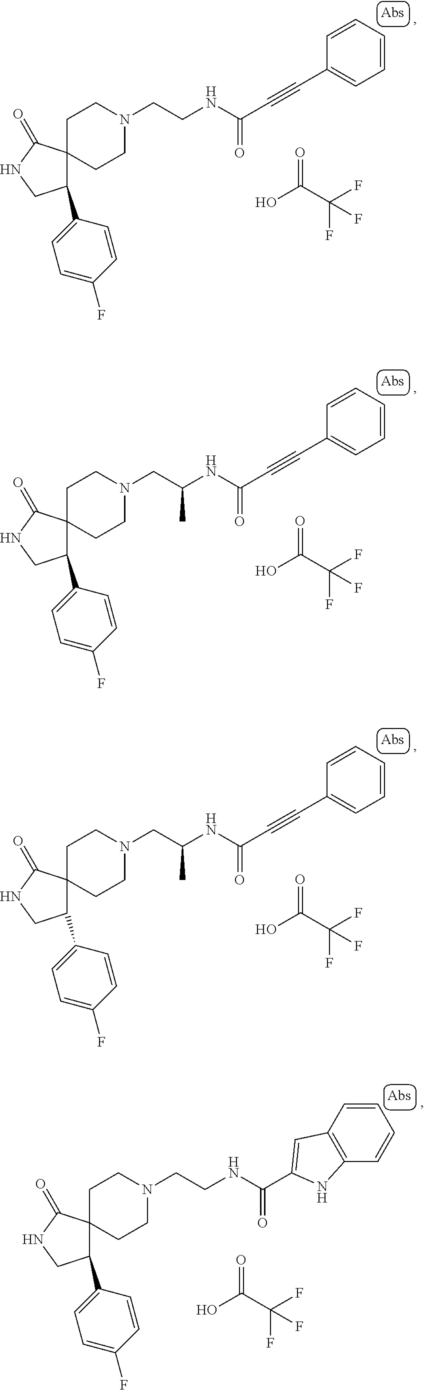

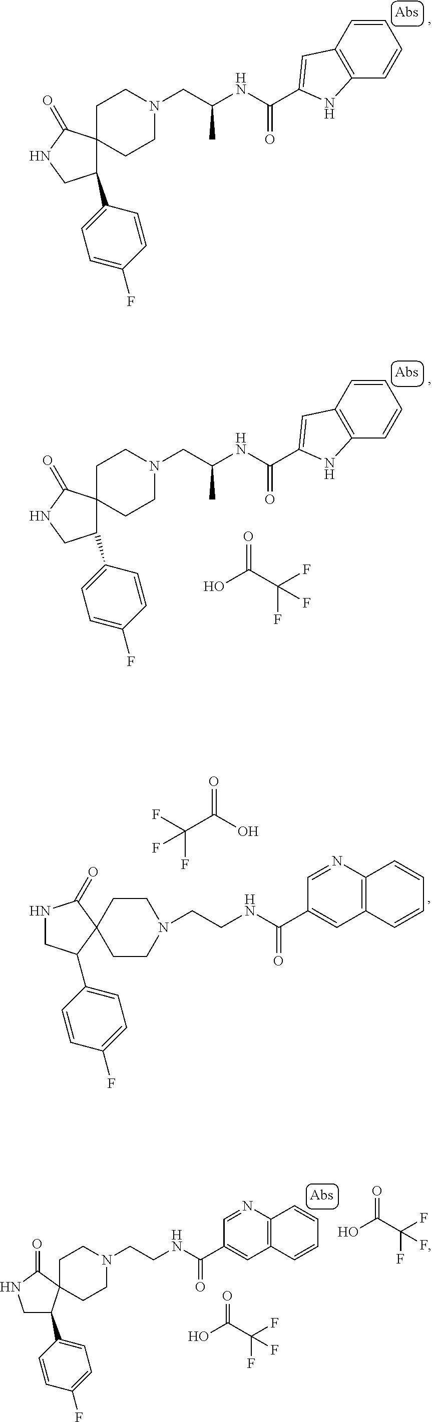

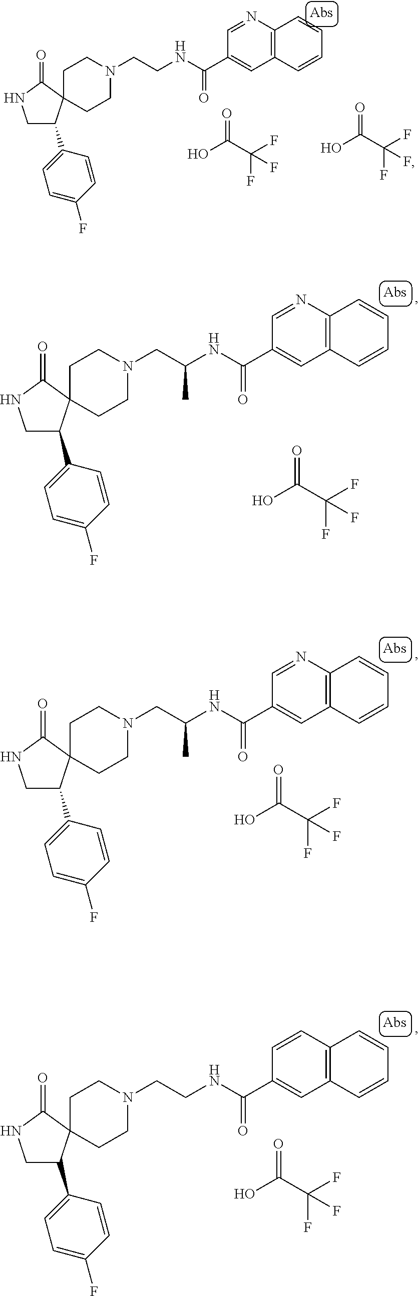

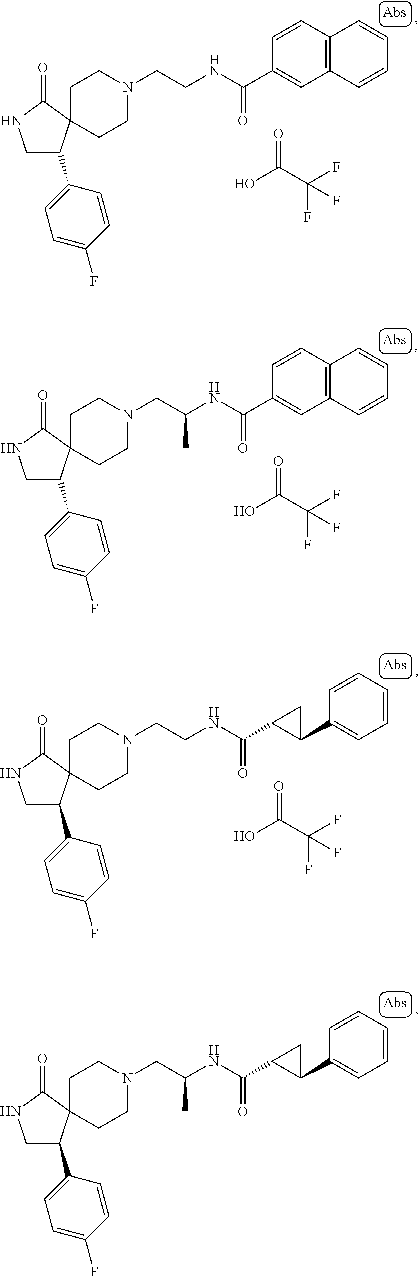

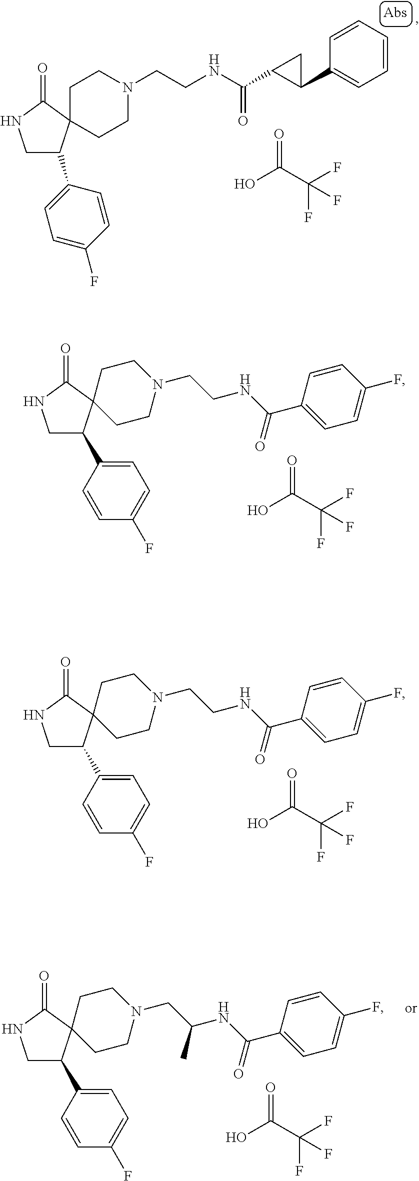

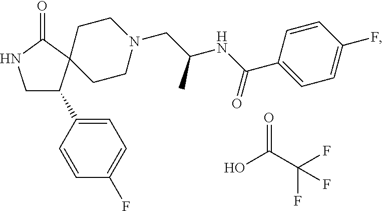

or a subgroup thereof.

### C. PHOSPHOLIPASE D INHIBITION ACTIVITY

In a further aspect, the invention relates to compounds that inhibit a phospholipase D selected from PLD1 and PLD2. In a still further aspect, the compounds inhibit PLD1. In a yet further aspect, the compounds inhibit PLD2. In an even further aspect, the compounds inhibit one or more PLD1 proteins selected from PLD1A, PLD1B, PLD1C, and PLD1D. In a yet further aspect, the compounds inhibit one or more PLD2 selected from PLD2A, PLD2B, and PLD2C.

In one aspect, the compound inhibits PLD activity, i.e. a compound can inhibit PLD 1 activity and/or PLD2 activity. In a further aspect, the compound inhibits PLD 1 response in Calu-1 cells. In a further aspect, the compound inhibits PLD2 response in HEK293gfpPLD2 cells. In a further aspect, the compound inhibits in vitro PLD1 response. In a further aspect, the compound inhibits in vitro PLD2 response. For example, the compound can have a PLD 1 IC₅₀ of less than about 10 μM, of less than about 5 μM, of less than about 1 μM, of less than about 500 nM, of less than about 100 nM, or of less than about 50 nM. As further examples, the compound can have a PLD2 IC₅₀ of less than about 10 μM, of less than about 5 μM, of less than about 1 μM, of less than about 500 nM, of less than about 100 nM, or of less than about 50 nM.

In a further aspect, the compound can have a PLD1 IC₅₀ of less than about 10 μM, of less than about 1 μM, of less than about 500 nM, of less than about 100 nM, of less than about 60 nM, or of less than about 20 nM. In a further aspect, the compound can have a PLD2 IC₅₀ of less than about 10 μM, of less than about 1 μM, of less than about 500 nM, of less than about 100 nM, of less than about 60 nM, or of less than about 20 nM.

### D. MODULATION OF AKT ACTIVITY

In a further aspect, the invention relates to modulation of Akt activity by compounds that inhibit a phospholipase D selected from PLD1 and PLD2. Without wishing to be bound by a particular theory, it is believed that the modulation of Akt activity by phospholipase D inhibitors is indirect. For example, again without wishing to be bound by a particular theory, phosphatidic acid binds to Akt and is involved with the level of Akt protein-protein interactions. Thus, inhibition of a PLD is associated with alteration of cellular pool of phosphatidic acid, resulting in modulation of Akt activity. Without wishing to be bound by a particular theory, both PIP3 and phosphatidic acid modulate the activity of Akt.

### E. METHODS OF MAKING THE COMPOUNDS

The compounds of this invention can be prepared by employing reactions as shown in the disclosed schemes below, in addition to other standard manipulations that are known in the literature, exemplified in the experimental sections or clear to one skilled in the art. For clarity, examples having a fewer substituent can be shown where multiple substituents are allowed under the definitions disclosed herein. The compounds of this invention can be prepared by employing reactions as disclosed in the references cited herein. For example, suitable methods for synthesizing the disclosed compounds are provided in WO/2011/011680; Scott, S., et al. (2009) Nat. Chem. Biol. 5(2):108-117; Lewis, J. A., et al. (2009) Bioorg. Med. Chem. 19:1916-1920; Lavieri, R., et al. (2009) Bioorg. Med. Chem. 19:2240-2243; and Lavieri, R. R., et al. (2010) J. Med. Chem. 53:6706-6719.

1. Route I

In one aspect, substituted 1-oxo-2,8-diazaspiro[4.5]decanyl analogs of the present invention can be prepared generically by the synthetic scheme as shown below.

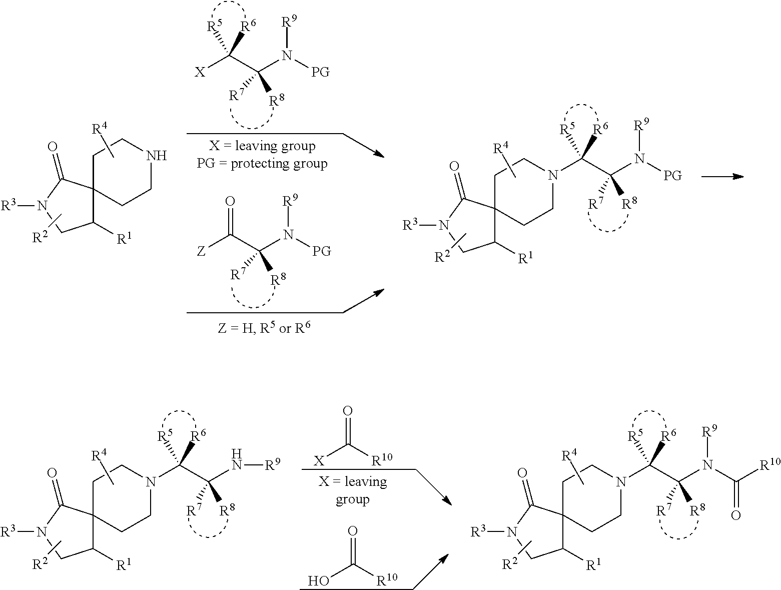

Compounds are represented in generic form, with substituents as noted in compound descriptions elsewhere herein. A more specific example is set forth below.

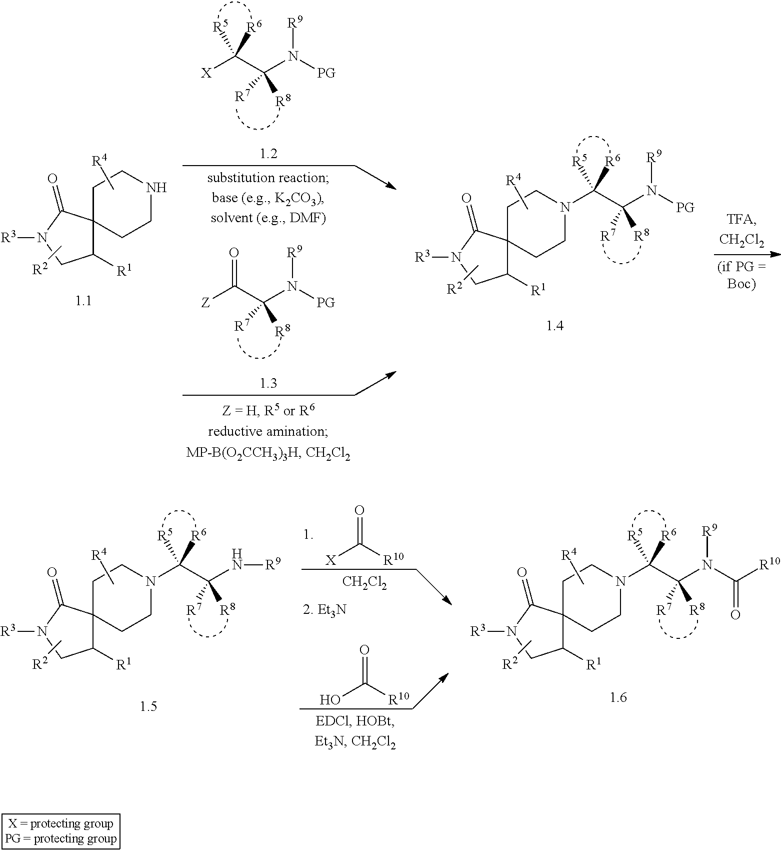

In one aspect, Route I begins with a suitable substituted 2,8-diazaspiro[4.5]decan-1-one (1.1). A suitable 2,8-diazaspiro[4.5]decan-1-one (1.1) is commercially available or can be readily prepared by one skilled in the art. The first reaction of 1.1 and a suitable substituted N-protected amino derivative (1.2) involves a nucleophilic substitution reaction resulting in a N-protected product (1.4). Alternately, the reaction of 1.1 and compound 1.3 [e.g., where R⁵ or R⁶═H, alkyl group, or aryl group] is a reductive amination reaction resulting in a N-protected product (1.4).

In one aspect, the reaction of 1.1 and 1.2 is typically carried out under a suitable reaction atmosphere and in a suitable solvent that supports substitution reactions such as DMF in the presence of an appropriate base such as K₂CO₃. The reaction is conducted at a suitable temperature and for a time sufficient to complete the reaction and to provide compounds of type 1.4 as shown above. The product, a compound of type 1.4, is isolated by methods known to one skilled in the art (e.g., extraction, washing, drying, and concentration under a vacuum; followed by purification, e.g., chromatography, if necessary).

In one aspect, the reaction of 1.1 and 1.3 is typically carried out under a suitable reaction condition that supports reductive amination of carbonyl compounds known to one skilled in the art to give products of type 1.4. Reaction components 1.1 and 1.3 are dissolved in a suitable solvent, e.g., dichloromethane, and stirred at ambient temperature (about 15-30° C.) for about 15 min. Then, the reducing agent, [e.g., macroporous polystyrene triacetoxyborohydride, MP—B(O₂CCH₃)₃H. or other suitable reducing agent] is added to the reaction mixture. The reaction is carried out for a time sufficient to complete the reaction, e.g., overnight (about 8-18 h), to provide compounds of type 1.4 as shown above. The product, a compound of type 1.4, is isolated by methods known to one skilled in the art (e.g., filtered, and concentration under a vacuum; followed by purification, e.g., chromatography, if necessary).

In one aspect, compounds of type 1.5 can be prepared by the conversion of the N-protected compound (e.g., N-Boc compound type 1.4) to the corresponding amine derivative (1.5). For example, a reaction of this type is commonly carried out by dissolving the N-Boc derivative (1.4) in a suitable solvent, e.g., CH₂Cl₂, and then TFA is added. The mixture is stirred for a time sufficient, e.g., about overnight (8-18 h), at ambient room temperature (about 15-30° C.) to complete the reaction. The product (1.8) is isolated by methods known to one skilled in the art (e.g., concentration under a vacuum; followed by purification, e.g., chromatography, if necessary).

In one aspect, compounds of type 1.6 can be prepared by the acylation of 1.5 with an appropriate acid halide of type R¹⁰C(O)X under a standard amine acylation procedure known to one skilled in the art. In an example, R¹⁰C(O)X and the appropriate amine of type 1.5, dissolved in a suitable solvent such as dichloromethane, then an appropriate base, e.g., triethylamine, is added. The reaction is stirred at an appropriate temperature (about 0-30° C.) for about 24-36 h. The product (1.6) is isolated by methods known to one skilled in the art (e.g., concentration under a vacuum; followed by purification, e.g., chromatography, if necessary).

In one aspect, compounds of type 1.6 can be prepared by the acylation of 1.5 with an appropriate carboxylic acid of type R¹⁰CO₂H under a standard carboxylic acid and amine coupling procedure known to one skilled in the art. In an example, R¹⁰CO₂H, EDCI, HOBt, triethylamine are dissolved in a suitable solvent such as dichloromethane, and allowed to stir for a period of time, e.g., about 15 min. Then, a solution of 1.5, in a solvent, e.g., dichloromethane, is added to the reaction mixture, and the reaction is stirred at ambient temperature (about 15-30° C.) for about 24-36 h. The product (1.6) is isolated by methods known to one skilled in the art (e.g., concentration under a vacuum; followed by purification, e.g., chromatography, if necessary).

2. Route II

In one aspect, substituted 4-oxo-1,3,8-triazaspiro[4.5]decanyl analogs of the present invention can be prepared generically by the synthetic scheme as shown below.

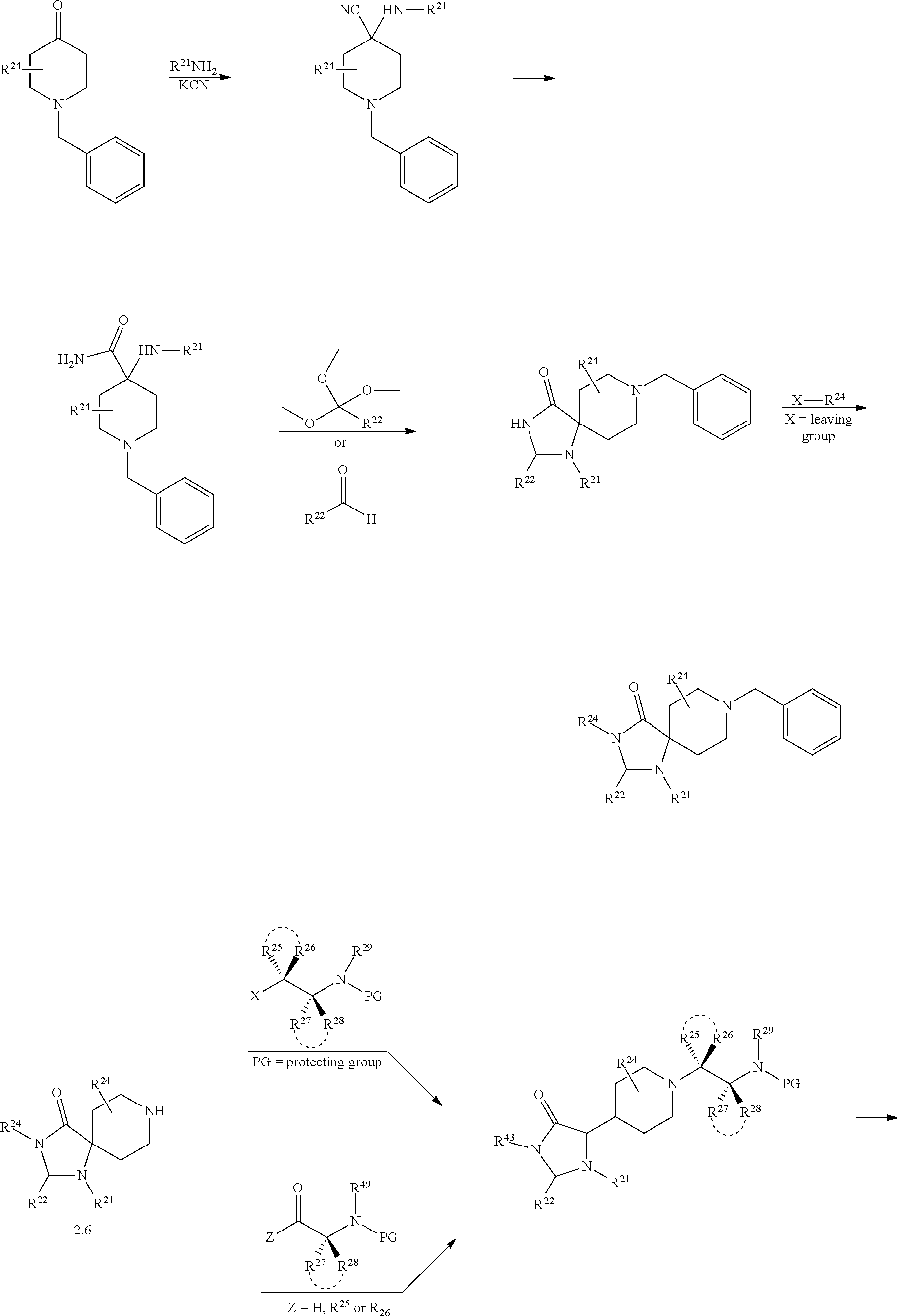

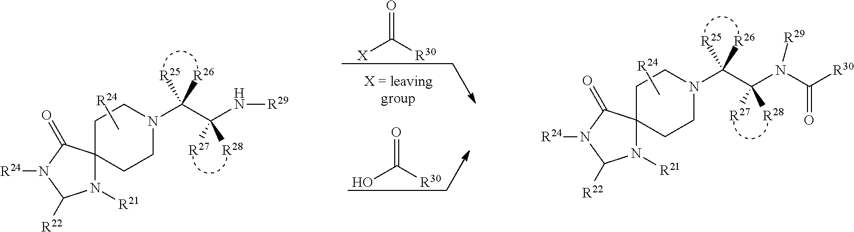

Compounds are represented in generic form, with substituents as noted in compound descriptions elsewhere herein. A more specific example is set forth below.

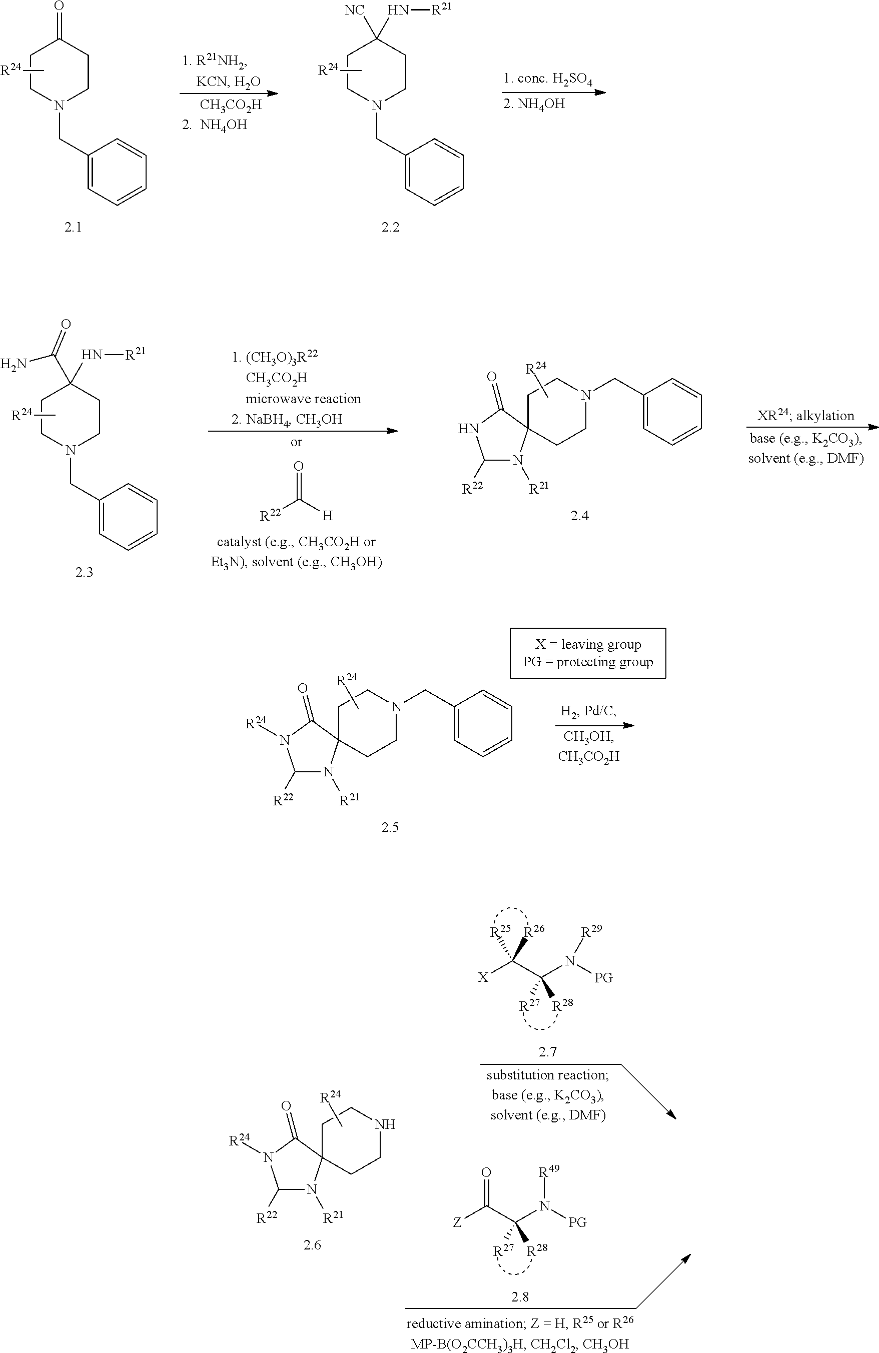

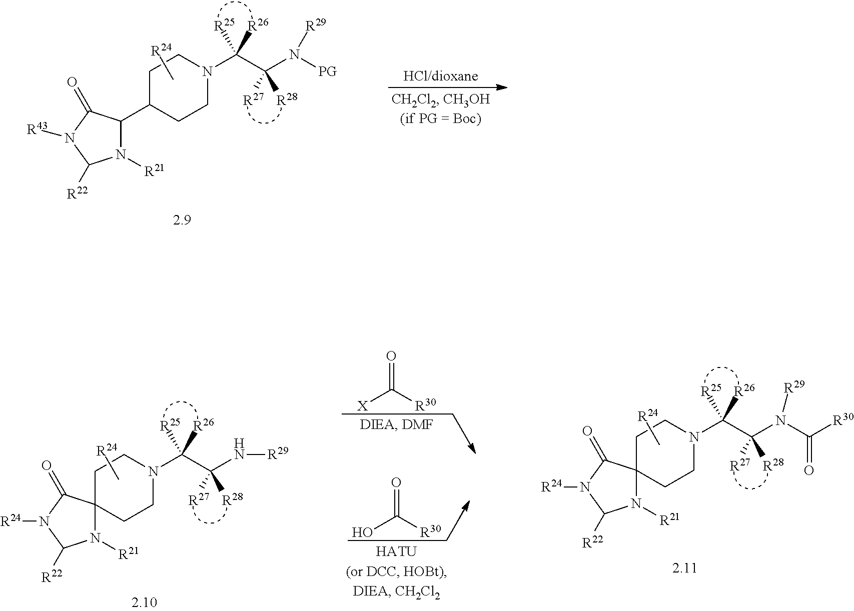

In one aspect, Route II begins with a suitable substituted 1-benzylpiperidine-4-one. A suitable 1-benzylpiperidine-4-one derivatives (2.1) are commercially available or can be readily prepared by one skilled in the art. To a solution of 2.1 in acetic acid and water at about 0° C. is added the amine, R²¹NH₂, and potassium cyanide. The reaction is allowed to warm to about ambient temperature (about 15-30° C.) and agitated/stirred for sufficient time to allow complete reaction to occur (e.g., about 12 h). The reaction is mixture is cooled to about 0° C. and concentrated ammonium hydroxide is added until about pH≧11 is reached. The product (2.2) is isolated by methods known to one skilled in the art (e.g., extraction, and concentration under a vacuum). Immediately following, the unpurified 2.2 is cooled to about 0° C. and concentrated sulfuric acid is added slowly. The reaction is allowed to warm to ambient temperature (about 15-30° C.) with stirring for about 12 h. The reaction is mixture is cooled to about 0° C. and concentrated ammonium hydroxide is added until about pH>11 is reached. The product (2.3) is isolated by methods known to one skilled in the art (e.g., extraction, and concentration under a vacuum, followed by purification, e.g., chromatography, if necessary).

In one aspect, compounds of type 2.4 can be prepared by the reaction of an appropriate orthoformate derivative [e.g., (CH₃O)₃R²²] and 2.3. Compound 2.3, (CH₃O)₃R²², and acetic acid are combined and subjected to microwave irradiation at an appropriate temperature to effect reaction, e.g., about 150° C., for about 15 min or sufficient time to complete the reaction. Then ammonium hydroxide is added until about pH=12 and extracted with dichloromethane and concentrated under vacuum. The resulting material is added to a suspension of sodium borohydride in methanol and stirred for about 3 h or sufficient time to complete the reaction. The reaction is quenched with water. The product (2.4) is isolated by methods known to one skilled in the art (e.g., extraction, and concentration under a vacuum, followed by purification, e.g., chromatography, if necessary).

In one aspect, compounds of type 2.4 can be prepared by the reaction of an appropriate aldehyde (R²²CHO) under in the presence of a suitable acid (e.g., acetic acid) or base (e.g., triethylamine) catalyst in a suitable solvent (e.g., methanol) at suitable reaction temperature and sufficient time to complete the reaction. The product (2.4) is isolated by methods known to one skilled in the art (e.g., extraction, washing, drying, filtering, and concentration under a vacuum, followed by purification, e.g., chromatography, if necessary).

In one aspect, compounds of type 2.5 can be prepared from 2.4 (where R²⁴═H) by alkylation with an appropriate alkyl halide (or similar XR²⁴ where X is an appropriate leaving group or other electrophile to afford the substituent, R²⁴). Compound 2.4 is reacted with an appropriate base (e.g., K₂CO₃) in an appropriate solvent (e.g., DMF) at a sufficient reaction temperature and for sufficient time to allow for complete reaction to afford a product (2.5). The product (2.5) is isolated by methods known to one skilled in the art (e.g., extraction, washing, drying, filtering, and concentration under a vacuum, followed by purification, e.g., chromatography, if necessary).

In one aspect, compounds of type 2.6 can be prepared from 2.5 by hydrogenation. Compound 2.5 is dissolved in an appropriate solvent(s) (e.g., methanol, acetic acid) and treated with an appropriate metal catalyst (e.g., Pd/C) under an atmosphere of hydrogen gas. The reaction is allowed to stir at an appropriate temperature and sufficient time (e.g., about 36 h) to allow for complete reaction to occur. The product (2.6) is isolated by methods known to one skilled in the art (e.g., filtering, adjusting the pH, washing, extraction, drying, filtering, and concentration under a vacuum, followed by purification, e.g., chromatography, if necessary).

In one aspect, the reaction of 2.6 and 2.7 is typically carried out under a suitable reaction atmosphere and in a suitable solvent that supports substitution reactions such as DMF in the presence of an appropriate base such as K₂CO₃. The reaction is conducted at a suitable temperature and for a time sufficient to complete the reaction, to provide compounds of type 2.9 as shown above. The product, a compound of type 2.9, is isolated by methods known to one skilled in the art (e.g., extraction, washing, drying, and concentration under a vacuum; followed by purification, e.g., chromatography, if necessary).

In one aspect, the reaction of 2.6 and 2.8 is typically carried out under a suitable reaction condition that supports reductive amination of carbonyl compounds known to one skilled in the art to give products of type 2.9. Reaction components 2.6 and 2.8 are dissolved in a suitable solvent, e.g., dichloromethane and stirred at ambient temperature (about 15 to 30° C.) for about 15 min. Then, the reducing agent, [e.g., macroporous polystyrene triacetoxyborohydride, MP—B(O₂CCH₃)₃H. or other suitable reducing agent] is added to the reaction mixture. The reaction is carried out for a time sufficient to complete the reaction, e.g., overnight (about 8-18 h), to provide compounds of type 2.9 as shown above. The product, a compound of type 2.9, is isolated by methods known to one skilled in the art (e.g., filtered, and concentration under a vacuum; followed by purification, e.g., chromatography, if necessary).

In one aspect, compounds of type 2.10 can be prepared by the conversion of the N-protected compound (e.g., N-Boc compound type 2.9) to the corresponding amine derivative (2.10). For example, a reaction of this type is commonly carried out by dissolving the N-Boc derivative (2.9) in a suitable solvent(s) (e.g., CH₂Cl₂, CH₃OH) and then HCl (e.g., 4 M HCl in dioxane) is added. The mixture is stirred for a time sufficient, e.g., about 36 h, at ambient room temperature (about 15 to 30° C.) to complete the reaction. The product (2.10) is isolated by methods known to one skilled in the art (e.g., concentration under a vacuum; followed by purification, e.g., chromatography, if necessary).

In one aspect, compounds of type 2.11 can be prepared by the acylation of 2.10 with an appropriate acid halide of type R³⁰C(O)X under a standard amine acylation procedure known to one skilled in the art. In an example, R³⁰C(O)X and the appropriate amine of type 2.10, dissolved in a suitable solvent such as DMF, then an appropriate base, e.g., N,N-diisopropylamine (DIEA), is added at an appropriate temperature (about 0° C.). The mixture is allowed to stir for about 12 h or sufficient time to complete the reaction while slowly warming to ambient temperature (about 15-30° C.). The product (2.11) is isolated by methods known to one skilled in the art (e.g., concentration under a vacuum; followed by purification, e.g., chromatography, if necessary).

In one aspect, compounds of type 2.11 can be prepared by the acylation of 2.10 with an appropriate carboxylic acid of type R³⁰CO₂H under a standard carboxylic acid and amine coupling procedure known to one skilled in the art. In an example, compound 2.10, R³⁰CO₂H, HATU (or other appropriate amine-carboxylic acid coupling agent, e.g., DCC or PS-DCC in the presence of HOBt) are combined, and then DIEA is added. The mixture is diluted with an appropriate solvent(s) (e.g., 2:1 CH₂Cl₂: DMF) to an appropriate solution concentration, and allowed to stir at ambient temperature (about 15-30° C.) for a period of time sufficient to complete the reaction, e.g., about 4 h. The product (2.11) is isolated by methods known to one skilled in the art (e.g., filtering by vacuum to collect the precipitated product; followed by purification, e.g., chromatography, if necessary).

3. Route III

In one aspect, substituted 2-oxo-2,3-dihydro-1H-benzo[d]imidazol-1-yl analogs of the present invention can be prepared generically by the synthetic scheme as shown below.

[symbol]

Compounds are represented in generic form, with substituents as noted in compound descriptions elsewhere herein. A more specific example is set forth below.

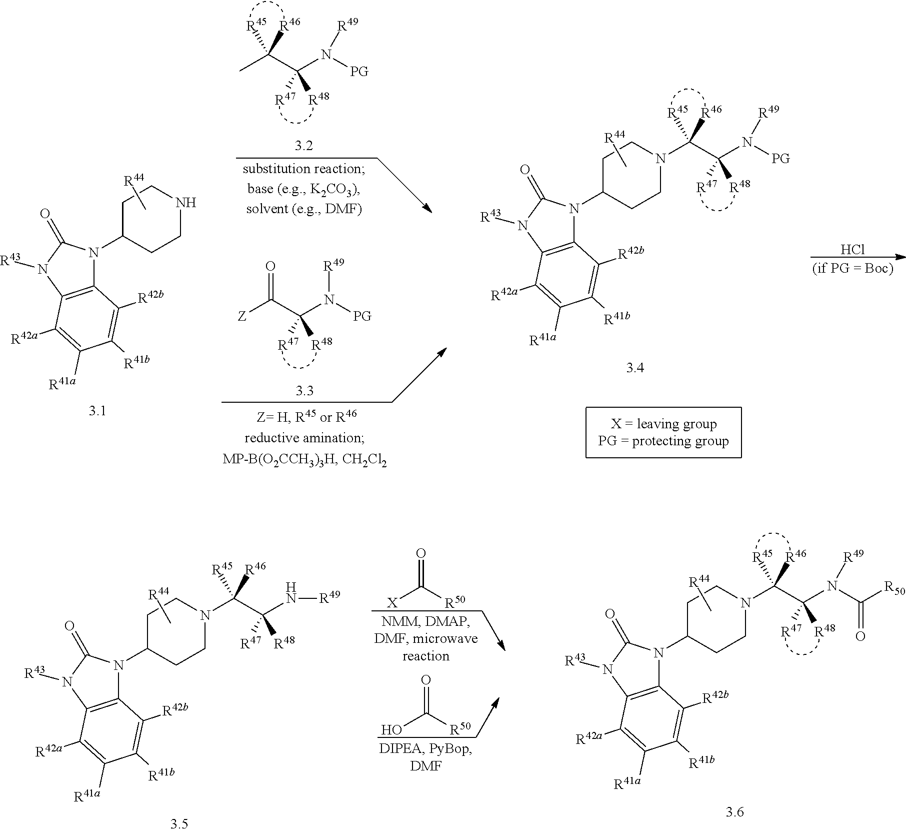

In one aspect, Route III begins with a suitable substituted compound of type 3.1. A suitable 1-(piperidin-4-yl)-1H-benzo[d]imidazol-2(3H)-one derivative (3.1) is commercially available or can be readily prepared by one skilled in the art. In one aspect, the reaction of 3.1 and 3.2 is typically carried out under a suitable reaction atmosphere and in a suitable solvent that supports substitution reactions such as DMF in the presence of an appropriate base such as K₂CO₃. The reaction is conducted at a suitable temperature and for a time sufficient to complete the reaction, to provide compounds of type 3.4 as shown above. The product, a compound of type 3.4, is isolated by methods known to one skilled in the art (e.g., extraction, washing, drying, and concentration under a vacuum; followed by purification, e.g., chromatography, if necessary).

In one aspect, the reaction of 3.1 and 3.3 is typically carried out under a suitable reaction condition that supports reductive amination of carbonyl compounds known to one skilled in the art to give products of type 3.4. Reaction components 3.1 and 3.3 are dissolved in a suitable solvent, e.g., dichloromethane and stirred Then, the reducing agent, [e.g., macroporous polystyrene triacetoxyborohydride, MP—B(O₂CCH₃)₃H. or other suitable reducing agent] is added to the reaction mixture. The reaction is carried out for a time sufficient to complete the reaction, e.g., 16 h, to provide compounds of type 3.4 as shown above. The product, a compound of type 3.4, is isolated by methods known to one skilled in the art (e.g., filtered, extracted, and concentration under a vacuum; followed by purification, e.g., chromatography, if necessary).

In one aspect, compounds of type 3.5 can be prepared by the conversion of the N-protected compound (e.g., N-Boc compound type 3.4) to the corresponding amine derivative (3.5). For example, a reaction of this type is commonly carried out by dissolving the N-Boc derivative (2.9) in a suitable solvent(s) (e.g., 1,2-dichloroethane/methanol) and then HCl (e.g., 4 M HCl in dioxane) is added. The mixture is stirred for a time sufficient, e.g., about 16 h, at ambient room temperature (about 15 to 30° C.) to complete the reaction. The product (3.5) is isolated by methods known to one skilled in the art (e.g., concentration under a vacuum; followed by purification, e.g., chromatography).

In one aspect, compounds of type 3.6 can be prepared by the acylation of 3.5 with an appropriate acid halide of type R⁵⁰C(O)X under a standard amine acylation procedure known to one skilled in the art. In an example, compound 3.5 is dissolved in a suitable solvent such as DMF; N-methylmorpholine is added, R⁵⁰C(O)X is added; and a catalytic amount of DMAP is added. The mixture is reacted under microwave irradiation for about 17 min or sufficient time and at an appropriate temperature (about 155° C.) to complete the reaction. The product (3.6) is isolated by methods known to one skilled in the art (e.g., concentration under a vacuum; followed by purification, e.g., chromatography).

In one aspect, compounds of type 3.6 can be prepared by the acylation of 3.5 with an appropriate carboxylic acid of type R⁵⁰CO₂H under a standard amine acylation procedure known to one skilled in the art. In an example, compound 3.5 is dissolved in a suitable solvent such as DMF; R⁵⁰CO₂H is added; an appropriate base, e.g., N,N-diisopropylamine (DIEA), is added; and (benzotriazol-1-lyoxy)tripyrrolidinophosphonium hexafluorophosphate (PyBOP) is added. The mixture is allowed to stir/rotate for about 16 h. or sufficient time and at ambient temperature (about 15-30° C.) to complete the reaction. The product (3.6) is isolated by methods known to one skilled in the art (e.g., concentration under a vacuum; followed by purification, e.g., chromatography).

It is understood that the disclosed methods of making can be used in connection with the disclosed compounds, compositions, kits, and uses.

### F. PHARMACEUTICAL COMPOSITIONS

In one aspect, the invention relates to pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In one aspect, the invention relates to pharmaceutical compositions comprising an effective amount of an Akt therapeutic agent inhibitor, or a pharmaceutically acceptable prodrug, salt, solvate, or polymorph thereof; an effective amount of an antiviral therapeutic agent; and a pharmaceutically acceptable carrier.

In a further aspect, the Akt therapeutic agent is an Akt inhibitor. It is understood that an Akt inhibitor can be a small molecule inhibitor (i.e. an organic compound), or a short peptide, including cyclic peptides, of about 2-10 amino acids. In a still further aspect, the Akt inhibitor binds to the pleckstrin homology domain. In a yet further aspect, the Akt inhibitor is an ATP-competitive inhibitor. In an even further aspect, the Akt inhibitor is an allosteric inhibitor. In a still further aspect, the Akt allosteric inhibitor is MK-2066. In a yet further aspect, the Akt inhibitor is a pan-Akt inhibitor. In an even further aspect, the Akt inhibitor inhibits Akt1, Akt2, or Akt3. In a still further aspect, the Akt inhibitor is an isoform-selective inhibitor. In an even further aspect, the Akt isoform-selective inhibitor selectively inhibits Akt1.

In a further aspect, the Akt therapeutic agent is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine. In a still further aspect, the Akt inhibitor is erucylphosphocholine. In a yet further aspect, the Akt inhibitor is GDC-0068. In an even further aspect, the Akt inhibitor is GSK-2141795. In a still further aspect, the Akt inhibitor is MK-2206. In a yet further aspect, the Akt inhibitor is perifosine. In an even further aspect, the Akt inhibitor is PHT-427.

In a further aspect, the Akt therapeutic agent is a siRNA. In a still further aspect, the Akt therapeutic agent is an antisense oligonucleotide. In a yet further aspect, the antisense oligonucleotide is RX-0201.

In a further aspect, the effective amount is a therapeutically effective amount. In a still further aspect, the effective amount is a prophylactically effective amount.

In a further aspect, the effective amount of the Akt inhibitor inhibits HIV infection. In a still further aspect, the effective amount of the Akt inhibitor inhibits HIV replication.

In a further aspect, the effective amount of the Akt inhibitor decreases cellular nucleotide pools.

In a further aspect, the antiviral therapeutic agent comprises at least one more HIV therapeutic agent selected from: a) a HIV fusion/lysis inhibitor, or a pharmaceutically acceptable prodrug, salt, solvate, or polymorph thereof; b) a HIV integrase inhibitor, or a pharmaceutically acceptable prodrug, salt, solvate, or polymorph thereof; c) a HIV non-nucleoside reverse transcriptase inhibitor, or a pharmaceutically acceptable prodrug, salt, solvate, or polymorph thereof; d) a HIV nucleoside reverse transcriptase inhibitor, or a pharmaceutically acceptable prodrug, salt, solvate, or polymorph thereof; and e) a HIV protease inhibitor, or a pharmaceutically acceptable prodrug, salt, solvate, or polymorph thereof.

In a further aspect, the HIV fusion/lysis inhibitor is selected from enfuvirtide, maraviroc, cenicriviroc, ibalizumab, BMS-663068, and PRO-140, or a pharmaceutically acceptable prodrug, salt, solvate, or polymorph thereof. In a still further aspect, the HIV fusion/lysis inhibitor is selected from enfuvirtide, maraviroc, cenicriviroc, and ibalizumab, or a pharmaceutically acceptable prodrug, salt, solvate, or polymorph thereof. In yet a further aspect, the HIV fusion/lysis inhibitor is enfuvirtide, or a pharmaceutically acceptable prodrug, salt, solvate, or polymorph thereof. In an even further aspect, the HIV fusion/lysis inhibitor is maraviroc, or a pharmaceutically acceptable prodrug, salt, solvate, or polymorph thereof. In a still further aspect, the HIV fusion/lysis inhibitor is cenicriviroc, or a pharmaceutically acceptable prodrug, salt, solvate, or polymorph thereof. In yet a further aspect, the HIV fusion/lysis inhibitor is ibalizumab, or a pharmaceutically acceptable prodrug, salt, solvate, or polymorph thereof.

In a further aspect, the HIV integrase inhibitor is selected from raltegravir, dolutegravir, and elvitegravir, or a pharmaceutically acceptable prodrug, salt, solvate, or polymorph thereof. In a still further aspect, the HIV integrase inhibitor is raltegravir, or a pharmaceutically acceptable prodrug, salt, solvate, or polymorph thereof. In yet a further aspect, the HIV integrase inhibitor is dolutegravir, or a pharmaceutically acceptable prodrug, salt, solvate, or polymorph thereof. In an even further aspect, the HIV integrase inhibitor is elvitegravir, or a pharmaceutically acceptable prodrug, salt, solvate, or polymorph thereof.

In a further aspect, the HIV non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In a still further aspect, the HIV non-nucleoside reverse transcriptase inhibitor is delavirdine, or a pharmaceutically acceptable prodrug, salt, solvate, or polymorph thereof. In yet a further aspect, the HIV non-nucleoside reverse transcriptase inhibitor is efavirenz, or a pharmaceutically acceptable prodrug, salt, solvate, or polymorph thereof. In an even further aspect, the HIV non-nucleoside reverse transcriptase inhibitor is etravirine, or a pharmaceutically acceptable prodrug, salt, solvate, or polymorph thereof. In a still further aspect, the HIV non-nucleoside reverse transcriptase inhibitor is lersivirine, or a pharmaceutically acceptable prodrug, salt, solvate, or polymorph thereof. In yet a further aspect, the HIV non-nucleoside reverse transcriptase inhibitor is nevirapine, or a pharmaceutically acceptable prodrug, salt, solvate, or polymorph thereof. In an even further aspect, the HIV non-nucleoside reverse transcriptase inhibitor is rilpivirine, or a pharmaceutically acceptable prodrug, salt, solvate, or polymorph thereof.

In a further aspect, the HIV nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In a still further aspect, the HIV nucleoside reverse transcriptase inhibitor is selected from abacavir, didansine, elvucitabine, emtricitabine, lamivudine, stavudine, tenofovir, and zidovudine, or a pharmaceutically acceptable prodrug, salt, solvate, or polymorph thereof. In yet a further aspect, the HIV nucleoside reverse transcriptase inhibitor is abacavir, or a pharmaceutically acceptable prodrug, salt, solvate, or polymorph thereof. In an even further aspect, the HIV nucleoside reverse transcriptase inhibitor is didansine, or a pharmaceutically acceptable prodrug, salt, solvate, or polymorph thereof. In a still further aspect, the HIV nucleoside reverse transcriptase inhibitor is elvucitabine, or a pharmaceutically acceptable prodrug, salt, solvate, or polymorph thereof. In yet a further aspect, the HIV nucleoside reverse transcriptase inhibitor is emtricitabine, or a pharmaceutically acceptable prodrug, salt, solvate, or polymorph thereof. In an even further aspect, the HIV nucleoside reverse transcriptase inhibitor is lamivudine, or a pharmaceutically acceptable prodrug, salt, solvate, or polymorph thereof. In a still further aspect, the HIV nucleoside reverse transcriptase inhibitor is stavudine, or a pharmaceutically acceptable prodrug, salt, solvate, or polymorph thereof. In yet a further aspect, the HIV nucleoside reverse transcriptase inhibitor is tenofovir, or a pharmaceutically acceptable prodrug, salt, solvate, or polymorph thereof. In an even further aspect, the HIV nucleoside reverse transcriptase inhibitor is zidovudine, or a pharmaceutically acceptable prodrug, salt, solvate, or polymorph thereof.

In a further aspect, the HIV protease inhibitor is selected from wherein the HIV protease inhibitor is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In a still further aspect, the HIV protease inhibitor is atazanir, or a pharmaceutically acceptable prodrug, salt, solvate, or polymorph thereof. In yet a further aspect, the HIV protease inhibitor is darunavir, or a pharmaceutically acceptable prodrug, salt, solvate, or polymorph thereof. In an even further aspect, the HIV protease inhibitor is fosamprenavir, or a pharmaceutically acceptable prodrug, salt, solvate, or polymorph thereof. In a still further aspect, the HIV protease inhibitor is indinavir, or a pharmaceutically acceptable prodrug, salt, solvate, or polymorph thereof. In yet a further aspect, the HIV protease inhibitor is lopinavir, or a pharmaceutically acceptable prodrug, salt, solvate, or polymorph thereof. In an even further aspect, the HIV protease inhibitor is nelfinavir, or a pharmaceutically acceptable prodrug, salt, solvate, or polymorph thereof. In a still further aspect, the HIV protease inhibitor is ritonavir, or a pharmaceutically acceptable prodrug, salt, solvate, or polymorph thereof. In yet a further aspect, the HIV protease inhibitor is saquinavir, or a pharmaceutically acceptable prodrug, salt, solvate, or polymorph thereof. In an even further aspect, the HIV protease inhibitor is tipranavir, or a pharmaceutically acceptable prodrug, salt, solvate, or polymorph thereof.

In a further aspect, the antiviral therapeutic agent comprises an effective amount of at least one influenza therapeutic agent selected from: a) a viral protein M2 ion channel inhibitor, or a pharmaceutically acceptable prodrug, salt, solvate, or polymorph thereof; b) a neuraminidase inhibitor, or a pharmaceutically acceptable prodrug, salt, solvate, or polymorph thereof; and c) a nucleoside analog, or a pharmaceutically acceptable prodrug, salt, solvate, or polymorph thereof. In a still further aspect, the effective amount of the at least one influenza therapeutic agent is a therapeutically effective amount. In yet a further aspect, the effective amount of the at least one influenza therapeutic agent is a prophylactically effective amount.

In a further aspect, the viral protein M2 ion channel inhibitor is an amino-adamantane compound. In a still further aspect, the amino-adamantane compound is selected from 1-amino-adamantane and 1-(1-aminoethyl)adamantane.

In a further aspect, the viral protein M2 ion channel inhibitor is selected from amantadine and rimantadine, or a pharmaceutically acceptable prodrug, salt, solvate, or polymorph thereof. In a still further aspect, the viral protein M2 ion channel inhibitor is an analog of amantadine or rimantadine. In yet a further aspect, the amantadine analog is selected from 1-amino-1,3,5-trimethylcyclohexane, 1-amino-1(trans),3(trans),5-trimethylcyclohexane, 1-amino-1(cis),3(cis),5-trimethylcyclohexane, 1-amino-1,3,3,5-tetramethylcyclohexane, 1-amino-1,3,3,5,5-pentamethylcyclohexane(neramexane), 1-amino-1,3,5,5-tetramethyl-3-ethylcyclohexane, 1-amino-1,5,5-trimethyl-3,3-diethylcyclohexane, 1-amino-1,5,5-trimethyl-cis-3-ethylcyclohexane, 1-amino-(1S,5S)cis-3-ethyl-1,5,5-trimethylcyclohexane, 1-amino-1,5,5-trimethyl-trans-3-ethylcyclohexane, 1-amino-(1R,5S)trans-3-ethyl-1,5,5-trimethylcyclohexane, 1-amino-1-ethyl-3,3,5,5-tetramethylcyclohexane, 1-amino-1-propyl-3,3,5,5-tetramethylcyclohexane, N-methyl-1-amino-1,3,3,5,5-pentamethylcyclohexane, N-ethyl-1-amino-1,3,3,5,5-pentamethyl-cyclohexane, N-(1,3,3,5,5-pentamethylcyclohexyl)pyrrolidine, 3,3,5,5-tetramethylcyclohexylmethylamine, 1-amino-1-propyl-3,3,5,5-tetramethylcyclohexane, 1 amino-1,3,3,5(trans)-tetramethylcyclohexane (axial amino group), 3-propyl-1,3,5,5-tetramethylcyclohexylamine semihydrate, 1-amino-1,3,5,5-tetramethyl-3-ethylcyclohexane, 1-amino-1,3,5-trimethylcyclohexane, 1-amino-1,3-dimethyl-3-propylcyclohexane, 1-amino-1,3 (trans),5(trans)-trimethyl-3(cis)-propylcyclohexane, 1-amino-1,3-dimethyl-3-ethylcyclohexane, 1-amino-1,3,3-trimethylcyclohexane, cis-3-ethyl-1(trans)-3 (trans)-5-trimethylcyclohexamine, 1-amino-1,3(trans)-dimethylcyclohexane, 1,3,3-trimethyl-5,5-dipropylcyclohexylamine, 1-amino-1-methyl-3 (trans)-propylcyclohexane, 1-methyl-3(cis)-propylcyclohexylamine, 1-amino-1-methyl-3(trans)-ethylcyclohexane, 1-amino-1,3,3-trimethyl-5(cis)-ethylcyclohexane, 1-amino-1,3,3-trimethyl-5(trans)-ethylcyclohexane, cis-3-propyl-1,5,5-trimethylcyclohexylamine, trans-3-propyl-1,5,5-trimethylcyclohexylamine, N-ethyl-1,3,3,5,5-pentamethylcyclohexylamine, N-methyl-1-amino-1,3,3,5,5-pentamethylcyclohexane, 1-amino-1-methylcyclohexane, N,N-dimethyl-1-amino-1,3,3,5,5-pentamethylcyclohexane, 2-(3,3,5,5-tetramethylcyclohexyl)ethylamine, 2-methyl-1-(3,3,5,5-tetramethylcyclohexyl)propyl-2-amine, 2-(1,3,3,5,5-pentamethylcyclohexyl-1)-ethylamine semihydrate, N-(1,3,3,5,5-pentamethylcyclohexyl)-pyrrolidine, 1-amino-1,3(trans),5(trans)-trimethylcyclohexane, 1-amino-1,3(cis),5 (cis)-trimethylcyclohexane, 1-amino-(1R,5S)trans-5-ethyl-1,3,3-trimethylcyclohexane, 1-amino-(1S,5S)cis-5-ethyl-1,3,3-trimethylcyclohexane, 1-amino-1,5,5-trimethyl-3(cis)-isopropyl-cyclohexane, 1-amino-1,5,5-trimethyl-3(trans)-isopropyl-cyclohexane, 1-amino-1-methyl-3 (cis)-ethyl-cyclohexane, 1-amino-1-methyl-3 (cis)-methyl-cyclohexane, 1-amino-5,5-diethyl-1,3,3-trimethyl-cyclohexane, 1-amino-1,3,3,5,5-pentamethylcyclohexane, 1-amino-1,5,5-trimethyl-3,3-diethylcyclohexane, 1-amino-1-ethyl-3,3,5,5-tetramethylcyclohexane, N-ethyl-1-amino-1,3,3,5,5-pentamethylcyclohexane, N-(1,3,5-trimethylcyclohexyl)pyrrolidine, N-(1,3,5-trimethylcyclohexyl)piperidine, N-[1,3(trans),5(trans)-trimethylcyclohexyl]pyrrolidine, N-[1,3(trans),5(trans)-trimethylcyclohexyl]piperidine, N-[1,3(cis),5(cis)-trimethylcyclohexyl]pyrrolidine, N-[1,3(cis),5 (cis)-trimethylcyclohexyl]piperidine, N-(1,3,3,5-tetramethylcyclohexyl)pyrrolidine, N-(1,3,3,5-tetramethylcyclohexyl)piperidine, N-(1,3,3,5,5-pentamethylcyclohexyl)pyrrolidine, N-(1,3,3,5,5-pentamethylcyclohexyl)piperidine, N-(1,3,5,5-tetramethyl-3-ethylcyclohexyl)pyrrolidine, N-(1,3,5,5-tetramethyl-3-ethylcyclohexyl)piperidine, N-(1,5,5-trimethyl-3,3-diethylcyclohexyl)pyrrolidine, N-(1,5,5-trimethyl-3,3-diethylcyclohexyl)piperidine, N-(1,3,3-trimethyl-cis-5-ethylcyclohexyl)pyrrolidine, N-(1,3,3-trimethyl-cis-5-ethylcyclohexyl)piperidine, N-[(1S,5S)cis-5-ethyl-1,3,3-trimethylcyclohexyl]pyrrolidine, N-[(1S,5S)cis-5-ethyl-1,3,3-trimethylcyclohexyl]piperidine, N-(1,3,3-trimethyl-trans-5-ethylcyclohexyl)pyrrolidine, N-(1,3,3-trimethyl-trans-5-ethylcyclohexyl)piperidine, N-[(1R,5S)trans-5-ethyl,3,3-trimethylcyclohexyl]pyrrolidine, N-[(1R,5S)trans-5-ethyl,3,3-trimethylcyclohexyl]piperidine, N-(1-ethyl-3,3,5,5-tetramethylyclohexyl)pyrrolidine, N-(1-ethyl-3,3,5,5-tetramethylyclohexyl)piperidine, N-(1-propyl-3,3,5,5-tetramethylcyclohexyl)pyrrolidine, N-(1-propyl-3,3,5,5-tetramethylcyclohexyl)piperidine, N-(1,3,3,5,5-pentamethylcyclohexyl)pyrrolidine, spiro[cyclopropane-1,2-adamantan]-2-amine, spiro[pyrrolidine-2,2′-adamantane], spiro[piperidine-2,2-adamantane], 2-(2-adamantyl)piperidine, 3-(2-adamantyl)pyrrolidine, 2-(1-adamantyl)piperidine, 2-(1-adamantyl)pyrrolidine, and 2-(1-adamantyl)-2-methyl-pyrrolidine. In an even further aspect, the amantadine analog is selected from 1-amino-3-phenyl adamantane, 1-amino-methyl adamantane, 1-amino-3-ethyl adamantane, 1-amino-3-isopropyl adamantane, 1-amino-3-n-butyl adamantane, 1-amino-3,5-diethyl adamantane, 1-amino-3,5-diisopropyl adamantane, 1-amino-3,5-di-n-butyl adamantane, 1-amino-3-methyl-5-ethyl adamantane, 1-N-methylamino-3,5-dimethyl adamantane, 1-N-ethylamino-3,5-dimethyl adamantane, 1-N-isopropyl-amino-3,5-dimethyl adamantane, 1-N,N-dimethyl-amino-3,5-dimethyl adamantane, 1-N-methyl-N-isopropyl-amino-3-methyl-5-ethyl adamantane, 1-amino-3-butyl-5-phenyl adamantane, 1-amino-3-pentyl adamantane, 1-amino-3,5-dipentyl adamantane, 1-amino-3-pentyl-5-hexyl adamantane, 1-amino-3-pentyl-5-cyclohexyl adamantane, 1-amino-3-pentyl-5-phenyl adamantane, 1-amino-3-hexyl adamantane, 1-amino-3,5-dihexyl adamantane, 1-amino-3-hexyl-5-cyclohexyl adamantane, 1-amino-3-hexyl-5-phenyl adamantane, 1-amino-3-cyclohexyl adamantane, 1-amino-3,5-dicyclohexyl adamantane, 1-amino-3-cyclohexyl-5-phenyl adamantane, 1-amino-3,5-diphenyl adamantane, 1-amino-3,5,7-trimethyl adamantane, 1-amino-3,5-dimethyl-7-ethyl adamantane, 1-amino-3,5-diethyl-7-methyl adamantane, 1-N-pyrrolidino and 1-N-piperidine derivatives, 1-amino-3-methyl-5-propyl adamantane, 1-amino-3-methyl-5-butyl adamantane, 1-amino-3-methyl-5-pentyl adamantane, 1-amino-3-methyl-5-hexyl adamantane, 1-amino-3-methyl-5-cyclohexyl adamantane, 1-amino-3-methyl-5-phenyl adamantane, 1-amino-3-ethyl-5-propyl adamantane, 1-amino-3-ethyl-5-butyl adamantane, 1-amino-3-ethyl-5-pentyl adamantane, 1-amino-3-ethyl-5-hexyl adamantane, 1-amino-3-ethyl-5-cyclohexyl adamantane, 1-amino-3-ethyl-5-phenyl adamantane, 1-amino-3-propyl-5-butyl adamantane, 1-amino-3-propyl-5-pentyl adamantane, 1-amino-3-propyl-5-hexyl adamantane, 1-amino-3-propyl-5-cyclohexyl adamantane, 1-amino-3-propyl-5-phenyl adamantane, 1-amino-3-butyl-5-pentyl adamantane, 1-amino-3-butyl-5-hexyl adamantane, and 1-amino-3-butyl-5-cyclohexyl adamantine.

In a further aspect, the neuraminidase inhibitor is selected from oseltamivir, zanamivir, peramivir, laninamivir octanoate, 2,3-didehydro-2-deoxy-N-acetylneuraminic acid (DANA), 2-deoxy-2,3-dehydro-N-trifluoroacetylneuraminic acid (FANA), N-[(1R,2S)-2-methoxy-2-methyl-1-[(2R,3S,5R)-5-(2-methylpropanoyl)-3-[(Z)-prop-1-enyl]pyrrolidin-2-yl]pentyl]acetamide (A-322278), and (2R,4S,5R)-5-[(1R,2S)-1-acetamido-2-methoxy-2-methylpentyl]-4-[(Z)-prop-1-enyl]pyrrolidine-2-carboxylic acid (A-315675), or a pharmaceutically acceptable salt, solvate, or polymorph thereof. In a still further aspect, the neuraminidase inhibitor is selected from oseltamivir, zanamivir, peramivir, laninamivir octanoate, or a pharmaceutically acceptable salt, solvate, or polymorph thereof. In yet a further aspect, the neuraminidase inhibitor is oseltamivir, oseltamivir phosphate, or oseltamivir carboxylate. In an even further aspect, the neuraminidase inhibitor is oseltamivir phosphate. In a still further aspect, the neuraminidase inhibitor is zanamivir. In yet a further aspect, the neuraminidase inhibitor is peramivir. In an even further aspect, the neuraminidase inhibitor is laninamivir octanoate.

In a further aspect, the nucleoside analog is selected from ribavirin, viramidine, 6-fluoro-3-hydroxy-2-pyrazinecarboxamide, 2′-deoxy-2′-fluoroguanosine, pyrazofurin, carbodine, and cyclopenenyl cytosine. In a still further aspect, the nucleoside analog is selected from ribavirin and viramidine. In yet a further aspect, the nucleoside analog is ribavirin. In an even further aspect, the nucleoside analog is viramidine.

In a further aspect, the composition further comprises a prostaglandin E2 receptor agonist, or a pharmaceutically acceptable prodrug, salt, solvate, or polymorph thereof. In a still further aspect, the prostaglandin E2 receptor agonist is selected from a prostaglandin E receptor 4 (subtype EP4) selective agonist, a prostaglandin E receptor 2 (subtype EP2) selective agonist, and a mixed agonist for prostaglandin E receptor 4 (subtype EP4) and prostaglandin E receptor 2 (subtype EP2). In yet a further aspect, the prostaglandin E2 receptor agonist is a prostaglandin E receptor 4 (subtype EP4) agonist. In an even further aspect, the prostaglandin E receptor 4 (subtype EP4) agonist is selected from beraprost, nileprost, iloprost, cicaprost, eptaloprost, and ciprosten, or a pharmaceutically acceptable prodrug, salt, solvate, or polymorph thereof. In a still further aspect, the prostaglandin E receptor 4 (subtype EP4) agonist is beraprost. In yet a further aspect, the prostaglandin E receptor 4 (subtype EP4) agonist is nileprost.

In a further aspect, the composition further comprises an interferon, or an isoform, mutein or fused protein thereof. In a still further aspect, the interferon is a pegylated interferon, a recombinant interferon, or a natural interferon. In yet a further aspect, the interferon is recombinant human interferon-beta, recombinant human interfone-beta which has a CHO cell-derived glycosylation, or consensus interferon-beta. In an even further aspect, the interferon is interferon is pegylated interferon-beta or interferon-beta Fc-fusion protein.

In a further aspect, the composition further comprises an effective amount of an antiviral agent selected from a replication inhibitor, an IMP dehydrogenase inhibitor, an RNA polymerase inhibitor, and an influenza-specific interfering oligonucleotide. In a still further aspect, the IMP dehydrogenase inhibitor is selected from ribavirin, viramidine, merimepodib (VX-497), mycophenolic acid, mycophenolate mofetil, benzamide riboside, tiazofurin, mizoribine, and 3-deazaguanosine. In yet a further aspect, the IMP dehydrogenase inhibitor is selected from ribavirin, viramidine, merimepodib (VX-497), mycophenolic acid, and mycophenolate mofetil. In an even further aspect, the IMP dehydrogenase inhibitor is selected from ribavirin, viramidine, mycophenolic acid, and mycophenolate mofetil. In a still further aspect, the RNA polymerase inhibitor is favipiravir.

In a further aspect, the composition further comprises an effective amount of an influenza virus absorption inhibitor selected from a hemagglutinin-specific antibody, a polyoxometalate, a sulfated polysaccharide, a sialidase fusion protein, and an O-glycoside of sialic acid. In a still further aspect, the influenza virus absorption inhibitor is a recombinant sialidase fusion protein. In yet a further aspect, the recombinant sialidase fusion protein is Fludase (DAS181).

In a further aspect, the composition further comprises an effective amount of a cysteamine compound. In a still further aspect, the cysteamine compound is selected from cysteamine, cysteamine salts, prodrugs of cysteamine, analogs of cysteamine, derivatives of cysteamine, conjugates of cysteamine, metabolic precursors of cysteamine, and metabolites of cysteamine. In yet a further aspect, the cysteamine salt is cysteamine hydrochloride. In an even further aspect, the metabolic precursor of cysteamine is selected from cysteine, cystamine, and pantethine In a still further aspect, the cysteamine metabolite is selected from taurine and hypotaurine.

In a further aspect, the composition further comprises an effective amount of a therapeutic agent selected from an antitussive, a mucolytic, an expectorant, an antipyretic, an analgesic, and a nasal decongestant.

In a further aspect, the composition further comprises an effective amount of an immunomodulator, or a pharmaceutically acceptable prodrug, salt, solvate, or polymorph thereof. In a still further aspect, an effective amount of an immunomodulator is an amount effective to reduce or inhibit one or more symptoms of inflammation of a subject. In yet a further aspect, the immunomodulator is polyoxidonium. In an even further aspect, the immunomodulator is an anti-inflammatory agent. In a still further aspect, the anti-inflammatory agent is non-steroidal, steroidal, or a combination thereof. In yet a further aspect, the anti-inflammatory agent is a non-steroidal anti-inflammatory agent. In an even further aspect, the non-steroidal anti-inflammatory agent is selected from a COX2 inhibitor, an aminosalicylate drug, a PPAR ligand. In a still further aspect, the non-steroidal anti-inflammatory agent is selected from an oxicam, a salicylate, an acetic acid derivative, a fenamate, a propionic acid derivative, and a pyrazole. In yet a further aspect, the non-steroidal anti-inflammatory agent comprises one or more of piroxicam, isoxicam, tenoxicam, sudoxicam, aspirin, disalcid, benorylate, trilisate, safapryn, solprin, diflunisal, fendosal, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, mefenamic acid, meclofenamic acid, flufenamic acid, niflumic acid, tolfenamic acid, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofenic, phenylbutazone, oxyphenbutazone, feprazone, azapropazone, and trimethazone.

In a further aspect, the non-steroidal anti-inflammatory agent is a COX2 inhibitor. In a still further aspect, the COX2 inhibitor is celecoxib. In yet a further aspect, the non-steroidal anti-inflammatory agent is an aminosalicylate. In an even further aspect, the aminosalicylate drug is selected from mesalazine and sulfasalazine.

In a further aspect, the non-steroidal anti-inflammatory agent is a PPAR ligand. In a still further aspect, the PPAR ligand is a fibrate. In yet a further aspect, the fibrate is selected from gemfibrozil, bezafibrate, ciprofibrate, clofibrate, and renofibrate, or combinations thereof.

In a further aspect, the anti-inflammatory agent is a steroidal anti-inflammatory agent. In a still further aspect, the steroidal anti-inflammatory agent is selected from hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, predisone, beclomethasone dipropionate, triamcinolone, and mixtures thereof.

In one aspect, the invention relates to pharmaceutical compositions comprising an effective amount of an Akt therapeutic agent, or a pharmaceutically acceptable prodrug, salt, solvate, or polymorph thereof; an effective amount of at least one antibacterial therapeutic agent; and a pharmaceutically acceptable carrier. In a further aspect, the Akt therapeutic agent is an Akt inhibitor.

In a further aspect, the Akt inhibitor binds to the pleckstrin homology domain.

In a further aspect, the Akt inhibitor is an ATP-competitive inhibitor.

In a further aspect, the Akt inhibitor is an allosteric inhibitor. In a still further aspect, the allosteric inhibitor is MK-2066.

In a further aspect, the Akt inhibitor is a pan-Akt inhibitor. In a still further aspect, the Akt inhibitor inhibits Akt1, Akt2, or Akt3. In yet a further aspect, the Akt inhibitor is an isoform-selective inhibitor. In an even further aspect, the isoform-selective inhibitor selectively inhibits Akt1.

In a further aspect, the Akt therapeutic agent is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine. In a still further aspect, the Akt therapeutic agent is erucylphosphocholine. In yet a further aspect, the Akt therapeutic agent is GDC-0068. In an even further aspect, the Akt therapeutic agent is GSK-2141795. In a still further aspect, the Akt therapeutic agent is MK-2206. In yet a further aspect, the Akt therapeutic agent is PHT-427.

In a further aspect, the Akt therapeutic agent is a siRNA.

In a further aspect, the Akt therapeutic agent is an antisense oligonucleotide. In a still further aspect, the antisense oligonucleotide is RX-0201.

In a further aspect, an effective amount of the Akt therapeutic agent is a therapeutically effective amount. In a still further aspect, an effective amount of the Akt therapeutic agent is a prophylactically effective amount.

In a further aspect, an effective amount of the at least one antibacterial therapeutic agent is a therapeutically effective amount. In a still further aspect, an effective amount of the at least one antibacterial therapeutic agent is a prophylactically effective amount.

In a further aspect, the at least one antibacterial therapeutic agent selected from amikacin, amoxicillin, amoxicillin/clavulanate, aztreonam, azithromycin, cefaclor, cefadroxil, cephalexin, cefazolin, cefixime, cefotaxime, cefotetan, cefoxitin, cefpodoxime, ceftaroline fosamil, ceftazidime, ceftriaxone, cefuroxime, cephalexin, cephradine, chloramphenicol, cilastatin/imipenem, ciprofloxacin, clavulanate/ticarcillin, clarithromycin, clindamycin, clofazimine, colistin, daptomycin, demeclocycline, doripenem, doxycycline, ertapenem, fosfomycin/trometamol, fusidic acid, gentamicin, grepafloxacin, kanamycin, levofloxacin, lincomycin, linezolid, lymecycline, meropenem, metronidazole, minocycline, moxifloxacin, nafcillin, nalidixic acid, netilmicin, nitrofuratoin, norfloxacin, ofloxacin, oxacillin, oxytetracycline, penicillin, phenoxymethylpenicillin, piperacillin, pivmecillinam, polymyxin B, rifaximin, streptomycin, sulfadiazine, sulfamethoxazole/trimethoprim, sulfisoxazole, telithromycin, tetracycline, tobramycin, trimethoprim/sulfamethoxazole, vancomycin, LFF571, MK-3415, MK-3415A, and MK-6072.

In a further aspect, the at least one antibacterial therapeutic agent is an antituberculosis agent. In a still further aspect, the antituberculosis agent is selected from capreomycin, clofazimine, cycloserine, ethambutol, ethionamide, isoniazid, pyrazinamide, rifabutin, rifampin, and rifapentine. In yet a further aspect, the antituberculosis therapeutic agent is selected from isoniazid, rifampin, ethambutol, and pyrazinamide.

In a further aspect, wherein the at least one antibacterial therapeutic agent comprises an effective amount of at least one antibacterial therapeutic agent selected from: a) an inhibitor of bacterial DNA synthesis, or a pharmaceutically acceptable prodrug, salt, solvate, or polymorph thereof; b) an inhibitor of bacterial RNA synthesis, or a pharmaceutically acceptable prodrug, salt, solvate, or polymorph thereof; c) an inhibitor of bacterial protein synthesis, or a pharmaceutically acceptable prodrug, salt, solvate, or polymorph thereof; d) an bacterial antimetabolite agent, or a pharmaceutically acceptable prodrug, salt, solvate, or polymorph thereof; and e) an inhibitor of bacterial cell wall synthesis, or a pharmaceutically acceptable prodrug, salt, solvate, or polymorph thereof.

In a further aspect, the inhibitor of bacterial DNA synthesis is selected from ciprofloxacin, clofazimine, enoxacin, gatifloxacin, gemifloxacin, levofloxacin, lomefloxacin, metronidazole, moxifloxacin, nalidixic acid, norfloxacin, and ofloxacin. In a still further aspect, the inhibitor of bacterial RNA synthesis is selected from rifampin, rifabutin, rifapentine, and rifampin/isoniazid/pyrazinamide.

In a further aspect, the inhibitor of bacterial protein synthesis is selected from amikacin, azithromycin, capreomycin, chloramphenicol, clarithromycin, clindamycin, demeclocycline, dirithromycin, doxycycline, erythromycin, ethionamide, gentamicin, kanamycin, lincomycin, linezolid, minocycline, neomycin, puromycin, quinupristin/dalfopristin, roxithromycin, spectinomycin, telithromycin, tetracycline, tigecycline, and tobramycin.

In a further aspect, the bacterial antimetabolite agent is selected from aminosalicylic acid, furazolidinone, nitrofurantoin, introfurazone, sulfacetamide, sulfabenzamide, sulfanilamide, sulfisoxazole, sulfathiazole, trimethoprim/polymyxin B, trimethoprim/sulfamethoxazole, and trimetrexate.

In a further aspect, the inhibitor of bacterial cell wall synthesis is selected from ampicillin, ampicillin/sulbactam, amoxicillin, amoxicillin/clavulanate, aztreonam, bacampicillin, carbenicillin, cefaclor, cefadroxil, cefazolin, cefdinir, cefditoren, cefepime, cefixime, cefoperazone, cefotaxime, cefotetan, cefoxitin, cefprozil, cefpirome, cefpodoxime, ceftibuten, ceftriaxone, cefuroxime, cephalexin, cephradine, cycloserine, dicloxacillin, doripenem, ertapenem, ethambutol, fosfomycin, imipenem, imipenem/cilastatin, isoniazid, loracarbef, meropenem, methicillin, mezlocillin, nafcillin, oxacillin, penicillin G, penicillin V, penicillin W, piperacillin, pipercillin/tazobactam, ticarcillin, ticarcillin/clavulanate, and vancomycin.

In a further aspect, the composition further comprises an effective amount of an mTor inhibitor. In a still further aspect, the effective amount of an mTor inhibitor is a therapeutically effective amount. In yet a further aspect, the effective amount of an mTor inhibitor is a prophylactically effective amount.

In a further aspect, the mTor inhibitor is selected from everolimus, rapamycin (sirolimus), temsirolimus, deforolimus, ridaforolimus, tacrolimus, zotarolimus, salirasib, curcumin, farnesylthiosalicylic acid, XL765, ABI-009, AP-23675, AP-23841, AP-23765, AZD-8055, AZD-2014, BEZ-235 (NVP-BEZ235), BGT226, GDC-0980, INK-128, KU-0063794, MK8669, MKC-1 (Ro 31-7453), NVP-BGT226, OSI-027, Palomid-529, PF-04691502, PKI-402, PKI-587, PP-242, PP-30, SB-1518, SB-2312, SF-1126, TAFA-93, TOP-216, Torin1, WAY-600, WYE-125132, WYE-354, WYE-687, and XL-765, or a pharmaceutically acceptable prodrug, salt, solvate, or polymorph thereof. In a still further aspect, the mTor inhibitor is selected from everolimus, rapamycin (sirolimus), and temsirolimus, or a pharmaceutically acceptable prodrug, salt, solvate, or polymorph thereof. In yet a further aspect, the mTor inhibitor is everolimus, or a pharmaceutically acceptable prodrug, salt, solvate, or polymorph thereof. In an even further aspect, the mTor inhibitor is rapamycin (sirolimus), or a pharmaceutically acceptable prodrug, salt, solvate, or polymorph thereof. In a still further aspect, the mTor inhibitor is temsiorlimus, or a pharmaceutically acceptable prodrug, salt, solvate, or polymorph thereof. In yet a further aspect, the mTor inhibitor is deforolimus, or a pharmaceutically acceptable prodrug, salt, solvate, or polymorph thereof. In an even further aspect, the mTor inhibitor is tacrolimus, or a pharmaceutically acceptable prodrug, salt, solvate, or polymorph thereof. In a still further aspect, the mTor inhibitor is zotarolimus, or a pharmaceutically acceptable prodrug, salt, solvate, or polymorph thereof. In yet a further aspect, the mTor inhibitor is salirasib, or a pharmaceutically acceptable prodrug, salt, solvate, or polymorph thereof. In an even further aspect, the mTor inhibitor is curcumin, or a pharmaceutically acceptable prodrug, salt, solvate, or polymorph thereof. In a still further aspect, the mTor inhibitor is farnesylthiosalicylic acid, or a pharmaceutically acceptable prodrug, salt, solvate, or polymorph thereof. In yet a further aspect, the mTor inhibitor is Torin1, or a pharmaceutically acceptable prodrug, salt, solvate, or polymorph thereof.

In a further aspect, the composition further comprises an effective amount of a PLD inhibitor. In a still further aspect, the effective amount of the PLD inhibitor is a therapeutically effective amount. In yet a further aspect, the effective amount of the PLD inhibitor is a prophylactically effective amount.

In a further aspect, the PLD inhibitor is a compound having a structure represented by a formula:

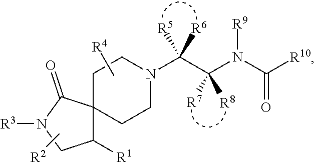

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, trifluoromethyl, carboxamido, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, trifluoromethyl, carboxamido, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof.

In a further aspect, the compound has a structure represented by a formula:

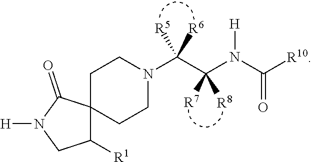

In a further aspect, the PLD inhibitor is a compound having a structure represented by a formula:

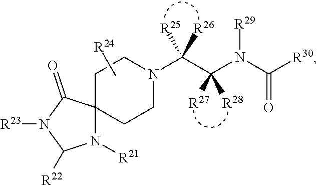

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, trifluoromethyl, carboxamido, alkylsulfonyl, an optionally substituted C1 to C6 alkyl, or an optionally substituted C3 to C6 cycloalkyl or R²⁵ and R²⁶, together with the intermediate carbon, comprise an optionally substituted C3 to C6 cycloalkyl; wherein each of R²⁷ and R²⁸ independently comprises hydrogen, trifluoromethyl, carboxamido, alkylsulfonyl, an optionally substituted C1 to C6 alkyl, or an optionally substituted C3 to C6 cycloalkyl or R²⁷ and R²⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof.

In a further aspect, the compound has a structure represented by a formula:

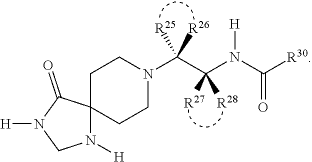

In a further aspect, the compound is:

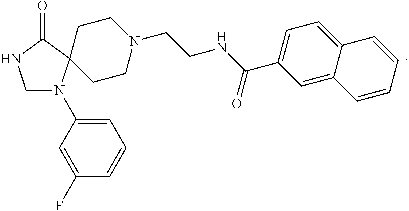 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

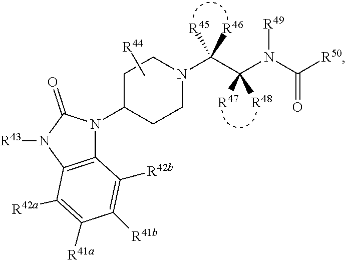

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, trifluoromethyl, carboxamido, alkylsulfonyl, an optionally substituted C1 to C6 alkyl, or an optionally substituted C3 to C6 cycloalkyl or R⁴⁵ and R⁴⁶, together with the intermediate carbon, comprise an optionally substituted C3 to C6 cycloalkyl; wherein each of R⁴⁷ and R⁴⁸ independently comprises hydrogen, trifluoromethyl, carboxamido, alkylsulfonyl, an optionally substituted C1 to C6 alkyl, or an optionally substituted C3 to C6 cycloalkyl or R⁴⁷ and R⁴⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof.

In a further aspect, the compound has a structure represented by a formula:

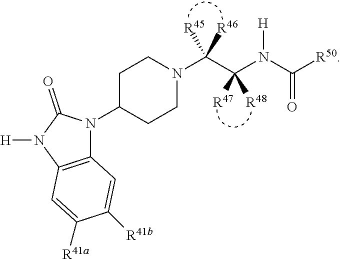

In a further aspect, the compound is:

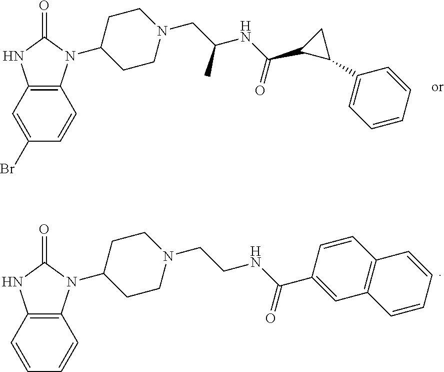

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxy tamoxifen; h) 5-fluoro-2-indoyl des-chlorohalopemide; and i) halopemide, or a pharmaceutically acceptable salt, hydrate, solvate, or polymorph thereof, thereby treating the subject for viral infection.

In a further aspect, the PLD inhibitor is selected from:

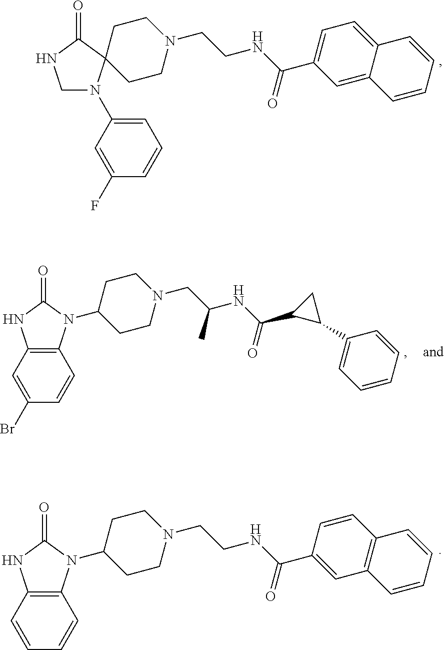

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the PLD inhibitor is selected from:

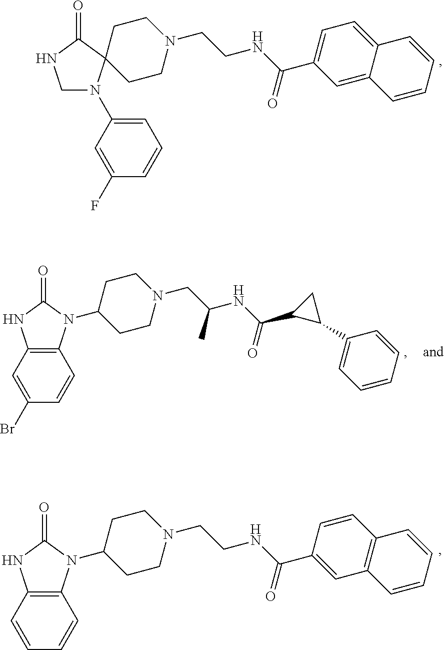

or a pharmaceutically acceptable salt, hydrate, solvate, or polymorph thereof.

In one aspect, the invention relates to pharmaceutical compositions comprising: a) a first therapeutic agent comprising an effective amount of a phospholipase D inhibitor, or a pharmaceutically acceptable prodrug, salt, solvate, or polymorph thereof; and b) a second therapeutic agent comprising an effective amount of a mTor inhibitor, or a pharmaceutically acceptable prodrug, salt, solvate, or polymorph thereof; and a pharmaceutically acceptable carrier. In a further aspect, an effective amount of a phospholipase D inhibitor is a therapeutically effective amount. In a still further aspect, an effective amount of a phospholipase D inhibitor is a prophylactically effective amount. In yet a further aspect, an effective amount of a mTor inhibitor is a therapeutically effective amount. In an even further aspect, an effective amount of a mTor inhibitor is a prophylactically effective amount.

In a further aspect, the PLD inhibitor is a compound having a structure represented by a formula:

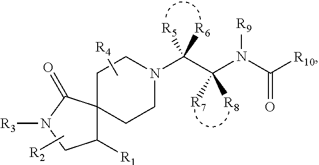

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

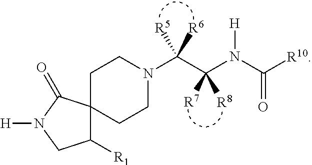

In a further aspect, the PLD inhibitor is a compound having a structure represented by a formula:

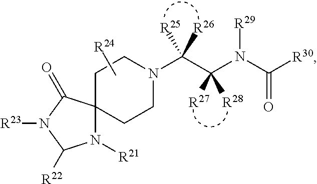

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

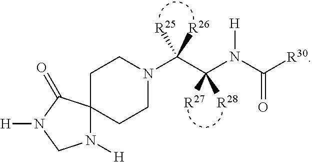

In a further aspect, the compound is:

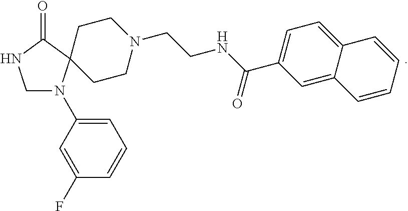 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

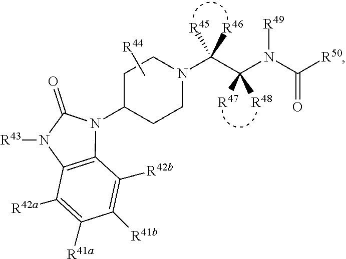

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

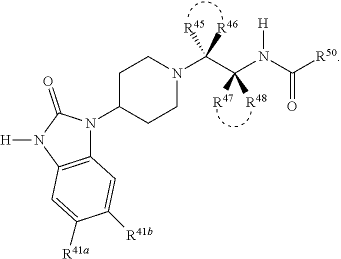

In a further aspect, the compound is:

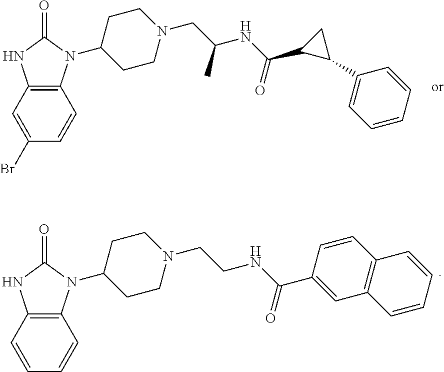

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof.

In a further aspect, the PLD inhibitor is a compound selected from:

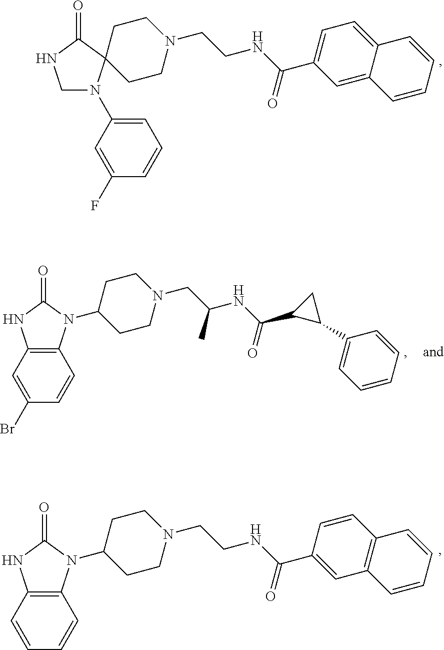

or a pharmaceutically acceptable salt, hydrate, solvate, or polymorph thereof.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the phospholipase D inhibitor is selected from:

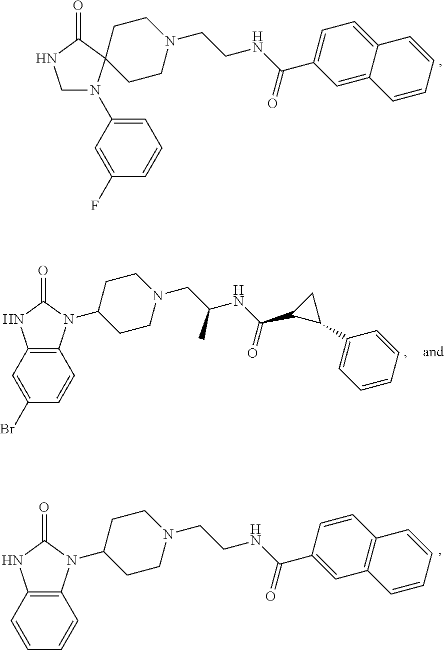

or a pharmaceutically acceptable salt, hydrate, solvate, or polymorph thereof.

In a further aspect, the mTor inhibitor is selected from everolimus, rapamycin (sirolimus), temsirolimus, deforolimus, ridaforolimus, tacrolimus, zotarolimus, salirasib, curcumin, farnesylthiosalicylic acid, XL765, ABI-009, AP-23675, AP-23841, AP-23765, AZD-8055, AZD-2014, BEZ-235 (NVP-BEZ235), BGT226, GDC-0980, INK-128, KU-0063794, MK8669, MKC-1 (Ro 31-7453), NVP-BGT226, OSI-027, Palomid-529, PF-04691502, PKI-402, PKI-587, PP-242, PP-30, SB-1518, SB-2312, SF-1126, TAFA-93, TOP-216, Torin1, WAY-600, WYE-125132, WYE-354, WYE-687, and XL-765, or a pharmaceutically acceptable prodrug, salt, solvate, or polymorph thereof. In a still further aspect, the mTor inhibitor is selected from everolimus, rapamycin (sirolimus), and temsirolimus, or a pharmaceutically acceptable prodrug, salt, solvate, or polymorph thereof. In yet a further aspect, the mTor inhibitor is everolimus, or a pharmaceutically acceptable prodrug, salt, solvate, or polymorph thereof. In an even further aspect, the mTor inhibitor is rapamycin (sirolimus), or a pharmaceutically acceptable prodrug, salt, solvate, or polymorph thereof. In a still further aspect, the mTor inhibitor is temsiorlimus, or a pharmaceutically acceptable prodrug, salt, solvate, or polymorph thereof. In yet a further aspect, the mTor inhibitor is deforolimus, or a pharmaceutically acceptable prodrug, salt, solvate, or polymorph thereof. In an even further aspect, the mTor inhibitor is tacrolimus, or a pharmaceutically acceptable prodrug, salt, solvate, or polymorph thereof. In a still further aspect, the mTor inhibitor is zotarolimus, or a pharmaceutically acceptable prodrug, salt, solvate, or polymorph thereof. In yet a further aspect, the mTor inhibitor is salirasib, or a pharmaceutically acceptable prodrug, salt, solvate, or polymorph thereof. In an even further aspect, the mTor inhibitor is curcumin, or a pharmaceutically acceptable prodrug, salt, solvate, or polymorph thereof. In a still further aspect, the mTor inhibitor is farnesylthiosalicylic acid, or a pharmaceutically acceptable prodrug, salt, solvate, or polymorph thereof. In yet a further aspect, the mTor inhibitor is Torin1, or a pharmaceutically acceptable prodrug, salt, solvate, or polymorph thereof.

In a further aspect, the composition further comprises an effective amount of an Akt therapeutic agent. In a still further aspect, the Akt therapeutic agent is an Akt inhibitor.

In a further aspect, the Akt inhibitor binds to the pleckstrin homology domain.

In a further aspect, the Akt inhibitor is an ATP-competitive inhibitor.

In a further aspect, the Akt inhibitor is an allosteric inhibitor. In a still further aspect, the allosteric inhibitor is MK-2066.

In a further aspect, the Akt inhibitor is a pan-Akt inhibitor.

In a further aspect, the Akt inhibitor is an isoform-selective inhibitor. In a still further aspect, the Akt inhibitor inhibits Akt1, Akt2, or Akt3. In yet a further aspect, the isoform-selective inhibitor selectively inhibits Akt1.

In a further aspect, the Akt therapeutic agent is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine. In a still further aspect, the Akt therapeutic agent is erucylphosphocholine. In yet a further aspect, the Akt therapeutic agent is GDC-0068. In an even further aspect, the Akt therapeutic agent is GSK-2141795. In a still further aspect, the Akt therapeutic agent is MK-2206. In yet a further aspect, the Akt therapeutic agent is perifosine. In an even further aspect, the therapeutic agent is PHT-427.

In a further aspect, the Akt therapeutic agent is a siRNA.

In a further aspect, the Akt therapeutic agent is an antisense oligonucleotide. In a still further aspect, the antisense oligonucleotide is RX-0201.

In a further aspect, the composition further comprises an effective amount of at least one HIV therapeutic agent selected from: a) a HIV fusion/lysis inhibitor, or a pharmaceutically acceptable prodrug, salt, solvate, or polymorph thereof b) a HIV integrase inhibitor, or a pharmaceutically acceptable prodrug, salt, solvate, or polymorph thereof; c) a HIV non-nucleoside reverse transcriptase inhibitor, or a pharmaceutically acceptable prodrug, salt, solvate, or polymorph thereof; d) a HIV nucleoside reverse transcriptase inhibitor, or a pharmaceutically acceptable prodrug, salt, solvate, or polymorph thereof; and e) a HIV protease inhibitor, or a pharmaceutically acceptable prodrug, salt, solvate. In a further aspect, the effective amount of at least one HIV therapeutic agent is a therapeutically effective amount. In a still further aspect, the effective amount of at least one HIV therapeutic agent is a prophylactically effective amount.

In a further aspect, the HIV fusion/lysis inhibitor is selected from enfuvirtide, maraviroc, cenicriviroc, ibalizumab, BMS-663068, and PRO-140, or a pharmaceutically acceptable prodrug, salt, solvate, or polymorph thereof. In a still further aspect, the HIV fusion/lysis inhibitor is selected from enfuvirtide, maraviroc, cenicriviroc, and ibalizumab, or a pharmaceutically acceptable prodrug, salt, solvate, or polymorph thereof. In yet a further aspect, the HIV fusion/lysis inhibitor is enfuvirtide, or a pharmaceutically acceptable prodrug, salt, solvate, or polymorph thereof. In an even further aspect, the HIV fusion/lysis inhibitor is maraviroc, or a pharmaceutically acceptable prodrug, salt, solvate, or polymorph thereof. In a still further aspect, the HIV fusion/lysis inhibitor is cenicriviroc, or a pharmaceutically acceptable prodrug, salt, solvate, or polymorph thereof. In yet a further aspect, the HIV fusion/lysis inhibitor is ibalizumab, or a pharmaceutically acceptable prodrug, salt, solvate, or polymorph thereof.

In a further aspect, the HIV integrase inhibitor is selected from raltegravir, dolutegravir, and elvitegravir, or a pharmaceutically acceptable prodrug, salt, solvate, or polymorph thereof. In a still further aspect, the HIV integrase inhibitor is raltegravir, or a pharmaceutically acceptable prodrug, salt, solvate, or polymorph thereof. In yet a further aspect, the HIV integrase inhibitor is dolutegravir, or a pharmaceutically acceptable prodrug, salt, solvate, or polymorph thereof. In an even further aspect, the HIV integrase inhibitor is elvitegravir, or a pharmaceutically acceptable prodrug, salt, solvate, or polymorph thereof.

In a further aspect, the HIV non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In a still further aspect, the HIV non-nucleoside reverse transcriptase inhibitor is delavirdine, or a pharmaceutically acceptable prodrug, salt, solvate, or polymorph thereof. In yet a further aspect, the HIV non-nucleoside reverse transcriptase inhibitor is efavirenz, or a pharmaceutically acceptable prodrug, salt, solvate, or polymorph thereof. In an even further aspect, the HIV non-nucleoside reverse transcriptase inhibitor is etravirine, or a pharmaceutically acceptable prodrug, salt, solvate, or polymorph thereof. In a still further aspect, the HIV non-nucleoside reverse transcriptase inhibitor is lersivirine, or a pharmaceutically acceptable prodrug, salt, solvate, or polymorph thereof. In yet a further aspect, the HIV non-nucleoside reverse transcriptase inhibitor is nevirapine, or a pharmaceutically acceptable prodrug, salt, solvate, or polymorph thereof. In an even further aspect, the HIV non-nucleoside reverse transcriptase inhibitor is rilpivirine, or a pharmaceutically acceptable prodrug, salt, solvate, or polymorph thereof.

In a further aspect, the HIV nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In a still further aspect, the HIV nucleoside reverse transcriptase inhibitor is selected from abacavir, didansine, elvucitabine, emtricitabine, lamivudine, stavudine, tenofovir, and zidovudine, or a pharmaceutically acceptable prodrug, salt, solvate, or polymorph thereof. In yet a further aspect, the HIV nucleoside reverse transcriptase inhibitor is abacavir, or a pharmaceutically acceptable prodrug, salt, solvate, or polymorph thereof. In an even further aspect, the HIV nucleoside reverse transcriptase inhibitor is didansine, or a pharmaceutically acceptable prodrug, salt, solvate, or polymorph thereof. In a still further aspect, the HIV nucleoside reverse transcriptase inhibitor is elvucitabine, or a pharmaceutically acceptable prodrug, salt, solvate, or polymorph thereof. In yet a further aspect, the HIV nucleoside reverse transcriptase inhibitor is emtricitabine, or a pharmaceutically acceptable prodrug, salt, solvate, or polymorph thereof. In an even further aspect, the HIV nucleoside reverse transcriptase inhibitor is lamivudine, or a pharmaceutically acceptable prodrug, salt, solvate, or polymorph thereof. In a still further aspect, the HIV nucleoside reverse transcriptase inhibitor is stavudine, or a pharmaceutically acceptable prodrug, salt, solvate, or polymorph thereof. In yet a further aspect, the HIV nucleoside reverse transcriptase inhibitor is tenofovir, or a pharmaceutically acceptable prodrug, salt, solvate, or polymorph thereof. In an even further aspect, the HIV nucleoside reverse transcriptase inhibitor is zidovudine, or a pharmaceutically acceptable prodrug, salt, solvate, or polymorph thereof.

In a further aspect, the HIV protease inhibitor is selected from wherein the HIV protease inhibitor is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In a still further aspect, the HIV protease inhibitor is atazanir, or a pharmaceutically acceptable prodrug, salt, solvate, or polymorph thereof. In yet a further aspect, the HIV protease inhibitor is darunavir, or a pharmaceutically acceptable prodrug, salt, solvate, or polymorph thereof. In an even further aspect, the HIV protease inhibitor is fosamprenavir, or a pharmaceutically acceptable prodrug, salt, solvate, or polymorph thereof. In a still further aspect, the HIV protease inhibitor is indinavir, or a pharmaceutically acceptable prodrug, salt, solvate, or polymorph thereof. In yet a further aspect, the HIV protease inhibitor is lopinavir, or a pharmaceutically acceptable prodrug, salt, solvate, or polymorph thereof. In an even further aspect, the HIV protease inhibitor is nelfinavir, or a pharmaceutically acceptable prodrug, salt, solvate, or polymorph thereof. In a still further aspect, the HIV protease inhibitor is ritonavir, or a pharmaceutically acceptable prodrug, salt, solvate, or polymorph thereof. In yet a further aspect, the HIV protease inhibitor is saquinavir, or a pharmaceutically acceptable prodrug, salt, solvate, or polymorph thereof. In an even further aspect, the HIV protease inhibitor is tipranavir, or a pharmaceutically acceptable prodrug, salt, solvate, or polymorph thereof.

In a further aspect, the composition further comprises an effective amount of at least one influenza therapeutic agent selected from: a) a viral protein M2 ion channel inhibitor, or a pharmaceutically acceptable prodrug, salt, solvate, or polymorph thereof; b) a neuraminidase inhibitor, or a pharmaceutically acceptable prodrug, salt, solvate, or polymorph thereof; and c) a nucleoside analog, or a pharmaceutically acceptable prodrug, salt, solvate, or polymorph thereof; and a pharmaceutically acceptable carrier. In a still further aspect, the effective amount of the at least one influenza therapeutic agent is a therapeutically effective amount. In yet a further aspect, the effective amount of the at least one influenza therapeutic agent is a prophylatically effective amount.

In a further aspect, the viral protein M2 ion channel inhibitor is an amino-adamantane compound. In a still further aspect, the amino-adamantane compound is selected from 1-amino-adamantane and 1-(1-aminoethyl)adamantane. In yet a further aspect, the viral protein M2 ion channel inhibitor is selected from amantadine and rimantadine, or a pharmaceutically acceptable prodrug, salt, solvate, or polymorph thereof. In an even further aspect, the viral protein M2 ion channel inhibitor is an analog of amantadine or rimantadine. In a still further aspect, the amantadine analog is selected from 1-amino-1,3,5-trimethylcyclohexane, 1-amino-1(trans),3(trans),5-trimethylcyclohexane, 1-amino-1(cis),3(cis),5-trimethylcyclohexane, 1-amino-1,3,3,5-tetramethylcyclohexane, 1-amino-1,3,3,5,5-pentamethylcyclohexane(neramexane), 1-amino-1,3,5,5-tetramethyl-3-ethylcyclohexane, 1-amino-1,5,5-trimethyl-3,3-diethylcyclohexane, 1-amino-1,5,5-trimethyl-cis-3-ethylcyclohexane, 1-amino-(1S,5S)cis-3-ethyl-1,5,5-trimethylcyclohexane, 1-amino-1,5,5-trimethyl-trans-3-ethylcyclohexane, 1-amino-(1R,5S)trans-3-ethyl-1,5,5-trimethylcyclohexane, 1-amino-1-ethyl-3,3,5,5-tetramethylcyclohexane, 1-amino-1-propyl-3,3,5,5-tetramethylcyclohexane, N-methyl-1-amino-1,3,3,5,5-pentamethylcyclohexane, N-ethyl-1-amino-1,3,3,5,5-pentamethyl-cyclohexane, N-(1,3,3,5,5-pentamethylcyclohexyl)pyrrolidine, 3,3,5,5-tetramethylcyclohexylmethylamine, 1-amino-1-propyl-3,3,5,5-tetramethylcyclohexane, 1 amino-1,3,3,5(trans)-tetramethylcyclohexane (axial amino group), 3-propyl-1,3,5,5-tetramethylcyclohexylamine semihydrate, 1-amino-1,3,5,5-tetramethyl-3-ethylcyclohexane, 1-amino-1,3,5-trimethylcyclohexane, 1-amino-1,3-dimethyl-3-propylcyclohexane, 1-amino-1,3(trans),5(trans)-trimethyl-3(cis)-propylcyclohexane, 1-amino-1,3-dimethyl-3-ethylcyclohexane, 1-amino-1,3,3-trimethylcyclohexane, cis-3-ethyl-1(trans)-3(trans)-5-trimethylcyclohexamine, 1-amino-1,3(trans)-dimethylcyclohexane, 1,3,3-trimethyl-5,5-dipropylcyclohexylamine, 1-amino-1-methyl-3(trans)-propylcyclohexane, 1-methyl-3 (cis)-propylcyclohexylamine, 1-amino-1-methyl-3(trans)-ethylcyclohexane, 1-amino-1,3,3-trimethyl-5(cis)-ethylcyclohexane, 1-amino-1,3,3-trimethyl-5(trans)-ethylcyclohexane, cis-3-propyl-1,5,5-trimethylcyclohexylamine, trans-3-propyl-1,5,5-trimethylcyclohexylamine, N-ethyl-1,3,3,5,5-pentamethylcyclohexylamine, N-methyl-1-amino-1,3,3,5,5-pentamethylcyclohexane, 1-amino-1-methylcyclohexane, N,N-dimethyl-1-amino-1,3,3,5,5-pentamethylcyclohexane, 2-(3,3,5,5-tetramethylcyclohexyl)ethylamine, 2-methyl-1-(3,3,5,5-tetramethylcyclohexyl)propyl-2-amine, 2-(1,3,3,5,5-pentamethylcyclohexyl-1)-ethylamine semihydrate, N-(1,3,3,5,5-pentamethylcyclohexyl)-pyrrolidine, 1-amino-1,3(trans),5(trans)-trimethylcyclohexane, 1-amino-1,3(cis),5(cis)-trimethylcyclohexane, 1-amino-(1R,5S)trans-5-ethyl-1,3,3-trimethylcyclohexane, 1-amino-(1S,5S)cis-5-ethyl-1,3,3-trimethylcyclohexane, 1-amino-1,5,5-trimethyl-3(cis)-isopropyl-cyclohexane, 1-amino-1,5,5-trimethyl-3(trans)-isopropyl-cyclohexane, 1-amino-1-methyl-3(cis)-ethyl-cyclohexane, 1-amino-1-methyl-3(cis)-methyl-cyclohexane, 1-amino-5,5-diethyl-1,3,3-trimethyl-cyclohexane, 1-amino-1,3,3,5,5-pentamethylcyclohexane, 1-amino-1,5,5-trimethyl-3,3-diethylcyclohexane, 1-amino-1-ethyl-3,3,5,5-tetramethylcyclohexane, N-ethyl-1-amino-1,3,3,5,5-pentamethylcyclohexane, N-(1,3,5-trimethylcyclohexyl)pyrrolidine, N-(1,3,5-trimethylcyclohexyl)piperidine, N-[1,3(trans),5(trans)-trimethylcyclohexyl]pyrrolidine, N-[1,3(trans),5(trans)-trimethylcyclohexyl]piperidine, N-[1,3(cis),5(cis)-trimethylcyclohexyl]pyrrolidine, N-[1,3(cis),5(cis)-trimethylcyclohexyl]piperidine, N-(1,3,3,5-tetramethylcyclohexyl)pyrrolidine, N-(1,3,3,5-tetramethylcyclohexyl)piperidine, N-(1,3,3,5,5-pentamethylcyclohexyl)pyrrolidine, N-(1,3,3,5,5-pentamethylcyclohexyl)piperidine, N-(1,3,5,5-tetramethyl-3-ethylcyclohexyl)pyrrolidine, N-(1,3,5,5-tetramethyl-3-ethylcyclohexyl)piperidine, N-(1,5,5-trimethyl-3,3-diethylcyclohexyl)pyrrolidine, N-(1,5,5-trimethyl-3,3-diethylcyclohexyl)piperidine, N-(1,3,3-trimethyl-cis-5-ethylcyclohexyl)pyrrolidine, N-(1,3,3-trimethyl-cis-5-ethylcyclohexyl)piperidine, N-[(1S,5S)cis-5-ethyl-1,3,3-trimethylcyclohexyl]pyrrolidine, N-[(1S,5S)cis-5-ethyl-1,3,3-trimethylcyclohexyl]piperidine, N-(1,3,3-trimethyl-trans-5-ethylcyclohexyl)pyrrolidine, N-(1,3,3-trimethyl-trans-5-ethylcyclohexyl)piperidine, N-[(1R,5S)trans-5-ethyl,3,3-trimethylcyclohexyl]pyrrolidine, N-[(1R,5S)trans-5-ethyl,3,3-trimethylcyclohexyl]piperidine, N-(1-ethyl-3,3,5,5-tetramethylyclohexyl)pyrrolidine, N-(1-ethyl-3,3,5,5-tetramethylyclohexyl)piperidine, N-(1-propyl-3,3,5,5-tetramethylcyclohexyl)pyrrolidine, N-(1-propyl-3,3,5,5-tetramethylcyclohexyl)piperidine, N-(1,3,3,5,5-pentamethylcyclohexyl)pyrrolidine, spiro[cyclopropane-1,2-adamantan]-2-amine, spiro[pyrrolidine-2,2′-adamantane], spiro[piperidine-2,2-adamantane], 2-(2-adamantyl)piperidine, 3-(2-adamantyl)pyrrolidine, 2-(1-adamantyl)piperidine, 2-(1-adamantyl)pyrrolidine, and 2-(1-adamantyl)-2-methyl-pyrrolidine. In yet a further aspect, the amantadine analog is selected from 1-amino-3-phenyl adamantane, 1-amino-methyl adamantane, 1-amino-3-ethyl adamantane, 1-amino-3-isopropyl adamantane, 1-amino-3-n-butyl adamantane, 1-amino-3,5-diethyl adamantane, 1-amino-3,5-diisopropyl adamantane, 1-amino-3,5-di-n-butyl adamantane, 1-amino-3-methyl-5-ethyl adamantane, 1-N-methylamino-3,5-dimethyl adamantane, 1-N-ethylamino-3,5-dimethyl adamantane, 1-N-isopropyl-amino-3,5-dimethyl adamantane, 1-N,N-dimethyl-amino-3,5-dimethyl adamantane, 1-N-methyl-N-isopropyl-amino-3-methyl-5-ethyl adamantane, 1-amino-3-butyl-5-phenyl adamantane, 1-amino-3-pentyl adamantane, 1-amino-3,5-dipentyl adamantane, 1-amino-3-pentyl-5-hexyl adamantane, 1-amino-3-pentyl-5-cyclohexyl adamantane, 1-amino-3-pentyl-5-phenyl adamantane, 1-amino-3-hexyl adamantane, 1-amino-3,5-dihexyl adamantane, 1-amino-3-hexyl-5-cyclohexyl adamantane, 1-amino-3-hexyl-5-phenyl adamantane, 1-amino-3-cyclohexyl adamantane, 1-amino-3,5-dicyclohexyl adamantane, 1-amino-3-cyclohexyl-5-phenyl adamantane, 1-amino-3,5-diphenyl adamantane, 1-amino-3,5,7-trimethyl adamantane, 1-amino-3,5-dimethyl-7-ethyl adamantane, 1-amino-3,5-diethyl-7-methyl adamantane, 1-N-pyrrolidino and 1-N-piperidine derivatives, 1-amino-3-methyl-5-propyl adamantane, 1-amino-3-methyl-5-butyl adamantane, 1-amino-3-methyl-5-pentyl adamantane, 1-amino-3-methyl-5-hexyl adamantane, 1-amino-3-methyl-5-cyclohexyl adamantane, 1-amino-3-methyl-5-phenyl adamantane, 1-amino-3-ethyl-5-propyl adamantane, 1-amino-3-ethyl-5-butyl adamantane, 1-amino-3-ethyl-5-pentyl adamantane, 1-amino-3-ethyl-5-hexyl adamantane, 1-amino-3-ethyl-5-cyclohexyl adamantane, 1-amino-3-ethyl-5-phenyl adamantane, 1-amino-3-propyl-5-butyl adamantane, 1-amino-3-propyl-5-pentyl adamantane, 1-amino-3-propyl-5-hexyl adamantane, 1-amino-3-propyl-5-cyclohexyl adamantane, 1-amino-3-propyl-5-phenyl adamantane, 1-amino-3-butyl-5-pentyl adamantane, 1-amino-3-butyl-5-hexyl adamantane, and 1-amino-3-butyl-5-cyclohexyl adamantine.

In a further aspect, the neuraminidase inhibitor is selected from oseltamivir, zanamivir, peramivir, laninamivir octanoate, 2,3-didehydro-2-deoxy-N-acetylneuraminic acid (DANA), 2-deoxy-2,3-dehydro-N-trifluoroacetylneuraminic acid (FANA), N-[(1R,2S)-2-methoxy-2-methyl-1-[(2R,3S,5R)-5-(2-methylpropanoyl)-3-[(Z)-prop-1-enyl]pyrrolidin-2-yl]pentyl]acetamide (A-322278), and (2R,4S,5R)-5-[(1R,2S)-1-acetamido-2-methoxy-2-methylpentyl]-4-[(Z)-prop-1-enyl]pyrrolidine-2-carboxylic acid (A-315675), or a pharmaceutically acceptable salt, solvate, or polymorph thereof. In a still further aspect, the neuraminidase inhibitor is selected from oseltamivir, zanamivir, peramivir, laninamivir octanoate, or a pharmaceutically acceptable salt, solvate, or polymorph thereof. In yet a further aspect, the neuraminidase inhibitor is oseltamivir, oseltamivir phosphate, or oseltamivir carboxylate. In an even further aspect, the neuraminidase inhibitor is oseltamivir phosphate. In a still further aspect, the neuraminidase inhibitor is zanamivir. In yet a further aspect, the neuraminidase inhibitor is peramivir. In an even further aspect, the neuraminidase inhibitor is laninamivir octanoate.

In a further aspect, the nucleoside analog is selected from ribavirin, viramidine, 6-fluoro-3-hydroxy-2-pyrazinecarboxamide, 2′-deoxy-2′-fluoroguanosine, pyrazofurin, carbodine, and cyclopenenyl cytosine. In a still further aspect, the nucleoside analog is selected from ribavirin and viramidine. In yet a further aspect, the nucleoside analog is ribavirin. In an even further aspect, the nucleoside analog is viramidine.

In a further aspect, the composition further comprises an effective amount of at least one anticancer agent selected from: a) a hormone therapy therapeutic agent, or a pharmaceutically acceptable prodrug, salt, solvate, or polymorph thereof; b) an alkylating therapeutic agent, or a pharmaceutically acceptable prodrug, salt, solvate, or polymorph thereof; c) an antineoplastic antimetabolite therapeutic agent, or a pharmaceutically acceptable prodrug, salt, solvate, or polymorph thereof; d) a mitotic inhibitor therapeutic agent, or a pharmaceutically acceptable prodrug, salt, solvate, or polymorph thereof; e) an antineoplastic antibiotic therapeutic agent, or a pharmaceutically acceptable prodrug, salt, solvate, or polymorph thereof; or f) other chemotherapeutic agent, or a pharmaceutically acceptable prodrug, salt, solvate, or polymorph thereof. In a further aspect, the effective amount of at least one anticancer agent is a therapeutically effective amount. In a still further aspect, the effective amount of at least one anticancer agent is a prophylactically effective amount.

In a further aspect, the hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt, hydrate, solvate, or polymorph thereof.

In a further aspect, the alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof.

In a further aspect, the antineoplastic antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt, hydrate, solvate, or polymorph thereof.

In a further aspect, the mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt, hydrate, solvate, or polymorph thereof.

In a further aspect, the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt, hydrate, solvate, or polymorph thereof.

The disclosed compounds can be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and can be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans. The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions encompass any composition made by admixing a compound of the present invention and a pharmaceutically acceptable carrier.

As used herein, the term “pharmaceutically acceptable salts” refers to salts prepared from pharmaceutically acceptable non-toxic bases or acids. When a disclosed compound is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids,” includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable derivatives thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

Pharmaceutical compositions suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carriers) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment of the disclosed conditions, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The disclosed pharmaceutical compositions can further comprise other therapeutically active compounds, as discussed further herein, which are usually applied in the treatment of the above mentioned pathological conditions.

In a further aspect, a pharmaceutical composition can comprise a therapeutically effective amount of any one or more disclosed compound and a pharmaceutically acceptable carrier. In a further aspect, a pharmaceutical composition can comprise a therapeutically effective amount of one or more product of any disclosed method and a pharmaceutically acceptable carrier. In one aspect, the invention relates to a method for manufacturing a medicament comprising combining at least one disclosed compound or at least one product of a disclosed method with a pharmaceutically acceptable carrier or diluent.

It is understood that the disclosed compositions can be prepared from the disclosed compounds. It is also understood that the disclosed compositions can be employed in the disclosed methods of using.

### G. METHODS OF USING THE COMPOUNDS AND COMPOSITIONS

Also provided is a method of use of a disclosed compound, composition, or medicament. In one aspect, the method of use is directed to the treatment of a disorder. In a further aspect, the disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The disclosed compounds can be used as single agents or in combination with one or more other drugs in the treatment, prevention, control, amelioration or reduction of risk of the aforementioned diseases, disorders and conditions for which compounds of formula I or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

1. Treatment Methods

The compounds disclosed herein are useful for treating, preventing, ameliorating, controlling or reducing the risk of a variety of viral infections or disorders of uncontrolled proliferation associated with phospholipase D and/or Akt dysfunction. Thus, provided is a method of treating or preventing a disorder in a subject comprising the step of administering to the subject at least one disclosed compound; at least one disclosed pharmaceutical composition; and/or at least one disclosed product in a dosage and amount effective to treat the disorder in the subject.

Also provided is a method for the treatment of one or more viral infections or disorders of uncontrolled proliferation associated with phospholipase D and/or Akt dysfunction in a subject comprising the step of administering to the subject at least one disclosed compound; at least one disclosed pharmaceutical composition; and/or at least one disclosed product in a dosage and amount effective to treat the disorder in the subject.

a. Treating an Infectious Disease

In one aspect, the invention relates to a method for treating a subject diagnosed with an infectious disease, the method comprising the step of administering to the subject an effective amount of an Akt therapeutic agent, thereby treating the subject for the infectious disease. In a further aspect, the subject is mammal. In a still further aspect, the mammal is a human.

In a further aspect, the infectious disease is associated with a viral infection. In a still further aspect, the method further comprises administering at least one anti-viral agent in combination with the therapeutic agent. In yet a further aspect, the subject has been diagnosed with a need for treatment of the viral infection prior to the administering step. In an even further aspect, the method further comprises the step of identifying a subject in need of treatment of the viral infection.

In a further aspect, the viral infection comprises infection with HIV. In a still further aspect, the HIV infection comprises a HIV-1 serotype virus. In yet a further aspect, the HIV-1 infection comprises a Group M, Group N, Group O, or Group P virus strain. In an even further aspect, the HIV-1 infection comprises a Group M virus strain. In a still further aspect, the HIV-1 Group M virus strain is selected from the subtypes A, B, C, D, F, G, H, J, and K. In yet a further aspect, the HIV-1 Group M virus strain subtype is subtype A. In an even further aspect, the HIV-1 Group M virus strain subtype is subtype B. In a still further aspect, the HIV-1 Group M virus strain subtype is subtype C. In yet a further aspect, the HIV-1 Group M virus strain subtype is subtype D. In an even further aspect, the HIV-1 Group M virus strain subtype is subtype H.

In a further aspect, the HIV-1 Group M virus strain subtype comprises a circulating recombinant form (“CRF”) comprising genetic material from one or more subtypes selected from subtypes A, B, C, D, F, G, H, J, and K. In a still further aspect, the circulating recombinant form is CRF A/E. In yet a further aspect, the circulating recombinant form is CRF A/G.

In a further aspect, the HIV infection comprises a HIV-2 serotype virus.

In a further aspect, the HIV infection is associated with a disease selected from AIDS, aspergillosis, atypical mycobacteriosis, bacillary angiomatosis, bacteremia, bacterial pneumonia, bacterial sinusitis, candidiasis, CMV, CMV retinitis, coccidioidomycosis, cryptococcosis, cryptosporidiosis-isosporiasis, non-specific enteritis, folliculitis, herpes, histoplasmosis, HIV dementia, HIV meningitis, leismaniasis, Mycobacterium avium complex disease, nocardiosis, pencilliosis, progressive multifocal leukoencephalopathy (PML; or HIV encephalitis), Pneumocystis carinii pneumonia (PCP), pneumonia, Pseudomonas pneumonia, toxoplasma encephalitis, toxoplasmosis, tuberculosis, Kaposi sarcoma, lymphoma, and squamous cell carcinoma. In a still further aspect, the lymphoma is selected from Non-Hodgkin's lymphoma, CNS lymphoma, primary lymphoma of the brain, and systemic lymphoma.

In a further aspect, the HIV infection is associated with a cancer. In a still further aspect, the cancer is selected from a lymphoma, sarcoma, and a carcinoma. In yet a further aspect, the carcinoma is a squamous cell carcinoma. In an even further aspect, the sarcoma is Kaposi sarcoma. In a still further aspect, the lymphoma is selected from Non-Hodgkin's lymphoma, CNS lymphoma, primary lymphoma of the brain, and systemic lymphoma.

In a further aspect, the HIV infection is associated with an opportunistic infection. In a still further aspect, the opportunistic infection is selected from aspergillosis, atypical mycobacteriosis, bacillary angiomatosis, bacteremia, bacterial pneumonia, bacterial sinusitis, candidiasis, CMV retinitis, coccidioidomycosis, cryptococcosis, cryptosporidiosis-isosporiasis, non-specific enteritis, folliculitis, herpes, histoplasmosis, HIV dementia, HIV meningitis, leismaniasis, Mycobacterium avium complex disease, nocardiosis, pencilliosis, progressive multifocal leukoencephalopathy (PML; or HIV encephalitis), Pneumocystis carinii pneumonia (PCP), pneumonia, Pseudomonas pneumonia, toxoplasma encephalitis, toxoplasmosis, and tuberculosis.

In a further aspect, the HIV infection is associated with an infection associated with Cryptosporidium muris, Isospora belli, Toxoplasma gondii, Candida sp., Coccidioides immitis, Histoplasma capsulatum, Pneumocystis carnii, Mycobacterium avium complex, Mycobacterium tuberculosis, Cytomegalovirus, Epstein-Barr virus, Herpes simplex virus, Papovirus J-C, or Varicella-zoster.

In a further aspect, the subject has been diagnosed with a need for treatment of an HIV infection prior to the administering step. In a still further aspect, the method further comprises the step of identifying a subject in need of treatment of the HIV infection.

In a further aspect, the HIV infection comprises an HIV virus that is resistant to treatment with a non-nucleoside reverse transcriptase inhibitor. In a still further aspect, the HIV virus resistant to treatment with a non-nucleoside reverse transcriptase inhibitor has at least one mutation in the HIV reverse transcriptase. In yet a further aspect, the at least one mutation in the HIV reverse transcriptase is selected from 100I, 103N, 106A, 106M, 108I, 181C, 181I, 188C, 188H, 188L, 190A, 190S, 225H, 230L, and 236L. In an even further aspect, the at least one mutation is at amino acid position 100, 103, 106, 108, 181, 188, 190, 225, 230, or 236 of the HIV reverse transcriptase.

In a further aspect, the HIV non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In a still further aspect, the HIV non-nucleoside reverse transcriptase inhibitor is delavirdine, or a pharmaceutically acceptable prodrug, salt, solvate, or polymorph thereof. In yet a further aspect, the HIV non-nucleoside reverse transcriptase inhibitor is efavirenz, or a pharmaceutically acceptable prodrug, salt, solvate, or polymorph thereof. In an even further aspect, the HIV non-nucleoside reverse transcriptase inhibitor is etravirine, or a pharmaceutically acceptable prodrug, salt, solvate, or polymorph thereof. In a still further aspect, the HIV non-nucleoside reverse transcriptase inhibitor is nevirapine, or a pharmaceutically acceptable prodrug, salt, solvate, or polymorph thereof. In yet a further aspect, the HIV non-nucleoside reverse transcriptase inhibitor is rilpivirine, or a pharmaceutically acceptable prodrug, salt, solvate, or polymorph thereof. In an even further aspect, the HIV non-nucleoside reverse transcriptase inhibitor is lersivirine, or a pharmaceutically acceptable prodrug, salt, solvate, or polymorph thereof.

In a further aspect, the HIV infection comprises an HIV virus that is resistant to treatment with an HIV nucleoside reverse transcriptase inhibitor. In a still further aspect, the HIV virus resistant to treatment with the HIV nucleoside reverse transcriptase inhibitor has at least one mutation in the HIV reverse transcriptase. In yet a further aspect, the at least one mutation in the HIV reverse transcriptase is selected from 41L, 44D, 62V, 65R, 67N, 69A, 69D, 69N, 69S, 69 insertion, 70R, 74V, 75I, 77L, 115F, 116Y, 118I, 151M, 184I, 184V, 210W, 215C, 215D, 215E, 215F, 215I, 215S, 215Y, 219E, and 219Q. In an even further aspect, the at least one mutation is at amino acid position 41, 44, 62, 65, 67, 69, 70, 74, 77, 115, 116, 118, 151, 184, 210, 215 or 219 of the HIV reverse transcriptase.

In a further aspect, the HIV nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In a still further aspect, the HIV nucleoside reverse transcriptase inhibitor is selected from abacavir, didansine, emtricitabine, lamivudine, stavudine, tenofovir, zidovudine, and elvucitabine, or a pharmaceutically acceptable prodrug, salt, solvate, or polymorph thereof. In yet a further aspect, the HIV nucleoside reverse transcriptase inhibitor is abacavir, or a pharmaceutically acceptable prodrug, salt, solvate, or polymorph thereof. In an even further aspect, the HIV nucleoside reverse transcriptase inhibitor is didansine, or a pharmaceutically acceptable prodrug, salt, solvate, or polymorph thereof. In a still further aspect, the HIV nucleoside reverse transcriptase inhibitor is emtricitabine, or a pharmaceutically acceptable prodrug, salt, solvate, or polymorph thereof. In yet a further aspect, the HIV nucleoside reverse transcriptase inhibitor is lamivudine, or a pharmaceutically acceptable prodrug, salt, solvate, or polymorph thereof. In an even further aspect, the HIV nucleoside reverse transcriptase inhibitor is stavudine, or a pharmaceutically acceptable prodrug, salt, solvate, or polymorph thereof. In a still further aspect, the HIV nucleoside reverse transcriptase inhibitor is tenofovir, or a pharmaceutically acceptable prodrug, salt, solvate, or polymorph thereof. In yet a further aspect, the HIV nucleoside reverse transcriptase inhibitor is zidovudine, or a pharmaceutically acceptable prodrug, salt, solvate, or polymorph thereof. In an even further aspect, the HIV nucleoside reverse transcriptase inhibitor is elvucitabine, or a pharmaceutically acceptable prodrug, salt, solvate, or polymorph thereof.

In a further aspect, the HIV infection comprises an HIV virus that is resistant to treatment with an HIV protease inhibitor. In a still further aspect, the HIV virus resistant to treatment with the HIV protease inhibitor has at least one mutation in the HIV protease. In yet a further aspect, the at least one mutation in the HIV protease is selected from 30N, 46I, 46L, 48V, 50V, 82A, 82F, 82S, 82T, 84V, and 90M. In an even further aspect, the at least one mutation is at amino acid position 30, 46, 48, 50, 82, 84, or 90 of the HIV protease.

In a further aspect, the HIV protease inhibitor is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In a still further aspect, the HIV protease inhibitor is atazanavir, or a pharmaceutically acceptable prodrug, salt, solvate, or polymorph thereof. In yet a further aspect, the HIV protease inhibitor is darunavir, or a pharmaceutically acceptable prodrug, salt, solvate, or polymorph thereof. In an even further aspect, the HIV protease inhibitor is fosamprenavir, or a pharmaceutically acceptable prodrug, salt, solvate, or polymorph thereof. In a still further aspect, the HIV protease inhibitor is indinavir, or a pharmaceutically acceptable prodrug, salt, solvate, or polymorph thereof. In yet a further aspect, the HIV protease inhibitor is lopinavir, or a pharmaceutically acceptable prodrug, salt, solvate, or polymorph thereof. In an even further aspect, the HIV protease inhibitor is nelfinavir, or a pharmaceutically acceptable prodrug, salt, solvate, or polymorph thereof. In a still further aspect, the HIV protease inhibitor is ritonavir, or a pharmaceutically acceptable prodrug, salt, solvate, or polymorph thereof. In yet a further aspect, the HIV protease inhibitor is saquinavir, or a pharmaceutically acceptable prodrug, salt, solvate, or polymorph thereof. In an even further aspect, the HIV protease inhibitor is tipranavir, or a pharmaceutically acceptable prodrug, salt, solvate, or polymorph thereof. In a still further aspect, the HIV protease inhibitor is lopinavir/ritonavir, or a pharmaceutically acceptable prodrug, salt, solvate, or polymorph thereof.

In a further aspect, the HIV infection comprises an HIV virus that is resistant to treatment with an HIV integrase inhibitor.

In a further aspect, the HIV integrase inhibitor is selected from raltegravir, dolutegravir, elvitegravir, and S/GSK1265744, or a pharmaceutically acceptable prodrug, salt, solvate, or polymorph thereof. In a still further aspect, the HIV integrase inhibitor is selected from raltegravir, dolutegravir, and elvitegravir, or a pharmaceutically acceptable prodrug, salt, solvate, or polymorph thereof. In yet a further aspect, the HIV integrase inhibitor is raltegravir, or a pharmaceutically acceptable prodrug, salt, solvate, or polymorph thereof. In an even further aspect, the HIV integrase inhibitor is dolutegravir, or a pharmaceutically acceptable prodrug, salt, solvate, or polymorph thereof. In a still further aspect, the HIV integrase inhibitor is elvitegravir, or a pharmaceutically acceptable prodrug, salt, solvate, or polymorph thereof.

In a further aspect, the HIV infection comprises an HIV virus that is resistant to treatment with an HIV fusion inhibitor.

In a further aspect, the HIV fusion inhibitor is selected from enfuvirtide, maraviroc, cenicriviroc, ibalizumab, BMS-663068, and PRO-140, or a pharmaceutically acceptable prodrug, salt, solvate, or polymorph thereof. In a still further aspect, the HIV fusion inhibitor is selected from enfuvirtide, maraviroc, cenicriviroc, and ibalizumab, or a pharmaceutically acceptable prodrug, salt, solvate, or polymorph thereof. In yet a further aspect, the HIV fusion inhibitor is enfuvirtide, or a pharmaceutically acceptable prodrug, salt, solvate, or polymorph thereof. In a still further aspect, the HIV fusion inhibitor is maraviroc, or a pharmaceutically acceptable prodrug, salt, solvate, or polymorph thereof. In yet a further aspect, the HIV fusion inhibitor is cenicriviroc, or a pharmaceutically acceptable prodrug, salt, solvate, or polymorph thereof. In an even further aspect, the HIV fusion inhibitor is ibalizumab, or a pharmaceutically acceptable prodrug, salt, solvate, or polymorph thereof.

In a further aspect, the method further comprises co-administering the Akt therapeutic agent with an effective amount of at least one HIV therapeutic agent selected from: a) a HIV fusion/lysis inhibitor, or a pharmaceutically acceptable prodrug, salt, solvate, or polymorph thereof b) a HIV integrase inhibitor, or a pharmaceutically acceptable prodrug, salt, solvate, or polymorph thereof; c) a HIV non-nucleoside reverse transcriptase inhibitor, or a pharmaceutically acceptable prodrug, salt, solvate, or polymorph thereof; d) a HIV nucleoside reverse transcriptase inhibitor, or a pharmaceutically acceptable prodrug, salt, solvate, or polymorph thereof; and e) a HIV protease inhibitor, or a pharmaceutically acceptable prodrug, salt, solvate.

In a further aspect, the effective amount of the least one HIV therapeutic agent is a therapeutically effective amount. In a still further aspect, the effective amount of the least one HIV therapeutic agent is a prophylactically effective amount.

In a further aspect, the HIV fusion/lysis inhibitor is selected from enfuvirtide, maraviroc, cenicriviroc, ibalizumab, BMS-663068, and PRO-140, or a pharmaceutically acceptable prodrug, salt, solvate, or polymorph thereof. In a still further aspect, the HIV fusion/lysis inhibitor is selected from enfuvirtide, maraviroc, cenicriviroc, and ibalizumab, or a pharmaceutically acceptable prodrug, salt, solvate, or polymorph thereof. In yet a further aspect, the HIV fusion/lysis inhibitor is enfuvirtide, or a pharmaceutically acceptable prodrug, salt, solvate, or polymorph thereof. In an even further aspect, the HIV fusion/lysis inhibitor is maraviroc, or a pharmaceutically acceptable prodrug, salt, solvate, or polymorph thereof. In a still further aspect, the HIV fusion/lysis inhibitor is cenicriviroc, or a pharmaceutically acceptable prodrug, salt, solvate, or polymorph thereof. In yet a further aspect, the HIV fusion/lysis inhibitor is ibalizumab, or a pharmaceutically acceptable prodrug, salt, solvate, or polymorph thereof.

In a further aspect, the HIV integrase inhibitor is selected from raltegravir, dolutegravir, elvitegravir, and S/GSK1265744, or a pharmaceutically acceptable prodrug, salt, solvate, or polymorph thereof. In a still further aspect, the HIV integrase inhibitor is selected from raltegravir, dolutegravir, and elvitegravir, or a pharmaceutically acceptable prodrug, salt, solvate, or polymorph thereof. In yet a further aspect, the HIV integrase inhibitor is raltegravir, or a pharmaceutically acceptable prodrug, salt, solvate, or polymorph thereof. In an even further aspect, the HIV integrase inhibitor is dolutegravir, or a pharmaceutically acceptable prodrug, salt, solvate, or polymorph thereof. In a still further aspect, the HIV integrase inhibitor is elvitegravir, or a pharmaceutically acceptable prodrug, salt, solvate, or polymorph thereof.

In a further aspect, the HIV non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In a still further aspect, the HIV non-nucleoside reverse transcriptase inhibitor is delavirdine, or a pharmaceutically acceptable prodrug, salt, solvate, or polymorph thereof. In yet a further aspect, the HIV non-nucleoside reverse transcriptase inhibitor is efavirenz, or a pharmaceutically acceptable prodrug, salt, solvate, or polymorph thereof. In an even further aspect, the HIV non-nucleoside reverse transcriptase inhibitor is etravirine, or a pharmaceutically acceptable prodrug, salt, solvate, or polymorph thereof. In a still further aspect, the HIV non-nucleoside reverse transcriptase inhibitor is nevirapine, or a pharmaceutically acceptable prodrug, salt, solvate, or polymorph thereof. In yet a further aspect, the HIV non-nucleoside reverse transcriptase inhibitor is rilpivirine, or a pharmaceutically acceptable prodrug, salt, solvate, or polymorph thereof.

In a further aspect, the HIV nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In a still further aspect, the HIV nucleoside reverse transcriptase inhibitor is abacavir, or a pharmaceutically acceptable prodrug, salt, solvate, or polymorph thereof. In yet a further aspect, the HIV nucleoside reverse transcriptase inhibitor is didansine, or a pharmaceutically acceptable prodrug, salt, solvate, or polymorph thereof. In an even further aspect, the HIV nucleoside reverse transcriptase inhibitor is elvucitabine, or a pharmaceutically acceptable prodrug, salt, solvate, or polymorph thereof. In a still further aspect, the HIV nucleoside reverse transcriptase inhibitor is emtricitabine, or a pharmaceutically acceptable prodrug, salt, solvate, or polymorph thereof. In yet a further aspect, the HIV nucleoside reverse transcriptase inhibitor is lamivudine, or a pharmaceutically acceptable prodrug, salt, solvate, or polymorph thereof. In an even further aspect, the HIV nucleoside reverse transcriptase inhibitor is stavudine, or a pharmaceutically acceptable prodrug, salt, solvate, or polymorph thereof. In a still further aspect, the HIV nucleoside reverse transcriptase inhibitor is tenofovir, or a pharmaceutically acceptable prodrug, salt, solvate, or polymorph thereof. In yet a further aspect, the HIV nucleoside reverse transcriptase inhibitor is zidovudine, or a pharmaceutically acceptable prodrug, salt, solvate, or polymorph thereof.

In a further aspect, the HIV protease inhibitor is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In a still further aspect, the HIV protease inhibitor is atazanir, or a pharmaceutically acceptable prodrug, salt, solvate, or polymorph thereof. In yet a further aspect, the HIV protease inhibitor is darunavir, or a pharmaceutically acceptable prodrug, salt, solvate, or polymorph thereof. In a still further aspect, the HIV protease inhibitor is fosamprenavir, or a pharmaceutically acceptable prodrug, salt, solvate, or polymorph thereof. In yet a further aspect, the HIV protease inhibitor is indinavir, or a pharmaceutically acceptable prodrug, salt, solvate, or polymorph thereof. In an even further aspect, the HIV protease inhibitor is lopinavir, or a pharmaceutically acceptable prodrug, salt, solvate, or polymorph thereof. In a still further aspect, the HIV protease inhibitor is nelfinavir, or a pharmaceutically acceptable prodrug, salt, solvate, or polymorph thereof. In yet a further aspect, the HIV protease inhibitor is ritonavir, or a pharmaceutically acceptable prodrug, salt, solvate, or polymorph thereof. In an even further aspect, the HIV protease inhibitor is saquinavir, or a pharmaceutically acceptable prodrug, salt, solvate, or polymorph thereof. In a still further aspect, the HIV protease inhibitor is tipranavir, or a pharmaceutically acceptable prodrug, salt, solvate, or polymorph thereof.

In a further aspect, the viral infection comprises infection with an influenza virus. In a still further aspect, the subject is a bird. In yet a further aspect, the subject is a mammal. In an even further aspect, the mammal is selected from a human, a swine, a horse, a cat, and a dog. In a still further aspect, the mammal is a human.

In a further aspect, the influenza virus is selected from a type A influenza virus, type B influenza virus, and type C influenza virus. In a still further aspect, the virus is a type A influenza virus. In yet a further aspect, the type A influenza virus is of subtype H1, H5, H7 or H9. In an even further aspect, the type A influenza virus is of subtype H1N1, H1N2, H2N2, H3N2, H3N8, H5N1, H5N2, H5N3, H5N8, H5N9, H7N1, H7N2, H7N3, H7N4, H7N7, H7N9, H9N2, or H10N7. In a still further aspect, the type A influenza virus is of subtype H1N1, H1N2, H2N2, H3N2, H5N1, H5N3, H7N2, H7N3, H7N7, H9N2, or H10N7. In yet a further aspect, the type A influenza virus is H5N1. In an even further aspect, the type A influenza virus is H1N1. In a yet further aspect, the type A influenza virus is H7N9. In a still further aspect, the virus is a type B influenza virus. In yet a further aspect, the virus is a type C influenza virus.

In a further aspect, the virus is oseltamivir resistant. In a still further aspect, the virus is not oseltamivir resistant.

In a further aspect, the virus is amantadine resistant. In a still further aspect, the virus is not amantadine resistant.

In a further aspect, the virus is rimantadine resistant. In a still further aspect, the virus is not rimantadine resistant.

In a further aspect, the method further comprises co-administering the Akt therapeutic agent with an effective amount of at least one influenza therapeutic agent selected from: a) a viral protein M2 ion channel inhibitor; b) a neuraminidase inhibitor; and c) a nucleoside analog.

In a further aspect, the effective amount of the at least one influenza therapeutic agent is a therapeutically effective amount. In a still further aspect, the effective amount of the at least one influenza therapeutic agent is a prophylactically effective amount.

In a further aspect, co-administration is administration in a substantially simultaneous manner. In a still further aspect, co-administration is administration in a substantially sequential manner.

In a further aspect, the viral protein M2 ion channel inhibitor is an amino-adamantane compound. In a still further aspect, the amino-adamantane compound is selected from 1-amino-adamantane and 1-(1-aminoethyl)adamantane.

In a further aspect, the viral protein M2 ion channel inhibitor is selected from amantadine and rimantadine, or a pharmaceutically acceptable prodrug, salt, solvate, or polymorph thereof. In a still further aspect, the viral protein M2 ion channel inhibitor is an analog of amantadine or rimantadine. In yet a further aspect, the amantadine analog is selected from 1-amino-1,3,5-trimethylcyclohexane, 1-amino-1(trans),3(trans),5-trimethylcyclohexane, 1-amino-1(cis),3(cis),5-trimethylcyclohexane, 1-amino-1,3,3,5-tetramethylcyclohexane, 1-amino-1,3,3,5,5-pentamethylcyclohexane(neramexane), 1-amino-1,3,5,5-tetramethyl-3-ethylcyclohexane, 1-amino-1,5,5-trimethyl-3,3-diethylcyclohexane, 1-amino-1,5,5-trimethyl-cis-3-ethylcyclohexane, 1-amino-(1S,5S)cis-3-ethyl-1,5,5-trimethylcyclohexane, 1-amino-1,5,5-trimethyl-trans-3-ethylcyclohexane, 1-amino-(1R,5S)trans-3-ethyl-1,5,5-trimethylcyclohexane, 1-amino-1-ethyl-3,3,5,5-tetramethylcyclohexane, 1-amino-1-propyl-3,3,5,5-tetramethylcyclohexane, N-methyl-1-amino-1,3,3,5,5-pentamethylcyclohexane, N-ethyl-1-amino-1,3,3,5,5-pentamethyl-cyclohexane, N-(1,3,3,5,5-pentamethylcyclohexyl)pyrrolidine, 3,3,5,5-tetramethylcyclohexylmethylamine, 1-amino-1-propyl-3,3,5,5-tetramethylcyclohexane, 1 amino-1,3,3,5(trans)-tetramethylcyclohexane (axial amino group), 3-propyl-1,3,5,5-tetramethylcyclohexylamine semihydrate, 1-amino-1,3,5,5-tetramethyl-3-ethylcyclohexane, 1-amino-1,3,5-trimethylcyclohexane, 1-amino-1,3-dimethyl-3-propylcyclohexane, 1-amino-1,3 (trans),5(trans)-trimethyl-3(cis)-propylcyclohexane, 1-amino-1,3-dimethyl-3-ethylcyclohexane, 1-amino-1,3,3-trimethylcyclohexane, cis-3-ethyl-1(trans)-3(trans)-5-trimethylcyclohexamine, 1-amino-1,3(trans)-dimethylcyclohexane, 1,3,3-trimethyl-5,5-dipropylcyclohexylamine, 1-amino-1-methyl-3(trans)-propylcyclohexane, 1-methyl-3(cis)-propylcyclohexylamine, 1-amino-1-methyl-3(trans)-ethylcyclohexane, 1-amino-1,3,3-trimethyl-5(cis)-ethylcyclohexane, 1-amino-1,3,3-trimethyl-5(trans)-ethylcyclohexane, cis-3-propyl-1,5,5-trimethylcyclohexylamine, trans-3-propyl-1,5,5-trimethylcyclohexylamine, N-ethyl-1,3,3,5,5-pentamethylcyclohexylamine, N-methyl-1-amino-1,3,3,5,5-pentamethylcyclohexane, 1-amino-1-methylcyclohexane, N,N-dimethyl-1-amino-1,3,3,5,5-pentamethylcyclohexane, 2-(3,3,5,5-tetramethylcyclohexyl)ethylamine, 2-methyl-1-(3,3,5,5-tetramethylcyclohexyl)propyl-2-amine, 2-(1,3,3,5,5-pentamethylcyclohexyl-1)-ethylamine semihydrate, N-(1,3,3,5,5-pentamethylcyclohexyl)-pyrrolidine, 1-amino-1,3(trans),5(trans)-trimethylcyclohexane, 1-amino-1,3(cis),5(cis)-trimethylcyclohexane, 1-amino-(1R,5S)trans-5-ethyl-1,3,3-trimethylcyclohexane, 1-amino-(1S,5S)cis-5-ethyl-1,3,3-trimethylcyclohexane, 1-amino-1,5,5-trimethyl-3(cis)-isopropyl-cyclohexane, 1-amino-1,5,5-trimethyl-3(trans)-isopropyl-cyclohexane, 1-amino-1-methyl-3 (cis)-ethyl-cyclohexane, 1-amino-1-methyl-3(cis)-methyl-cyclohexane, 1-amino-5,5-diethyl-1,3,3-trimethyl-cyclohexane, 1-amino-1,3,3,5,5-pentamethylcyclohexane, 1-amino-1,5,5-trimethyl-3,3-diethylcyclohexane, 1-amino-1-ethyl-3,3,5,5-tetramethylcyclohexane, N-ethyl-1-amino-1,3,3,5,5-pentamethylcyclohexane, N-(1,3,5-trimethylcyclohexyl)pyrrolidine, N-(1,3,5-trimethylcyclohexyl)piperidine, N-[1,3(trans),5(trans)-trimethylcyclohexyl]pyrrolidine, N-[1,3(trans),5(trans)-trimethylcyclohexyl]piperidine, N-[1,3(cis),5(cis)-trimethylcyclohexyl]pyrrolidine, N-[1,3(cis),5(cis)-trimethylcyclohexyl]piperidine, N-(1,3,3,5-tetramethylcyclohexyl)pyrrolidine, N-(1,3,3,5-tetramethylcyclohexyl)piperidine, N-(1,3,3,5,5-pentamethylcyclohexyl)pyrrolidine, N-(1,3,3,5,5-pentamethylcyclohexyl)piperidine, N-(1,3,5,5-tetramethyl-3-ethylcyclohexyl)pyrrolidine, N-(1,3,5,5-tetramethyl-3-ethylcyclohexyl)piperidine, N-(1,5,5-trimethyl-3,3-diethylcyclohexyl)pyrrolidine, N-(1,5,5-trimethyl-3,3-diethylcyclohexyl)piperidine, N-(1,3,3-trimethyl-cis-5-ethylcyclohexyl)pyrrolidine, N-(1,3,3-trimethyl-cis-5-ethylcyclohexyl)piperidine, N-[(1S,5S)cis-5-ethyl-1,3,3-trimethylcyclohexyl]pyrrolidine, N-[(1S,5S)cis-5-ethyl-1,3,3-trimethylcyclohexyl]piperidine, N-(1,3,3-trimethyl-trans-5-ethylcyclohexyl)pyrrolidine, N-(1,3,3-trimethyl-trans-5-ethylcyclohexyl)piperidine, N-[(1R,5S)trans-5-ethyl,3,3-trimethylcyclohexyl]pyrrolidine, N-[(1R,5S)trans-5-ethyl,3,3-trimethylcyclohexyl]piperidine, N-(1-ethyl-3,3,5,5-tetramethylyclohexyl)pyrrolidine, N-(1-ethyl-3,3,5,5-tetramethylyclohexyl)piperidine, N-(1-propyl-3,3,5,5-tetramethylcyclohexyl)pyrrolidine, N-(1-propyl-3,3,5,5-tetramethylcyclohexyl)piperidine, N-(1,3,3,5,5-pentamethylcyclohexyl)pyrrolidine, spiro[cyclopropane-1,2-adamantan]-2-amine, spiro[pyrrolidine-2,2′-adamantane], spiro[piperidine-2,2-adamantane], 2-(2-adamantyl)piperidine, 3-(2-adamantyl)pyrrolidine, 2-(1-adamantyl)piperidine, 2-(1-adamantyl)pyrrolidine, and 2-(1-adamantyl)-2-methyl-pyrrolidine. In an even further aspect, the amantadine analog is selected from 1-amino-3-phenyl adamantane, 1-amino-methyl adamantane, 1-amino-3-ethyl adamantane, 1-amino-3-isopropyl adamantane, 1-amino-3-n-butyl adamantane, 1-amino-3,5-diethyl adamantane, 1-amino-3,5-diisopropyl adamantane, 1-amino-3,5-di-n-butyl adamantane, 1-amino-3-methyl-5-ethyl adamantane, 1-N-methylamino-3,5-dimethyl adamantane, 1-N-ethylamino-3,5-dimethyl adamantane, 1-N-isopropyl-amino-3,5-dimethyl adamantane, 1-N,N-dimethyl-amino-3,5-dimethyl adamantane, 1-N-methyl-N-isopropyl-amino-3-methyl-5-ethyl adamantane, 1-amino-3-butyl-5-phenyl adamantane, 1-amino-3-pentyl adamantane, 1-amino-3,5-dipentyl adamantane, 1-amino-3-pentyl-5-hexyl adamantane, 1-amino-3-pentyl-5-cyclohexyl adamantane, 1-amino-3-pentyl-5-phenyl adamantane, 1-amino-3-hexyl adamantane, 1-amino-3,5-dihexyl adamantane, 1-amino-3-hexyl-5-cyclohexyl adamantane, 1-amino-3-hexyl-5-phenyl adamantane, 1-amino-3-cyclohexyl adamantane, 1-amino-3,5-dicyclohexyl adamantane, 1-amino-3-cyclohexyl-5-phenyl adamantane, 1-amino-3,5-diphenyl adamantane, 1-amino-3,5,7-trimethyl adamantane, 1-amino-3,5-dimethyl-7-ethyl adamantane, 1-amino-3,5-diethyl-7-methyl adamantane, 1-N-pyrrolidino and 1-N-piperidine derivatives, 1-amino-3-methyl-5-propyl adamantane, 1-amino-3-methyl-5-butyl adamantane, 1-amino-3-methyl-5-pentyl adamantane, 1-amino-3-methyl-5-hexyl adamantane, 1-amino-3-methyl-5-cyclohexyl adamantane, 1-amino-3-methyl-5-phenyl adamantane, 1-amino-3-ethyl-5-propyl adamantane, 1-amino-3-ethyl-5-butyl adamantane, 1-amino-3-ethyl-5-pentyl adamantane, 1-amino-3-ethyl-5-hexyl adamantane, 1-amino-3-ethyl-5-cyclohexyl adamantane, 1-amino-3-ethyl-5-phenyl adamantane, 1-amino-3-propyl-5-butyl adamantane, 1-amino-3-propyl-5-pentyl adamantane, 1-amino-3-propyl-5-hexyl adamantane, 1-amino-3-propyl-5-cyclohexyl adamantane, 1-amino-3-propyl-5-phenyl adamantane, 1-amino-3-butyl-5-pentyl adamantane, 1-amino-3-butyl-5-hexyl adamantane, and 1-amino-3-butyl-5-cyclohexyl adamantine.

In a further aspect, the neuraminidase inhibitor is selected from oseltamivir, zanamivir, peramivir, laninamivir octanoate, 2,3-didehydro-2-deoxy-N-acetylneuraminic acid (DANA), 2-deoxy-2,3-dehydro-N-trifluoroacetylneuraminic acid (FANA), N-[(1R,2S)-2-methoxy-2-methyl-1-[(2R,3S,5R)-5-(2-methylpropanoyl)-3-[(Z)-prop-1-enyl]pyrrolidin-2-yl]pentyl]acetamide (A-322278), and (2R,4S,5R)-5-[(1R,2S)-1-acetamido-2-methoxy-2-methylpentyl]-4-[(Z)-prop-1-enyl]pyrrolidine-2-carboxylic acid (A-315675), or a pharmaceutically acceptable salt, solvate, or polymorph thereof. In a still further aspect, the neuraminidase inhibitor is selected from oseltamivir, zanamivir, peramivir, laninamivir octanoate, or a pharmaceutically acceptable salt, solvate, or polymorph thereof. In yet a further aspect, the neuraminidase inhibitor is oseltamivir, oseltamivir phosphate, or oseltamivir carboxylate. In an even further aspect, the neuraminidase inhibitor is oseltamivir phosphate. In a still further aspect, the neuraminidase inhibitor is zanamivir. In yet a further aspect, the neuraminidase inhibitor is peramivir. In an even further aspect, the neuraminidase inhibitor is laninamivir octanoate.

In a further aspect, the nucleoside analog is selected from ribavirin, viramidine, 6-fluoro-3-hydroxy-2-pyrazinecarboxamide, 2′-deoxy-2′-fluoroguanosine, pyrazofurin, carbodine, and cyclopenenyl cytosine. In a still further aspect, the nucleoside analog is selected from ribavirin and viramidine. In yet a further aspect, the nucleoside analog is ribavirin. In an even further aspect, the nucleoside analog is viramidine.

In a further aspect, the method further comprises a prostaglandin E2 receptor agonist, or a pharmaceutically acceptable prodrug, salt, solvate, or polymorph thereof. In a still further aspect, the prostaglandin E2 receptor agonist is selected from a prostaglandin E receptor 4 (subtype EP4) selective agonist, a prostaglandin E receptor 2 (subtype EP2) selective agonist, and a mixed agonist for prostaglandin E receptor 4 (subtype EP4) and prostaglandin E receptor 2 (subtype EP2). In yet a further aspect, the prostaglandin E2 receptor agonist is a prostaglandin E receptor 4 (subtype EP4) agonist. In an even further aspect, the prostaglandin E receptor 4 (subtype EP4) agonist is selected from beraprost, nileprost, iloprost, cicaprost, eptaloprost, and ciprosten, or a pharmaceutically acceptable prodrug, salt, solvate, or polymorph thereof. In a still further aspect, the prostaglandin E receptor 4 (subtype EP4) agonist is beraprost. In yet a further aspect, the prostaglandin E receptor 4 (subtype EP4) agonist is nileprost.

In a further aspect, the method further comprises an interferon, or an isoform, mutein or fused protein thereof. In a still further aspect, the interferon is a pegylated interferon, a recombinant interferon, or a natural interferon. In yet a further aspect, the interferon is recombinant human interferon-beta, recombinant human interfone-beta which has a CHO cell-derived glycosylation, or consensus interferon-beta. In an even further aspect, the interferon is interferon is pegylated interferon-beta or interferon-beta Fc-fusion protein.

In a further aspect, the method further comprises an effective amount of an antiviral agent selected from a replication inhibitor, an IMP dehydrogenase inhibitor, an RNA polymerase inhibitor, and an influenza-specific interfering oligonucleotide. In a still further aspect, the IMP dehydrogenase inhibitor is selected from ribavirin, viramidine, merimepodib (VX-497), mycophenolic acid, mycophenolate mofetil, benzamide riboside, tiazofurin, mizoribine, and 3-deazaguanosine. In yet a further aspect, the IMP dehydrogenase inhibitor is selected from ribavirin, viramidine, merimepodib (VX-497), mycophenolic acid, and mycophenolate mofetil. In an even further aspect, the IMP dehydrogenase inhibitor is selected from ribavirin, viramidine, mycophenolic acid, and mycophenolate mofetil.

In a further aspect, the RNA polymerase inhibitor is favipiravir.

In a further aspect, the method further comprises an effective amount of an influenza virus absorption inhibitor selected from a hemagglutinin-specific antibody, a polyoxometalate, a sulfated polysaccharide, a sialidase fusion protein, and an O-glycoside of sialic acid. In a still further aspect, the influenza virus absorption inhibitor is a recombinant sialidase fusion protein. In yet a further aspect, the recombinant sialidase fusion protein is Fludase (DAS181).

In a further aspect, the method further comprises an effective amount of a cysteamine compound. In a still further aspect, the cysteamine compound is selected from cysteamine, cysteamine salts, prodrugs of cysteamine, analogs of cysteamine, derivatives of cysteamine, conjugates of cysteamine, metabolic precursors of cysteamine, and metabolites of cysteamine. In a still further aspect, the cysteamine salt is cysteamine hydrochloride. In yet a further aspect, the metabolic precursor of cysteamine is selected from cysteine, cystamine, and pantethine. In an even further aspect, the cysteamine metabolite is selected from taurine and hypotaurine.

In a further aspect, the method further comprises an effective amount of a therapeutic agent selected from an antitussive, a mucolytic, an expectorant, an antipyretic, an analgesic, and a nasal decongestant.

In a further aspect, the method further comprises an effective amount of an immunomodulator, or a pharmaceutically acceptable prodrug, salt, solvate, or polymorph thereof. In a still further aspect, an effective amount of an immunomodulator is an amount effective to reduce or inhibit one or more symptoms of inflammation of a subject.

In a further aspect, the immunomodulator is polyoxidonium. In a still further aspect, the immunomodulator is an anti-inflammatory agent. In yet a further aspect, the anti-inflammatory agent is non-steroidal, steroidal, or a combination thereof. In an even further aspect, the anti-inflammatory agent is a non-steroidal anti-inflammatory agent. In a still further aspect, the non-steroidal anti-inflammatory agent is selected from a COX2 inhibitor, an aminosalicylate drug, a PPAR ligand. In yet a further aspect, the non-steroidal anti-inflammatory agent is selected from an oxicam, a salicylate, an acetic acid derivative, a fenamate, a propionic acid derivative, and a pyrazole. In an even further aspect, the non-steroidal anti-inflammatory agent comprises one or more of piroxicam, isoxicam, tenoxicam, sudoxicam, aspirin, disalcid, benorylate, trilisate, safapryn, solprin, diflunisal, fendosal, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, mefenamic acid, meclofenamic acid, flufenamic acid, niflumic acid, tolfenamic acid, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofenic, phenylbutazone, oxyphenbutazone, feprazone, azapropazone, and trimethazone.

In a further aspect, the non-steroidal anti-inflammatory agent is a COX2 inhibitor. In a still further aspect, the COX2 inhibitor is celecoxib.

In a further aspect, the non-steroidal anti-inflammatory agent is an aminosalicylate. In a still further aspect, the aminosalicylate drug is selected from mesalazine and sulfasalazine.

In a further aspect, the non-steroidal anti-inflammatory agent is a PPAR ligand. In a further aspect, the PPAR ligand is a fibrate. In a still further aspect, the fibrate is selected from gemfibrozil, bezafibrate, ciprofibrate, clofibrate, and renofibrate, or combinations thereof.

In a further aspect, the anti-inflammatory agent is a steroidal anti-inflammatory agent. In a still further aspect, the steroidal anti-inflammatory agent is selected from hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, predisone, beclomethasone dipropionate, triamcinolone, and mixtures thereof.

In a further aspect, the treatment comprises prophylactic treatment.

In a further aspect, the infectious disease is associated with a bacterial infection. In a still further aspect, the bacterial infection comprises infection with a gram negative bacteria. In yet a further aspect, the gram negative bacteria is a Salmonella species. In an even further aspect, the Salmonella species is Salmonella typhimurium.

In a further aspect, the bacterial infection comprises infection with a non-gram negative, non-gram positive bacteria. In a still further aspect, the non-gram negative, non-gram positive bacteria is a Mycobacterium species. In yet a further aspect, the Mycobacterium species is Mycobacterium tuberculosis.

In a further aspect, the method further comprises co-administering the Akt therapeutic agent with at least one antituberculosis therapeutic agent selected from capreomycin, clofazimine, cycloserine, ethambutol, ethionamide, isoniazid, pyrazinamide, rifabutin, rifampin, and rifapentine. In a still further aspect, the antituberculosis therapeutic agent is selected from isoniazid, rifampin, ethambutol, and pyrazinamide.

In a further aspect, the method further comprises co-administering the Akt therapeutic agent with at least one antibacterial therapeutic agent selected from: a) an inhibitor of bacterial DNA synthesis, or a pharmaceutically acceptable prodrug, salt, solvate, or polymorph thereof b) an inhibitor of bacterial RNA synthesis, or a pharmaceutically acceptable prodrug, salt, solvate, or polymorph thereof c) an inhibitor of bacterial protein synthesis, or a pharmaceutically acceptable prodrug, salt, solvate, or polymorph thereof d) an bacterial antimetabolite agent, or a pharmaceutically acceptable prodrug, salt, solvate, or polymorph thereof; and e) an inhibitor of bacterial cell wall synthesis, or a pharmaceutically acceptable prodrug, salt, solvate, or polymorph thereof.

In a further aspect, the inhibitor of bacterial DNA synthesis is selected from ciprofloxacin, clofazimine, enoxacin, gatifloxacin, gemifloxacin, levofloxacin, lomefloxacin, metronidazole, moxifloxacin, nalidixic acid, norfloxacin, and ofloxacin.

In a further aspect, the inhibitor of bacterial RNA synthesis is selected from rifampin, rifabutin, rifapentine, and rifampin/isoniazid/pyrazinamide.

In a further aspect, the inhibitor of bacterial protein synthesis is selected from amikacin, azithromycin, capreomycin, chloramphenicol, clarithromycin, clindamycin, demeclocycline, dirithromycin, doxycycline, erythromycin, ethionamide, gentamicin, kanamycin, lincomycin, linezolid, minocycline, neomycin, puromycin, quinupristin/dalfopristin, roxithromycin, spectinomycin, telithromycin, tetracycline, tigecycline, and tobramycin.

In a further aspect, the bacterial antimetabolite agent is selected from aminosalicylic acid, furazolidinone, nitrofurantoin, introfurazone, sulfacetamide, sulfabenzamide, sulfanilamide, sulfisoxazole, sulfathiazole, trimethoprim/polymyxin B, trimethoprim/sulfamethoxazole, and trimetrexate.

In a further aspect, the inhibitor of bacterial cell wall synthesis is selected from ampicillin, ampicillin/sulbactam, amoxicillin, amoxicillin/clavulanate, aztreonam, bacampicillin, carbenicillin, cefaclor, cefadroxil, cefazolin, cefdinir, cefditoren, cefepime, cefixime, cefoperazone, cefotaxime, cefotetan, cefoxitin, cefprozil, cefpirome, cefpodoxime, ceftibuten, ceftriaxone, cefuroxime, cephalexin, cephradine, cycloserine, dicloxacillin, doripenem, ertapenem, ethambutol, fosfomycin, imipenem, imipenem/cilastatin, isoniazid, loracarbef, meropenem, methicillin, mezlocillin, nafcillin, oxacillin, penicillin G, penicillin V, penicillin W, piperacillin, pipercillin/tazobactam, ticarcillin, ticarcillin/clavulanate, and vancomycin.

In a further aspect, the method further comprises co-administering the Akt therapeutic agent with at least one antibacterial therapeutic agent selected from amikacin, amoxicillin, amoxicillin/clavulanate, aztreonam, azithromycin, cefaclor, cefadroxil, cephalexin, cefazolin, cefixime, cefotaxime, cefotetan, cefoxitin, cefpodoxime, ceftaroline fosamil, ceftazidime, ceftriaxone, cefuroxime, cephalexin, cephradine, chloramphenicol, cilastatin/imipenem, ciprofloxacin, clavulanate/ticarcillin, clarithromycin, clindamycin, clofazimine, colistin, daptomycin, demeclocycline, doripenem, doxycycline, ertapenem, fosfomycin/trometamol, fusidic acid, gentamicin, grepafloxacin, kanamycin, levofloxacin, lincomycin, linezolid, lymecycline, meropenem, metronidazole, minocycline, moxifloxacin, nafcillin, nalidixic acid, netilmicin, nitrofuratoin, norfloxacin, ofloxacin, oxacillin, oxytetracycline, penicillin, phenoxymethylpenicillin, piperacillin, pivmecillinam, polymyxin B, rifaximin, streptomycin, sulfadiazine, sulfamethoxazole/trimethoprim, sulfisoxazole, telithromycin, tetracycline, tobramycin, trimethoprim/sulfamethoxazole, vancomycin, LFF571, MK-3415, MK-3415A, and MK-6072.

In a further aspect, the method further comprises co-administration of an mTor inhibitor. In a still further aspect, the mTor inhibitor is selected from everolimus, rapamycin (sirolimus), temsirolimus, deforolimus, ridaforolimus, tacrolimus, zotarolimus, salirasib, curcumin, farnesylthiosalicylic acid, XL765, ABI-009, AP-23675, AP-23841, AP-23765, AZD-8055, AZD-2014, BEZ-235 (NVP-BEZ235), BGT226, GDC-0980, INK-128, KU-0063794, MK8669, MKC-1 (Ro 31-7453), NVP-BGT226, OSI-027, Palomid-529, PF-04691502, PKI-402, PKI-587, PP-242, PP-30, SB-1518, SB-2312, SF-1126, TAFA-93, TOP-216, Torin1, WAY-600, WYE-125132, WYE-354, WYE-687, and XL-765, or a pharmaceutically acceptable prodrug, salt, solvate, or polymorph thereof. In yet a further aspect, the mTor inhibitor is selected from everolimus, rapamycin (sirolimus), and temsirolimus, or a pharmaceutically acceptable prodrug, salt, solvate, or polymorph thereof. In an even further aspect, the mTor inhibitor is everolimus, or a pharmaceutically acceptable prodrug, salt, solvate, or polymorph thereof. In a still further aspect, the mTor inhibitor is rapamycin (sirolimus), or a pharmaceutically acceptable prodrug, salt, solvate, or polymorph thereof. In yet a further aspect, the mTor inhibitor is temsiorlimus, or a pharmaceutically acceptable prodrug, salt, solvate, or polymorph thereof. In an even further aspect, the mTor inhibitor is deforolimus, or a pharmaceutically acceptable prodrug, salt, solvate, or polymorph thereof. In a still further aspect, the mTor inhibitor is tacrolimus, or a pharmaceutically acceptable prodrug, salt, solvate, or polymorph thereof. In yet a further aspect, the mTor inhibitor is zotarolimus, or a pharmaceutically acceptable prodrug, salt, solvate, or polymorph thereof. In an even further aspect, the mTor inhibitor is salirasib, or a pharmaceutically acceptable prodrug, salt, solvate, or polymorph thereof. In a still further aspect, the mTor inhibitor is curcumin, or a pharmaceutically acceptable prodrug, salt, solvate, or polymorph thereof. In yet a further aspect, the mTor inhibitor is farnesylthiosalicylic acid, or a pharmaceutically acceptable prodrug, salt, solvate, or polymorph thereof. In an even further aspect, the mTor inhibitor is Torin1, or a pharmaceutically acceptable prodrug, salt, solvate, or polymorph thereof.

In a further aspect, the method further comprises co-administration of an effective amount of a PLD inhibitor. In a still further aspect, the PLD inhibitor is a compound having a structure represented by a formula:

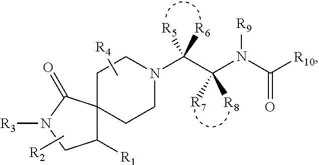

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof. In yet a further aspect, the compound has a structure represented by a formula:

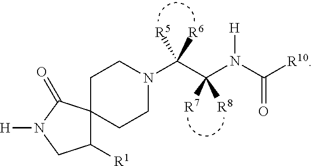

In a further aspect, the PLD inhibitor is a compound having a structure represented by a formula:

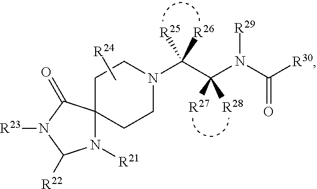

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof. In a still further aspect, the compound has a structure represented by a formula:

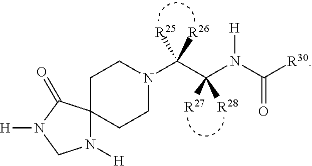

In a further aspect, the compound is:

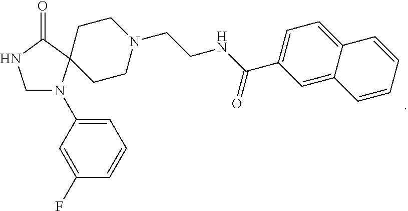 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

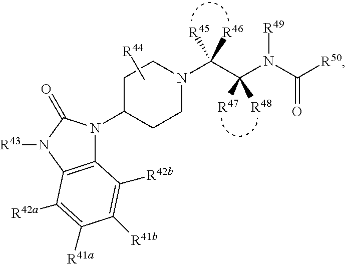

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof. In a still further aspect, the compound has a structure represented by a formula:

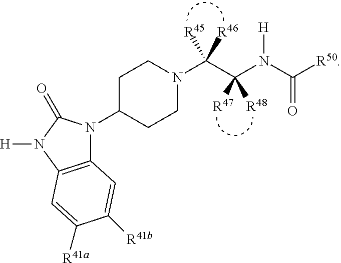

In a further aspect, the compound is:

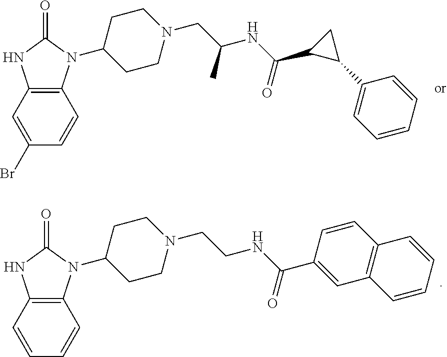

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof.

In a further aspect, the PLD inhibitor is a compound selected from:

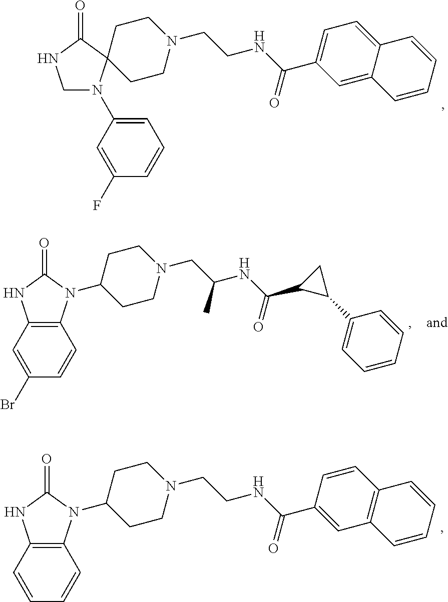

or a pharmaceutically acceptable salt, hydrate, solvate, or polymorph thereof.

In a further aspect, the effective amount of a phospholipase D inhibitor inhibits HIV replication. In a still further aspect, the effective amount of a phospholipase D inhibitor inhibits HIV integration.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, an effective amount of the Akt therapeutic agent is a therapeutically effective amount. In a still further aspect, an effective amount of the Akt therapeutic agent is a prophylactically effective amount.

In a further aspect, the Akt therapeutic agent is an Akt inhibitor. In a still further aspect, the Akt inhibitor binds to the pleckstrin homology domain.

In a further aspect, the Akt inhibitor is an ATP-competitive inhibitor.

In a further aspect, the Akt inhibitor is an allosteric inhibitor. In a still further aspect, the allosteric inhibitor is MK-2066.

In a further aspect, the Akt inhibitor is a pan-Akt inhibitor.

In a further aspect, the Akt inhibitor inhibits Akt1, Akt2, or Akt3.

In a further aspect, the Akt inhibitor is an isoform-selective inhibitor. In a still further aspect, the isoform-selective inhibitor selectively inhibits Akt1.

In a further aspect, the Akt therapeutic agent is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine. In a still further aspect, the Akt therapeutic agent is erucylphosphocholine. In yet a further aspect, the Akt therapeutic agent is GDC-0068. In an even further aspect, the Akt therapeutic agent is GSK-2141795. In a still further aspect, the Akt therapeutic agent is MK-2206. In yet a further aspect, the Akt therapeutic agent is perifosine. In an even further aspect, the Akt therapeutic agent is PHT-427.

In a further aspect, the Akt therapeutic agent is a siRNA.

In a further aspect, the Akt therapeutic agent is an antisense oligonucleotide. In a still further aspect, the antisense oligonucleotide is RX-0201.

b. Treating a Viral Infection

In one aspect, the invention relates to a method for treating a subject for a viral infection comprising the step of co-administering to the subject an effective amount of: a) a phospholipase D inhibitor, or a pharmaceutically acceptable prodrug, salt, solvate, or polymorph thereof; and b) a mTor inhibitor, or a pharmaceutically acceptable prodrug, salt, solvate, or polymorph thereof.

In a further aspect, the subject has been diagnosed with a need for treatment of the viral infection prior to the administering step. In a still further aspect, the method further comprises the step of identifying a subject in need of treatment of the viral infection.

In a further aspect, the PLD inhibitor is a compound having a structure represented by a formula:

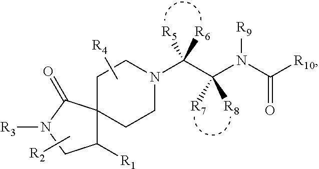

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

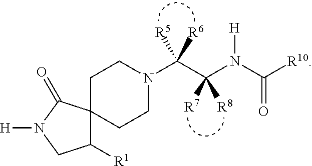

In a further aspect, the PLD inhibitor is a compound having a structure represented by a formula:

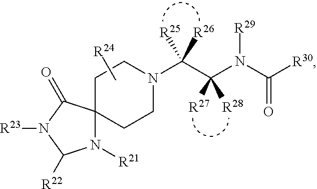

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

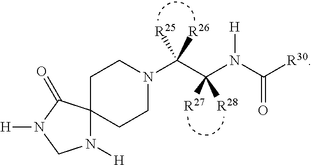

In a further aspect, the compound is:

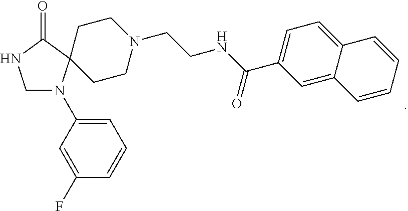 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

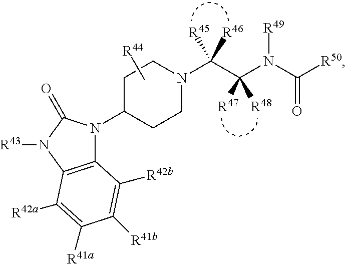

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

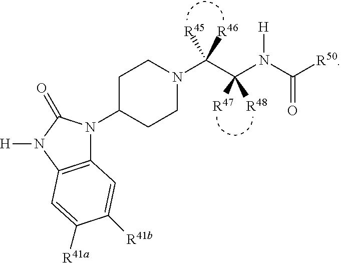

In a further aspect, the compound is:

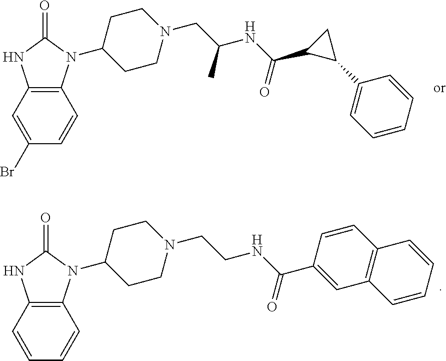

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof.

In a further aspect, the PLD inhibitor is a compound selected from:

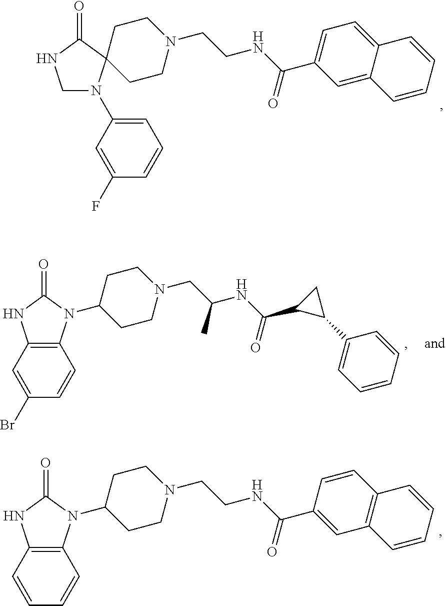

or a pharmaceutically acceptable salt, hydrate, solvate, or polymorph thereof.

In a further aspect, the effective amount of a phospholipase D inhibitor inhibits HIV replication. In a still further aspect, the effective amount of a phospholipase D inhibitor inhibits HIV integration.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the phospholipase D inhibitor is selected from:

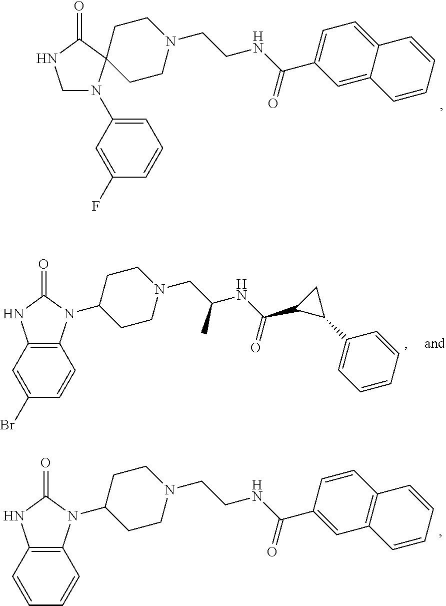

or a pharmaceutically acceptable salt, hydrate, solvate, or polymorph thereof.

In a further aspect, the mTor inhibitor is selected from everolimus, rapamycin (sirolimus), temsirolimus, deforolimus, ridaforolimus, tacrolimus, zotarolimus, salirasib, curcumin, farnesylthiosalicylic acid, XL765, ABI-009, AP-23675, AP-23841, AP-23765, AZD-8055, AZD-2014, BEZ-235 (NVP-BEZ235), BGT226, GDC-0980, INK-128, KU-0063794, MK8669, MKC-1 (Ro 31-7453), NVP-BGT226, OSI-027, Palomid-529, PF-04691502, PKI-402, PKI-587, PP-242, PP-30, SB-1518, SB-2312, SF-1126, TAFA-93, TOP-216, Torin1, WAY-600, WYE-125132, WYE-354, WYE-687, and XL-765, or a pharmaceutically acceptable prodrug, salt, solvate, or polymorph thereof. In a still further aspect, the mTor inhibitor is selected from everolimus, rapamycin (sirolimus), and temsirolimus, or a pharmaceutically acceptable prodrug, salt, solvate, or polymorph thereof. In yet a further aspect, the mTor inhibitor is everolimus, or a pharmaceutically acceptable prodrug, salt, solvate, or polymorph thereof. In an even further aspect, the mTor inhibitor is rapamycin (sirolimus), or a pharmaceutically acceptable prodrug, salt, solvate, or polymorph thereof. In a still further aspect, the mTor inhibitor is temsiorlimus, or a pharmaceutically acceptable prodrug, salt, solvate, or polymorph thereof. In yet a further aspect, the mTor inhibitor is deforolimus, or a pharmaceutically acceptable prodrug, salt, solvate, or polymorph thereof. In an even further aspect, the mTor inhibitor is tacrolimus, or a pharmaceutically acceptable prodrug, salt, solvate, or polymorph thereof. In a still further aspect, the mTor inhibitor is zotarolimus, or a pharmaceutically acceptable prodrug, salt, solvate, or polymorph thereof. In yet a further aspect, the mTor inhibitor is salirasib, or a pharmaceutically acceptable prodrug, salt, solvate, or polymorph thereof. In an even further aspect, the mTor inhibitor is curcumin, or a pharmaceutically acceptable prodrug, salt, solvate, or polymorph thereof. In a still further aspect, the mTor inhibitor is farnesylthiosalicylic acid, or a pharmaceutically acceptable prodrug, salt, solvate, or polymorph thereof. In yet a further aspect, the mTor inhibitor is Torin1, or a pharmaceutically acceptable prodrug, salt, solvate, or polymorph thereof.

In a further aspect, the method further comprises an effective amount of an Akt therapeutic agent. In a still further aspect, the Akt therapeutic agent is an Akt inhibitor.

In a further aspect, the Akt inhibitor binds to the pleckstrin homology domain.

In a further aspect, the Akt inhibitor is an ATP-competitive inhibitor.

In a further aspect, the Akt inhibitor is an allosteric inhibitor. In a still further aspect, the allosteric inhibitor is MK-2066.

In a further aspect, the Akt inhibitor is a pan-Akt inhibitor.

In a further aspect, the Akt inhibitor is an isoform-selective inhibitor. In a still further aspect, the Akt inhibitor inhibits Akt1, Akt2, or Akt3. In yet a further aspect, the isoform-selective inhibitor selectively inhibits Akt1.

In a further aspect, the Akt therapeutic agent is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine. In a still further aspect, the Akt inhibitor is erucylphosphocholine. In yet a further aspect, the Akt inhibitor is GDC-0068. In an even further aspect, the Akt inhibitor is GSK-2141795. In a still further aspect, the Akt inhibitor is MK-2206. In yet a further aspect, the Akt inhibitor is perifosine. In an even further aspect, the Akt inhibitor is PHT-427.

In a further aspect, the Akt therapeutic agent is a siRNA.

In a further aspect, the Akt therapeutic agent is an antisense oligonucleotide. In a still further aspect, the antisense oligonucleotide is RX-0201.

In a further aspect, the viral infection comprises infection with HIV. In a still further aspect, the subject has been diagnosed with a need for treatment of the HIV infection prior to the administering step. In yet a further aspect, the method further comprises the step of identifying a subject in need of treatment of the HIV infection.

In a further aspect, the HIV infection comprises a HIV-1 serotype virus. In a still further aspect, the HIV-1 infection comprises a Group M, Group N, Group O, or Group P virus strain. In yet a further aspect, the HIV-1 infection comprises a Group M virus strain. In an even further aspect, the HIV-1 Group M virus strain is selected from the subtypes A, B, C, D, F, G, H, J, and K. In a still further aspect, the HIV-1 Group M virus strain subtype is subtype A. In yet a further aspect, the HIV-1 Group M virus strain subtype is subtype B. In an even further aspect, the HIV-1 Group M virus strain subtype is subtype C. In a still further aspect, the HIV-1 Group M virus strain subtype is subtype D. In yet a further aspect, the HIV-1 Group M virus strain subtype is subtype H.

In a further aspect, the HIV-1 Group M virus strain subtype comprises a circulating recombinant form (“CRF”) comprising genetic material from one or more subtypes selected from subtypes A, B, C, D, F, G, H, J, and K. In a still further aspect, the circulating recombinant form is CRF A/E. In yet a further aspect, the circulating recombinant form is CRF A/G.

In a further aspect, the HIV infection comprises a HIV-2 serotype virus.

In a further aspect, the HIV infection is associated with a disease selected from AIDS, aspergillosis, atypical mycobacteriosis, bacillary angiomatosis, bacteremia, bacterial pneumonia, bacterial sinusitis, candidiasis, CMV, CMV retinitis, coccidioidomycosis, cryptococcosis, cryptosporidiosis-isosporiasis, non-specific enteritis, folliculitis, herpes, histoplasmosis, HIV dementia, HIV meningitis, leismaniasis, Mycobacterium avium complex disease, nocardiosis, pencilliosis, progressive multifocal leukoencephalopathy (PML; or HIV encephalitis), Pneumocystis carinii pneumonia (PCP), pneumonia, Pseudomonas pneumonia, toxoplasma encephalitis, toxoplasmosis, tuberculosis, Kaposi sarcoma, lymphoma, and squamous cell carcinoma. In a still further aspect, the lymphoma is selected from Non-Hodgkin's lymphoma, CNS lymphoma, primary lymphoma of the brain, and systemic lymphoma.

In a further aspect, the HIV infection is associated with a cancer. In a still further aspect, the cancer is selected from a lymphoma, sarcoma, and a carcinoma. In yet a further aspect, the carcinoma is a squamous cell carcinoma. In an even further aspect, the sarcoma is Kaposi sarcoma. In a still further aspect, the lymphoma is selected from Non-Hodgkin's lymphoma, CNS lymphoma, primary lymphoma of the brain, and systemic lymphoma.

In a further aspect, the HIV infection is associated with an opportunistic infection. In a still further aspect, the opportunistic infection is selected from aspergillosis, atypical mycobacteriosis, bacillary angiomatosis, bacteremia, bacterial pneumonia, bacterial sinusitis, candidiasis, CMV retinitis, coccidioidomycosis, cryptococcosis, cryptosporidiosis-isosporiasis, non-specific enteritis, folliculitis, herpes, histoplasmosis, HIV dementia, HIV meningitis, leismaniasis, Mycobacterium avium complex disease, nocardiosis, pencilliosis, progressive multifocal leukoencephalopathy (PML; or HIV encephalitis), Pneumocystis carinii pneumonia (PCP), pneumonia, Pseudomonas pneumonia, toxoplasma encephalitis, toxoplasmosis, and tuberculosis. In yet a further aspect, the HIV infection is associated with an infection associated with Cryptosporidium muris, Isospora belli, Toxoplasma gondii, Candida sp., Coccidioides immitis, Histoplasma capsulatum, Pneumocystis carnii, Mycobacterium avium complex, Mycobacterium tuberculosis, Cytomegalovirus, Epstein-Barr virus, Herpes simplex virus, Papovirus J-C, or Varicella-zoster.

In a further aspect, the subject has been diagnosed with a need for treatment of an HIV infection prior to the administering step. In a still further aspect, the method further comprises the step of identifying a subject in need of treatment of the HIV infection.

In a further aspect, the HIV infection comprises an HIV virus that is resistant to treatment with an HIV non-nucleoside reverse transcriptase inhibitor. In a still further aspect, the HIV virus resistant to treatment with a non-nucleoside reverse transcriptase inhibitor has at least one mutation in the HIV reverse transcriptase. In yet a further aspect, the at least one mutation in the HIV reverse transcriptase is selected from 100I, 103N, 106A, 106M, 108I, 181C, 181I, 188C, 188H, 188L, 190A, 190S, 225H, 230L, and 236L. In an even further aspect, the at least one mutation is at amino acid position 100, 103, 106, 108, 181, 188, 190, 225, 230, or 236 of the HIV reverse transcriptase.

In a further aspect, the HIV non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In a still further aspect, the HIV non-nucleoside reverse transcriptase inhibitor is delavirdine, or a pharmaceutically acceptable prodrug, salt, solvate, or polymorph thereof. In yet a further aspect, the HIV non-nucleoside reverse transcriptase inhibitor is efavirenz, or a pharmaceutically acceptable prodrug, salt, solvate, or polymorph thereof. In an even further aspect, the HIV non-nucleoside reverse transcriptase inhibitor is etravirine, or a pharmaceutically acceptable prodrug, salt, solvate, or polymorph thereof. In a still further aspect, the HIV non-nucleoside reverse transcriptase inhibitor is nevirapine, or a pharmaceutically acceptable prodrug, salt, solvate, or polymorph thereof. In yet a further aspect, the HIV non-nucleoside reverse transcriptase inhibitor is rilpivirine, or a pharmaceutically acceptable prodrug, salt, solvate, or polymorph thereof. In an even further aspect, the HIV non-nucleoside reverse transcriptase inhibitor is lersivirine, or a pharmaceutically acceptable prodrug, salt, solvate, or polymorph thereof.

In a further aspect, the HIV infection comprises an HIV virus that is resistant to treatment with an HIV nucleoside reverse transcriptase inhibitor. In a still further aspect, the HIV virus resistant to treatment with a nucleoside reverse transcriptase inhibitor has at least one mutation in the HIV reverse transcriptase. In yet a further aspect, the at least one mutation in the HIV reverse transcriptase is selected from 41L, 44D, 62V, 65R, 67N, 69A, 69D, 69N, 69S, 69 insertion, 70R, 74V, 75I, 77L, 115F, 116Y, 118I, 151M, 184I, 184V, 210W, 215C, 215D, 215E, 215F, 215I, 215S, 215Y, 219E, and 219Q. In an even further aspect, the at least one mutation is at amino acid position 41, 44, 62, 65, 67, 69, 70, 74, 77, 115, 116, 118, 151, 184, 210, 215 or 219 of the HIV reverse transcriptase.

In a further aspect, the HIV nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In a still further aspect, the HIV nucleoside reverse transcriptase inhibitor is selected from abacavir, didansine, emtricitabine, lamivudine, stavudine, tenofovir, zidovudine, and elvucitabine, or a pharmaceutically acceptable prodrug, salt, solvate, or polymorph thereof. In yet a further aspect, the HIV nucleoside reverse transcriptase inhibitor is abacavir, or a pharmaceutically acceptable prodrug, salt, solvate, or polymorph thereof. In an even further aspect, the HIV nucleoside reverse transcriptase inhibitor is didansine, or a pharmaceutically acceptable prodrug, salt, solvate, or polymorph thereof. In a still further aspect, the HIV nucleoside reverse transcriptase inhibitor is emtricitabine, or a pharmaceutically acceptable prodrug, salt, solvate, or polymorph thereof. In yet a further aspect, the HIV nucleoside reverse transcriptase inhibitor is lamivudine, or a pharmaceutically acceptable prodrug, salt, solvate, or polymorph thereof. In an even further aspect, the HIV nucleoside reverse transcriptase inhibitor is stavudine, or a pharmaceutically acceptable prodrug, salt, solvate, or polymorph thereof. In a still further aspect, the HIV nucleoside reverse transcriptase inhibitor is tenofovir, or a pharmaceutically acceptable prodrug, salt, solvate, or polymorph thereof. In yet a further aspect, the HIV nucleoside reverse transcriptase inhibitor is zidovudine, or a pharmaceutically acceptable prodrug, salt, solvate, or polymorph thereof. In an even further aspect, the HIV nucleoside reverse transcriptase inhibitor is elvucitabine, or a pharmaceutically acceptable prodrug, salt, solvate, or polymorph thereof.

In a further aspect, the HIV infection comprises an HIV virus that is resistant to treatment with a protease inhibitor. In a still further aspect, the HIV virus resistant to treatment with a protease inhibitor has at least one mutation in the HIV protease. In yet a further aspect, the at least one mutation in the HIV protease is selected from 30N, 46I, 46L, 48V, 50V, 82A, 82F, 82S, 82T, 84V, and 90M. In an even further aspect, the at least one mutation is at amino acid position 30, 46, 48, 50, 82, 84, or 90 of the HIV protease.

In a further aspect, the HIV protease inhibitor is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In a still further aspect, the HIV protease inhibitor is atazanavir, or a pharmaceutically acceptable prodrug, salt, solvate, or polymorph thereof. In yet a further aspect, the HIV protease inhibitor is darunavir, or a pharmaceutically acceptable prodrug, salt, solvate, or polymorph thereof. In an even further aspect, the HIV protease inhibitor is fosamprenavir, or a pharmaceutically acceptable prodrug, salt, solvate, or polymorph thereof. In a still further aspect, the HIV protease inhibitor is indinavir, or a pharmaceutically acceptable prodrug, salt, solvate, or polymorph thereof. In yet a further aspect, the HIV protease inhibitor is lopinavir, or a pharmaceutically acceptable prodrug, salt, solvate, or polymorph thereof. In an even further aspect, the HIV protease inhibitor is nelfinavir, or a pharmaceutically acceptable prodrug, salt, solvate, or polymorph thereof. In a still further aspect, the HIV protease inhibitor is ritonavir, or a pharmaceutically acceptable prodrug, salt, solvate, or polymorph thereof. In yet a further aspect, the HIV protease inhibitor is saquinavir, or a pharmaceutically acceptable prodrug, salt, solvate, or polymorph thereof. In an even further aspect, the HIV protease inhibitor is tipranavir, or a pharmaceutically acceptable prodrug, salt, solvate, or polymorph thereof. In a still further aspect, the HIV protease inhibitor is lopinavir/ritonavir, or a pharmaceutically acceptable prodrug, salt, solvate, or polymorph thereof.

In a further aspect, the HIV infection comprises an HIV virus that is resistant to treatment with an HIV integrase inhibitor. In a still further aspect, the HIV integrase inhibitor is selected from raltegravir, dolutegravir, elvitegravir, and S/GSK1265744, or a pharmaceutically acceptable prodrug, salt, solvate, or polymorph thereof. In yet a further aspect, the HIV integrase inhibitor is selected from raltegravir, dolutegravir, and elvitegravir, or a pharmaceutically acceptable prodrug, salt, solvate, or polymorph thereof. In an even further aspect, the HIV integrase inhibitor is raltegravir, or a pharmaceutically acceptable prodrug, salt, solvate, or polymorph thereof. In a still further aspect, the HIV integrase inhibitor is dolutegravir, or a pharmaceutically acceptable prodrug, salt, solvate, or polymorph thereof. In yet a further aspect, the HIV integrase inhibitor is elvitegravir, or a pharmaceutically acceptable prodrug, salt, solvate, or polymorph thereof.

In a further aspect, the HIV infection comprises an HIV virus that is resistant to treatment with an HIV fusion inhibitor. In a still further aspect, the HIV fusion inhibitor is selected from enfuvirtide, maraviroc, cenicriviroc, ibalizumab, BMS-663068, and PRO-140, or a pharmaceutically acceptable prodrug, salt, solvate, or polymorph thereof. In yet a further aspect, the HIV fusion inhibitor is selected from enfuvirtide, maraviroc, cenicriviroc, and ibalizumab, or a pharmaceutically acceptable prodrug, salt, solvate, or polymorph thereof. In an even further aspect, the HIV fusion inhibitor is enfuvirtide, or a pharmaceutically acceptable prodrug, salt, solvate, or polymorph thereof. In a still further aspect, the HIV fusion inhibitor is maraviroc, or a pharmaceutically acceptable prodrug, salt, solvate, or polymorph thereof. In yet a further aspect, the HIV fusion inhibitor is cenicriviroc, or a pharmaceutically acceptable prodrug, salt, solvate, or polymorph thereof. In an even further aspect, the HIV fusion inhibitor is ibalizumab, or a pharmaceutically acceptable prodrug, salt, solvate, or polymorph thereof.

In a further aspect, the method further comprises co-administering the Akt therapeutic agent with an effective amount of at least one HIV therapeutic agent selected from: a) a HIV fusion/lysis inhibitor, or a pharmaceutically acceptable prodrug, salt, solvate, or polymorph thereof; b) a HIV integrase inhibitor, or a pharmaceutically acceptable prodrug, salt, solvate, or polymorph thereof; c) a HIV non-nucleoside reverse transcriptase inhibitor, or a pharmaceutically acceptable prodrug, salt, solvate, or polymorph thereof; d) a HIV nucleoside reverse transcriptase inhibitor, or a pharmaceutically acceptable prodrug, salt, solvate, or polymorph thereof; and e) a HIV protease inhibitor, or a pharmaceutically acceptable prodrug, salt, solvate. In a still further aspect, the effective amount of the least one HIV therapeutic agent is a therapeutically effective amount. In yet a further aspect, the effective amount of the least one HIV therapeutic agent is a prophylactically effective amount.

In a further aspect, the HIV fusion/lysis inhibitor is selected from enfuvirtide, maraviroc, cenicriviroc, ibalizumab, BMS-663068, and PRO-140, or a pharmaceutically acceptable prodrug, salt, solvate, or polymorph thereof. In a still further aspect, the HIV fusion/lysis inhibitor is selected from enfuvirtide, maraviroc, cenicriviroc, and ibalizumab, or a pharmaceutically acceptable prodrug, salt, solvate, or polymorph thereof. In yet a further aspect, the HIV fusion/lysis inhibitor is enfuvirtide, or a pharmaceutically acceptable prodrug, salt, solvate, or polymorph thereof. In an even further aspect, the HIV fusion/lysis inhibitor is maraviroc, or a pharmaceutically acceptable prodrug, salt, solvate, or polymorph thereof. In a still further aspect, the HIV fusion/lysis inhibitor is cenicriviroc, or a pharmaceutically acceptable prodrug, salt, solvate, or polymorph thereof. In yet a further aspect, the HIV fusion/lysis inhibitor is ibalizumab, or a pharmaceutically acceptable prodrug, salt, solvate, or polymorph thereof.

In a further aspect, the HIV integrase inhibitor is selected from raltegravir, dolutegravir, elvitegravir, and S/GSK1265744, or a pharmaceutically acceptable prodrug, salt, solvate, or polymorph thereof. In a still further aspect, the HIV integrase inhibitor is selected from raltegravir, dolutegravir, and elvitegravir, or a pharmaceutically acceptable prodrug, salt, solvate, or polymorph thereof. In yet a further aspect, the HIV integrase inhibitor is raltegravir, or a pharmaceutically acceptable prodrug, salt, solvate, or polymorph thereof. In an even further aspect, the HIV integrase inhibitor is dolutegravir, or a pharmaceutically acceptable prodrug, salt, solvate, or polymorph thereof. In a still further aspect, the HIV integrase inhibitor is elvitegravir, or a pharmaceutically acceptable prodrug, salt, solvate, or polymorph thereof.

In a further aspect, the HIV non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In a still further aspect, the HIV non-nucleoside reverse transcriptase inhibitor is delavirdine, or a pharmaceutically acceptable prodrug, salt, solvate, or polymorph thereof. In yet a further aspect, the HIV non-nucleoside reverse transcriptase inhibitor is efavirenz, or a pharmaceutically acceptable prodrug, salt, solvate, or polymorph thereof. In an even further aspect, the HIV non-nucleoside reverse transcriptase inhibitor is etravirine, or a pharmaceutically acceptable prodrug, salt, solvate, or polymorph thereof. In a still further aspect, the HIV non-nucleoside reverse transcriptase inhibitor is nevirapine, or a pharmaceutically acceptable prodrug, salt, solvate, or polymorph thereof. In yet a further aspect, the HIV non-nucleoside reverse transcriptase inhibitor is rilpivirine, or a pharmaceutically acceptable prodrug, salt, solvate, or polymorph thereof.

In a further aspect, the HIV nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In a still further aspect, the HIV nucleoside reverse transcriptase inhibitor is selected from abacavir, didansine, elvucitabine, emtricitabine, lamivudine, stavudine, tenofovir, and zidovudine, or a pharmaceutically acceptable prodrug, salt, solvate, or polymorph thereof. In yet a further aspect, the HIV nucleoside reverse transcriptase inhibitor is abacavir, or a pharmaceutically acceptable prodrug, salt, solvate, or polymorph thereof. In an even further aspect, the HIV nucleoside reverse transcriptase inhibitor is didansine, or a pharmaceutically acceptable prodrug, salt, solvate, or polymorph thereof. In a still further aspect, the HIV nucleoside reverse transcriptase inhibitor is elvucitabine, or a pharmaceutically acceptable prodrug, salt, solvate, or polymorph thereof. In yet a further aspect, the HIV nucleoside reverse transcriptase inhibitor is emtricitabine, or a pharmaceutically acceptable prodrug, salt, solvate, or polymorph thereof. In an even further aspect, the HIV nucleoside reverse transcriptase inhibitor is lamivudine, or a pharmaceutically acceptable prodrug, salt, solvate, or polymorph thereof. In a still further aspect, the HIV nucleoside reverse transcriptase inhibitor is stavudine, or a pharmaceutically acceptable prodrug, salt, solvate, or polymorph thereof. In yet a further aspect, the HIV nucleoside reverse transcriptase inhibitor is tenofovir, or a pharmaceutically acceptable prodrug, salt, solvate, or polymorph thereof. In an even further aspect, the HIV nucleoside reverse transcriptase inhibitor is zidovudine, or a pharmaceutically acceptable prodrug, salt, solvate, or polymorph thereof.

In a further aspect, the HIV protease inhibitor is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In a still further aspect, the HIV protease inhibitor is atazanir, or a pharmaceutically acceptable prodrug, salt, solvate, or polymorph thereof. In yet a further aspect, the HIV protease inhibitor is darunavir, or a pharmaceutically acceptable prodrug, salt, solvate, or polymorph thereof. In an even further aspect, the HIV protease inhibitor is fosamprenavir, or a pharmaceutically acceptable prodrug, salt, solvate, or polymorph thereof. In a still further aspect, the HIV protease inhibitor is indinavir, or a pharmaceutically acceptable prodrug, salt, solvate, or polymorph thereof. In yet a further aspect, the HIV protease inhibitor is lopinavir, or a pharmaceutically acceptable prodrug, salt, solvate, or polymorph thereof. In an even further aspect, the HIV protease inhibitor is nelfinavir, or a pharmaceutically acceptable prodrug, salt, solvate, or polymorph thereof. In a still further aspect, the HIV protease inhibitor is ritonavir, or a pharmaceutically acceptable prodrug, salt, solvate, or polymorph thereof. In yet a further aspect, the HIV protease inhibitor is saquinavir, or a pharmaceutically acceptable prodrug, salt, solvate, or polymorph thereof. In an even further aspect, the HIV protease inhibitor is tipranavir, or a pharmaceutically acceptable prodrug, salt, solvate, or polymorph thereof.

In a further aspect, the viral infection comprises an infection with an influenza virus. In a still further aspect, the subject has been diagnosed with a need for treatment of the influenza infection prior to the administering step. In yet a further aspect, the method further comprises the step of identifying a subject in need of treatment of the influenza infection.

In a further aspect, the subject is a bird. In a still further aspect, the subject is a mammal. In yet a further aspect, the mammal is selected from a human, a swine, a horse, a cat, and a dog. In an even further aspect, the mammal is a human.

In a further aspect, the influenza virus is selected from a type A influenza virus, type B influenza virus, and type C influenza virus. In a still further aspect, the virus is a type A influenza virus. In yet a further aspect, the type A influenza virus is of subtype H1, H5, H7 or H9. In an even further aspect, the type A influenza virus is of subtype H1N1, H1N2, H2N2, H3N2, H3N8, H5N1, H5N2, H5N3, H5N8, H5N9, H7N1, H7N2, H7N3, H7N4, H7N7, H7N9, H9N2, or H10N7. In a still further aspect, the type A influenza virus is of subtype H1N1, H1N2, H2N2, H3N2, H5N1, H5N3, H7N2, H7N3, H7N7, H9N2, or H10N7. In yet a further aspect, the type A influenza virus is H5N1. In an even further aspect, the type A influenza virus is H1N1. In a yet further aspect, the type A influenza virus is H7N9.

In a further aspect, the virus is a type B influenza virus.

In a further aspect, the virus is a type C influenza virus.

In a further aspect, the virus is oseltamivir resistant. In a still further aspect, the virus is not oseltamivir resistant.

In a further aspect, the virus is amantadine resistant. In a still further aspect, the virus is not amantadine resistant.

In a further aspect, the virus is rimantadine resistant. In a still further aspect, the virus is not rimantadine resistant.

In a further aspect, the method further comprises co-administering at least one influenza therapeutic agent selected from: a) a viral protein M2 ion channel inhibitor, or a pharmaceutically acceptable prodrug, salt, solvate, or polymorph thereof; b) a neuraminidase inhibitor, or a pharmaceutically acceptable prodrug, salt, solvate, or polymorph thereof; and c) a nucleoside analog, or a pharmaceutically acceptable prodrug, salt, solvate, or polymorph thereof. In a still further aspect, the effective amount of the at least one influenza therapeutic agent is a therapeutically effective amount. In yet a further aspect, the effective amount of the at least one influenza therapeutic agent is a prophylactically effective amount.

In a further aspect, co-administration is administration in a substantially simultaneous manner. In a still further aspect, co-administration is administration in a substantially sequential manner.

In a further aspect, the viral protein M2 ion channel inhibitor is an amino-adamantane compound. In a still further aspect, the amino-adamantane compound is selected from 1-amino-adamantane and 1-(1-aminoethyl)adamantane.

In a further aspect, the viral protein M2 ion channel inhibitor is selected from amantadine and rimantadine, or a pharmaceutically acceptable prodrug, salt, solvate, or polymorph thereof. In a still further aspect, the viral protein M2 ion channel inhibitor is an analog of amantadine or rimantadine. In yet a further aspect, the amantadine analog is selected from 1-amino-1,3,5-trimethylcyclohexane, 1-amino-1(trans),3(trans),5-trimethylcyclohexane, 1-amino-1(cis),3(cis),5-trimethylcyclohexane, 1-amino-1,3,3,5-tetramethylcyclohexane, 1-amino-1,3,3,5,5-pentamethylcyclohexane(neramexane), 1-amino-1,3,5,5-tetramethyl-3-ethylcyclohexane, 1-amino-1,5,5-trimethyl-3,3-diethylcyclohexane, 1-amino-1,5,5-trimethyl-cis-3-ethylcyclohexane, 1-amino-(1S,5S)cis-3-ethyl-1,5,5-trimethylcyclohexane, 1-amino-1,5,5-trimethyl-trans-3-ethylcyclohexane, 1-amino-(1R,5S)trans-3-ethyl-1,5,5-trimethylcyclohexane, 1-amino-1-ethyl-3,3,5,5-tetramethylcyclohexane, 1-amino-1-propyl-3,3,5,5-tetramethylcyclohexane, N-methyl-1-amino-1,3,3,5,5-pentamethylcyclohexane, N-ethyl-1-amino-1,3,3,5,5-pentamethyl-cyclohexane, N-(1,3,3,5,5-pentamethylcyclohexyl)pyrrolidine, 3,3,5,5-tetramethylcyclohexylmethylamine, 1-amino-1-propyl-3,3,5,5-tetramethylcyclohexane, 1 amino-1,3,3,5 (trans)-tetramethylcyclohexane (axial amino group), 3-propyl-1,3,5,5-tetramethylcyclohexylamine semihydrate, 1-amino-1,3,5,5-tetramethyl-3-ethylcyclohexane, 1-amino-1,3,5-trimethylcyclohexane, 1-amino-1,3-dimethyl-3-propylcyclohexane, 1-amino-1,3 (trans),5(trans)-trimethyl-3 (cis)-propylcyclohexane, 1-amino-1,3-dimethyl-3-ethylcyclohexane, 1-amino-1,3,3-trimethylcyclohexane, cis-3-ethyl-1(trans)-3 (trans)-5-trimethylcyclohexamine, 1-amino-1,3(trans)-dimethylcyclohexane, 1,3,3-trimethyl-5,5-dipropylcyclohexylamine, 1-amino-1-methyl-3 (trans)-propylcyclohexane, 1-methyl-3 (cis)-propylcyclohexylamine, 1-amino-1-methyl-3(trans)-ethylcyclohexane, 1-amino-1,3,3-trimethyl-5(cis)-ethylcyclohexane, 1-amino-1,3,3-trimethyl-5(trans)-ethylcyclohexane, cis-3-propyl-1,5,5-trimethylcyclohexylamine, trans-3-propyl-1,5,5-trimethylcyclohexylamine, N-ethyl-1,3,3,5,5-pentamethylcyclohexylamine, N-methyl-1-amino-1,3,3,5,5-pentamethylcyclohexane, 1-amino-1-methylcyclohexane, N,N-dimethyl-1-amino-1,3,3,5,5-pentamethylcyclohexane, 2-(3,3,5,5-tetramethylcyclohexyl)ethylamine, 2-methyl-1-(3,3,5,5-tetramethylcyclohexyl)propyl-2-amine, 2-(1,3,3,5,5-pentamethylcyclohexyl-1)-ethylamine semihydrate, N-(1,3,3,5,5-pentamethylcyclohexyl)-pyrrolidine, 1-amino-1,3(trans),5(trans)-trimethylcyclohexane, 1-amino-1,3 (cis),5 (cis)-trimethylcyclohexane, 1-amino-(1R,5S)trans-5-ethyl-1,3,3-trimethylcyclohexane, 1-amino-(1S,5S)cis-5-ethyl-1,3,3-trimethylcyclohexane, 1-amino-1,5,5-trimethyl-3 (cis)-isopropyl-cyclohexane, 1-amino-1,5,5-trimethyl-3 (trans)-isopropyl-cyclohexane, 1-amino-1-methyl-3 (cis)-ethyl-cyclohexane, 1-amino-1-methyl-3 (cis)-methyl-cyclohexane, 1-amino-5,5-diethyl-1,3,3-trimethyl-cyclohexane, 1-amino-1,3,3,5,5-pentamethylcyclohexane, 1-amino-1,5,5-trimethyl-3,3-diethylcyclohexane, 1-amino-1-ethyl-3,3,5,5-tetramethylcyclohexane, N-ethyl-1-amino-1,3,3,5,5-pentamethylcyclohexane, N-(1,3,5-trimethylcyclohexyl)pyrrolidine, N-(1,3,5-trimethylcyclohexyl)piperidine, N-[1,3 (trans),5(trans)-trimethylcyclohexyl]pyrrolidine, N-[1,3 (trans),5 (trans)-trimethylcyclohexyl]piperidine, N-[1,3(cis),5(cis)-trimethylcyclohexyl]pyrrolidine, N-[1,3 (cis),5 (cis)-trimethylcyclohexyl]piperidine, N-(1,3,3,5-tetramethylcyclohexyl)pyrrolidine, N-(1,3,3,5-tetramethylcyclohexyl)piperidine, N-(1,3,3,5,5-pentamethylcyclohexyl)pyrrolidine, N-(1,3,3,5,5-pentamethylcyclohexyl)piperidine, N-(1,3,5,5-tetramethyl-3-ethylcyclohexyl)pyrrolidine, N-(1,3,5,5-tetramethyl-3-ethylcyclohexyl)piperidine, N-(1,5,5-trimethyl-3,3-diethylcyclohexyl)pyrrolidine, N-(1,5,5-trimethyl-3,3-diethylcyclohexyl)piperidine, N-(1,3,3-trimethyl-cis-5-ethylcyclohexyl)pyrrolidine, N-(1,3,3-trimethyl-cis-5-ethylcyclohexyl)piperidine, N-[(1S,5S)cis-5-ethyl-1,3,3-trimethylcyclohexyl]pyrrolidine, N-[(1S,5S)cis-5-ethyl-1,3,3-trimethylcyclohexyl]piperidine, N-(1,3,3-trimethyl-trans-5-ethylcyclohexyl)pyrrolidine, N-(1,3,3-trimethyl-trans-5-ethylcyclohexyl)piperidine, N-[(1R,5S)trans-5-ethyl,3,3-trimethylcyclohexyl]pyrrolidine, N-[(1R,5S)trans-5-ethyl,3,3-trimethylcyclohexyl]piperidine, N-(1-ethyl-3,3,5,5-tetramethylyclohexyl)pyrrolidine, N-(1-ethyl-3,3,5,5-tetramethylyclohexyl)piperidine, N-(1-propyl-3,3,5,5-tetramethylcyclohexyl)pyrrolidine, N-(1-propyl-3,3,5,5-tetramethylcyclohexyl)piperidine, N-(1,3,3,5,5-pentamethylcyclohexyl)pyrrolidine, spiro[cyclopropane-1,2-adamantan]-2-amine, spiro[pyrrolidine-2,2′-adamantane], spiro[piperidine-2,2-adamantane], 2-(2-adamantyl)piperidine, 3-(2-adamantyl)pyrrolidine, 2-(1-adamantyl)piperidine, 2-(1-adamantyl)pyrrolidine, and 2-(1-adamantyl)-2-methyl-pyrrolidine. In an even further aspect, the amantadine analog is selected from 1-amino-3-phenyl adamantane, 1-amino-methyl adamantane, 1-amino-3-ethyl adamantane, 1-amino-3-isopropyl adamantane, 1-amino-3-n-butyl adamantane, 1-amino-3,5-diethyl adamantane, 1-amino-3,5-diisopropyl adamantane, 1-amino-3,5-di-n-butyl adamantane, 1-amino-3-methyl-5-ethyl adamantane, 1-N-methylamino-3,5-dimethyl adamantane, 1-N-ethylamino-3,5-dimethyl adamantane, 1-N-isopropyl-amino-3,5-dimethyl adamantane, 1-N,N-dimethyl-amino-3,5-dimethyl adamantane, 1-N-methyl-N-isopropyl-amino-3-methyl-5-ethyl adamantane, 1-amino-3-butyl-5-phenyl adamantane, 1-amino-3-pentyl adamantane, 1-amino-3,5-dipentyl adamantane, 1-amino-3-pentyl-5-hexyl adamantane, 1-amino-3-pentyl-5-cyclohexyl adamantane, 1-amino-3-pentyl-5-phenyl adamantane, 1-amino-3-hexyl adamantane, 1-amino-3,5-dihexyl adamantane, 1-amino-3-hexyl-5-cyclohexyl adamantane, 1-amino-3-hexyl-5-phenyl adamantane, 1-amino-3-cyclohexyl adamantane, 1-amino-3,5-dicyclohexyl adamantane, 1-amino-3-cyclohexyl-5-phenyl adamantane, 1-amino-3,5-diphenyl adamantane, 1-amino-3,5,7-trimethyl adamantane, 1-amino-3,5-dimethyl-7-ethyl adamantane, 1-amino-3,5-diethyl-7-methyl adamantane, 1-N-pyrrolidino and 1-N-piperidine derivatives, 1-amino-3-methyl-5-propyl adamantane, 1-amino-3-methyl-5-butyl adamantane, 1-amino-3-methyl-5-pentyl adamantane, 1-amino-3-methyl-5-hexyl adamantane, 1-amino-3-methyl-5-cyclohexyl adamantane, 1-amino-3-methyl-5-phenyl adamantane, 1-amino-3-ethyl-5-propyl adamantane, 1-amino-3-ethyl-5-butyl adamantane, 1-amino-3-ethyl-5-pentyl adamantane, 1-amino-3-ethyl-5-hexyl adamantane, 1-amino-3-ethyl-5-cyclohexyl adamantane, 1-amino-3-ethyl-5-phenyl adamantane, 1-amino-3-propyl-5-butyl adamantane, 1-amino-3-propyl-5-pentyl adamantane, 1-amino-3-propyl-5-hexyl adamantane, 1-amino-3-propyl-5-cyclohexyl adamantane, 1-amino-3-propyl-5-phenyl adamantane, 1-amino-3-butyl-5-pentyl adamantane, 1-amino-3-butyl-5-hexyl adamantane, and 1-amino-3-butyl-5-cyclohexyl adamantine.

In a further aspect, the neuraminidase inhibitor is selected from oseltamivir, zanamivir, peramivir, laninamivir octanoate, 2,3-didehydro-2-deoxy-N-acetylneuraminic acid (DANA), 2-deoxy-2,3-dehydro-N-trifluoroacetylneuraminic acid (FANA), N-[(1R,2S)-2-methoxy-2-methyl-1-[(2R,3S,5R)-5-(2-methylpropanoyl)-3-[(Z)-prop-1-enyl]pyrrolidin-2-yl]pentyl]acetamide (A-322278), and (2R,4S,5R)-5-[(1R,2S)-1-acetamido-2-methoxy-2-methylpentyl]-4-[(Z)-prop-1-enyl]pyrrolidine-2-carboxylic acid (A-315675), or a pharmaceutically acceptable salt, solvate, or polymorph thereof. In a still further aspect, the neuraminidase inhibitor is selected from oseltamivir, zanamivir, peramivir, laninamivir octanoate, or a pharmaceutically acceptable salt, solvate, or polymorph thereof. In yet a further aspect, the neuraminidase inhibitor is oseltamivir, oseltamivir phosphate, or oseltamivir carboxylate. In an even further aspect, the neuraminidase inhibitor is oseltamivir phosphate. In a still further aspect, the neuraminidase inhibitor is zanamivir. In yet a further aspect, the neuraminidase inhibitor is peramivir. In an even further aspect, the neuraminidase inhibitor is laninamivir octanoate.

In a further aspect, the nucleoside analog is selected from ribavirin, viramidine, 6-fluoro-3-hydroxy-2-pyrazinecarboxamide, 2′-deoxy-2′-fluoroguanosine, pyrazofurin, carbodine, and cyclopenenyl cytosine. In a still further aspect, the nucleoside analog is selected from ribavirin and viramidine. In yet a further aspect, the nucleoside analog is ribavirin. In an even further aspect, the nucleoside analog is viramidine.

In a further aspect, the method further comprises a prostaglandin E2 receptor agonist, or a pharmaceutically acceptable prodrug, salt, solvate, or polymorph thereof. In a still further aspect, the prostaglandin E2 receptor agonist is selected from a prostaglandin E receptor 4 (subtype EP4) selective agonist, a prostaglandin E receptor 2 (subtype EP2) selective agonist, and a mixed agonist for prostaglandin E receptor 4 (subtype EP4) and prostaglandin E receptor 2 (subtype EP2). In yet a further aspect, the prostaglandin E2 receptor agonist is a prostaglandin E receptor 4 (subtype EP4) agonist. In an even further aspect, the prostaglandin E receptor 4 (subtype EP4) agonist is selected from beraprost, nileprost, iloprost, cicaprost, eptaloprost, and ciprosten, or a pharmaceutically acceptable prodrug, salt, solvate, or polymorph thereof. In a still further aspect, the prostaglandin E receptor 4 (subtype EP4) agonist is beraprost. In yet a further aspect, the prostaglandin E receptor 4 (subtype EP4) agonist is nileprost.

In a further aspect, the method further comprises an interferon, or an isoform, mutein or fused protein thereof. In a still further aspect, the interferon is a pegylated interferon, a recombinant interferon, or a natural interferon. In yet a further aspect, the interferon is recombinant human interferon-beta, recombinant human interfone-beta which has a CHO cell-derived glycosylation, or consensus interferon-beta. In an even further aspect, the interferon is interferon is pegylated interferon-beta or interferon-beta Fc-fusion protein.

In a further aspect, the method further comprises an effective amount of an antiviral agent selected from a replication inhibitor, an IMP dehydrogenase inhibitor, an RNA polymerase inhibitor, and an influenza-specific interfering oligonucleotide. In a still further aspect, the IMP dehydrogenase inhibitor is selected from ribavirin, viramidine, merimepodib (VX-497), mycophenolic acid, mycophenolate mofetil, benzamide riboside, tiazofurin, mizoribine, and 3-deazaguanosine. In yet a further aspect, the IMP dehydrogenase inhibitor is selected from ribavirin, viramidine, merimepodib (VX-497), mycophenolic acid, and mycophenolate mofetil. In an even further aspect, the IMP dehydrogenase inhibitor is selected from ribavirin, viramidine, mycophenolic acid, and mycophenolate mofetil.

In a further aspect, the RNA polymerase inhibitor is favipiravir.

In a further aspect, the method further comprises an effective amount of an influenza virus absorption inhibitor selected from a hemagglutinin-specific antibody, a polyoxometalate, a sulfated polysaccharide, a sialidase fusion protein, and an O-glycoside of sialic acid. In a still further aspect, the influenza virus absorption inhibitor is a recombinant sialidase fusion protein. In yet a further aspect, the recombinant sialidase fusion protein is Fludase (DAS181).

In a further aspect, the method further comprises an effective amount of a cysteamine compound. In a still further aspect, the cysteamine compound is selected from cysteamine, cysteamine salts, prodrugs of cysteamine, analogs of cysteamine, derivatives of cysteamine, conjugates of cysteamine, metabolic precursors of cysteamine, and metabolites of cysteamine. In a still further aspect, the cysteamine salt is cysteamine hydrochloride. In yet a further aspect, the metabolic precursor of cysteamine is selected from cysteine, cystamine, and pantethine. In an even further aspect, the cysteamine metabolite is selected from taurine and hypotaurine.

In a further aspect, the method further comprises an effective amount of a therapeutic agent selected from an antitussive, a mucolytic, an expectorant, an antipyretic, an analgesic, and a nasal decongestant.

In a further aspect, the method further comprises an effective amount of an immunomodulator, or a pharmaceutically acceptable prodrug, salt, solvate, or polymorph thereof. In a still further aspect, an effective amount of an immunomodulator is an amount effective to reduce or inhibit one or more symptoms of inflammation of a subject. In yet a further aspect, the immunomodulator is polyoxidonium.

In a further aspect, the immunomodulator is an anti-inflammatory agent. In a still further aspect, the anti-inflammatory agent is non-steroidal, steroidal, or a combination thereof. In yet a further aspect, the anti-inflammatory agent is a non-steroidal anti-inflammatory agent. In an even further aspect, the non-steroidal anti-inflammatory agent is selected from a COX2 inhibitor, an aminosalicylate drug, a PPAR ligand. In a still further aspect, the non-steroidal anti-inflammatory agent is selected from a COX2 inhibitor, an aminosalicylate drug, a PPAR ligand. In yet a further aspect, the non-steroidal anti-inflammatory agent is selected from an oxicam, a salicylate, an acetic acid derivative, a fenamate, a propionic acid derivative, and a pyrazole. In an even further aspect, the non-steroidal anti-inflammatory agent comprises one or more of piroxicam, isoxicam, tenoxicam, sudoxicam, aspirin, disalcid, benorylate, trilisate, safapryn, solprin, diflunisal, fendosal, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, mefenamic acid, meclofenamic acid, flufenamic acid, niflumic acid, tolfenamic acid, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofenic, phenylbutazone, oxyphenbutazone, feprazone, azapropazone, and trimethazone. In a still further aspect, the non-steroidal anti-inflammatory agent is a COX2 inhibitor. In yet a further aspect, the COX2 inhibitor is celecoxib.

In a further aspect, the non-steroidal anti-inflammatory agent is an aminosalicylate. In a still further aspect, the aminosalicylate drug is selected from mesalazine and sulfasalazine.

In a further aspect, the non-steroidal anti-inflammatory agent is a PPAR ligand. In a still further aspect, the PPAR ligand is a fibrate. In yet a further aspect, the fibrate is selected from gemfibrozil, bezafibrate, ciprofibrate, clofibrate, and renofibrate, or combinations thereof.

In a further aspect, the anti-inflammatory agent is a steroidal anti-inflammatory agent. In a still further aspect, the steroidal anti-inflammatory agent is selected from hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, predisone, beclomethasone dipropionate, triamcinolone, and mixtures thereof.

In a further aspect, treatment comprises prophylactic treatment.

c. Treating a Disorder of Uncontrolled Cellular Proliferation

In one aspect, the invention relates to a method treating a subject for disorder of uncontrolled cellular proliferation, the method comprising the step of administering to the subject an effective amount of a phospholipase D inhibitor, wherein the subject has been identified to have a mutation associated with activation of Akt, thereby treating the subject for the disorder of uncontrolled cellular proliferation. In a further aspect, the disorder of uncontrolled cellular proliferation is a cancer. In a still further aspect, the cancer is associated with activation of Akt. In yet a further aspect, the cancer is a hematological cancer. In an even further aspect, the hematological cancer is selected from a leukemia, lymphoma, chronic myeloproliferative disorder, myelodysplastic syndrome, myeloproliferative neoplasm, plasma cell neoplasm (myeloma), solid tumor, sarcoma, and carcinoma. In a still further aspect, the hematological cancer is a leukemia. In yet a further aspect, the leukemia is selected from acute leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, and chronic lymphocytic leukemia.

In a further aspect, the hematological cancer is a lymphoma. In a still further aspect, the lymphoma is selected from AIDS-Related lymphoma, cutaneous T-Cell lymphoma, Hodgkin lymphoma, non-Hodgkin lymphoma, primary central nervous system lymphoma, mycosis fungoides and the Sézary Syndrome, heavy chain disease, and Waldenström macroglobulinemia. In yet a further aspect, the lymphoma is Hodgkin's lymphoma. In an even further aspect, the lymphoma is non-Hodgkin's lymphoma.

In a further aspect, the cancer is a solid tumor. In a still further aspect, the cancer is selected from a cancer of the brain, genitourinary tract, gastrointestinal tract, colon, rectum, breast, kidney, lymphatic system, stomach, lung, pancreas, and skin. In yet a further aspect, the cancer is selected from prostate cancer, glioblastoma multiforme, endometrial cancer, breast cancer, and colon cancer. In an even further aspect, the cancer is selected from synovioma, mesothelioma, Ewing's tumor, pancreatic cancer, breast cancer, ovarian cancer, prostate cancer, hepatoma, Wilms' tumor, cervical cancer, testicular cancer, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In a further aspect, the cancer is a sarcoma. In a still further aspect, the sarcoma is selected from fibrosarcoma, myxosarcoma, liposarcoma, chondrosarcoma, osteogenic sarcoma, chordoma, angiosarcoma, endotheliosarcoma, lymphangiosarcoma, leiomyosarcoma, rhabdomyosarcoma, and lymphangioendotheliosarcoma.

In a further aspect, the cancer is a carcinoma. In a still further aspect, the carcinoma is selected from colon carcinoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, bile duct carcinoma, choriocarcinoma, seminoma, embryonal carcinoma, lung carcinoma, small cell lung carcinoma, bladder carcinoma, and epithelial carcinoma.

In a further aspect, the method further comprises the step of identifying a subject with a mutation associated with activation of Akt. In a still further aspect, the mutation associated with activation of Akt is in the PTEN gene. In yet a further aspect, the mutation is selected from c.17_{—}18delAA, c.955_{—}958delACTT, c.741_{—}742insA, c.968_{—}969insA, c.742_{—}743insC, c.742_{—}743insA, c.389G>A, c.202T>C, c.518G>A, c.517C>T, c.511C>T, c.640C>T, c.1003C>T, c.388C>T, c.697C>T, c.389G>T, c.388C>G, c.1002_{—}1003CC>TT, c.968delA, c.800delA, c.867delA, c.389delG, c.723_{—}724insTT, and c.969delT. In an even further aspect, the mutation in the PTEN gene is associated with a mutation in the PTEN protein selected from p.E242fs*15, p.K267fs*9, p.K6fs*4, p.N323fs*2, p.N323fs*21, p.P248fs*5, p.Q171*, p.Q214*, p.R130*, p.R130fs*4, p.R130G, p.R130L, p.R130Q, p.R173c, p.R173H, p.R233*, p.R335*, p.T319fs*1, p.V290fs*1, and p.Y68H.

In a further aspect, the mutation associated with activation of Akt is in the PIK3CA gene. In a still further aspect, the mutation is selected from c.3149G>A, c.3194A>T, c.3012G>T, c.1638G>T, c.3141T>G, c.3146G>C, c.323G>A, c.353G>A, c.3127A>G, c.113G>A, c.333G>C, c.331A>G, c.277C>T, c.1634A>T, c.1035T>A, c.1258T>C, c.1616C>G, c.1624G>A, c.1625A>T, c.1634A>G, c.1636C>A, c.1637A>C, c.3129G>T, c.3139C>T, c.3140A>T, c.3145G>A, c.2102A>C, c.1634A>C, c.1637A>G, c.3062A>G, c.1624G>C, c.1633G>C, c.1635G>C, c.3073A>G, c.1635G>Tc.3204_{—}3205insA, c.1633G>A, c.3140A>G, c.1636C>G, c.3145G>C, c.263G>A, c.1637A>T, c.317G>T, and c.3068G>A. In yet a further aspect, the mutation in the PIK3CA gene is associated with a mutation in the PIK3CA protein selected from p.C420R, p.E542K, p.E542Q, p.E542V, p.E545A, p.E545D, p.E545G, p.E545K, p.E545Q, p.E545V, p.G1049A, p.G1049R, p.G1049S, p.G1050D, p.G106V, p.G118D, p.H1047L, p.H1047Q, p.H1047R, p.H1047Y, p.H1065L, p.H701P, p.K111E, p.K111N, p.M1004I, p.M1043I, p.M1043V, p.N1068fs*4, p.N345K, p.P539R, p.Q546E, p.Q546H, p.Q546K, p.Q546L, p.Q546P, p.Q546R, p.R1023Q, p.R108H, p.R38H, p.R88Q, p.R93W, p.T1025A, and p.Y1021C.

In a further aspect, an effective amount of the PLD inhibitor is a therapeutically effective amount. In a still further aspect, an effective amount of the PLD inhibitor is a prophylactically effective amount.

In a further aspect, the PLD inhibitor is a compound having a structure represented by a formula:

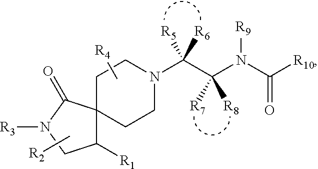

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

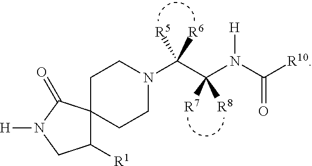

In a further aspect, the PLD inhibitor is a compound having a structure represented by a formula:

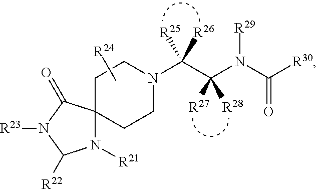

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

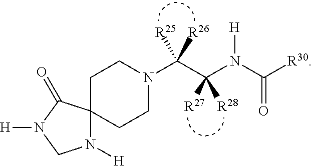

In a further aspect, the compound is:

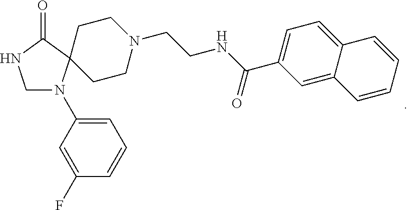 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

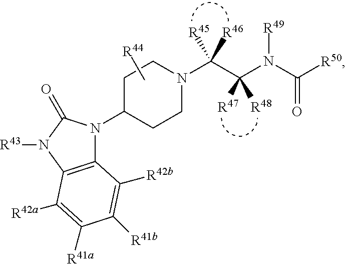

wherein each ----- independently comprises an optional covalent bond; wherein each of ea and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

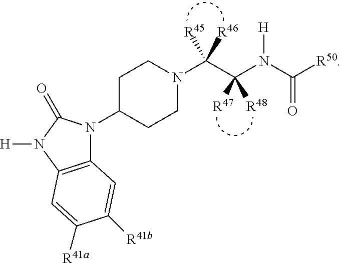

In a further aspect, the compound is:

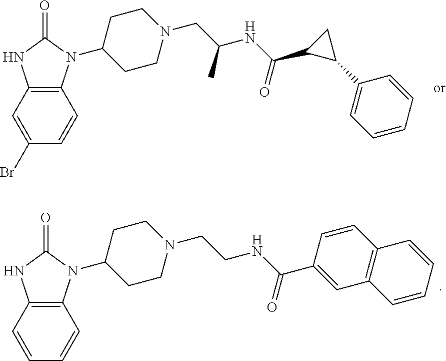

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof.

In a further aspect, the PLD inhibitor is a compound selected from:

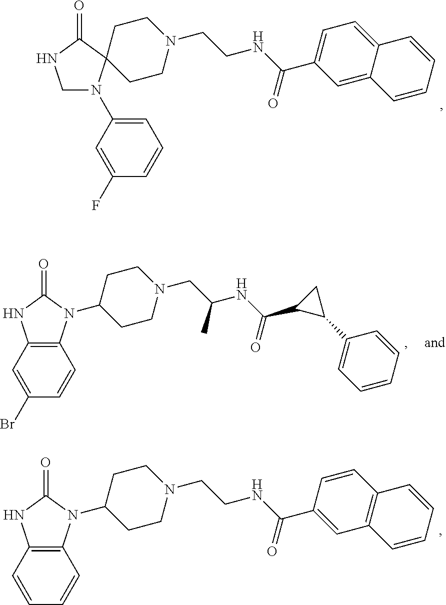

or a pharmaceutically acceptable salt, hydrate, solvate, or polymorph thereof.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the phospholipase D inhibitor is selected from:

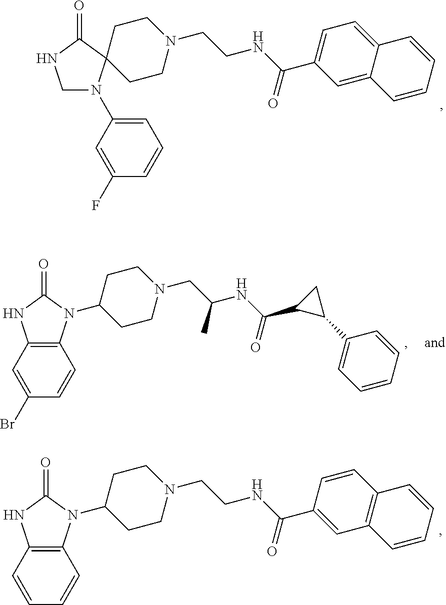

or a pharmaceutically acceptable salt, hydrate, solvate, or polymorph thereof

d. Modulating Autophagy in Cells

In one aspect, the invention relates to a method for modulating autophagy in at least one cell, comprising the step of contacting the cell with an effective amount of a phospholipase D inhibitor, thereby modulating autophagy in the cell. In a further aspect, an effective amount of the PLD inhibitor is a therapeutically effective amount. In a still further aspect, an effective amount of the PLD inhibitor is a prophylactically effective amount. In various aspects, modulating autophagy is decreasing net flux of autophagy in at least one cell. In a further aspect, decreasing net flux of autophagy in at least one cell effectively provides increased autophagy in the cell.

In a further aspect, the PLD inhibitor is a compound having a structure represented by a formula:

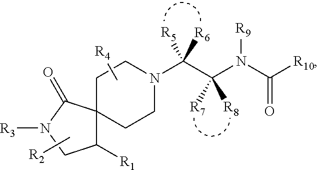

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

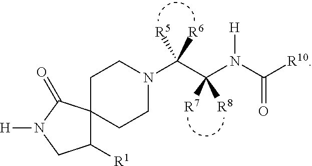

In a further aspect, the PLD inhibitor is a compound having a structure represented by a formula:

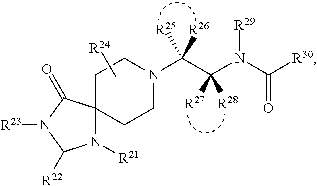

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

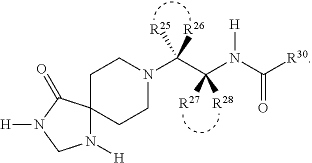

In a further aspect, the compound is:

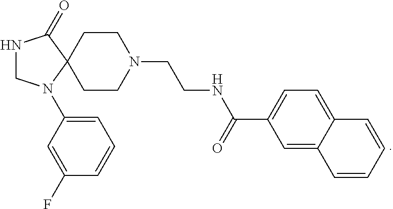 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

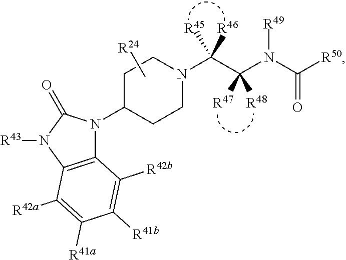

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

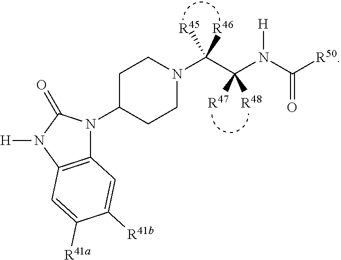

In a further aspect, the compound is:

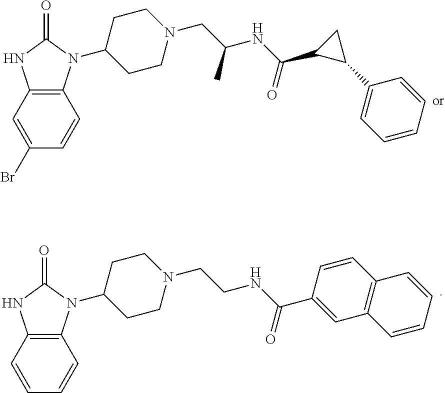

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof.

In a further aspect, the PLD inhibitor is a compound selected from:

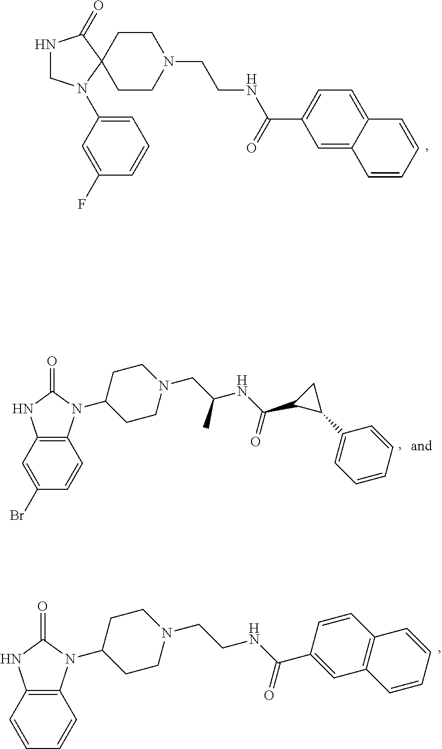

or a pharmaceutically acceptable salt, hydrate, solvate, or polymorph thereof.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the phospholipase D inhibitor is selected from:

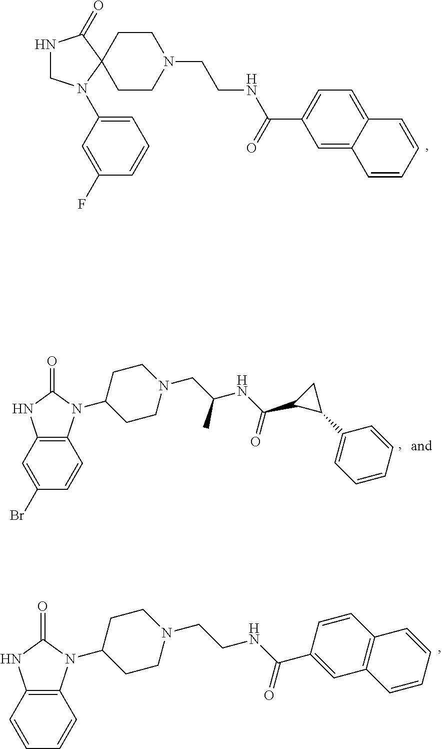

or a pharmaceutically acceptable salt, hydrate, solvate, or polymorph thereof.

e. Treating a Disorder

In one aspect, the invention relates to a method for treating a disorder in a subject, comprising the step of co-administering to the subject an Akt therapeutic agent and a phospholipase D inhibitor, thereby treating the disorder in the subject. In a further aspect, the amount of the Akt therapeutic agent co-administered with the phospholipase D inhibitor is less than the amount of the Akt therapeutic agent administered in the absence of the phospholipase D inhibitor in order to achieve substantially the same therapeutic effect in the subject. In a still further aspect, the disorder is associated with a viral infection. In yet a further aspect, the disorder is associated with a bacterial infection. In an even further aspect, the disorder is a disorder of uncontrolled cellular proliferation.

In a further aspect, an effective amount of the PLD inhibitor is a therapeutically effective amount. In a still further aspect, an effective amount of the PLD inhibitor is a prophylactically effective amount.

In a further aspect, the PLD inhibitor is a compound having a structure represented by a formula:

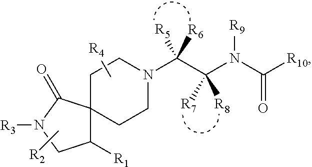

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

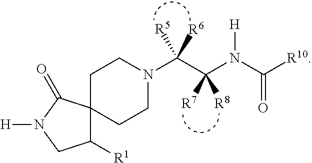

In a further aspect, the PLD inhibitor is a compound having a structure represented by a formula:

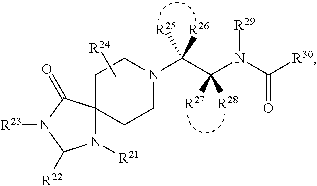

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

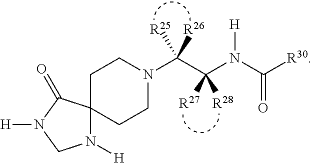

In a further aspect, the compound is:

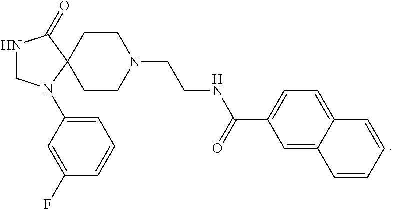 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

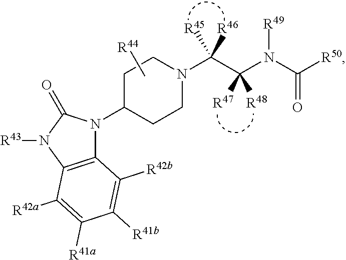

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

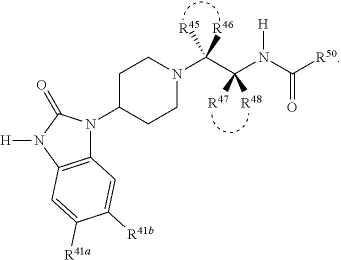

In a further aspect, the compound is:

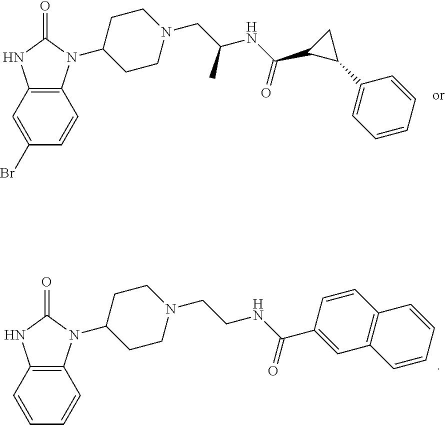

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof.

In a further aspect, the PLD inhibitor is a compound selected from:

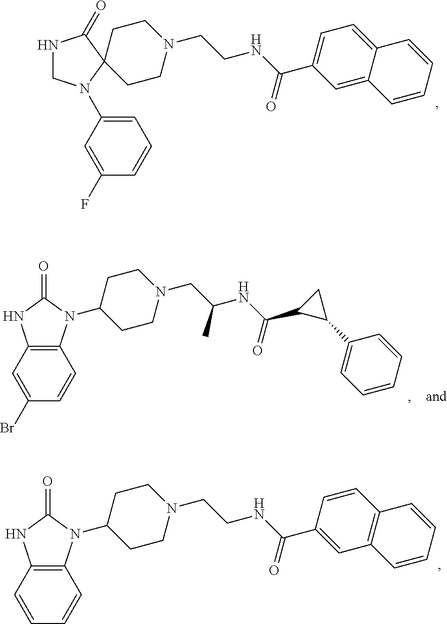

or a pharmaceutically acceptable salt, hydrate, solvate, or polymorph thereof.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the phospholipase D inhibitor is selected from:

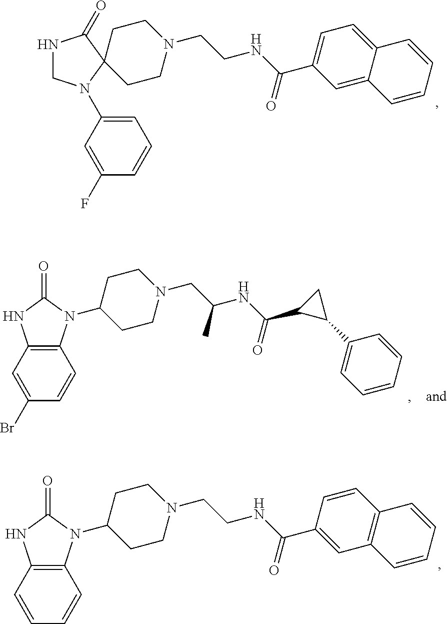

or a pharmaceutically acceptable salt, hydrate, solvate, or polymorph thereof.

In a further aspect, an effective amount of the Akt therapeutic agent is a therapeutically effective amount. In a still further aspect, an effective amount of the Akt therapeutic agent is a prophylatically effective amount.

In a further aspect, the Akt therapeutic agent is an Akt inhibitor. In a still further aspect, the Akt inhibitor binds to the pleckstrin homology domain. In yet a further aspect, the Akt inhibitor is an ATP-competitive inhibitor. In an even further aspect, the Akt inhibitor is an allosteric inhibitor. In a still further aspect, the allosteric inhibitor is MK-2066.

In a further aspect, the Akt inhibitor is a pan-Akt inhibitor.

In a further aspect, the Akt inhibitor inhibits Akt1, Akt2, or Akt3. In a still further aspect, the Akt inhibitor is an isoform-selective inhibitor. In yet a further aspect, the isoform-selective inhibitor selectively inhibits Akt1.

In a further aspect, the Akt therapeutic agent is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine. In a still further aspect, the Akt therapeutic agent is erucylphosphocholine. In yet a further aspect, the Akt therapeutic agent is GDC-0068. In an even further aspect, the Akt therapeutic agent is GSK-2141795. In a still further aspect, the Akt therapeutic agent is MK-2206. In a yet further aspect, the Akt therapeutic agent is perifosine. In an even further aspect, the Akt therapeutic agent is PHT-427.

In a further aspect, the Akt therapeutic agent is a siRNA.

In a further aspect, the Akt therapeutic agent is an antisense oligonucleotide. In a still further aspect, the antisense oligonucleotide is RX-0201.

2. Use of Compounds

In a further aspect, the invention relates to use of at least one disclosed compound in the manufacture of a medicament for the treatment of a viral infection. In a further aspect, the use is in the manufacture of a medicament for the treatment of a viral infection in a mammal.

In a further aspect, the invention relates to use of at least one disclosed compound in the manufacture of a medicament for the treatment of a disorder of uncontrolled cellular proliferation. In a further aspect, the use is in the manufacture of a medicament for the treatment of a disorder of uncontrolled cellular proliferation in a mammal.

In a further aspect, the medicament is formulated for inhalation or oral administration. In a still further aspect, the medicament is formulated for intravenous or intra-arterial injection.

It is understood that the disclosed uses can be employed in connection with the disclosed compounds, methods, compositions, and kits.

3. Manufacture of a Medicament

In one aspect, the invention relates to the manufacture of a medicament comprising combining at least one disclosed compound or at least one disclosed product with a pharmaceutically acceptable carrier or diluent.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

4. Kits

In one aspect, the invention relates to a kit comprising an Akt therapeutic agent, or a pharmaceutically acceptable prodrug, salt, solvate, or polymorph thereof, and one or more of: a) at least one therapeutic agent known to treat an HIV infection; b) at least one therapeutic agent known to treat an opportunistic infection associated with an HIV infection; c) instructions for treating an HIV infection; d) instructions for treating an opportunistic infection associated with an HIV infection; e) instructions for administering the Akt therapeutic agent in connection with treating an HIV infection; or f) instructions for administering the Akt therapeutic agent in connection with reducing the risk of HIV infection.

In a further aspect, the Akt therapeutic agent and the at least one therapeutic agent known to treat an HIV infection are co-packaged. In a still further aspect, the Akt therapeutic agent and the at least one therapeutic agent known to treat an HIV infection are co-packaged.

In a further aspect, the Akt therapeutic agent and the at least one therapeutic agent known to treat an opportunistic infection associated with an HIV infection are co-packaged. In a still further aspect, the Akt therapeutic agent and the at least one therapeutic agent known to treat an opportunistic infection associated with an HIV infection are co-formulated.

In a further aspect, the kit further comprises a plurality of dosage forms, the plurality comprising one or more doses; wherein each dose comprises an effective amount of the Akt therapeutic agent and the at least one therapeutic agent known to treat an HIV infection. In a still further aspect, the effective amount is a therapeutically effective amount. In yet a further aspect, the effective amount is a prophylactically effective amount.

In a further aspect, each dose of the Akt therapeutic agent and the at least one therapeutic agent known to treat an HIV infection are co-formulated. In a still further aspect, each dose of the Akt therapeutic agent and the at least one therapeutic agent known to treat an HIV infection are co-packaged.

In a further aspect, the dosage forms are formulated for oral administration and/or intravenous administration. In a still further aspect, the dosage forms are formulated for oral administration. In yet a further aspect, the dosage forms are formulated for intravenous administration. In an even further aspect, the dosage form for the Akt therapeutic agent is formulated for oral administration and the dosage for the at least one therapeutic agent known to treat an HIV infection is formulated for intravenous administration. In a still further aspect, the dosage form for the Akt therapeutic agent is formulated for intravenous administration and the dosage for the at least one therapeutic agent known to treat an HIV infection is formulated for oral administration.

In a further aspect, the Akt therapeutic agent is an Akt inhibitor. In a still further aspect, the Akt inhibitor binds to the pleckstrin homology domain. In yet a further aspect, the Akt inhibitor is an ATP-competitive inhibitor.

In a further aspect, the Akt inhibitor is an allosteric inhibitor. In a still further aspect, the allosteric inhibitor is MK-2066.

In a further aspect, the Akt inhibitor is a pan-Akt inhibitor

In a further aspect, the Akt inhibitor is an isoform-selective inhibitor. In a still further aspect, the Akt inhibitor inhibits Akt1, Akt2, or Akt3. In yet a further aspect, the isoform-selective inhibitor selectively inhibits Akt1.

In a further aspect, the Akt therapeutic agent is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine. In a still further aspect, the Akt therapeutic agent is erucylphosphocholine. In yet a further aspect, the Akt therapeutic agent is GDC-0068. In an even further aspect, the Akt therapeutic agent is GSK-2141795. In a still further aspect, the Akt therapeutic agent is MK-2206. In yet a further aspect, the Akt therapeutic agent is perifosine. In an even further aspect, the Akt therapeutic agent is PHT-427.

In a further aspect, the Akt therapeutic agent is a siRNA.

In a further aspect, the Akt therapeutic agent is an antisense oligonucleotide. In a still further aspect, the antisense oligonucleotide is RX-0201.

In a further aspect, the least one therapeutic agent known to treat an HIV infection comprises at least one more HIV therapeutic agent selected from: a) a HIV fusion/lysis inhibitor, or a pharmaceutically acceptable prodrug, salt, solvate, or polymorph thereof; b) a HIV integrase inhibitor, or a pharmaceutically acceptable prodrug, salt, solvate, or polymorph thereof; c) a HIV non-nucleoside reverse transcriptase inhibitor, or a pharmaceutically acceptable prodrug, salt, solvate, or polymorph thereof; d) a HIV nucleoside reverse transcriptase inhibitor, or a pharmaceutically acceptable prodrug, salt, solvate, or polymorph thereof; and e) a HIV protease inhibitor, or a pharmaceutically acceptable prodrug, salt, solvate, or polymorph thereof.

In a further aspect, the HIV fusion/lysis inhibitor is selected from enfuvirtide, maraviroc, cenicriviroc, ibalizumab, BMS-663068, and PRO-140, or a pharmaceutically acceptable prodrug, salt, solvate, or polymorph thereof. In a still further aspect, the HIV fusion/lysis inhibitor is selected from enfuvirtide, maraviroc, cenicriviroc, and ibalizumab, or a pharmaceutically acceptable prodrug, salt, solvate, or polymorph thereof. In yet a further aspect, the HIV fusion/lysis inhibitor is enfuvirtide, or a pharmaceutically acceptable prodrug, salt, solvate, or polymorph thereof. In an even further aspect, the HIV fusion/lysis inhibitor is maraviroc, or a pharmaceutically acceptable prodrug, salt, solvate, or polymorph thereof. In a still further aspect, the HIV fusion/lysis inhibitor is cenicriviroc, or a pharmaceutically acceptable prodrug, salt, solvate, or polymorph thereof. In yet a further aspect, the HIV fusion/lysis inhibitor is ibalizumab, or a pharmaceutically acceptable prodrug, salt, solvate, or polymorph thereof.

In a further aspect, the HIV integrase inhibitor is selected from raltegravir, dolutegravir, and elvitegravir, or a pharmaceutically acceptable prodrug, salt, solvate, or polymorph thereof. In a still further aspect, the HIV integrase inhibitor is raltegravir, or a pharmaceutically acceptable prodrug, salt, solvate, or polymorph thereof. In yet a further aspect, the HIV integrase inhibitor is dolutegravir, or a pharmaceutically acceptable prodrug, salt, solvate, or polymorph thereof. In an even further aspect, the HIV integrase inhibitor is elvitegravir, or a pharmaceutically acceptable prodrug, salt, solvate, or polymorph thereof.

In a further aspect, the HIV non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In a still further aspect, the HIV non-nucleoside reverse transcriptase inhibitor is delavirdine, or a pharmaceutically acceptable prodrug, salt, solvate, or polymorph thereof. In yet a further aspect, the HIV non-nucleoside reverse transcriptase inhibitor is efavirenz, or a pharmaceutically acceptable prodrug, salt, solvate, or polymorph thereof. In an even further aspect, the HIV non-nucleoside reverse transcriptase inhibitor is etravirine, or a pharmaceutically acceptable prodrug, salt, solvate, or polymorph thereof. In a still further aspect, the HIV non-nucleoside reverse transcriptase inhibitor is lersivirine, or a pharmaceutically acceptable prodrug, salt, solvate, or polymorph thereof. In yet a further aspect, the HIV non-nucleoside reverse transcriptase inhibitor is nevirapine, or a pharmaceutically acceptable prodrug, salt, solvate, or polymorph thereof. In an even further aspect, the HIV non-nucleoside reverse transcriptase inhibitor is rilpivirine, or a pharmaceutically acceptable prodrug, salt, solvate, or polymorph thereof.

In a further aspect, the HIV nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In a still further aspect, the HIV nucleoside reverse transcriptase inhibitor is selected from abacavir, didansine, elvucitabine, emtricitabine, lamivudine, stavudine, tenofovir, and zidovudine, or a pharmaceutically acceptable prodrug, salt, solvate, or polymorph thereof. In yet a further aspect, the HIV nucleoside reverse transcriptase inhibitor is abacavir, or a pharmaceutically acceptable prodrug, salt, solvate, or polymorph thereof. In an even further aspect, the HIV nucleoside reverse transcriptase inhibitor is didansine, or a pharmaceutically acceptable prodrug, salt, solvate, or polymorph thereof. In a still further aspect, the HIV nucleoside reverse transcriptase inhibitor is elvucitabine, or a pharmaceutically acceptable prodrug, salt, solvate, or polymorph thereof. In yet a further aspect, the HIV nucleoside reverse transcriptase inhibitor is emtricitabine, or a pharmaceutically acceptable prodrug, salt, solvate, or polymorph thereof. In an even further aspect, the HIV nucleoside reverse transcriptase inhibitor is lamivudine, or a pharmaceutically acceptable prodrug, salt, solvate, or polymorph thereof. In a still further aspect, the HIV nucleoside reverse transcriptase inhibitor is stavudine, or a pharmaceutically acceptable prodrug, salt, solvate, or polymorph thereof. In yet a further aspect, the HIV nucleoside reverse transcriptase inhibitor is tenofovir, or a pharmaceutically acceptable prodrug, salt, solvate, or polymorph thereof. In an even further aspect, the HIV nucleoside reverse transcriptase inhibitor is zidovudine, or a pharmaceutically acceptable prodrug, salt, solvate, or polymorph thereof.

In a further aspect, the HIV protease inhibitor is selected from wherein the HIV protease inhibitor is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In a still further aspect, the HIV protease inhibitor is atazanir, or a pharmaceutically acceptable prodrug, salt, solvate, or polymorph thereof. In yet a further aspect, the HIV protease inhibitor is darunavir, or a pharmaceutically acceptable prodrug, salt, solvate, or polymorph thereof. In an even further aspect, the HIV protease inhibitor is fosamprenavir, or a pharmaceutically acceptable prodrug, salt, solvate, or polymorph thereof. In a still further aspect, the HIV protease inhibitor is indinavir, or a pharmaceutically acceptable prodrug, salt, solvate, or polymorph thereof. In yet a further aspect, the HIV protease inhibitor is lopinavir, or a pharmaceutically acceptable prodrug, salt, solvate, or polymorph thereof. In an even further aspect, the HIV protease inhibitor is nelfinavir, or a pharmaceutically acceptable prodrug, salt, solvate, or polymorph thereof. In a still further aspect, the HIV protease inhibitor is ritonavir, or a pharmaceutically acceptable prodrug, salt, solvate, or polymorph thereof. In yet a further aspect, the HIV protease inhibitor is saquinavir, or a pharmaceutically acceptable prodrug, salt, solvate, or polymorph thereof. In an even further aspect, the HIV protease inhibitor is tipranavir, or a pharmaceutically acceptable prodrug, salt, solvate, or polymorph thereof.

In a further aspect, the kit further comprises an effective amount of an mTor inhibitor. In a still further aspect, the effective amount of an mTor inhibitor is a therapeutically effective amount. In yet a further aspect, the effective amount of an mTor inhibitor is a prophylactically effective amount.

In a further aspect, the mTor inhibitor is selected from everolimus, rapamycin (sirolimus), temsirolimus, deforolimus, ridaforolimus, tacrolimus, zotarolimus, salirasib, curcumin, farnesylthiosalicylic acid, XL765, ABI-009, AP-23675, AP-23841, AP-23765, AZD-8055, AZD-2014, BEZ-235 (NVP-BEZ235), BGT226, GDC-0980, INK-128, KU-0063794, MK8669, MKC-1 (Ro 31-7453), NVP-BGT226, OSI-027, Palomid-529, PF-04691502, PKI-402, PKI-587, PP-242, PP-30, SB-1518, SB-2312, SF-1126, TAFA-93, TOP-216, Torin1, WAY-600, WYE-125132, WYE-354, WYE-687, and XL-765, or a pharmaceutically acceptable prodrug, salt, solvate, or polymorph thereof. In a still further aspect, the mTor inhibitor is selected from everolimus, rapamycin (sirolimus), and temsirolimus, or a pharmaceutically acceptable prodrug, salt, solvate, or polymorph thereof. In yet a further aspect, the mTor inhibitor is everolimus, or a pharmaceutically acceptable prodrug, salt, solvate, or polymorph thereof. In an even further aspect, the mTor inhibitor is rapamycin (sirolimus), or a pharmaceutically acceptable prodrug, salt, solvate, or polymorph thereof. In a still further aspect, the mTor inhibitor is temsiorlimus, or a pharmaceutically acceptable prodrug, salt, solvate, or polymorph thereof. In yet a further aspect, the mTor inhibitor is deforolimus, or a pharmaceutically acceptable prodrug, salt, solvate, or polymorph thereof. In an even further aspect, the mTor inhibitor is tacrolimus, or a pharmaceutically acceptable prodrug, salt, solvate, or polymorph thereof. In a still further aspect, the mTor inhibitor is zotarolimus, or a pharmaceutically acceptable prodrug, salt, solvate, or polymorph thereof. In yet a further aspect, the mTor inhibitor is salirasib, or a pharmaceutically acceptable prodrug, salt, solvate, or polymorph thereof. In an even further aspect, the mTor inhibitor is curcumin, or a pharmaceutically acceptable prodrug, salt, solvate, or polymorph thereof. In a still further aspect, the mTor inhibitor is farnesylthiosalicylic acid, or a pharmaceutically acceptable prodrug, salt, solvate, or polymorph thereof. In yet a further aspect, the mTor inhibitor is farnesylthiosalicylic acid, or a pharmaceutically acceptable prodrug, salt, solvate, or polymorph thereof.

In a further aspect, the kit further comprises an effective amount of a PLD inhibitor. In a still further aspect, the effective amount of the PLD inhibitor is a therapeutically effective amount. In yet a further aspect, the effective amount of the PLD inhibitor is a prophylactically effective amount.

In a further aspect, the PLD inhibitor is a compound having a structure represented by a formula:

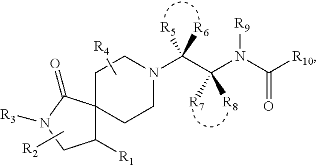

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

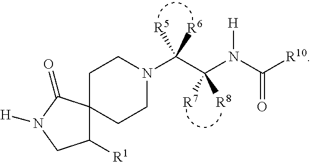

In a further aspect, the PLD inhibitor is a compound having a structure represented by a formula:

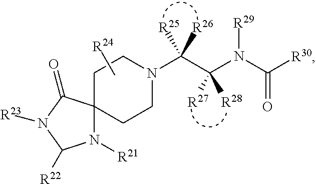

wherein each ----- independently comprises an optional covalent bond; wherein R″ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

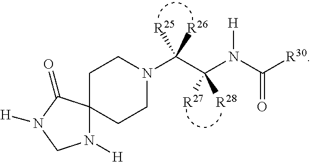

In a further aspect, the compound is:

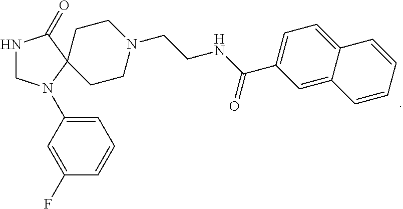 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

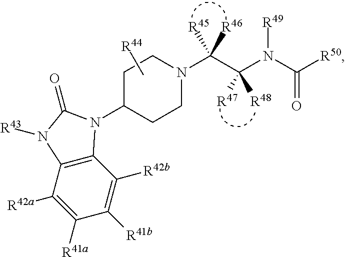

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

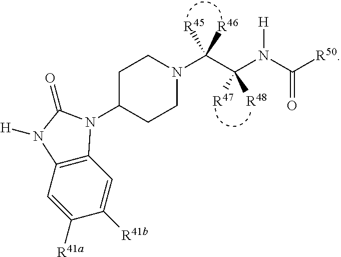

In a further aspect, the compound is:

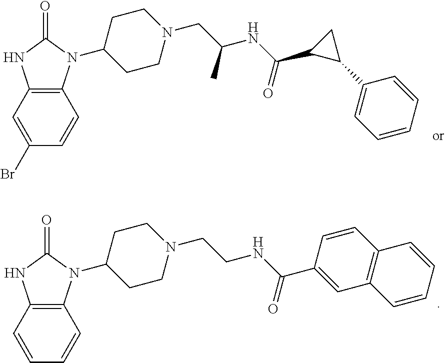

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide; or a pharmaceutically acceptable salt, hydrate, solvate, or polymorph thereof.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the phospholipase D inhibitor is selected from:

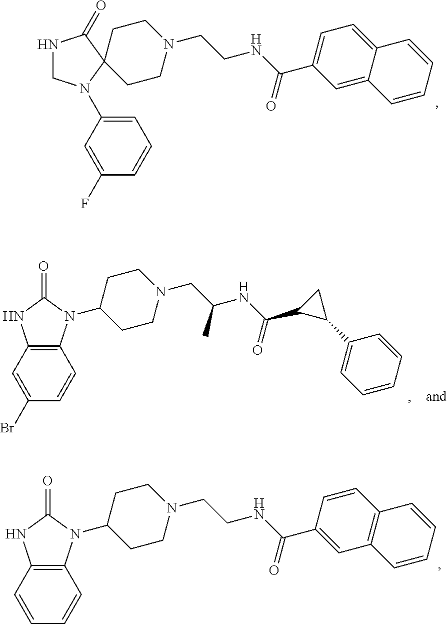

or a pharmaceutically acceptable salt, hydrate, solvate, or polymorph thereof.

In one aspect, the invention relates to a kit comprising an Akt therapeutic agent, or pharmaceutically acceptable salt, solvate, or polymorph thereof, and one or more of: a) at least one therapeutic agent known to decrease the severity of symptoms associated with an influenza infection; b) at least one therapeutic agent known to treat an influenza infection; c) instructions for treating an influenza infection; d) instructions for administering the Akt therapeutic agent in connection with treating an influenza infection; or e) instructions for administering the Akt therapeutic agent in connection with reducing the risk of influenza infection.

In a further aspect, the Akt therapeutic agent and the at least one therapeutic agent known to treat an influenza infection are co-packaged. In a still further aspect, the Akt therapeutic agent and the at least one therapeutic agent known to treat an influenza infection are co-formulated.

In a further aspect, the Akt therapeutic agent and the at least one therapeutic agent known to decrease the severity of symptoms associated with an influenza infection are co-packaged. In a still further aspect, the Akt therapeutic agent and the at least one therapeutic agent known to decrease the severity of symptoms associated with an influenza infection are co-formulated.

In a further aspect, the kit further comprises a plurality of dosage forms, the plurality comprising one or more doses; wherein each dose comprises an effective amount of the Akt therapeutic agent and the at least one therapeutic agent known to treat an influenza infection. In a still further aspect, the effective amount is a therapeutically effective amount. In yet a further aspect, the effective amount is a prophylactically effective amount.

In a further aspect, each dose of the Akt therapeutic agent and the at least one therapeutic agent known to treat an influenza infection are co-formulated. In a still further aspect, each dose of the Akt therapeutic agent and the at least one therapeutic agent known to treat an influenza infection are co-packaged.

In a further aspect, the dosage forms are formulated for oral administration and/or intravenous administration. In a still further aspect, the dosage forms are formulated for oral administration. In yet a further aspect, the dosage forms are formulated for intravenous administration. In an even further aspect, the dosage form for the Akt therapeutic agent is formulated for oral administration and the dosage for the at least one therapeutic agent known to treat an influenza infection is formulated for intravenous administration. In a still further aspect, the dosage form for the Akt therapeutic agent is formulated for intravenous administration and the dosage for the at least one therapeutic agent known to treat an influenza infection is formulated for oral administration.

In a further aspect, the Akt therapeutic agent is an Akt inhibitor. In a still further aspect, the Akt inhibitor binds to the pleckstrin homology domain.

In a further aspect, the Akt inhibitor is an ATP-competitive inhibitor.

In a further aspect, the Akt inhibitor is an allosteric inhibitor. In a still further aspect, the allosteric inhibitor is MK-2066.

In a further aspect, the Akt inhibitor is a pan-Akt inhibitor.

In a further aspect, the Akt inhibitor is an isoform-selective inhibitor. In a still further aspect, the Akt inhibitor inhibits Akt1, Akt2, or Akt3. In yet a further aspect, the isoform-selective inhibitor selectively inhibits Akt1.

In a further aspect, the Akt therapeutic agent is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine. In a still further aspect, the Akt therapeutic agent is erucylphosphocholine. In yet a further aspect, the Akt therapeutic agent is GDC-0068. In an even further aspect, the Akt therapeutic agent is GSK-2141795. In a still further aspect, the Akt therapeutic agent is MK-2206. In yet a further aspect, the Akt therapeutic agent is perifosine. In an even further aspect, the Akt therapeutic agent is PHT-427.

In a further aspect, the Akt therapeutic agent is a siRNA.

In a further aspect, the Akt therapeutic agent is an antisense oligonucleotide. In a still further aspect, the antisense oligonucleotide is RX-0201.

In a further aspect, the at least one therapeutic agent known to treat an influenza infection an effective amount of at least one influenza therapeutic agent selected from: a) a viral protein M2 ion channel inhibitor, or a pharmaceutically acceptable prodrug, salt, solvate, or polymorph thereof; b) a neuraminidase inhibitor, or a pharmaceutically acceptable prodrug, salt, solvate, or polymorph thereof; and c) a nucleoside analog, or a pharmaceutically acceptable prodrug, salt, solvate, or polymorph thereof.

In a further aspect, the effective amount of the at least one influenza therapeutic agent is a therapeutically effective amount. In a still further aspect, the effective amount of the at least one influenza therapeutic agent is a prophylactically effective amount.

In a further aspect, the viral protein M2 ion channel inhibitor is an amino-adamantane compound. In a still further aspect, the amino-adamantane compound is selected from 1-amino-adamantane and 1-(1-aminoethyl)adamantane. In yet a further aspect, the viral protein M2 ion channel inhibitor is selected from amantadine and rimantadine, or a pharmaceutically acceptable prodrug, salt, solvate, or polymorph thereof. In an even further aspect, the viral protein M2 ion channel inhibitor is an analog of amantadine or rimantadine. In a still further aspect, the amantadine analog is selected from 1-amino-1,3,5-trimethylcyclohexane, 1-amino-1(trans),3(trans),5-trimethylcyclohexane, 1-amino-1(cis),3(cis),5-trimethylcyclohexane, 1-amino-1,3,3,5-tetramethylcyclohexane, 1-amino-1,3,3,5,5-pentamethylcyclohexane(neramexane), 1-amino-1,3,5,5-tetramethyl-3-ethylcyclohexane, 1-amino-1,5,5-trimethyl-3,3-diethylcyclohexane, 1-amino-1,5,5-trimethyl-cis-3-ethylcyclohexane, 1-amino-(1S,5S)cis-3-ethyl-1,5,5-trimethylcyclohexane, 1-amino-1,5,5-trimethyl-trans-3-ethylcyclohexane, 1-amino-(1R,5S)trans-3-ethyl-1,5,5-trimethylcyclohexane, 1-amino-1-ethyl-3,3,5,5-tetramethylcyclohexane, 1-amino-1-propyl-3,3,5,5-tetramethylcyclohexane, N-methyl-1-amino-1,3,3,5,5-pentamethylcyclohexane, N-ethyl-1-amino-1,3,3,5,5-pentamethyl-cyclohexane, N-(1,3,3,5,5-pentamethylcyclohexyl)pyrrolidine, 3,3,5,5-tetramethylcyclohexylmethylamine, 1-amino-1-propyl-3,3,5,5-tetramethylcyclohexane, 1 amino-1,3,3,5(trans)-tetramethylcyclohexane (axial amino group), 3-propyl-1,3,5,5-tetramethylcyclohexylamine semihydrate, 1-amino-1,3,5,5-tetramethyl-3-ethylcyclohexane, 1-amino-1,3,5-trimethylcyclohexane, 1-amino-1,3-dimethyl-3-propylcyclohexane, 1-amino-1,3(trans),5(trans)-trimethyl-3(cis)-propylcyclohexane, 1-amino-1,3-dimethyl-3-ethylcyclohexane, 1-amino-1,3,3-trimethylcyclohexane, cis-3-ethyl-1(trans)-3(trans)-5-trimethylcyclohexamine, 1-amino-1,3(trans)-dimethylcyclohexane, 1,3,3-trimethyl-5,5-dipropylcyclohexylamine, 1-amino-1-methyl-3(trans)-propylcyclohexane, 1-methyl-3 (cis)-propylcyclohexylamine, 1-amino-1-methyl-3(trans)-ethylcyclohexane, 1-amino-1,3,3-trimethyl-5(cis)-ethylcyclohexane, 1-amino-1,3,3-trimethyl-5(trans)-ethylcyclohexane, cis-3-propyl-1,5,5-trimethylcyclohexylamine, trans-3-propyl-1,5,5-trimethylcyclohexylamine, N-ethyl-1,3,3,5,5-pentamethylcyclohexylamine, N-methyl-1-amino-1,3,3,5,5-pentamethylcyclohexane, 1-amino-1-methylcyclohexane, N,N-dimethyl-1-amino-1,3,3,5,5-pentamethylcyclohexane, 2-(3,3,5,5-tetramethylcyclohexyl)ethylamine, 2-methyl-1-(3,3,5,5-tetramethylcyclohexyl)propyl-2-amine, 2-(1,3,3,5,5-pentamethylcyclohexyl-1)-ethylamine semihydrate, N-(1,3,3,5,5-pentamethylcyclohexyl)-pyrrolidine, 1-amino-1,3(trans),5(trans)-trimethylcyclohexane, 1-amino-1,3(cis),5(cis)-trimethylcyclohexane, 1-amino-(1R,5S)trans-5-ethyl-1,3,3-trimethylcyclohexane, 1-amino-(1S,5S)cis-5-ethyl-1,3,3-trimethylcyclohexane, 1-amino-1,5,5-trimethyl-3(cis)-isopropyl-cyclohexane, 1-amino-1,5,5-trimethyl-3(trans)-isopropyl-cyclohexane, 1-amino-1-methyl-3 (cis)-ethyl-cyclohexane, 1-amino-1-methyl-3(cis)-methyl-cyclohexane, 1-amino-5,5-diethyl-1,3,3-trimethyl-cyclohexane, 1-amino-1,3,3,5,5-pentamethylcyclohexane, 1-amino-1,5,5-trimethyl-3,3-diethylcyclohexane, 1-amino-1-ethyl-3,3,5,5-tetramethylcyclohexane, N-ethyl-1-amino-1,3,3,5,5-pentamethylcyclohexane, N-(1,3,5-trimethylcyclohexyl)pyrrolidine, N-(1,3,5-trimethylcyclohexyl)piperidine, N-[1,3(trans),5(trans)-trimethylcyclohexyl]pyrrolidine, N-[1,3(trans),5(trans)-trimethylcyclohexyl]piperidine, N-[1,3(cis),5(cis)-trimethylcyclohexyl]pyrrolidine, N-[1,3(cis),5(cis)-trimethylcyclohexyl]piperidine, N-(1,3,3,5-tetramethylcyclohexyl)pyrrolidine, N-(1,3,3,5-tetramethylcyclohexyl)piperidine, N-(1,3,3,5,5-pentamethylcyclohexyl)pyrrolidine, N-(1,3,3,5,5-pentamethylcyclohexyl)piperidine, N-(1,3,5,5-tetramethyl-3-ethylcyclohexyl)pyrrolidine, N-(1,3,5,5-tetramethyl-3-ethylcyclohexyl)piperidine, N-(1,5,5-trimethyl-3,3-diethylcyclohexyl)pyrrolidine, N-(1,5,5-trimethyl-3,3-diethylcyclohexyl)piperidine, N-(1,3,3-trimethyl-cis-5-ethylcyclohexyl)pyrrolidine, N-(1,3,3-trimethyl-cis-5-ethylcyclohexyl)piperidine, N-[(1S,5S)cis-5-ethyl-1,3,3-trimethylcyclohexyl]pyrrolidine, N-[(1S,5S)cis-5-ethyl-1,3,3-trimethylcyclohexyl]piperidine, N-(1,3,3-trimethyl-trans-5-ethylcyclohexyl)pyrrolidine, N-(1,3,3-trimethyl-trans-5-ethylcyclohexyl)piperidine, N-[(1R,5S)trans-5-ethyl,3,3-trimethylcyclohexyl]pyrrolidine, N-[(1R,5S)trans-5-ethyl,3,3-trimethylcyclohexyl]piperidine, N-(1-ethyl-3,3,5,5-tetramethylyclohexyl)pyrrolidine, N-(1-ethyl-3,3,5,5-tetramethylyclohexyl)piperidine, N-(1-propyl-3,3,5,5-tetramethylcyclohexyl)pyrrolidine, N-(1-propyl-3,3,5,5-tetramethylcyclohexyl)piperidine, N-(1,3,3,5,5-pentamethylcyclohexyl)pyrrolidine, spiro[cyclopropane-1,2-adamantan]-2-amine, spiro[pyrrolidine-2,2′-adamantane], spiro[piperidine-2,2-adamantane], 2-(2-adamantyl)piperidine, 3-(2-adamantyl)pyrrolidine, 2-(1-adamantyl)piperidine, 2-(1-adamantyl)pyrrolidine, and 2-(1-adamantyl)-2-methyl-pyrrolidine. In yet a further aspect, the amantadine analog is selected from 1-amino-3-phenyl adamantane, 1-amino-methyl adamantane, 1-amino-3-ethyl adamantane, 1-amino-3-isopropyl adamantane, 1-amino-3-n-butyl adamantane, 1-amino-3,5-diethyl adamantane, 1-amino-3,5-diisopropyl adamantane, 1-amino-3,5-di-n-butyl adamantane, 1-amino-3-methyl-5-ethyl adamantane, 1-N-methylamino-3,5-dimethyl adamantane, 1-N-ethylamino-3,5-dimethyl adamantane, 1-N-isopropyl-amino-3,5-dimethyl adamantane, 1-N,N-dimethyl-amino-3,5-dimethyl adamantane, 1-N-methyl-N-isopropyl-amino-3-methyl-5-ethyl adamantane, 1-amino-3-butyl-5-phenyl adamantane, 1-amino-3-pentyl adamantane, 1-amino-3,5-dipentyl adamantane, 1-amino-3-pentyl-5-hexyl adamantane, 1-amino-3-pentyl-5-cyclohexyl adamantane, 1-amino-3-pentyl-5-phenyl adamantane, 1-amino-3-hexyl adamantane, 1-amino-3,5-dihexyl adamantane, 1-amino-3-hexyl-5-cyclohexyl adamantane, 1-amino-3-hexyl-5-phenyl adamantane, 1-amino-3-cyclohexyl adamantane, 1-amino-3,5-dicyclohexyl adamantane, 1-amino-3-cyclohexyl-5-phenyl adamantane, 1-amino-3,5-diphenyl adamantane, 1-amino-3,5,7-trimethyl adamantane, 1-amino-3,5-dimethyl-7-ethyl adamantane, 1-amino-3,5-diethyl-7-methyl adamantane, 1-N-pyrrolidino and 1-N-piperidine derivatives, 1-amino-3-methyl-5-propyl adamantane, 1-amino-3-methyl-5-butyl adamantane, 1-amino-3-methyl-5-pentyl adamantane, 1-amino-3-methyl-5-hexyl adamantane, 1-amino-3-methyl-5-cyclohexyl adamantane, 1-amino-3-methyl-5-phenyl adamantane, 1-amino-3-ethyl-5-propyl adamantane, 1-amino-3-ethyl-5-butyl adamantane, 1-amino-3-ethyl-5-pentyl adamantane, 1-amino-3-ethyl-5-hexyl adamantane, 1-amino-3-ethyl-5-cyclohexyl adamantane, 1-amino-3-ethyl-5-phenyl adamantane, 1-amino-3-propyl-5-butyl adamantane, 1-amino-3-propyl-5-pentyl adamantane, 1-amino-3-propyl-5-hexyl adamantane, 1-amino-3-propyl-5-cyclohexyl adamantane, 1-amino-3-propyl-5-phenyl adamantane, 1-amino-3-butyl-5-pentyl adamantane, 1-amino-3-butyl-5-hexyl adamantane, and 1-amino-3-butyl-5-cyclohexyl adamantine.

In a further aspect, the neuraminidase inhibitor is selected from oseltamivir, zanamivir, peramivir, laninamivir octanoate, 2,3-didehydro-2-deoxy-N-acetylneuraminic acid (DANA), 2-deoxy-2,3-dehydro-N-trifluoroacetylneuraminic acid (FANA), N-[(1R,2S)-2-methoxy-2-methyl-1-[(2R,3S,5R)-5-(2-methylpropanoyl)-3-[(Z)-prop-1-enyl]pyrrolidin-2-yl]pentyl]acetamide (A-322278), and (2R,4S,5R)-5-[(1R,2S)-1-acetamido-2-methoxy-2-methylpentyl]-4-[(Z)-prop-1-enyl]pyrrolidine-2-carboxylic acid (A-315675), or a pharmaceutically acceptable salt, solvate, or polymorph thereof. In a still further aspect, the neuraminidase inhibitor is selected from oseltamivir, zanamivir, peramivir, laninamivir octanoate, or a pharmaceutically acceptable salt, solvate, or polymorph thereof. In yet a further aspect, the neuraminidase inhibitor is oseltamivir, oseltamivir phosphate, or oseltamivir carboxylate. In an even further aspect, the neuraminidase inhibitor is oseltamivir phosphate. In a still further aspect, the neuraminidase inhibitor is zanamivir. In yet a further aspect, the neuraminidase inhibitor is peramivir. In an even further aspect, the neuraminidase inhibitor is laninamivir octanoate.

In a further aspect, the nucleoside analog is selected from ribavirin, viramidine, 6-fluoro-3-hydroxy-2-pyrazinecarboxamide, 2′-deoxy-2′-fluoroguanosine, pyrazofurin, carbodine, and cyclopenenyl cytosine. In a still further aspect, the nucleoside analog is selected from ribavirin and viramidine. In yet a further aspect, the nucleoside analog is ribavirin. In an even further aspect, the nucleoside analog is viramidine.

In a further aspect, the kit further comprises an effective amount of an mTor inhibitor. In a still further aspect, the effective amount of an mTor inhibitor is a therapeutically effective amount. In yet a further aspect, the effective amount of an mTor inhibitor is a prophylactically effective amount.

In a further aspect, the mTor inhibitor is selected from everolimus, rapamycin (sirolimus), temsirolimus, deforolimus, ridaforolimus, tacrolimus, zotarolimus, salirasib, curcumin, farnesylthiosalicylic acid, XL765, ABI-009, AP-23675, AP-23841, AP-23765, AZD-8055, AZD-2014, BEZ-235 (NVP-BEZ235), BGT226, GDC-0980, INK-128, KU-0063794, MK8669, MKC-1 (Ro 31-7453), NVP-BGT226, OSI-027, Palomid-529, PF-04691502, PKI-402, PKI-587, PP-242, PP-30, SB-1518, SB-2312, SF-1126, TAFA-93, TOP-216, Torin1, WAY-600, WYE-125132, WYE-354, WYE-687, and XL-765, or a pharmaceutically acceptable prodrug, salt, solvate, or polymorph thereof. In a still further aspect, the mTor inhibitor is selected from everolimus, rapamycin (sirolimus), and temsirolimus, or a pharmaceutically acceptable prodrug, salt, solvate, or polymorph thereof. In yet a further aspect, the mTor inhibitor is everolimus, or a pharmaceutically acceptable prodrug, salt, solvate, or polymorph thereof. In an even further aspect, the mTor inhibitor is rapamycin (sirolimus), or a pharmaceutically acceptable prodrug, salt, solvate, or polymorph thereof. In a still further aspect, the mTor inhibitor is temsiorlimus, or a pharmaceutically acceptable prodrug, salt, solvate, or polymorph thereof. In yet a further aspect, the mTor inhibitor is deforolimus, or a pharmaceutically acceptable prodrug, salt, solvate, or polymorph thereof. In an even further aspect, the mTor inhibitor is tacrolimus, or a pharmaceutically acceptable prodrug, salt, solvate, or polymorph thereof. In a still further aspect, the mTor inhibitor is zotarolimus, or a pharmaceutically acceptable prodrug, salt, solvate, or polymorph thereof. In yet a further aspect, the mTor inhibitor is salirasib, or a pharmaceutically acceptable prodrug, salt, solvate, or polymorph thereof. In an even further aspect, the mTor inhibitor is curcumin, or a pharmaceutically acceptable prodrug, salt, solvate, or polymorph thereof. In a still further aspect, the mTor inhibitor is farnesylthiosalicylic acid, or a pharmaceutically acceptable prodrug, salt, solvate, or polymorph thereof. In yet a further aspect, the mTor inhibitor is Torin1, or a pharmaceutically acceptable prodrug, salt, solvate, or polymorph thereof.

In a further aspect, the kit further comprises an effective amount of a PLD inhibitor. In a still further aspect, the effective amount of the PLD inhibitor is a therapeutically effective amount. In yet a further aspect, the effective amount of the PLD inhibitor is a prophylactically effective amount.

In a further aspect, the PLD inhibitor is a compound having a structure represented by a formula:

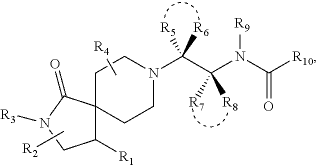

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

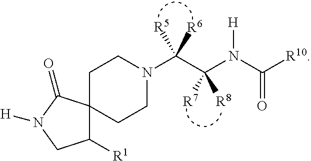

In a further aspect, the PLD inhibitor is a compound having a structure represented by a formula:

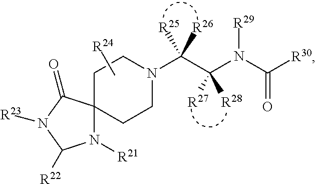

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

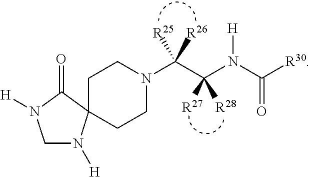

In a further aspect, the compound is:

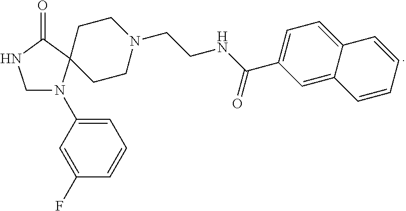 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

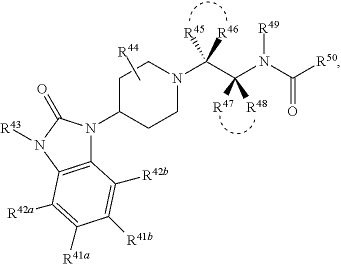

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

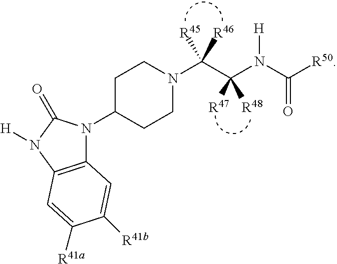

In a further aspect, the compound is:

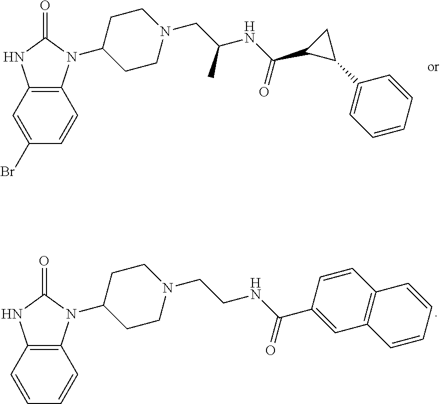

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide; or a pharmaceutically acceptable salt, hydrate, solvate, or polymorph thereof.

In a further aspect, the PLD inhibitor is a compound selected from:

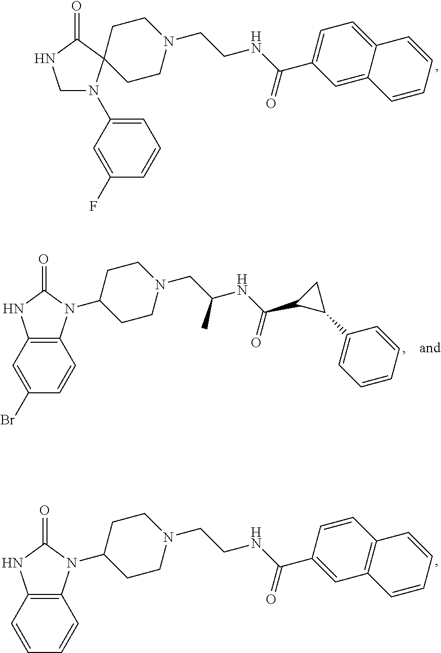

or a pharmaceutically acceptable salt, hydrate, solvate, or polymorph thereof.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the phospholipase D inhibitor is selected from:

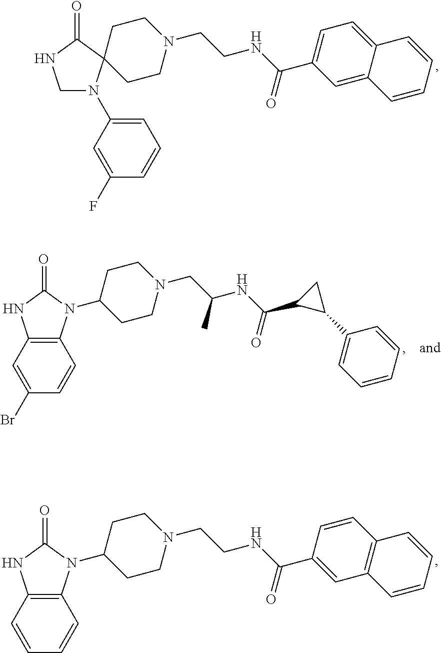

or a pharmaceutically acceptable salt, hydrate, solvate, or polymorph thereof.

In one aspect, the invention relates to a kit comprising an effective amount of at least one phospholipase D inhibitor, or a pharmaceutically acceptable prodrug, salt, solvate, or polymorph thereof; an effective amount of at least one mTor inhibitor, or a pharmaceutically acceptable prodrug, salt, solvate, or polymorph thereof; and one or more of: a) an effective amount of at least one agent known to treat an HIV infection; b) an effective amount of at least one agent known to treat an opportunistic infection associated with an HIV infection; c) instructions for treating an HIV infection; d) instructions for treating an opportunistic infection associated with an HIV infection; e) instructions for administering the phospholipase D inhibitor in connection with treating an HIV infection; or f) instructions for administering the phospholipase D inhibitor in connection with reducing the risk of HIV infection. In a further aspect, the effective amount of the PLD inhibitor is a therapeutically effective amount. In a still further aspect, the effective amount of the PLD inhibitor is a prophylatically effective amount.

In a further aspect, the PLD inhibitor is a compound having a structure represented by a formula:

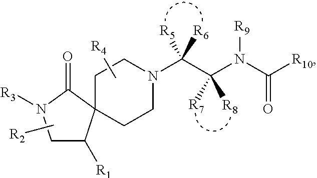

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

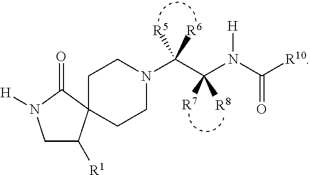

In a further aspect, the PLD inhibitor is a compound having a structure represented by a formula:

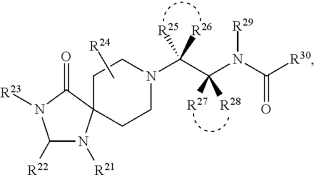

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

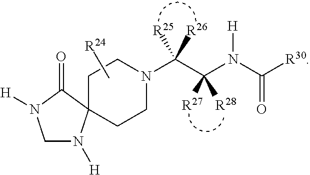

In a further aspect, the compound is:

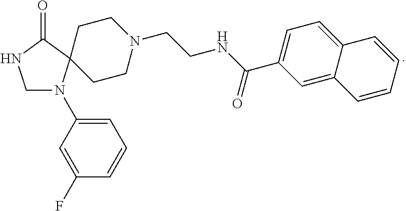 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

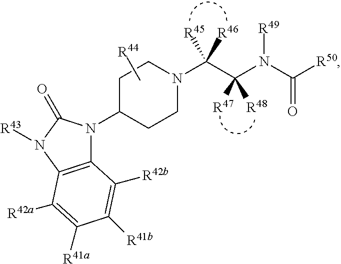

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

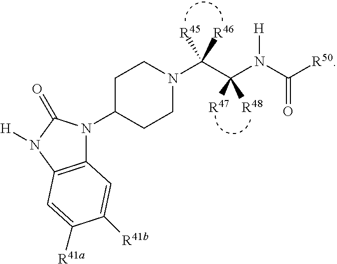

In a further aspect, the compound is:

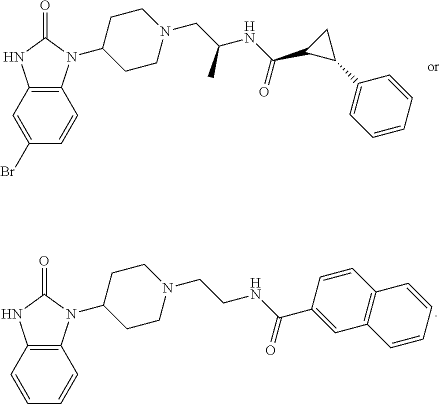

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof.

In a further aspect, the PLD inhibitor is a compound selected from:

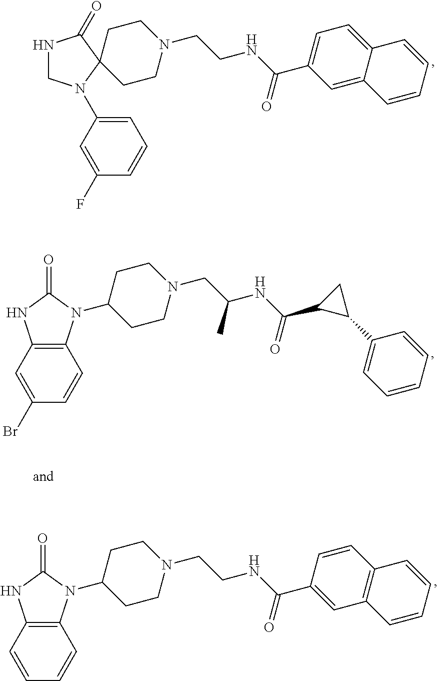

or a pharmaceutically acceptable salt, hydrate, solvate, or polymorph thereof.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the phospholipase D inhibitor is selected from:

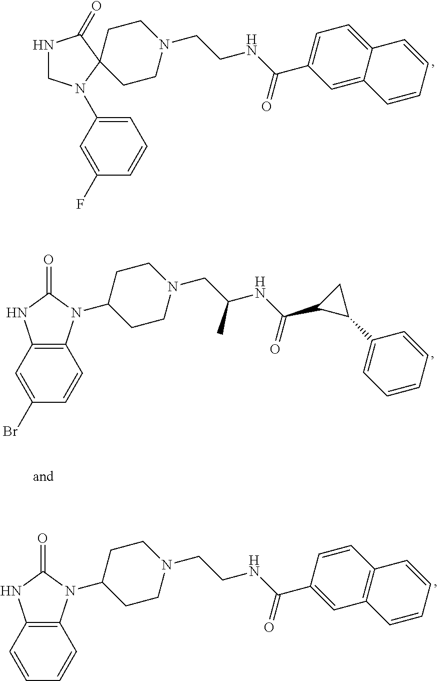

or a pharmaceutically acceptable salt, hydrate, solvate, or polymorph thereof.

In a further aspect, the effective amount of an mTor inhibitor is a therapeutically effective amount. In a still further aspect, the effective amount of an mTor inhibitor is a prophylatically effective amount.

In a further aspect, the mTor inhibitor is selected from everolimus, rapamycin (sirolimus), temsirolimus, deforolimus, ridaforolimus, tacrolimus, zotarolimus, salirasib, curcumin, farnesylthiosalicylic acid, XL765, ABI-009, AP-23675, AP-23841, AP-23765, AZD-8055, AZD-2014, BEZ-235 (NVP-BEZ235), BGT226, GDC-0980, INK-128, KU-0063794, MK8669, MKC-1 (Ro 31-7453), NVP-BGT226, OSI-027, Palomid-529, PF-04691502, PKI-402, PKI-587, PP-242, PP-30, SB-1518, SB-2312, SF-1126, TAFA-93, TOP-216, Torin1, WAY-600, WYE-125132, WYE-354, WYE-687, and XL-765, or a pharmaceutically acceptable prodrug, salt, solvate, or polymorph thereof. In a still further aspect, the mTor inhibitor is selected from everolimus, rapamycin (sirolimus), and temsirolimus, or a pharmaceutically acceptable prodrug, salt, solvate, or polymorph thereof. In yet a further aspect, the mTor inhibitor is everolimus, or a pharmaceutically acceptable prodrug, salt, solvate, or polymorph thereof. In an even further aspect, the mTor inhibitor is rapamycin (sirolimus), or a pharmaceutically acceptable prodrug, salt, solvate, or polymorph thereof. In a still further aspect, the mTor inhibitor is temsiorlimus, or a pharmaceutically acceptable prodrug, salt, solvate, or polymorph thereof. In yet a further aspect, the mTor inhibitor is deforolimus, or a pharmaceutically acceptable prodrug, salt, solvate, or polymorph thereof. In an even further aspect, the mTor inhibitor is tacrolimus, or a pharmaceutically acceptable prodrug, salt, solvate, or polymorph thereof. In a still further aspect, the mTor inhibitor is zotarolimus, or a pharmaceutically acceptable prodrug, salt, solvate, or polymorph thereof. In yet a further aspect, the mTor inhibitor is salirasib, or a pharmaceutically acceptable prodrug, salt, solvate, or polymorph thereof. In an even further aspect, the mTor inhibitor is curcumin, or a pharmaceutically acceptable prodrug, salt, solvate, or polymorph thereof. In a still further aspect, the mTor inhibitor is farnesylthiosalicylic acid, or a pharmaceutically acceptable prodrug, salt, solvate, or polymorph thereof. In yet a further aspect, the mTor inhibitor is Torin1, or a pharmaceutically acceptable prodrug, salt, solvate, or polymorph thereof.

In a further aspect, the least one agent known to treat an HIV infection comprises at least one more HIV therapeutic agent selected from: a) a HIV fusion/lysis inhibitor, or a pharmaceutically acceptable prodrug, salt, solvate, or polymorph thereof; b) a HIV integrase inhibitor, or a pharmaceutically acceptable prodrug, salt, solvate, or polymorph thereof; c) a HIV non-nucleoside reverse transcriptase inhibitor, or a pharmaceutically acceptable prodrug, salt, solvate, or polymorph thereof; d) a HIV nucleoside reverse transcriptase inhibitor, or a pharmaceutically acceptable prodrug, salt, solvate, or polymorph thereof; and e) a HIV protease inhibitor, or a pharmaceutically acceptable prodrug, salt, solvate, or polymorph thereof.

In a further aspect, the HIV fusion/lysis inhibitor is selected from enfuvirtide, maraviroc, cenicriviroc, ibalizumab, BMS-663068, and PRO-140, or a pharmaceutically acceptable prodrug, salt, solvate, or polymorph thereof. In a still further aspect, the HIV fusion/lysis inhibitor is selected from enfuvirtide, maraviroc, cenicriviroc, and ibalizumab, or a pharmaceutically acceptable prodrug, salt, solvate, or polymorph thereof. In yet a further aspect, the HIV fusion/lysis inhibitor is enfuvirtide, or a pharmaceutically acceptable prodrug, salt, solvate, or polymorph thereof. In an even further aspect, the HIV fusion/lysis inhibitor is maraviroc, or a pharmaceutically acceptable prodrug, salt, solvate, or polymorph thereof. In a still further aspect, the HIV fusion/lysis inhibitor is cenicriviroc, or a pharmaceutically acceptable prodrug, salt, solvate, or polymorph thereof. In yet a further aspect, the HIV fusion/lysis inhibitor is ibalizumab, or a pharmaceutically acceptable prodrug, salt, solvate, or polymorph thereof.

In a further aspect, the HIV integrase inhibitor is selected from raltegravir, dolutegravir, and elvitegravir, or a pharmaceutically acceptable prodrug, salt, solvate, or polymorph thereof. In a still further aspect, the HIV integrase inhibitor is raltegravir, or a pharmaceutically acceptable prodrug, salt, solvate, or polymorph thereof. In yet a further aspect, the HIV integrase inhibitor is dolutegravir, or a pharmaceutically acceptable prodrug, salt, solvate, or polymorph thereof. In an even further aspect, the HIV integrase inhibitor is elvitegravir, or a pharmaceutically acceptable prodrug, salt, solvate, or polymorph thereof.

In a further aspect, the HIV non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In a still further aspect, the HIV non-nucleoside reverse transcriptase inhibitor is delavirdine, or a pharmaceutically acceptable prodrug, salt, solvate, or polymorph thereof. In yet a further aspect, the HIV non-nucleoside reverse transcriptase inhibitor is efavirenz, or a pharmaceutically acceptable prodrug, salt, solvate, or polymorph thereof. In an even further aspect, the HIV non-nucleoside reverse transcriptase inhibitor is etravirine, or a pharmaceutically acceptable prodrug, salt, solvate, or polymorph thereof. In a still further aspect, the HIV non-nucleoside reverse transcriptase inhibitor is lersivirine, or a pharmaceutically acceptable prodrug, salt, solvate, or polymorph thereof. In yet a further aspect, the HIV non-nucleoside reverse transcriptase inhibitor is nevirapine, or a pharmaceutically acceptable prodrug, salt, solvate, or polymorph thereof. In an even further aspect, the HIV non-nucleoside reverse transcriptase inhibitor is rilpivirine, or a pharmaceutically acceptable prodrug, salt, solvate, or polymorph thereof.

In a further aspect, the HIV nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In a still further aspect, the HIV nucleoside reverse transcriptase inhibitor is selected from abacavir, didansine, elvucitabine, emtricitabine, lamivudine, stavudine, tenofovir, and zidovudine, or a pharmaceutically acceptable prodrug, salt, solvate, or polymorph thereof. In yet a further aspect, the HIV nucleoside reverse transcriptase inhibitor is abacavir, or a pharmaceutically acceptable prodrug, salt, solvate, or polymorph thereof. In an even further aspect, the HIV nucleoside reverse transcriptase inhibitor is didansine, or a pharmaceutically acceptable prodrug, salt, solvate, or polymorph thereof. In a still further aspect, the HIV nucleoside reverse transcriptase inhibitor is elvucitabine, or a pharmaceutically acceptable prodrug, salt, solvate, or polymorph thereof. In yet a further aspect, the HIV nucleoside reverse transcriptase inhibitor is emtricitabine, or a pharmaceutically acceptable prodrug, salt, solvate, or polymorph thereof. In an even further aspect, the HIV nucleoside reverse transcriptase inhibitor is lamivudine, or a pharmaceutically acceptable prodrug, salt, solvate, or polymorph thereof. In a still further aspect, the HIV nucleoside reverse transcriptase inhibitor is stavudine, or a pharmaceutically acceptable prodrug, salt, solvate, or polymorph thereof. In yet a further aspect, the HIV nucleoside reverse transcriptase inhibitor is tenofovir, or a pharmaceutically acceptable prodrug, salt, solvate, or polymorph thereof. In an even further aspect, the HIV nucleoside reverse transcriptase inhibitor is zidovudine, or a pharmaceutically acceptable prodrug, salt, solvate, or polymorph thereof.

In a further aspect, the HIV protease inhibitor is selected from wherein the HIV protease inhibitor is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In a still further aspect, the HIV protease inhibitor is atazanir, or a pharmaceutically acceptable prodrug, salt, solvate, or polymorph thereof. In yet a further aspect, the HIV protease inhibitor is darunavir, or a pharmaceutically acceptable prodrug, salt, solvate, or polymorph thereof. In an even further aspect, the HIV protease inhibitor is fosamprenavir, or a pharmaceutically acceptable prodrug, salt, solvate, or polymorph thereof. In a still further aspect, the HIV protease inhibitor is indinavir, or a pharmaceutically acceptable prodrug, salt, solvate, or polymorph thereof. In yet a further aspect, the HIV protease inhibitor is lopinavir, or a pharmaceutically acceptable prodrug, salt, solvate, or polymorph thereof. In an even further aspect, the HIV protease inhibitor is nelfinavir, or a pharmaceutically acceptable prodrug, salt, solvate, or polymorph thereof. In a still further aspect, the HIV protease inhibitor is ritonavir, or a pharmaceutically acceptable prodrug, salt, solvate, or polymorph thereof. In yet a further aspect, the HIV protease inhibitor is saquinavir, or a pharmaceutically acceptable prodrug, salt, solvate, or polymorph thereof. In an even further aspect, the HIV protease inhibitor is tipranavir, or a pharmaceutically acceptable prodrug, salt, solvate, or polymorph thereof.

In a further aspect, the kit further comprises an effective amount of an Akt therapeutic agent. In a still further aspect, the effective amount of the Akt therapeutic agent is a therapeutically effective amount. In yet a further aspect, the effective amount of the Akt therapeutic agent is a prophylactically effective amount.

In a further aspect, the Akt therapeutic agent is an Akt inhibitor. In a still further aspect, the Akt inhibitor binds to the pleckstrin homology domain. In yet a further aspect, the Akt inhibitor is an ATP-competitive inhibitor. In an even further aspect, the Akt inhibitor is an allosteric inhibitor. In a still further aspect, the allosteric inhibitor is MK-2066.

In a further aspect, the Akt inhibitor is a pan-Akt inhibitor.

In a further aspect, the Akt inhibitor inhibits Akt1, Akt2, or Akt3. In a still further aspect, the Akt inhibitor is an isoform-selective inhibitor. In yet a further aspect, the isoform-selective inhibitor selectively inhibits Akt1.

In a further aspect, the Akt therapeutic agent is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine. In a still further aspect, the Akt inhibitor is erucylphosphocholine. In yet a further aspect, the Akt inhibitor is GDC-0068. In an even further aspect, the Akt inhibitor is GSK-2141795. In a still further aspect, the Akt inhibitor is MK-2206. In yet a further aspect, the Akt inhibitor is perifosine. In an even further aspect, the Akt inhibitor is PHT-427.

In a further aspect, the Akt therapeutic agent is a siRNA.

In a further aspect, the Akt therapeutic agent is an antisense oligonucleotide. In a still further aspect, the antisense oligonucleotide is RX-0201.

In one aspect, the invention relates to a kit comprising a phospholipase D inhibitor, or a pharmaceutically acceptable prodrug, salt, solvate, or polymorph thereof; a mTor inhibitor, or a pharmaceutically acceptable prodrug, salt, solvate, or polymorph thereof; and one or more of: a) at least one agent known to decrease the severity of symptoms associated with an influenza infection; b) at least one agent known to treat an influenza infection; c) instructions for treating an influenza infection; d) instructions for administering the Akt inhibitor in connection with treating an influenza infection; or e) instructions for administering the Akt inhibitor in connection with reducing the risk of influenza infection. In a further aspect, the effective amount of the PLD inhibitor is a therapeutically effective amount. In a still further aspect, the effective amount of the PLD inhibitor is a prophylatically effective amount.

In a further aspect, the PLD inhibitor is a compound having a structure represented by a formula:

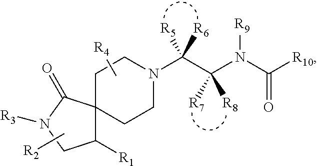

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

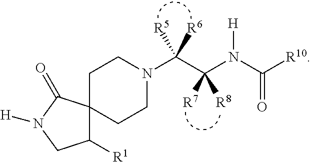

In a further aspect, the PLD inhibitor is a compound having a structure represented by a formula:

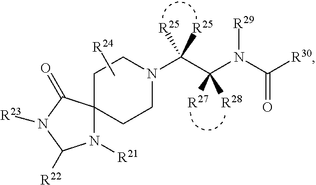

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

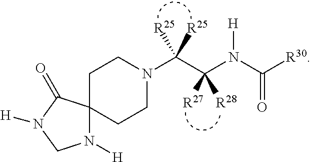

In a further aspect, the compound is:

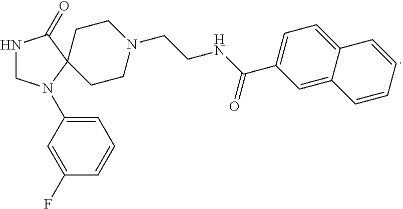 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

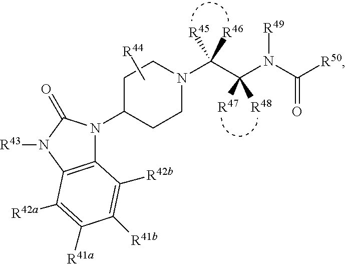

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

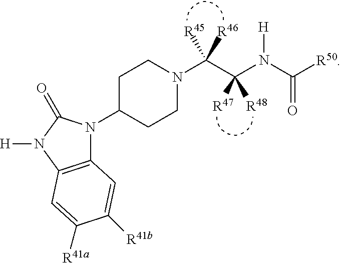

In a further aspect, the compound is:

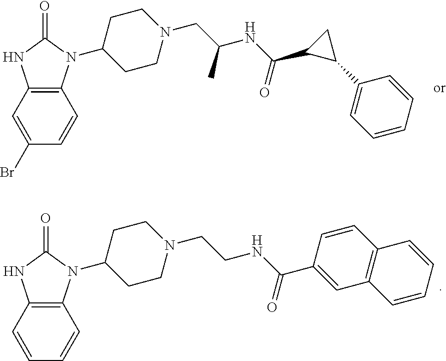

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof.

In a further aspect, the PLD inhibitor is a compound selected from:

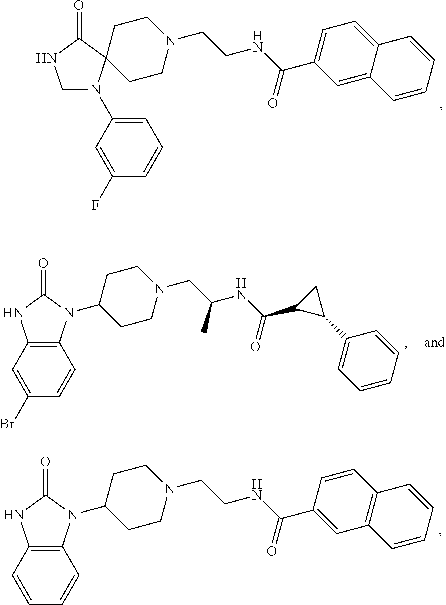

or a pharmaceutically acceptable salt, hydrate, solvate, or polymorph thereof.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the phospholipase D inhibitor is selected from:

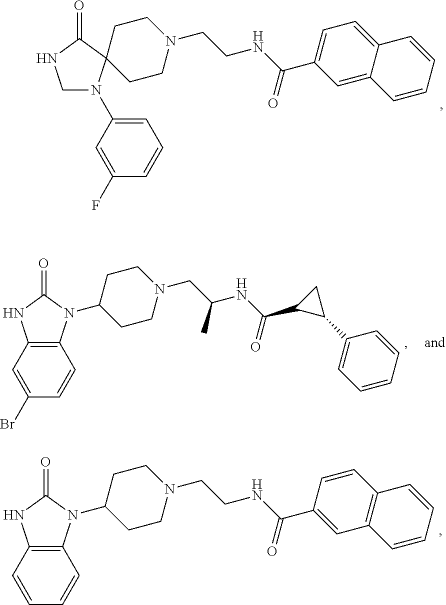

or a pharmaceutically acceptable salt, hydrate, solvate, or polymorph thereof.

In a further aspect, the effective amount of an mTor inhibitor is a therapeutically effective amount. In a still further aspect, the effective amount of an mTor inhibitor is a prophylatically effective amount.

In a further aspect, the mTor inhibitor is selected from everolimus, rapamycin (sirolimus), temsirolimus, deforolimus, ridaforolimus, tacrolimus, zotarolimus, salirasib, curcumin, farnesylthiosalicylic acid, XL765, ABI-009, AP-23675, AP-23841, AP-23765, AZD-8055, AZD-2014, BEZ-235 (NVP-BEZ235), BGT226, GDC-0980, INK-128, KU-0063794, MK8669, MKC-1 (Ro 31-7453), NVP-BGT226, OSI-027, Palomid-529, PF-04691502, PKI-402, PKI-587, PP-242, PP-30, SB-1518, SB-2312, SF-1126, TAFA-93, TOP-216, Torin1, WAY-600, WYE-125132, WYE-354, WYE-687, and XL-765, or a pharmaceutically acceptable prodrug, salt, solvate, or polymorph thereof. In a still further aspect, the mTor inhibitor is selected from everolimus, rapamycin (sirolimus), and temsirolimus, or a pharmaceutically acceptable prodrug, salt, solvate, or polymorph thereof. In yet a further aspect, the mTor inhibitor is everolimus, or a pharmaceutically acceptable prodrug, salt, solvate, or polymorph thereof. In an even further aspect, the mTor inhibitor is rapamycin (sirolimus), or a pharmaceutically acceptable prodrug, salt, solvate, or polymorph thereof. In a still further aspect, the mTor inhibitor is temsiorlimus, or a pharmaceutically acceptable prodrug, salt, solvate, or polymorph thereof. In yet a further aspect, the mTor inhibitor is deforolimus, or a pharmaceutically acceptable prodrug, salt, solvate, or polymorph thereof. In an even further aspect, the mTor inhibitor is tacrolimus, or a pharmaceutically acceptable prodrug, salt, solvate, or polymorph thereof. In a still further aspect, the mTor inhibitor is zotarolimus, or a pharmaceutically acceptable prodrug, salt, solvate, or polymorph thereof. In yet a further aspect, the mTor inhibitor is salirasib, or a pharmaceutically acceptable prodrug, salt, solvate, or polymorph thereof. In an even further aspect, the mTor inhibitor is curcumin, or a pharmaceutically acceptable prodrug, salt, solvate, or polymorph thereof. In a still further aspect, the mTor inhibitor is farnesylthiosalicylic acid, or a pharmaceutically acceptable prodrug, salt, solvate, or polymorph thereof. In yet a further aspect, the mTor inhibitor is Torin1, or a pharmaceutically acceptable prodrug, salt, solvate, or polymorph thereof.

In a further aspect, the at least one agent known to treat an influenza infection comprises an effective of at least one influenza therapeutic agent selected from: a) a viral protein M2 ion channel inhibitor, or a pharmaceutically acceptable prodrug, salt, solvate, or polymorph thereof; b) a neuraminidase inhibitor, or a pharmaceutically acceptable prodrug, salt, solvate, or polymorph thereof; and c) a nucleoside analog, or a pharmaceutically acceptable prodrug, salt, solvate, or polymorph thereof. In a still further aspect, the effective amount of the at least one influenza therapeutic agent is a therapeutically effective amount. In yet a further aspect, the effective amount of the at least one influenza therapeutic agent is a prophylatically effective amount.

In a further aspect, the viral protein M2 ion channel inhibitor is an amino-adamantane compound. In a still further aspect, the amino-adamantane compound is selected from 1-amino-adamantane and 1-(1-aminoethyl)adamantane.

In a further aspect, the viral protein M2 ion channel inhibitor is selected from amantadine and rimantadine, or a pharmaceutically acceptable prodrug, salt, solvate, or polymorph thereof.

In a further aspect, the viral protein M2 ion channel inhibitor is an analog of amantadine or rimantadine. In a still further aspect, the amantadine analog is selected from 1-amino-1,3,5-trimethylcyclohexane, 1-amino-1(trans),3(trans),5-trimethylcyclohexane, 1-amino-1(cis),3 (cis),5-trimethylcyclohexane, 1-amino-1,3,3,5-tetramethylcyclohexane, 1-amino-1,3,3,5,5-pentamethylcyclohexane(neramexane), 1-amino-1,3,5,5-tetramethyl-3-ethylcyclohexane, 1-amino-1,5,5-trimethyl-3,3-diethylcyclohexane, 1-amino-1,5,5-trimethyl-cis-3-ethylcyclohexane, 1-amino-(1S,5S)cis-3-ethyl-1,5,5-trimethylcyclohexane, 1-amino-1,5,5-trimethyl-trans-3-ethylcyclohexane, 1-amino-(1R,5S)trans-3-ethyl-1,5,5-trimethylcyclohexane, 1-amino-1-ethyl-3,3,5,5-tetramethylcyclohexane, 1-amino-1-propyl-3,3,5,5-tetramethylcyclohexane, N-methyl-1-amino-1,3,3,5,5-pentamethylcyclohexane, N-ethyl-1-amino-1,3,3,5,5-pentamethyl-cyclohexane, N-(1,3,3,5,5-pentamethylcyclohexyl)pyrrolidine, 3,3,5,5-tetramethylcyclohexylmethylamine, 1-amino-1-propyl-3,3,5,5-tetramethylcyclohexane, 1 amino-1,3,3,5(trans)-tetramethylcyclohexane (axial amino group), 3-propyl-1,3,5,5-tetramethylcyclohexylamine semihydrate, 1-amino-1,3,5,5-tetramethyl-3-ethylcyclohexane, 1-amino-1,3,5-trimethylcyclohexane, 1-amino-1,3-dimethyl-3-propylcyclohexane, 1-amino-1,3(trans),5(trans)-trimethyl-3(cis)-propylcyclohexane, 1-amino-1,3-dimethyl-3-ethylcyclohexane, 1-amino-1,3,3-trimethylcyclohexane, cis-3-ethyl-1(trans)-3(trans)-5-trimethylcyclohexamine, 1-amino-1,3(trans)-dimethylcyclohexane, 1,3,3-trimethyl-5,5-dipropylcyclohexylamine, 1-amino-1-methyl-3(trans)-propylcyclohexane, 1-methyl-3 (cis)-propylcyclohexylamine, 1-amino-1-methyl-3(trans)-ethylcyclohexane, 1-amino-1,3,3-trimethyl-5(cis)-ethylcyclohexane, 1-amino-1,3,3-trimethyl-5(trans)-ethylcyclohexane, cis-3-propyl-1,5,5-trimethylcyclohexylamine, trans-3-propyl-1,5,5-trimethylcyclohexylamine, N-ethyl-1,3,3,5,5-pentamethylcyclohexylamine, N-methyl-1-amino-1,3,3,5,5-pentamethylcyclohexane, 1-amino-1-methylcyclohexane, N,N-dimethyl-1-amino-1,3,3,5,5-pentamethylcyclohexane, 2-(3,3,5,5-tetramethylcyclohexyl)ethylamine, 2-methyl-1-(3,3,5,5-tetramethylcyclohexyl)propyl-2-amine, 2-(1,3,3,5,5-pentamethylcyclohexyl-1)-ethylamine semihydrate, N-(1,3,3,5,5-pentamethylcyclohexyl)-pyrrolidine, 1-amino-1,3(trans),5(trans)-trimethylcyclohexane, 1-amino-1,3(cis),5(cis)-trimethylcyclohexane, 1-amino-(1R,5S)trans-5-ethyl-1,3,3-trimethylcyclohexane, 1-amino-(1S,5S)cis-5-ethyl-1,3,3-trimethylcyclohexane, 1-amino-1,5,5-trimethyl-3(cis)-isopropyl-cyclohexane, 1-amino-1,5,5-trimethyl-3(trans)-isopropyl-cyclohexane, 1-amino-1-methyl-3 (cis)-ethyl-cyclohexane, 1-amino-1-methyl-3(cis)-methyl-cyclohexane, 1-amino-5,5-diethyl-1,3,3-trimethyl-cyclohexane, 1-amino-1,3,3,5,5-pentamethylcyclohexane, 1-amino-1,5,5-trimethyl-3,3-diethylcyclohexane, 1-amino-1-ethyl-3,3,5,5-tetramethylcyclohexane, N-ethyl-1-amino-1,3,3,5,5-pentamethylcyclohexane, N-(1,3,5-trimethylcyclohexyl)pyrrolidine, N-(1,3,5-trimethylcyclohexyl)piperidine, N-[1,3(trans),5(trans)-trimethylcyclohexyl]pyrrolidine, N-[1,3(trans),5(trans)-trimethylcyclohexyl]piperidine, N-[1,3(cis),5(cis)-trimethylcyclohexyl]pyrrolidine, N-[1,3(cis),5(cis)-trimethylcyclohexyl]piperidine, N-(1,3,3,5-tetramethylcyclohexyl)pyrrolidine, N-(1,3,3,5-tetramethylcyclohexyl)piperidine, N-(1,3,3,5,5-pentamethylcyclohexyl)pyrrolidine, N-(1,3,3,5,5-pentamethylcyclohexyl)piperidine, N-(1,3,5,5-tetramethyl-3-ethylcyclohexyl)pyrrolidine, N-(1,3,5,5-tetramethyl-3-ethylcyclohexyl)piperidine, N-(1,5,5-trimethyl-3,3-diethylcyclohexyl)pyrrolidine, N-(1,5,5-trimethyl-3,3-diethylcyclohexyl)piperidine, N-(1,3,3-trimethyl-cis-5-ethylcyclohexyl)pyrrolidine, N-(1,3,3-trimethyl-cis-5-ethylcyclohexyl)piperidine, N-[(1S,5S)cis-5-ethyl-1,3,3-trimethylcyclohexyl]pyrrolidine, N-[(1S,5S)cis-5-ethyl-1,3,3-trimethylcyclohexyl]piperidine, N-(1,3,3-trimethyl-trans-5-ethylcyclohexyl)pyrrolidine, N-(1,3,3-trimethyl-trans-5-ethylcyclohexyl)piperidine, N-[(1R,5S)trans-5-ethyl,3,3-trimethylcyclohexyl]pyrrolidine, N-[(1R,5S)trans-5-ethyl,3,3-trimethylcyclohexyl]piperidine, N-(1-ethyl-3,3,5,5-tetramethylyclohexyl)pyrrolidine, N-(1-ethyl-3,3,5,5-tetramethylyclohexyl)piperidine, N-(1-propyl-3,3,5,5-tetramethylcyclohexyl)pyrrolidine, N-(1-propyl-3,3,5,5-tetramethylcyclohexyl)piperidine, N-(1,3,3,5,5-pentamethylcyclohexyl)pyrrolidine, spiro[cyclopropane-1,2-adamantan]-2-amine, spiro[pyrrolidine-2,2′-adamantane], spiro[piperidine-2,2-adamantane], 2-(2-adamantyl)piperidine, 3-(2-adamantyl)pyrrolidine, 2-(1-adamantyl)piperidine, 2-(1-adamantyl)pyrrolidine, and 2-(1-adamantyl)-2-methyl-pyrrolidine. In yet a further aspect, the amantadine analog is selected from 1-amino-3-phenyl adamantane, 1-amino-methyl adamantane, 1-amino-3-ethyl adamantane, 1-amino-3-isopropyl adamantane, 1-amino-3-n-butyl adamantane, 1-amino-3,5-diethyl adamantane, 1-amino-3,5-diisopropyl adamantane, 1-amino-3,5-di-n-butyl adamantane, 1-amino-3-methyl-5-ethyl adamantane, 1-N-methylamino-3,5-dimethyl adamantane, 1-N-ethylamino-3,5-dimethyl adamantane, 1-N-isopropyl-amino-3,5-dimethyl adamantane, 1-N,N-dimethyl-amino-3,5-dimethyl adamantane, 1-N-methyl-N-isopropyl-amino-3-methyl-5-ethyl adamantane, 1-amino-3-butyl-5-phenyl adamantane, 1-amino-3-pentyl adamantane, 1-amino-3,5-dipentyl adamantane, 1-amino-3-pentyl-5-hexyl adamantane, 1-amino-3-pentyl-5-cyclohexyl adamantane, 1-amino-3-pentyl-5-phenyl adamantane, 1-amino-3-hexyl adamantane, 1-amino-3,5-dihexyl adamantane, 1-amino-3-hexyl-5-cyclohexyl adamantane, 1-amino-3-hexyl-5-phenyl adamantane, 1-amino-3-cyclohexyl adamantane, 1-amino-3,5-dicyclohexyl adamantane, 1-amino-3-cyclohexyl-5-phenyl adamantane, 1-amino-3,5-diphenyl adamantane, 1-amino-3,5,7-trimethyl adamantane, 1-amino-3,5-dimethyl-7-ethyl adamantane, 1-amino-3,5-diethyl-7-methyl adamantane, 1-N-pyrrolidino and 1-N-piperidine derivatives, 1-amino-3-methyl-5-propyl adamantane, 1-amino-3-methyl-5-butyl adamantane, 1-amino-3-methyl-5-pentyl adamantane, 1-amino-3-methyl-5-hexyl adamantane, 1-amino-3-methyl-5-cyclohexyl adamantane, 1-amino-3-methyl-5-phenyl adamantane, 1-amino-3-ethyl-5-propyl adamantane, 1-amino-3-ethyl-5-butyl adamantane, 1-amino-3-ethyl-5-pentyl adamantane, 1-amino-3-ethyl-5-hexyl adamantane, 1-amino-3-ethyl-5-cyclohexyl adamantane, 1-amino-3-ethyl-5-phenyl adamantane, 1-amino-3-propyl-5-butyl adamantane, 1-amino-3-propyl-5-pentyl adamantane, 1-amino-3-propyl-5-hexyl adamantane, 1-amino-3-propyl-5-cyclohexyl adamantane, 1-amino-3-propyl-5-phenyl adamantane, 1-amino-3-butyl-5-pentyl adamantane, 1-amino-3-butyl-5-hexyl adamantane, and 1-amino-3-butyl-5-cyclohexyl adamantine.

In a further aspect, the neuraminidase inhibitor is selected from oseltamivir, zanamivir, peramivir, laninamivir octanoate, 2,3-didehydro-2-deoxy-N-acetylneuraminic acid (DANA), 2-deoxy-2,3-dehydro-N-trifluoroacetylneuraminic acid (FANA), N-[(1R,2S)-2-methoxy-2-methyl-1-[(2R,3S,5R)-5-(2-methylpropanoyl)-3-[(Z)-prop-1-enyl]pyrrolidin-2-yl]pentyl]acetamide (A-322278), and (2R,4S,5R)-5-[(1R,2S)-1-acetamido-2-methoxy-2-methylpentyl]-4-[(Z)-prop-1-enyl]pyrrolidine-2-carboxylic acid (A-315675), or a pharmaceutically acceptable salt, solvate, or polymorph thereof. In a still further aspect, the neuraminidase inhibitor is selected from oseltamivir, zanamivir, peramivir, laninamivir octanoate, or a pharmaceutically acceptable salt, solvate, or polymorph thereof. In yet a further aspect, the neuraminidase inhibitor is oseltamivir, oseltamivir phosphate, or oseltamivir carboxylate. In an even further aspect, the neuraminidase inhibitor is oseltamivir phosphate. In a still further aspect, the neuraminidase inhibitor is zanamivir. In yet a further aspect, the neuraminidase inhibitor is peramivir. In an even further aspect, the neuraminidase inhibitor is laninamivir octanoate.

In a further aspect, the nucleoside analog is selected from ribavirin, viramidine, 6-fluoro-3-hydroxy-2-pyrazinecarboxamide, 2′-deoxy-2′-fluoroguanosine, pyrazofurin, carbodine, and cyclopenenyl cytosine. In a still further aspect, the nucleoside analog is selected from ribavirin and viramidine. In yet a further aspect, the nucleoside analog is ribavirin. In an even further aspect, the nucleoside analog is viramidine.

In a further aspect, the kit further comprises an effective amount of an Akt therapeutic agent. In a still further aspect, the effective amount of the Akt therapeutic agent is a therapeutically effective amount. In yet a further aspect, the effective amount of the Akt therapeutic agent is a prophylactically effective amount.

In a further aspect, the Akt therapeutic agent is an Akt inhibitor. In a still further aspect, the Akt inhibitor binds to the pleckstrin homology domain. In yet a further aspect, the Akt inhibitor is an ATP-competitive inhibitor. In an even further aspect, the Akt inhibitor is an allosteric inhibitor. In a still further aspect, the allosteric inhibitor is MK-2066.

In a further aspect, the Akt inhibitor is a pan-Akt inhibitor.

In a further aspect, the Akt inhibitor inhibits Akt1, Akt2, or Akt3. In a still further aspect, the Akt inhibitor is an isoform-selective inhibitor. In yet a further aspect, the isoform-selective inhibitor selectively inhibits Akt1.

In a further aspect, the Akt therapeutic agent is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine. In a still further aspect, the Akt therapeutic agent is erucylphosphocholine. In yet a further aspect, the Akt therapeutic agent is GDC-0068. In an even further aspect, the Akt therapeutic agent is GSK-2141795. In a still further aspect, the Akt therapeutic agent is MK-2206. In yet a further aspect, the Akt therapeutic agent is perifosine. In an even further aspect, the Akt therapeutic agent is PHT-427.

In a further aspect, the Akt therapeutic agent is a siRNA.

In a further aspect, the Akt therapeutic agent is an antisense oligonucleotide. In a still further aspect, the antisense oligonucleotide is RX-0201.

In one aspect, the invention relates to a kit comprising an effective amount of a phospholipase D inhibitor, or a pharmaceutically acceptable prodrug, salt, solvate, or polymorph thereof; an effective amount of a mTor inhibitor, or a pharmaceutically acceptable prodrug, salt, solvate, or polymorph thereof; and one or more of: a) an effective amount of at least one agent known to treat a disorder of uncontrolled cellular proliferation; b) an effective amount of an Akt therapeutic agent; c) at least one agent known to increase Akt activity; or d) instructions for treating a disorder of uncontrolled cellular proliferation. In a further aspect, the effective amount of the PLD inhibitor is a therapeutically effective amount. In a still further aspect, the effective amount of the PLD inhibitor is a prophylatically effective amount.

In a further aspect, the PLD inhibitor is a compound having a structure represented by a formula:

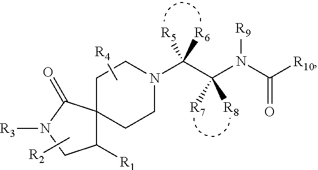

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

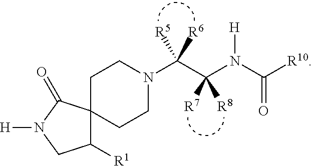

In a further aspect, the PLD inhibitor is a compound having a structure represented by a formula:

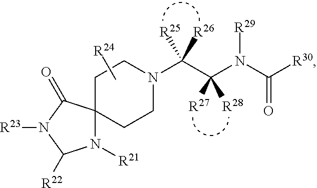

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

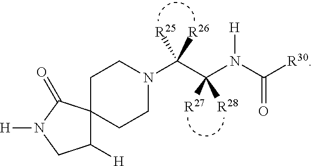

In a further aspect, the compound is:

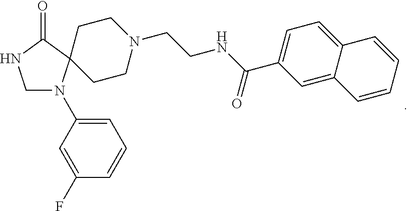 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

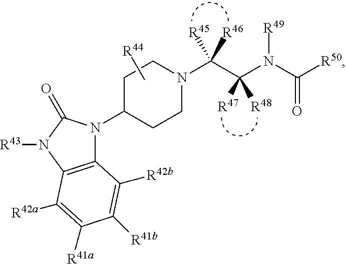

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

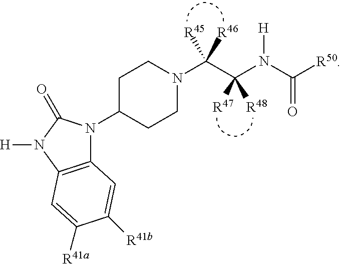

In a further aspect, the compound is:

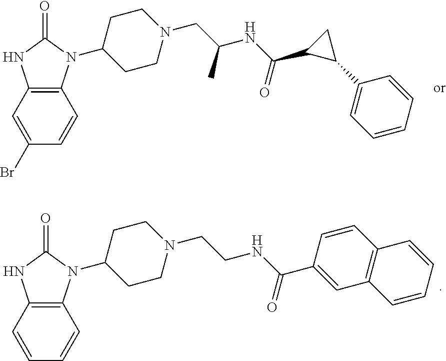

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof.

In a further aspect, the PLD inhibitor is a compound selected from:

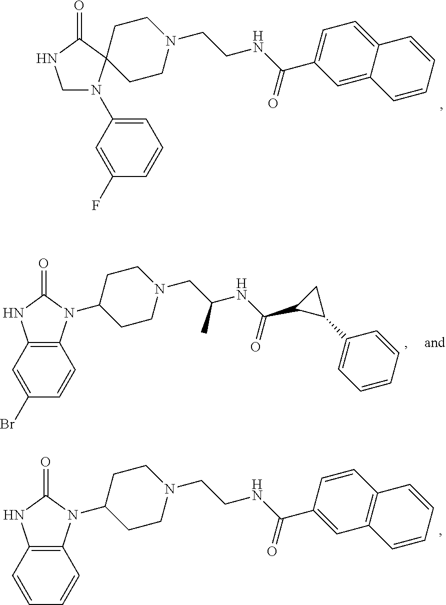

or a pharmaceutically acceptable salt, hydrate, solvate, or polymorph thereof.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the phospholipase D inhibitor is selected from:

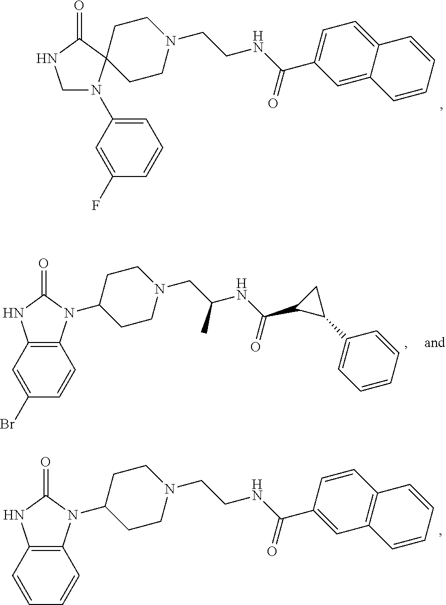

or a pharmaceutically acceptable salt, hydrate, solvate, or polymorph thereof.

In a further aspect, the effective amount of an mTor inhibitor is a therapeutically effective amount. In a still further aspect, the effective amount of an mTor inhibitor is a prophylatically effective amount.

In a further aspect, the mTor inhibitor is selected from everolimus, rapamycin (sirolimus), temsirolimus, deforolimus, ridaforolimus, tacrolimus, zotarolimus, salirasib, curcumin, farnesylthiosalicylic acid, XL765, ABI-009, AP-23675, AP-23841, AP-23765, AZD-8055, AZD-2014, BEZ-235 (NVP-BEZ235), BGT226, GDC-0980, INK-128, KU-0063794, MK8669, MKC-1 (Ro 31-7453), NVP-BGT226, OSI-027, Palomid-529, PF-04691502, PKI-402, PKI-587, PP-242, PP-30, SB-1518, SB-2312, SF-1126, TAFA-93, TOP-216, Torin1, WAY-600, WYE-125132, WYE-354, WYE-687, and XL-765, or a pharmaceutically acceptable prodrug, salt, solvate, or polymorph thereof. In a still further aspect, the mTor inhibitor is selected from everolimus, rapamycin (sirolimus), and temsirolimus, or a pharmaceutically acceptable prodrug, salt, solvate, or polymorph thereof. In yet a further aspect, the mTor inhibitor is everolimus, or a pharmaceutically acceptable prodrug, salt, solvate, or polymorph thereof. In an even further aspect, the mTor inhibitor is rapamycin (sirolimus), or a pharmaceutically acceptable prodrug, salt, solvate, or polymorph thereof. In a still further aspect, the mTor inhibitor is temsiorlimus, or a pharmaceutically acceptable prodrug, salt, solvate, or polymorph thereof. In yet a further aspect, the mTor inhibitor is deforolimus, or a pharmaceutically acceptable prodrug, salt, solvate, or polymorph thereof. In an even further aspect, the mTor inhibitor is tacrolimus, or a pharmaceutically acceptable prodrug, salt, solvate, or polymorph thereof. In a still further aspect, the mTor inhibitor is zotarolimus, or a pharmaceutically acceptable prodrug, salt, solvate, or polymorph thereof. In yet a further aspect, the mTor inhibitor is salirasib, or a pharmaceutically acceptable prodrug, salt, solvate, or polymorph thereof. In an even further aspect, the mTor inhibitor is curcumin, or a pharmaceutically acceptable prodrug, salt, solvate, or polymorph thereof. In a still further aspect, the mTor inhibitor is farnesylthiosalicylic acid, or a pharmaceutically acceptable prodrug, salt, solvate, or polymorph thereof. In yet a further aspect, the mTor inhibitor is Torin1, or a pharmaceutically acceptable prodrug, salt, solvate, or polymorph thereof.

In a further aspect, the effective amount of the Akt therapeutic agent is a therapeutically effective amount. In a still further aspect, the effective amount of the Akt therapeutic agent is a prophylactically effective amount.

In a further aspect, the Akt therapeutic agent is an Akt inhibitor. In a still further aspect, the Akt inhibitor binds to the pleckstrin homology domain. In yet a further aspect, the Akt inhibitor is an ATP-competitive inhibitor. In an even further aspect, the Akt inhibitor is an allosteric inhibitor. In a still further aspect, the allosteric inhibitor is MK-2066.

In a further aspect, the Akt inhibitor is a pan-Akt inhibitor.

In a further aspect, the Akt inhibitor inhibits Akt1, Akt2, or Akt3. In a still further aspect, the Akt inhibitor is an isoform-selective inhibitor. In yet a further aspect, the isoform-selective inhibitor selectively inhibits Akt1.

In a further aspect, the Akt therapeutic agent is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine. In a still further aspect, the Akt therapeutic agent is erucylphosphocholine. In yet a further aspect, the Akt therapeutic agent is GDC-0068. In an even further aspect, the Akt therapeutic agent is GSK-2141795. In a still further aspect, the Akt therapeutic agent is MK-2206. In yet a further aspect, the Akt therapeutic agent is perifosine. In an even further aspect, the Akt therapeutic agent is PHT-427.

In a further aspect, the Akt therapeutic agent is a siRNA.

In a further aspect, the Akt therapeutic agent is an antisense oligonucleotide. In a still further aspect, the antisense oligonucleotide is RX-0201.

In a further aspect, the effective amount of at least one agent known to treat a disorder of uncontrolled cellular proliferation is an effective amount of at least one anticancer agent. In a still further aspect, the one anticancer agent selected from: a) a hormone therapy therapeutic agent, or a pharmaceutically acceptable prodrug, salt, solvate, or polymorph thereof b) an alkylating therapeutic agent, or a pharmaceutically acceptable prodrug, salt, solvate, or polymorph thereof; c) an antineoplastic antimetabolite therapeutic agent, or a pharmaceutically acceptable prodrug, salt, solvate, or polymorph thereof; d) a mitotic inhibitor therapeutic agent, or a pharmaceutically acceptable prodrug, salt, solvate, or polymorph thereof; e) an antineoplastic antibiotic therapeutic agent, or a pharmaceutically acceptable prodrug, salt, solvate, or polymorph thereof; or f) other chemotherapeutic agent, or a pharmaceutically acceptable prodrug, salt, solvate, or polymorph thereof. In yet a further aspect, the effective amount of at least one anticancer agent is a therapeutically effective amount. In an even further aspect, the effective amount of at least one anticancer agent is a prophylactically effective amount.

In a further aspect, the hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt, hydrate, solvate, or polymorph thereof.

In a further aspect, the alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof.

In a further aspect, the antineoplastic antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt, hydrate, solvate, or polymorph thereof.

In a further aspect, the mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt, hydrate, solvate, or polymorph thereof.

In a further aspect, the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt, hydrate, solvate, or polymorph thereof.

In a further aspect, the instructions for treating a disorder of uncontrolled cellular proliferation are instructions for treating a cancer. In a still further aspect, the cancer is a hematological cancer. In yet a further aspect, the hematological cancer is selected from a leukemia, lymphoma, chronic myeloproliferative disorder, myelodysplastic syndrome, myeloproliferative neoplasm, plasma cell neoplasm (myeloma), solid tumor, sarcoma, and carcinoma.

In a further aspect, the cancer is a leukemia. In a still further aspect, the leukemia is selected from acute leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, and chronic lymphocytic leukemia.

In a further aspect, the cancer is a lymphoma. In a still further aspect, the lymphoma is selected from AIDS-Related lymphoma, cutaneous T-Cell lymphoma, Hodgkin lymphoma, non-Hodgkin lymphoma, primary central nervous system lymphoma, mycosis fungoides and the Sézary Syndrome, heavy chain disease, and Waldenström macroglobulinemia. In yet a further aspect, the lymphoma is Hodgkin's lymphoma. In an even further aspect, the lymphoma is non-Hodgkin's lymphoma.

In a further aspect, the cancer is a solid tumor. In a still further aspect, the cancer is selected from a cancer of the brain, genitourinary tract, gastrointestinal tract, colon, rectum, breast, kidney, lymphatic system, stomach, lung, pancreas, and skin. In yet a further aspect, the cancer is selected from prostate cancer, glioblastoma multiforme, endometrial cancer, breast cancer, and colon cancer. In an even further aspect, the cancer is selected from synovioma, mesothelioma, Ewing's tumor, pancreatic cancer, breast cancer, ovarian cancer, prostate cancer, hepatoma, Wilms' tumor, cervical cancer, testicular cancer, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In a further aspect, the cancer is a sarcoma. In a still further aspect, the sarcoma is selected from fibrosarcoma, myxosarcoma, liposarcoma, chondrosarcoma, osteogenic sarcoma, chordoma, angiosarcoma, endotheliosarcoma, lymphangiosarcoma, leiomyosarcoma, rhabdomyosarcoma, and lymphangioendotheliosarcoma.

In a further aspect, the cancer is a carcinoma. In a still further aspect, the carcinoma is selected from colon carcinoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, bile duct carcinoma, choriocarcinoma, seminoma, embryonal carcinoma, lung carcinoma, small cell lung carcinoma, bladder carcinoma, and epithelial carcinoma.

In a further aspect, the cancer is associated with a loss of PTEN function. In a still further aspect, the loss of PTEN function is associated with a mutation is in the PTEN gene. In yet a further aspect, the mutation is selected from c.17_{—}18delAA, c.955_{—}958delACTT, c.741_{—}742insA, c.968_{—}969insA, c.742_{—}743insC, c.742_{—}743insA, c.389G>A, c.202T>C, c.518G>A, c.517C>T, c.511C>T, c.640C>T, c.1003C>T, c.388C>T, c.697C>T, c.389G>T, c.388C>G, c.1002_{—}1003CC>TT, c.968delA, c.800delA, c.867delA, c.389delG, c.723_{—}724insTT, and c.969delT. In an even further aspect, the mutation in the PTEN gene is associated with a mutation in the PTEN protein selected from p.E242fs*15, p.K267fs*9, p.K6fs*4, p.N323fs*2, p.N323fs*21, p.P248fs*5, p.Q171*, p.Q214*, p.R130*, p.R130fs*4, p.R130G, p.R130L, p.R130Q, p.R173c, p.R173H, p.R233*, p.R335*, p.T319fs*1, p.V290fs*1, and p.Y68H.

In a further aspect, the cancer is associated with PI3K activation. In a still further aspect, the PI3K activation is associated with a mutation is in the PIK3CA gene. In yet a further aspect, the mutation is selected from c.3149G>A, c.3194A>T, c.3012G>T, c.1638G>T, c.3141T>G, c.3146G>C, c.323G>A, c.353G>A, c.3127A>G, c.113G>A, c.333G>C, c.331A>G, c.277C>T, c.1634A>T, c.1035T>A, c.1258T>C, c.1616C>G, c.1624G>A, c.1625A>T, c.1634A>G, c.1636C>A, c.1637A>C, c.3129G>T, c.3139C>T, c.3140A>T, c.3145G>A, c.2102A>C, c.1634A>C, c.1637A>G, c.3062A>G, c.1624G>C, c.1633G>C, c.1635G>C, c.3073A>G, c.1635G>Tc.3204_{—}3205insA, c.1633G>A, c.3140A>G, c.1636C>G, c.3145G>C, c.263G>A, c.1637A>T, c.317G>T, and c.3068G>A. In an even further aspect, the mutation in the PIK3CA gene is associated with a mutation in the PIK3CA protein selected from p.C420R, p.E542K, p.E542Q, p.E542V, p.E545A, p.E545D, p.E545G, p.E545K, p.E545Q, p.E545V, p.G1049A, p.G1049R, p.G1049S, p.G1050D, p.G106V, p.G118D, p.H1047L, p.H1047Q, p.H1047R, p.H1047Y, p.H1065L, p.H701P, p.K111E, p.K111N, p.M1004I, p.M1043I, p.M1043V, p.N1068fs*4, p.N345K, p.P539R, p.Q546E, p.Q546H, p.Q546K, p.Q546L, p.Q546P, p.Q546R, p.R1023Q, p.R108H, p.R38H, p.R88Q, p.R93W, p.T1025A, and p.Y1021C.

In one aspect, the invention relates to a kit comprising an effective amount of at least one phospholipase D inhibitor, or a pharmaceutically acceptable prodrug, salt, solvate, or polymorph thereof; instructions for administering the phospholipase D inhibitor to a subject identified with a mutation associated with activation of Akt; and one or more of: a) at least one anticancer therapeutic agent; b) an effective amount of an Akt therapeutic agent; c) at least one agent known to increase Akt activity; d) instructions for treating a disorder of uncontrolled cellular proliferation; or e) instructions for administering the phospholipase D inhibitor with the anticancer therapeutic agent and/or Akt therapeutic agent. In a further aspect, the effective amount of the PLD inhibitor is a therapeutically effective amount. In a still further aspect, the effective amount of the PLD inhibitor is a prophylatically effective amount.

In a further aspect, the PLD inhibitor is a compound having a structure represented by a formula:

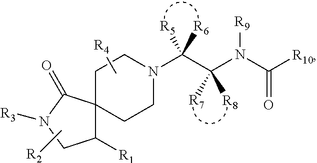

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

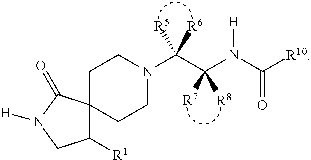

In a further aspect, the PLD inhibitor is a compound having a structure represented by a formula:

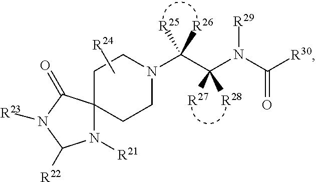

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

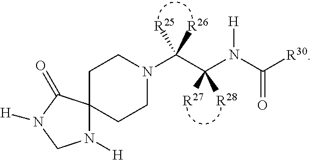

In a further aspect, the compound is:

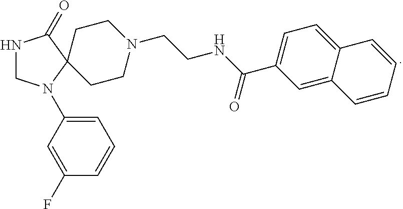 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

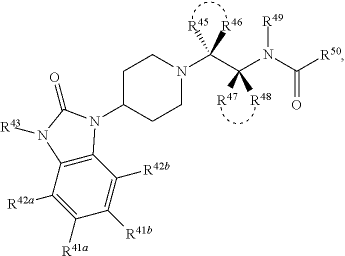

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

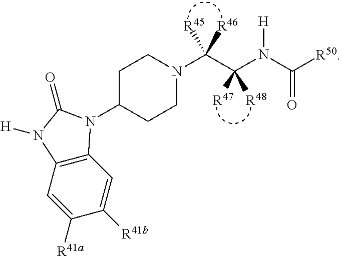

In a further aspect, the compound is:

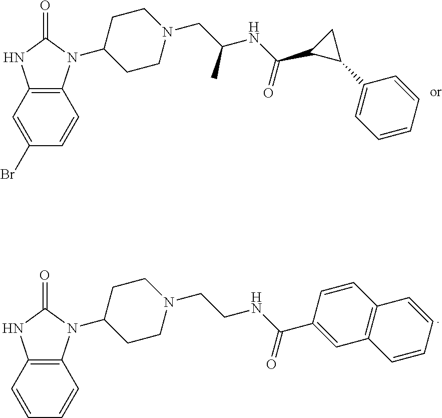

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof.

In a further aspect, the PLD inhibitor is a compound selected from:

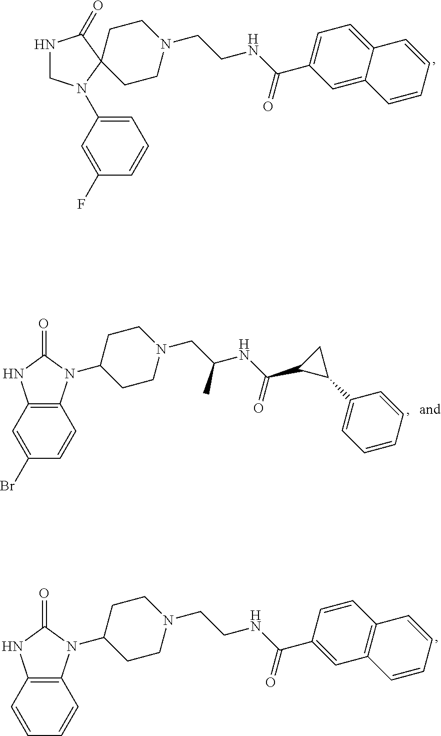

or a pharmaceutically acceptable salt, hydrate, solvate, or polymorph thereof.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the phospholipase D inhibitor is selected from:

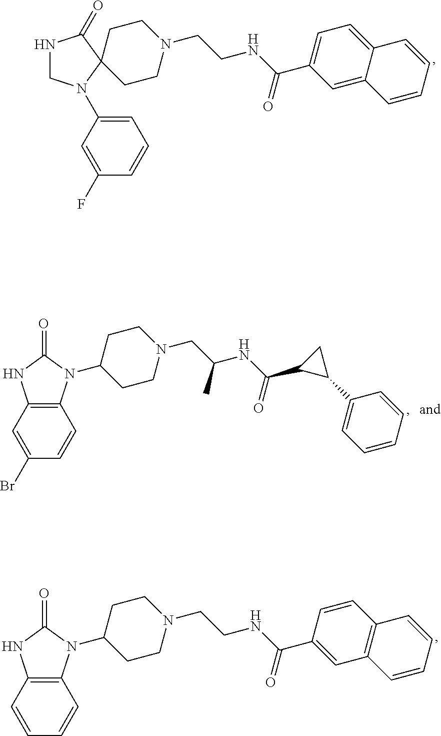

or a pharmaceutically acceptable salt, hydrate, solvate, or polymorph thereof.

In a further aspect, the effective amount of the Akt therapeutic agent is a therapeutically effective amount. In a still further aspect, the effective amount of the Akt therapeutic agent is a prophylactically effective amount.

In a further aspect, the Akt therapeutic agent is an Akt inhibitor. In a still further aspect, the Akt inhibitor binds to the pleckstrin homology domain. In yet a further aspect, the Akt inhibitor is an ATP-competitive inhibitor. In an even further aspect, the Akt inhibitor is an allosteric inhibitor. In a still further aspect, the allosteric inhibitor is MK-2066.

In a further aspect, the Akt inhibitor is a pan-Akt inhibitor.

In a further aspect, the Akt inhibitor inhibits Akt1, Akt2, or Akt3. In a still further aspect, the Akt inhibitor is an isoform-selective inhibitor. In yet a further aspect, the isoform-selective inhibitor selectively inhibits Akt1.

In a further aspect, the Akt therapeutic agent is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine. In a still further aspect, the Akt therapeutic agent is erucylphosphocholine. In yet a further aspect, the Akt therapeutic agent is GDC-0068. In an even further aspect, the Akt therapeutic agent is GSK-2141795. In a still further aspect, the Akt therapeutic agent is MK-2206. In yet a further aspect, the Akt therapeutic agent is perifosine. In an even further aspect, the Akt therapeutic agent is PHT-427.

In a further aspect, the Akt therapeutic agent is a siRNA.

In a further aspect, the Akt therapeutic agent is an antisense oligonucleotide. In a still further aspect, the antisense oligonucleotide is RX-0201.

In a further aspect, the effective amount of at least one anticancer therapeutic agent is a therapeutically effective amount. In a still further aspect, the effective amount of at least one anticancer therapeutic agent is a prophylactically effective amount.

In a further aspect, the one anticancer therapeutic agent selected from: a) a hormone therapy therapeutic agent, or a pharmaceutically acceptable prodrug, salt, solvate, or polymorph thereof; b) an alkylating therapeutic agent, or a pharmaceutically acceptable prodrug, salt, solvate, or polymorph thereof; c) an antineoplastic antimetabolite therapeutic agent, or a pharmaceutically acceptable prodrug, salt, solvate, or polymorph thereof; d) a mitotic inhibitor therapeutic agent, or a pharmaceutically acceptable prodrug, salt, solvate, or polymorph thereof; e) an antineoplastic antibiotic therapeutic agent, or a pharmaceutically acceptable prodrug, salt, solvate, or polymorph thereof; or f) other chemotherapeutic agent, or a pharmaceutically acceptable prodrug, salt, solvate, or polymorph thereof.

In a further aspect, the hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt, hydrate, solvate, or polymorph thereof.

In a further aspect, the alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof.

In a further aspect, the antineoplastic antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt, hydrate, solvate, or polymorph thereof.

In a further aspect, the mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt, hydrate, solvate, or polymorph thereof.

In a further aspect, the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt, hydrate, solvate, or polymorph thereof.

In a further aspect, the instructions for treating a disorder of uncontrolled cellular proliferation are instructions for treating a cancer. In a still further aspect, the cancer is a hematological cancer. In yet a further aspect, the hematological cancer is selected from a leukemia, lymphoma, chronic myeloproliferative disorder, myelodysplastic syndrome, myeloproliferative neoplasm, plasma cell neoplasm (myeloma), solid tumor, sarcoma, and carcinoma. In an even further aspect, the hematological cancer is a leukemia.

In a further aspect, the leukemia is selected from acute leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, and chronic lymphocytic leukemia.

In a further aspect, the cancer is a lymphoma. In a still further aspect, the lymphoma is selected from AIDS-Related lymphoma, cutaneous T-Cell lymphoma, Hodgkin lymphoma, non-Hodgkin lymphoma, primary central nervous system lymphoma, mycosis fungoides and the Sézary Syndrome, heavy chain disease, and Waldenström macroglobulinemia. In yet a further aspect, the lymphoma is Hodgkin's lymphoma. In an even further aspect, the lymphoma is non-Hodgkin's lymphoma.

In a further aspect, the cancer is a solid tumor. In a still further aspect, the cancer is selected from a cancer of the brain, genitourinary tract, gastrointestinal tract, colon, rectum, breast, kidney, lymphatic system, stomach, lung, pancreas, and skin. In yet a further aspect, the cancer is selected from prostate cancer, glioblastoma multiforme, endometrial cancer, breast cancer, and colon cancer. In an even further aspect, the cancer is selected from synovioma, mesothelioma, Ewing's tumor, pancreatic cancer, breast cancer, ovarian cancer, prostate cancer, hepatoma, Wilms' tumor, cervical cancer, testicular cancer, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In a further aspect, the cancer is a sarcoma. In a still further aspect, the sarcoma is selected from fibrosarcoma, myxosarcoma, liposarcoma, chondrosarcoma, osteogenic sarcoma, chordoma, angiosarcoma, endotheliosarcoma, lymphangiosarcoma, leiomyosarcoma, rhabdomyosarcoma, and lymphangioendotheliosarcoma.

In a further aspect, the cancer is a carcinoma. In a still further aspect, the carcinoma is selected from colon carcinoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, bile duct carcinoma, choriocarcinoma, seminoma, embryonal carcinoma, lung carcinoma, small cell lung carcinoma, bladder carcinoma, and epithelial carcinoma.

In a further aspect, the cancer is associated with a loss of PTEN function. In a still further aspect, the loss of PTEN function is associated with a mutation is in the PTEN gene. In yet a further aspect, the mutation is selected from c.17_{—}18delAA, c.955_{—}958delACTT, c.741_{—}742insA, c.968_{—}969insA, c.742_{—}743insC, c.742_{—}743insA, c.389G>A, c.202T>C, c.518G>A, c.517C>T, c.511C>T, c.640C>T, c.1003C>T, c.388C>T, c.697C>T, c.389G>T, c.388C>G, c.1002_{—}1003CC>TT, c.968delA, c.800delA, c.867delA, c.389delG, c.723_{—}724insTT, and c.969delT. In an even further aspect, the mutation in the PTEN gene is associated with a mutation in the PTEN protein selected from p.E242fs*15, p.K267fs*9, p.K6fs*4, p.N323fs*2, p.N323fs*21, p.P248fs*5, p.Q171*, p.Q214*, p.R130*, p.R130fs*4, p.R130G, p.R130L, p.R130Q, p.R173c, p.R173H, p.R233*, p.R335*, p.T319fs*1, p.V290fs*1, and p.Y68H.

In a further aspect, the cancer is associated with PI3K activation. In a still further aspect, the PI3K activation is associated with a mutation is in the PIK3CA gene. In yet a further aspect, the mutation is selected from c.3149G>A, c.3194A>T, c.3012G>T, c.1638G>T, c.3141T>G, c.3146G>C, c.323G>A, c.353G>A, c.3127A>G, c.113G>A, c.333G>C, c.331A>G, c.277C>T, c.1634A>T, c.1035T>A, c.1258T>C, c.1616C>G, c.1624G>A, c.1625A>T, c.1634A>G, c.1636C>A, c.1637A>C, c.3129G>T, c.3139C>T, c.3140A>T, c.3145G>A, c.2102A>C, c.1634A>C, c.1637A>G, c.3062A>G, c.1624G>C, c.1633G>C, c.1635G>C, c.3073A>G, c.1635G>Tc.3204_{—}3205insA, c.1633G>A, c.3140A>G, c.1636C>G, c.3145G>C, c.263G>A, c.1637A>T, c.317G>T, and c.3068G>A. In an even further aspect, the mutation in the PIK3CA gene is associated with a mutation in the PIK3CA protein selected from p.C420R, p.E542K, p.E542Q, p.E542V, p.E545A, p.E545D, p.E545G, p.E545K, p.E545Q, p.E545V, p.G1049A, p.G1049R, p.G1049S, p.G1050D, p.G106V, p.G118D, p.H1047L, p.H1047Q, p.H1047R, p.H1047Y, p.H1065L, p.H701P, p.K111E, p.K111N, p.M1004I, p.M1043I, p.M1043V, p.N1068fs*4, p.N345K, p.P539R, p.Q546E, p.Q546H, p.Q546K, p.Q546L, p.Q546P, p.Q546R, p.R1023Q, p.R108H, p.R38H, p.R88Q, p.R93W, p.T1025A, and p.Y1021C.

In a further aspect, the kit further comprises an effective amount of an mTor inhibitor. In a still further aspect, the effective amount of an mTor inhibitor is a therapeutically effective amount. In yet a further aspect, the effective amount of an mTor inhibitor is a prophylatically effective amount.

In a further aspect, the mTor inhibitor is selected from everolimus, rapamycin (sirolimus), temsirolimus, deforolimus, ridaforolimus, tacrolimus, zotarolimus, salirasib, curcumin, farnesylthiosalicylic acid, XL765, ABI-009, AP-23675, AP-23841, AP-23765, AZD-8055, AZD-2014, BEZ-235 (NVP-BEZ235), BGT226, GDC-0980, INK-128, KU-0063794, MK8669, MKC-1 (Ro 31-7453), NVP-BGT226, OSI-027, Palomid-529, PF-04691502, PKI-402, PKI-587, PP-242, PP-30, SB-1518, SB-2312, SF-1126, TAFA-93, TOP-216, Torin1, WAY-600, WYE-125132, WYE-354, WYE-687, and XL-765, or a pharmaceutically acceptable prodrug, salt, solvate, or polymorph thereof. In a still further aspect, the mTor inhibitor is selected from everolimus, rapamycin (sirolimus), and temsirolimus, or a pharmaceutically acceptable prodrug, salt, solvate, or polymorph thereof. In yet a further aspect, the mTor inhibitor is everolimus, or a pharmaceutically acceptable prodrug, salt, solvate, or polymorph thereof. In an even further aspect, the mTor inhibitor is rapamycin (sirolimus), or a pharmaceutically acceptable prodrug, salt, solvate, or polymorph thereof. In a still further aspect, the mTor inhibitor is temsiorlimus, or a pharmaceutically acceptable prodrug, salt, solvate, or polymorph thereof. In yet a further aspect, the mTor inhibitor is deforolimus, or a pharmaceutically acceptable prodrug, salt, solvate, or polymorph thereof. In an even further aspect, the mTor inhibitor is tacrolimus, or a pharmaceutically acceptable prodrug, salt, solvate, or polymorph thereof. In a still further aspect, the mTor inhibitor is zotarolimus, or a pharmaceutically acceptable prodrug, salt, solvate, or polymorph thereof. In yet a further aspect, the mTor inhibitor is salirasib, or a pharmaceutically acceptable prodrug, salt, solvate, or polymorph thereof. In an even further aspect, the mTor inhibitor is curcumin, or a pharmaceutically acceptable prodrug, salt, solvate, or polymorph thereof. In a still further aspect, the mTor inhibitor is farnesylthiosalicylic acid, or a pharmaceutically acceptable prodrug, salt, solvate, or polymorph thereof. In yet a further aspect, the mTor inhibitor is Torin1, or a pharmaceutically acceptable prodrug, salt, solvate, or polymorph thereof.

In one aspect, the invention relates to a kit comprising an effective amount of a phospholipase D inhibitor, or a pharmaceutically acceptable prodrug, salt, solvate, or polymorph thereof; an effective amount of an autophagy inducer, or a pharmaceutically acceptable prodrug, salt, solvate, or polymorph thereof; and one or more of: a) at least one agent known to decrease the severity of symptoms associated with an infectious disease; b) at least one agent known to treat an infectious disease; c) instructions for treating an infectious disease; d) instructions for administering the phospholipase D inhibitor and autophagy inducer in connection with treating an infectious disease; or e) instructions for administering the phospholipase D inhibitor and autophagy inducer in connection with reducing the risk of an infectious disease. In a further aspect, an effective amount of the PLD inhibitor is a therapeutically effective amount. In a still further aspect, an effective amount of the PLD inhibitor is a prophylactically effective amount.

In a further aspect, the PLD inhibitor is a compound having a structure represented by a formula:

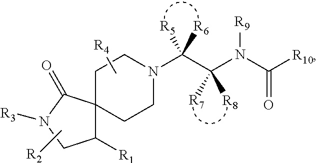

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

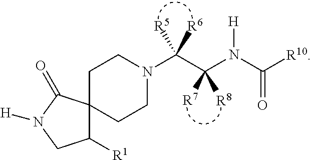

In a further aspect, the PLD inhibitor is a compound having a structure represented by a formula:

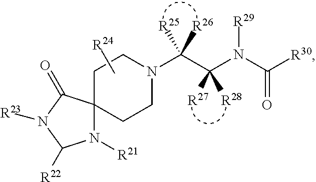

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

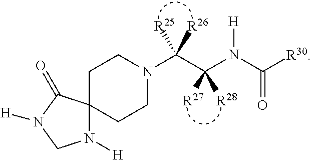

In a further aspect, the compound is:

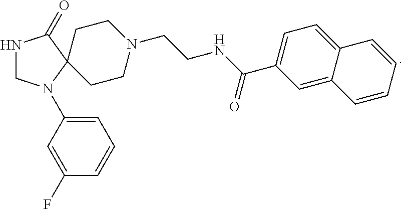 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

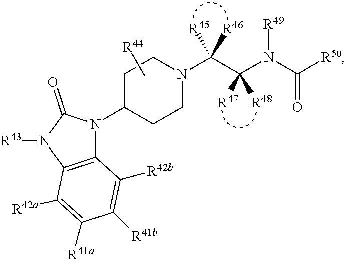

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

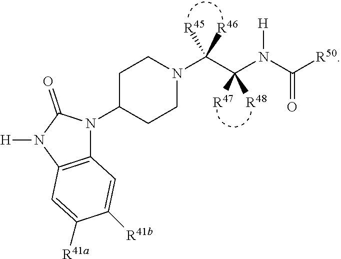

In a further aspect, the compound is:

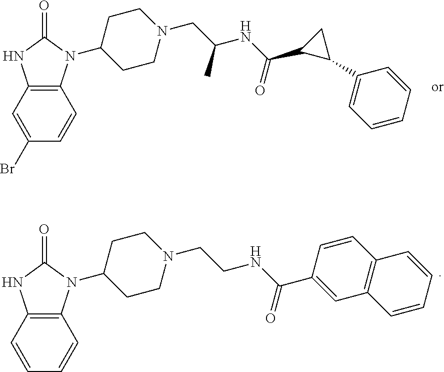

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof.

In a further aspect, the PLD inhibitor is a compound selected from:

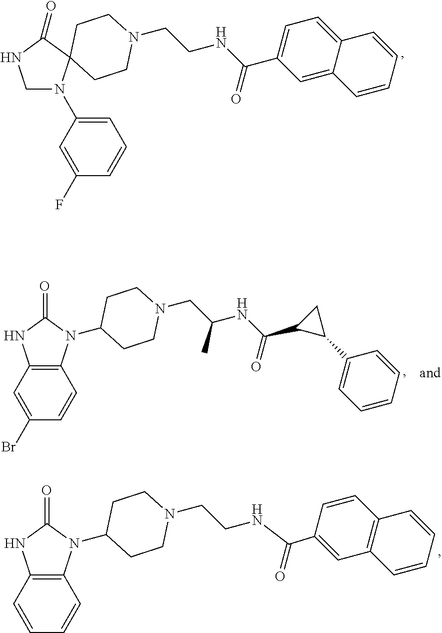

or a pharmaceutically acceptable salt, hydrate, solvate, or polymorph thereof.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the phospholipase D inhibitor is selected from:

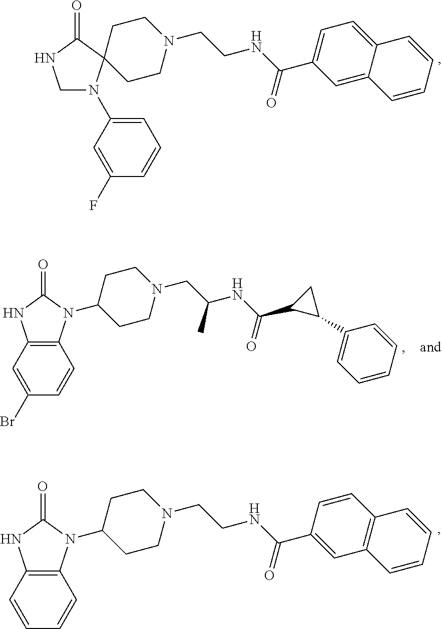

or a pharmaceutically acceptable salt, hydrate, solvate, or polymorph thereof.

In a further aspect, the autophagy inducer is an effective amount of an mTor inhibitor. In a still further aspect, the effective amount of an mTor inhibitor is a therapeutically effective amount. In yet a further aspect, the effective amount of an mTor inhibitor is a prophylatically effective amount.

In a further aspect, the mTor inhibitor is selected from everolimus, rapamycin (sirolimus), temsirolimus, deforolimus, ridaforolimus, tacrolimus, zotarolimus, salirasib, curcumin, farnesylthiosalicylic acid, XL765, ABI-009, AP-23675, AP-23841, AP-23765, AZD-8055, AZD-2014, BEZ-235 (NVP-BEZ235), BGT226, GDC-0980, INK-128, KU-0063794, MK8669, MKC-1 (Ro 31-7453), NVP-BGT226, OSI-027, Palomid-529, PF-04691502, PKI-402, PKI-587, PP-242, PP-30, SB-1518, SB-2312, SF-1126, TAFA-93, TOP-216, Torin1, WAY-600, WYE-125132, WYE-354, WYE-687, and XL-765, or a pharmaceutically acceptable prodrug, salt, solvate, or polymorph thereof. In a still further aspect, the mTor inhibitor is selected from everolimus, rapamycin (sirolimus), and temsirolimus, or a pharmaceutically acceptable prodrug, salt, solvate, or polymorph thereof. In yet a further aspect, the mTor inhibitor is everolimus, or a pharmaceutically acceptable prodrug, salt, solvate, or polymorph thereof. In an even further aspect, the mTor inhibitor is rapamycin (sirolimus), or a pharmaceutically acceptable prodrug, salt, solvate, or polymorph thereof. In a still further aspect, the mTor inhibitor is temsiorlimus, or a pharmaceutically acceptable prodrug, salt, solvate, or polymorph thereof. In yet a further aspect, the mTor inhibitor is deforolimus, or a pharmaceutically acceptable prodrug, salt, solvate, or polymorph thereof. In an even further aspect, the mTor inhibitor is tacrolimus, or a pharmaceutically acceptable prodrug, salt, solvate, or polymorph thereof. In a still further aspect, the mTor inhibitor is zotarolimus, or a pharmaceutically acceptable prodrug, salt, solvate, or polymorph thereof. In yet a further aspect, the mTor inhibitor is salirasib, or a pharmaceutically acceptable prodrug, salt, solvate, or polymorph thereof. In an even further aspect, the mTor inhibitor is curcumin, or a pharmaceutically acceptable prodrug, salt, solvate, or polymorph thereof. In a still further aspect, the mTor inhibitor is farnesylthiosalicylic acid, or a pharmaceutically acceptable prodrug, salt, solvate, or polymorph thereof. In yet a further aspect, the mTor inhibitor is Torin1, or a pharmaceutically acceptable prodrug, salt, solvate, or polymorph thereof.

In one aspect, the invention relates to a kit comprising an effective amount of a phospholipase D inhibitor, or a pharmaceutically acceptable prodrug, salt, solvate, or polymorph thereof; and one or more of: a) at least one agent known to increase Akt activity; b) at least one agent known to decrease Akt activity; c) instructions for treating an infectious disease; or d) instructions for administering the phospholipase D inhibitor in connection with treating a disorder associated with an increase in Akt activity. In a further aspect, an effective amount of the PLD inhibitor is a therapeutically effective amount. In a still further aspect, an effective amount of the PLD inhibitor is a prophylactically effective amount.

In a further aspect, the PLD inhibitor is a compound having a structure represented by a formula:

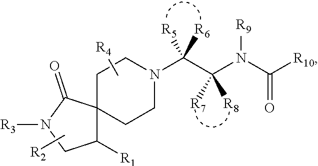

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

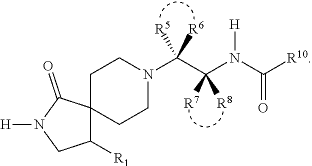

In a further aspect, the PLD inhibitor is a compound having a structure represented by a formula:

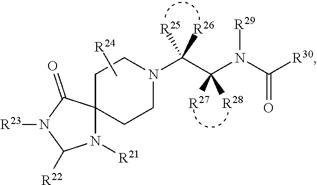

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

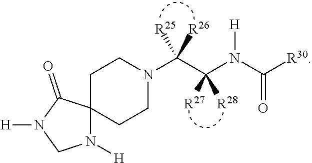

In a further aspect, the compound is:

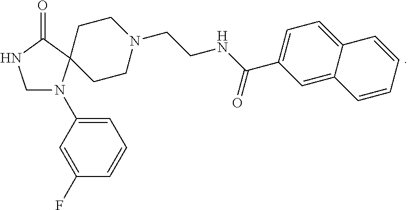 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

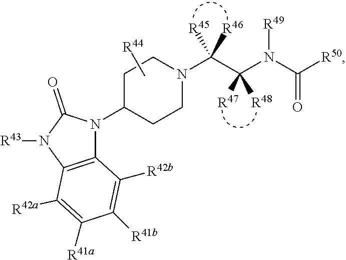

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

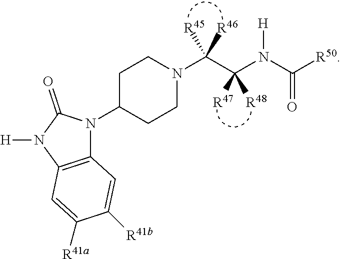

In a further aspect, the compound is:

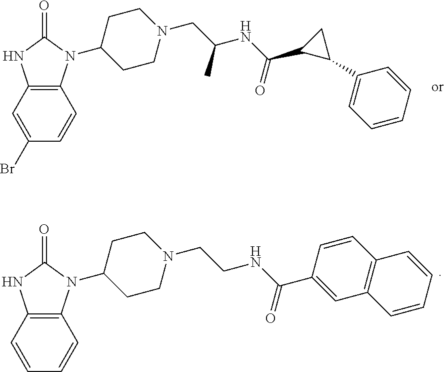

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof.

In a further aspect, the PLD inhibitor is a compound selected from:

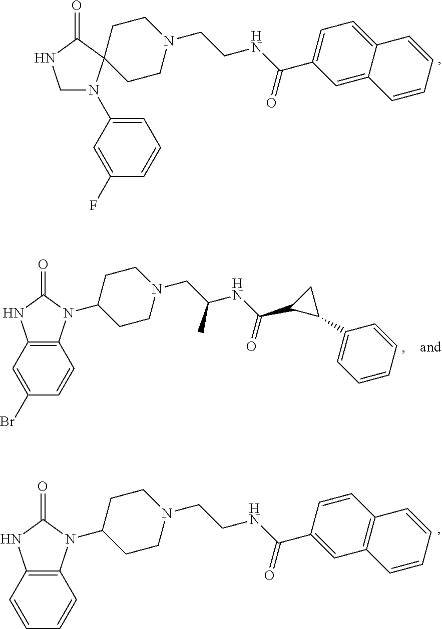

or a pharmaceutically acceptable salt, hydrate, solvate, or polymorph thereof.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the phospholipase D inhibitor is selected from:

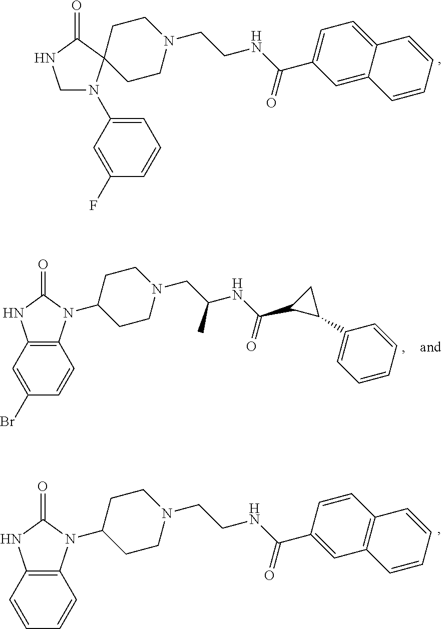

or a pharmaceutically acceptable salt, hydrate, solvate, or polymorph thereof.

In a further aspect, the at least one agent known to decrease Akt activity is an Akt inhibitor. In a still further aspect, the Akt inhibitor binds to the pleckstrin homology domain. In yet a further aspect, the Akt inhibitor is an ATP-competitive inhibitor. In an even further aspect, the Akt inhibitor is an allosteric inhibitor. In a still further aspect, the allosteric inhibitor is MK-2066.

In a further aspect, the Akt inhibitor is a pan-Akt inhibitor.

In a further aspect, the Akt inhibitor inhibits Akt1, Akt2, or Akt3. In a still further aspect, the Akt inhibitor is an isoform-selective inhibitor. In yet a further aspect, the isoform-selective inhibitor selectively inhibits Akt1.

In a further aspect, the Akt therapeutic agent is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine. In a still further aspect, the Akt therapeutic agent is erucylphosphocholine. In yet a further aspect, the Akt therapeutic agent is GDC-0068. In an even further aspect, the Akt therapeutic agent is GSK-2141795. In a still further aspect, the Akt therapeutic agent is MK-2206. In yet a further aspect, the Akt therapeutic agent is perifosine. In an even further aspect, the Akt therapeutic agent is PHT-427.

In a further aspect, the at least one agent known to decrease Akt activity is a siRNA.

In a further aspect, the at least one agent known to decrease Akt activity is an antisense oligonucleotide. In a still further aspect, the antisense oligonucleotide is RX-0201.

In one aspect, the invention relates to a kit comprising an effective amount of a phospholipase D inhibitor, or a pharmaceutically acceptable prodrug, salt, solvate, or polymorph thereof; an effective amount of an autophagy inducer, or a pharmaceutically acceptable prodrug, salt, solvate, or polymorph thereof; and one or more of: a) at least one agent known to decrease the severity of symptoms associated with an neurodegenerative disease; b) at least one agent known to treat to a neurodegenerative disorder; c) instructions for administering the phospholipase D inhibitor and autophagy inducer in connection with treating an neurodegenerative disorder; or e) instructions for administering the phospholipase D inhibitor and autophagy inducer in connection with reducing the severity of symptoms associated with a neurodegenerative disorder.

In a further aspect, an effective amount of the PLD inhibitor is a therapeutically effective amount. In a still further aspect, an effective amount of the PLD inhibitor is a prophylactically effective amount.

In a further aspect, the PLD inhibitor is a compound having a structure represented by a formula:

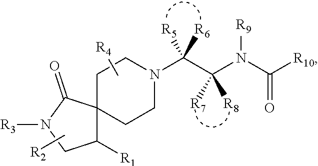

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; and wherein R¹⁰ comprises an optionally substituted C1 to C12 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

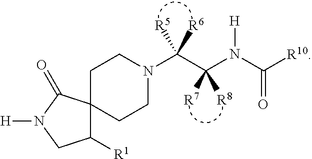

In a further aspect, the PLD inhibitor is a compound having a structure represented by a formula:

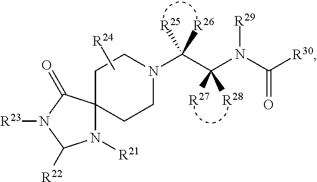

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; and wherein R³⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

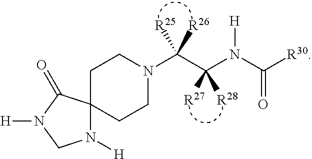

In a further aspect, the compound is:

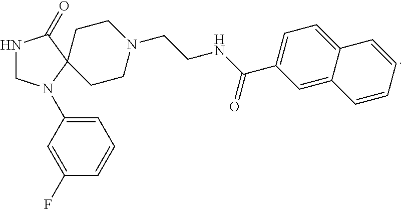 [SMILES: O=C(NCCN1CCC2(CC1)C(=O)NCN2c1cccc(F)c1)c1ccc2ccccc2c1]

In a further aspect, the PLD inhibitor is a compound having a structure represented by a formula:

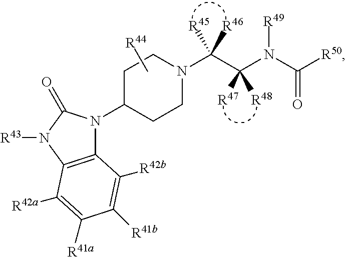

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; and wherein R⁵⁰ comprises an optionally substituted C1 to C16 organic residue selected from alkyl, aryl, heteroaryl, cycloalkyl, heterocycloalkyl, cycloalkenyl, and heterocycloalkenyl; or a pharmaceutically acceptable salt, hydrate, solvate, or polymorph thereof.

In a further aspect, the compound has a structure represented by a formula:

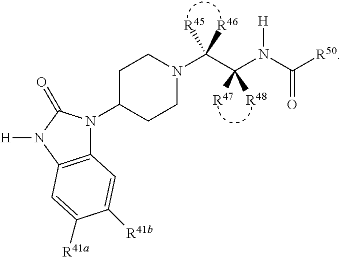

In a further aspect, the compound is:

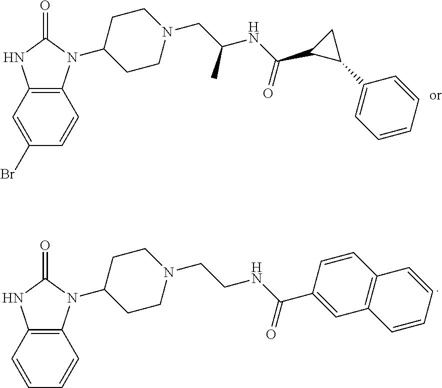

In a further aspect, the PLD inhibitor is a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof.

In a further aspect, the PLD inhibitor is a compound selected from:

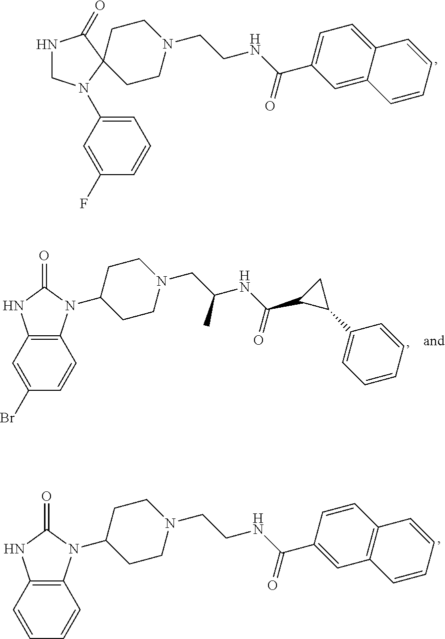

or a pharmaceutically acceptable salt, hydrate, solvate, or polymorph thereof.

In a further aspect, the phospholipase D inhibitor is a disclosed phospholipase D inhibitor.

In a further aspect, the phospholipase D inhibitor inhibits PLD1 and/or PLD2. In a still further aspect, the phospholipase D inhibitor inhibits PLD1. In yet a further aspect, the phospholipase D inhibitor inhibits PLD2.

In a further aspect, the phospholipase D inhibitor is selected from:

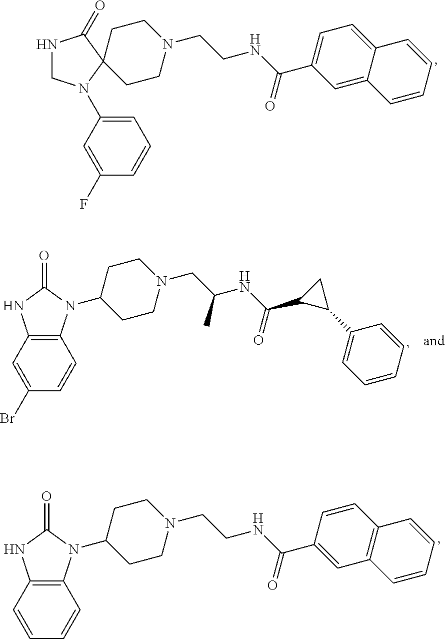

or a pharmaceutically acceptable salt, hydrate, solvate, or polymorph thereof.

In a further aspect, kit further comprises the at least one agent known to decrease Akt activity. In a yet further aspect, the agent known to decrease Akt activity is an Akt inhibitor. In a still further aspect, the Akt inhibitor binds to the pleckstrin homology domain. In yet a further aspect, the Akt inhibitor is an ATP-competitive inhibitor. In an even further aspect, the Akt inhibitor is an allosteric inhibitor. In a still further aspect, the allosteric inhibitor is MK-2066.

In a further aspect, the Akt inhibitor is a pan-Akt inhibitor.

In a further aspect, the Akt inhibitor inhibits Akt1, Akt2, or Akt3. In a still further aspect, the Akt inhibitor is an isoform-selective inhibitor. In yet a further aspect, the isoform-selective inhibitor selectively inhibits Akt1.

In a further aspect, the Akt therapeutic agent is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine. In a still further aspect, the Akt therapeutic agent is erucylphosphocholine. In yet a further aspect, the Akt therapeutic agent is GDC-0068. In an even further aspect, the Akt therapeutic agent is GSK-2141795. In a still further aspect, the Akt therapeutic agent is MK-2206. In yet a further aspect, the Akt therapeutic agent is perifosine. In an even further aspect, the Akt therapeutic agent is PHT-427.

In a further aspect, the at least one agent known to decrease Akt activity is a siRNA.

In a further aspect, the at least one agent known to decrease Akt activity is an antisense oligonucleotide. In a still further aspect, the antisense oligonucleotide is RX-0201.

In a further aspect, the neurodegenerative disorder is selected from the neurodegenerative disorders are selected from the group of Parkinson's disease; Huntington's disease; dementia such as for example Alzheimer's disease; multi-infarct dementia; AIDS-related dementia or frontotemporal dementia; the disorders or conditions comprising as a symptom a deficiency in attention and/or cognition are selected from the group of dementia, such as Alzheimer's disease; multi-infarct dementia; dementia due to Lewy body disease; alcoholic dementia or substance-induced persisting dementia; dementia associated with intracranial tumors or cerebral trauma; dementia associated with Huntington's disease; dementia associated with Parkinson's disease; AIDS-related dementia; dementia due to Pick's disease; dementia due to Creutzfeldt-Jakob disease; delirium; amnestic disorder; post-traumatic stress disorder; stroke; progressive supranuclear palsy; mental retardation; a learning disorder; attention-deficit/hyperactivity disorder (ADHD); mild cognitive disorder; Asperger's syndrome; and age-related cognitive impairment.

In a further aspect, the agent known to treat a neurodegenerative disorder is selected from an anti-Alzheimer's agent, beta-secretase inhibitor, gamma-secretase inhibitor, muscarinic agonist, muscarinic potentiator, HMG-CoA reductase inhibitor, NSAID and anti-amyloid antibodies.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

5. Non-Medical Uses

Also provided are the uses of the disclosed compounds and products as pharmacological tools in the development and standardization of in vitro and in vivo test systems for the evaluation of the effects of modulators of phospholipase D related activity in laboratory animals such as cats, dogs, rabbits, monkeys, rats and mice, as part of the search for new therapeutic agents of phospholipase D and/or Akt. In a further aspect, the invention relates to the use of a disclosed compound or a disclosed product as pharmacological tools in the development and standardization of in vitro and in vivo test systems for the evaluation of the effects of modulators of phospholipase D related activity in laboratory animals such as cats, dogs, rabbits, monkeys, rats and mice, as part of the search for new therapeutic agents of phospholipase D1 and/or Akt. In a still further aspect, the invention relates to the use of a disclosed compound or a disclosed product as pharmacological tools in the development and standardization of in vitro and in vivo test systems for the evaluation of the effects of modulators of phospholipase D related activity in laboratory animals such as cats, dogs, rabbits, monkeys, rats and mice, as part of the search for new therapeutic agents of phospholipase D2 and/or Akt.

It is contemplated that the disclosed kits can be used in connection with the disclosed methods of making, the disclosed methods of using, and/or the disclosed compositions.

### H. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Methods

a. PLD Inhibitor Compounds

The representative PLD inhibitor compounds used in the various studies described herein below are shown below in Table 1, and were synthesized as previously described (Lavieri et al. (2010) J. Med. Chem. 53 6709; and Scott, S. A., et al. (2009) Nat. Chem. Biol. 5:108-117). The inhibitor activity of these representative PLD inhibitors is provided in Table 2.

TABLE 1

| | | Reference | |
|---|---|---|---|
| No. | Structure | Codes | Chemical Name |
| 1 | | JWJ; | N-(2-(1-(3-fluorophenyl)- |
| | | VU0364739 | 4-oxo-1,3,8-triazaspiro[4.5]decan- |
| | | | 8-yl)ethyl)-2- |
| | | | naphthamide |
| 2 | | EVJ; | (1R,2S)-N-((S)-1-(4-(5- |
| | | VU0359595 | bromo-2-oxo-2,3- |
| | | | dihydro-1H- |
| | | | benzo[d]imidazol-1- |
| | | | yl)piperidin-1- |
| | | | yl)propan-2-yl)-2- |
| | | | phenylcyclopropane |
| | | | carboxamide |
| 3 | | 5WO; | N-(2-(4-(2-oxo-2,3- |
| | | VU0155056 | dihydro-1H- |
| | | | benzo[d]imidazol-1- |
| | | | yl)piperidin-1-yl)ethyl)- |
| | | | 2-naphthamide |

TABLE 2

| | Reference | PLD1* | PLD2* | PLD1** | PLD2** |
|---|---|---|---|---|---|
| No. | Codes | (IC₅₀, nM) | (IC₅₀, nM) | (IC₅₀, nM) | (IC₅₀, nM) |
| 1 | JWJ; | 1,500 | 20 | 7,400 | 100 |
| | VU0364739 | | | | |
| 2 | EVJ; | 3.7 | 6,400 | 15 | 1,100 |
| | VU0359595 | | | | |
| 3 | 5WO; | 21 | 380 | 80 | 240 |
| | VU0155056 | | | | |

*Cellular assay
**In vitro enzyme assay

b. In Vitro PLD Activity Assay

In vitro PLD activity is measured with an exogenous substrate assay as previously described (Brown, H. A. et al. (1993) Cell 75:1137-1144). Briefly, PLD activity was measured as the release of [methyl-3H]choline from [choline-methyl-³H]dipalmitoylphosphatidylcholine. PLD enzyme (PLD1=3 nM, or PLD2=15 nM) is reconstituted with phospholipid vesicle substrates. Lipid solutions were dried and resuspended, and small unilamellar vesicles were prepared by bath sonication. All assays were performed at 37° C. with agitation for 30 min. Reactions were stopped by addition of trichloroacetic acid and bovine serum albumin. Free [methyl-³H]choline was separated from precipitated lipids and proteins by centrifugation and analyzed by liquid scintillation counting. Raw data were normalized and are presented as percent total activity. Experiments were performed in triplicate.

c. Cell Culture

U87MG and U118MG cells (ATCC) and HEK293-TREx (Life technologies) were maintained in DMEM (Life technologies)+10% fetal bovine serum (Atlanta biologicals)+1% Penicillin/Streptomycin (PS) (Life technologies). myrAkt1 U87MG cells were maintained in DMEM+10% tetracycline-free fetal bovine serum (Atlanta biologicals)+1% PS. CD133+ glioma stem cells were cultured as described in Wang 2010. Stem cells were maintained in neurobasal media containing glutamine, B27, sodium pyruvate (all from Life technologies), 20 ng/ml fibroblast growth factor and epidermal growth factor (Peprotech). All human cells were maintained at 37° C. in a humidified incubator with 5% CO₂.

Sf21 insect cells were obtained from Orbigen and maintained in Grace's Media (Life technologies)+10% fetal bovine serum. All insect cells were maintained at 27 degrees C.

d. Plasmids and Baculovirus Production

The following plasmids were obtained from Addgene: pcDNA3 T7 Akt1 (PI: William Sellers, Ramaswamy 1999, plasmid 9003), pcDNA3 myr HA Akt1 (PI: William sellers, Ramaswamy 1999, plasmid 1036), ptfLC3 (PI: Tamotsu Yoshimori, Kimura 2007, plasmid 20174), and pcDNA4 Beclin1-HA (PI: Qing Zhong, Sun 2008, plasmid 24399).

FLAG-PLD1 and FLAG-PLD2 was created by PCR amplification of the PLD open reading frames (PLD1 cDNA was obtained from Open Biosystems MGC collection, cloe #6068382 and PLD2 cDNA was a generous gift from Dr. David Lambeth at Emory University) using forward primer containing FLAG epitope sequence and ligating into pcDNAS/TO (Life Technologies). To create the ProteinA-tec-Strep-tagged PLD2 construct (PtS-PLD2), the PtS tag from p31-N-PtS (a kind gift from Dr. Yisong Wang, Giannone 2007) was shuttled into pcDNAS/TO to create PtS-pcDNAS/TO and the PLD2 ORF was subsequently ligated 3′ of the PtS ORF into PtS-pcDNAS to create a PLD2 construct with an N-terminal PtS tag. To create the PtS-PLD2 baculovirus, the PtS-PLD2 ORF was ligated into pENTR1A (Life Technologies). After LR recombination into pDEST8 (Life Technologies), baculovirus was produced according to manufacturer's instructions. A bacterial expression vector for the PtS tag was created by amplification of the PtS tag from PtS-pcDNAS/TO and ligated into pET16b (EMD Millipore).

For 6×His-Akt1 baculovirus production, the Akt1 ORF was amplified from pcDNA3 myr HA Akt1 and ligated into pENTR3C (Life technologies). pENTR3C was LR recombined into pDEST10 (Life technologies) to generate a 6×His-Akt1 construct. The baculovirus was produced according to manufacturer instructions.

For ProteinA-tev-Strep (PtS) PLD2 baculovirus production, the PtS tag from p31-N-PtS into the BamHI/HindIII sites of pcDNAS/TO (forward primer: 5′-atggatccGCAACAACCTGGACAGCA-3′ reverse primer: 5′-aataagctttaagCCATGGTGGACAACAAATTC-3′) to create PtS-pcDNAS/TO. PLD2 was subsequently amplified (forward primer: 5′-ataagaatgcggccgcATGACGGCGACCCCTGAG -3′, reverse primer: 5′-gctctagaCAACTATGTCCACACTTCTAG-3′) and ligated into the NotI/XbaI sites of PtS-pcDNAS. PtS-PLD2 was then amplified (forward primer: 5′-acgcgtcgacGCCATGGTGGACAACAAATTC-3′, reverse primer: 5′-ataagaatgcggccgCAACTATGTCCACACTTCTAG-3′) and ligated into the SalI/NotI sites of pENTR1A (Life technologies). PtS-PLD2 in pENTR1A was LR recombined into pDEST8 (Life technologies). Bacmids were produced by transformation into DH10Bac E. coli as described by the manufacturer instructions. Baculovirus was produced by transfecting bacmids into Sf21 insect cells using Cellfectin II (Life technologies) transfection reagent and harvesting media 72 h post transfection.

e. Endogenous PLD Activity Assay

Endogenous PLD activity was determined using a modified in vivo deuterated 1-butanol PLD assay ((Brown, H. A. et al. Methods Enzymol. (2007) 434:49-87). Depending on the experimental paradigm, cells were treated with 0.3% deuterated n-butanol-d₁₀ for 30 minutes prior to phospholipid extraction. Internal standards were added before drying samples and resuspending in 9:1 methanol:chloroform. Samples were injected into a Finnigan TSQ Quantum triple quadrupole mass spectrometer and phosphatidylbutanol peaks were detected in negative ion mode. Background peaks were subtracted from cells not treated with n-butanol. Data is presented as the ratio of the predominant phosphatidylbutanol peaks to the internal standard. To generate concentration response curves, U87MG cells were serum starved for approximately 24 hours. Cells were treated with the indicated concentration of PLD inhibitor for 15 minutes prior to addition of n-butanol containing media and phospholipid extraction.

f. Transfection

Mammalian cells were transfected using Fugene 6 (Roche) according to the manufacturer's instructions using the standard volumes of transfection reagent and DNA quantities. Cells were harvested approximately 48 hours post transfection.

g. RNAi

All siRNA was obtained from Dharmacon as a pool of 4 oliogonucleotide targeting sequences per relevant target (ON-TARGETplus). For PLD siRNA transfections, U87MG cells were seeded in 6-well plates at approximately 100,000 cells/well. The following day, cells were transfected with 100 nM siRNA per the manufacturer's instructions using the Dharmafect 1 transfection reagent in antibiotic and serum-free DMEM. Complete media was added the following day. Approximately 48 hours post transfection, cells were serum starved overnight before assay to allow siRNA approximately 72 h to successfully degrade target transcripts.

For viability assays, U87MG cells were seeded in 60 mm tissue culture plates at 540,000 cells/plate and transfected with 100 nM siRNA. The following cells were split into 96-well plates, allowed to attach overnight, and serum starved in the presence of inhibitors the following day. Viability was assessed approximately 72 h post siRNA transfection.

h. Cell Viability Assays

Cells were seeded into clear-bottom, black-welled 96-well tissue culture plates and allowed to adhere overnight to achieve approximately 60% confluence the following day. Cells were serum-starved in the presence of indicated inhibitors overnight. Viability was measured by addition of the WST-1 reagent (Roche) and measuring absorbance at 450 nM. Time of WST-1 incubation was cell-line dependent and ranged from 30 minutes to 2 h.

To measure viability following RNAi treatment, cells were seeded in 60 mm tissue culture plates at 540,000 cells/plate and transfected with 100 nM siRNA. The next day, cells were split into 96-well plates, allowed to adhere overnight, and serum starved in the presence of inhibitors the following day. Viability was assessed approximately 72 h post siRNA transfection.

i. Anchorage-Independent Growth Assays

Base layers of 0.5 mL of 0.7% agarose (SeaKem GTG agarose, Cambrex Bio Science, Rockland, Me.) containing complete neurobasal growth media were prepared in 12-well tissue culture plates. PLD inhibitors or DMSO vehicle controls were incorporated into the base layers. A 0.5 mL overlayer of 0.35% agarose containing CD133+ cells (5.0×10³) in complete growth medium with PLD inhibitors was applied. Each condition was plated in triplicate wells. Plates were incubated at 37° C. in a humidified atmosphere of 5% CO₂ in air. Cells were fed every 2-3 days with complete growth media plus PLD inhibitor. Colony formation progressed for 8 weeks. Crystal Violet (0.005%) was used to stain colonies. Large colonies (>50 cells) were scored at 10× magnification with an inverted phase microscope using the average of 4 random fields per sample.

j. Immunoprecipitation

Cells were scraped in ice-cold PBS then centrifuged at 1,000 g for 5 minutes to pellet cells. Cells were lysed by suspension in lysis buffer (50 mM Tris pH 8.0, 15 0 mM NaCl, 0.5% NP-40, 40 mM beta-glycerophosphate, 20 mM sodium pyrophosphate, 1 mM Na₃VO₄, 2 mM EDTA, 2 mM EGTA, 5 mM NaF, 1 mM DTT, and Roche complete protease inhibitor cocktail) and then subjected to 3 freeze/thaw cycles in dry ice/ethanol. Between each thaw cycle, lysates were drawn up and down through 25G syringe needles. Lysates were clarified by centrifugation at 10,000×g for 10 minutes at 4° C. In order to remove non-specific proteins, lysates were incubated with protein-G agarose (Millipore) for 2 h at 4° C. Beads were removed by centrifugation at 1,000×g for 1 min. Supernatants were transferred to new tubes and immunoprecipitating antibodies were allowed to incubate with pre-cleared lysates overnight at 4° C. Antibodies were captured using protein-G agarose for 2 h. Beads were washed 3× in lysis buffer containing 300 mM NaCl and complexes eluted by boiling in 2×SDS-PAGE loading buffer. For immunoprecipitation of endogenous proteins, normal IgG (Santa Cruz) was used as a nonspecific control.

k. Immunoblotting

Lysates were prepared by incubating cell pellets in lysis buffer (see immunoprecipitation procedure) for 30 minutes at 4° C. Protein concentrations were measuring using the Bio-Rad protein assay reagent. The antibodies used in the various studies as described herein, along with their vendors and catalog numbers are provided in Table 3.

TABLE 3

| Antibody | Vendor | Catalog Number |
|---|---|---|
| Pan-Akt | Cell signaling | 9272 |
| Akt-S473 | Cell signaling | 4058 |
| Akt-T308 | Cell signaling | 4056 |
| Beclin-1 | Cell signaling | 3738 |
| Akt1 | Cell signaling | 2938 |
| Akt1-S473 | Cell signaling | 9018 |
| β-actin | Sigma | A5316 |
| PLD1 | Santa Cruz | sc-28314 |
| PLD2 | Abgent | AT3337a |
| FLAG (M2) | Sigma | F-3165 |
| Syndecan-4 | Santa Cruz | sc-15350 |
| Calnexin | BD Transduction | 610523 |
| | Laboratories | |
| ATP synthase a | Santa Cruz | sc-58613 |
| GAPDH | Santa Cruz | sc-25778 |
| LC3 | Novus | NB100-2220 |
| p62 | Cell signaling | 5114 |
| Atg7 | Cell signaling | 8558 |
| p70s6-kinase T389 | Cell signaling | 9234 |
| p70s6-kinase | Cell signaling | 9202 |
| AMPK a-T172 | Cell signaling | 2535 |
| AMPK a | Cell signaling | 5831 |
| ACC-S79 | Cell signaling | 3661 |
| ACC | Cell signaling | 3676 |
| ULK1-S555 | Cell signaling | 5869 |
| ULK1 | Cell signaling | 8054 |
| pan-cadherin | Abcam | ab6529 |
| rubicon | Abcam | ab92388 |
| HA-tag | Covance | MMS-101P |
| strep-tag | Qiagen | 34850 |

l. Protein-Lipid Binding

The general procedure for measuring Akt to lipid spots on nitrocellulose membranes was described in Dowler 2002. Briefly, lipids (18:0-18:0 PA or 18:1-18:1 PIP₃) were diluted to 1 mM in 1:1 methanol:chloroform then further diluted to the final working concentrations in 2:1:0.8 methanol:chloroform:water. 1 mL of each concentration was spotted onto nitrocellulose and allowed to dry for 1 h. Membranes were blocked for 1 h in 3 mg/mL fatty-acid free BSA in Tris buffered saline+0.1% Tween-20 (TBST). Recombinant protein (3 nM) was incubated with the membranes overnight in blocking buffer, and then washed the following with TBST. Washed membranes were incubated with primary antibody overnight in blocking buffer. The following morning, membranes were washed, and incubated with secondary antibody for 1 h. Membranes were washed and bound protein detected using enhanced chemiluminescence (ECL, Pierce). The membrane containing various classes of lipids was obtained from Avanti Polar Lipids and contained 2 μg of the indicated lipid per spot. The snooper membrane was obtained from Avanti polar lipids and contains 2 mg of the indicated lipid per spot.

For vesicle competition assays, vesicles were prepared by drying lipids under N₂ gas and resuspending in 50 mM Tris pH 7.4, 150 mM NaCl, and 2 mM EGTA. Lipids were vortexed and sonicated until in solution. The vesicles were composed of either 100% 32:0 PC (DPPC) or 95% DPPC+5% 32:0 DPPA (mol percent). Recombinant Akt was incubated for 2 h with 200 μM bulk vesicles in TBST and the mixture was incubated with nitrocellulose membranes overnight. Lipid-bound Akt was determined using a total Akt antibody and chemiluminescence.

m. Exogenous Phosphatidic Acid Rescue

Lipids were obtained as chloroform suspensions from Avanti Polar Lipids. Lipids were dried under N₂ gas in glass Pyrex® tubes. Dried lipid film was vortexed in DMEM+0.25 mg/mL fatty-acid free BSA then sonicated for 10 minutes. The sonicated lipid mixture was further diluted in the DMEM/BSA mixture to a final concentration of approximately 1 mM and cells were treated for the duration of time indicated in the text.

n. Membrane Isolation from U87Mg Cells

U87MG cells were seeded in 150 mm tissue culture plates at 2.6×10⁶ cells/plate (4 plates per condition) and allowed to adhere overnight. The following day, cells were washed and media replaced with DMEM+indicated inhibitor or vehicle and cells were treated for up to 12 h. Cells were washed twice in 1×PBS then scraped in homogenization buffer (20 mM HEPES pH 7.4, 1 mM EDTA, 250 mM sucrose). Cells were pelleted by centrifugation at 1,000×g for 4 minutes at 4° C. Cell pellets were resuspended in homogenization buffer containing Roche complete protease inhibitor cocktail, 40 mM beta-glycerophosphate, 20 mM sodium pyrophosphate, 1 mM Na₃VO₄, and 5 mM NaF and lysed by nitrogen cavitation (1000 psi for 5 min, 4° C.). Lysate was collected dropwise then centrifuged at 2,000×g for 10 min to pellet unbroken cells, nuclei, and heavy debris. The supernatant was subsequently centrifuged at 100,000×g for 60 min. The supernatant was saved as the cytosolic fraction and the 100,000×g pellet was washed once by resuspension in lysis buffer then centrifuged again under the same conditions.

A stock Iodixanol (Optiprep, Axis-Shield) gradient solutions as prepared by diluting the 60% iodixanol solution from the manufacturer in dilution buffer (120 mM HEPES pH7.4, 250 mM sucrose, 6 mM EDTA, 240 mM beta-glycerophosphate, 6 mM Na₃VO₄, and 120 mM sodium pyrophosphate) in a ratio of 5 parts 60% iodixanol to 1 part diltion buffer to create a 50% iodixanol working solution. 2.5%, 10%, 17.5%, 25%, and 30% iodixanol solutions were prepared by mixing the appropriate ratios of 50% iodixanol with homogenization buffer.

Washed membranes from the second 100,000×g spin were resuspended in 30% iodixanol (approximately 500 mL) and added to 11 mL polycarbonate ultracentrifuge tubes (Beckman Coulter). Equal volumes of 25%, 17.5%, 10%, 2.5% iodixanol were layered on top of the membrane suspension and tubes were centrifuged for 3.5 h at 165,000×g in a swinging bucket rotor. 1 mL fractions were collected from the bottom by introducing a small hole with a 25G syringe needle and collecting droplets. Samples were then boiled in 6×SDS-PAGE loading buffer prior to immunoblotting. Membranes predominantly banded at the 10%/17.5% iodixanol interface.

o. Protein Purification

6×His-Akt1 was produced by infecting monolayer cultures of Sf21 cells with baculovirus for 60 h. 4 hours prior to cell harvest, 100 nM okadaic acid (EMD Millipore Corporation, formerly, Calbiochem, Billerica, Massachussetts) was added to cell media.

PtS-PLD2 was produced by infecting monolayer Sf21 cells for 72 h. Cells were harvested and collected by centrifugation at 500×g for 5 minutes. Cells were lysed by sonication in lysis buffer (50 mM Tris pH 8.0, 500 mM NaCl, 0.5% NP-40, 2.5 mM EDTA, 50 mg/ml Avidin, 1 mM DTT, complete protease inhibitor tablet (Roche), and 1 mM PMSF added immediately prior to sonication). Lysate was cleared by centrifugation at 12,000×g for 10 minutes. Cleared lysate was incubated with strep-tactin affinity resin (IBA) overnight at 4° C. Beads were washed 3× with 10 mL wash buffer (lysis buffer with 0.01% NP-40), with rotation of beads and wash buffer for 10 minutes per wash). PtS-PLD2 was eluted by incubation of beads with 5 mM desthiobiotin (Sigma) in wash buffer for 10 minutes, centrifugation at 1,000×g, and collecting supernatants containing soluble PtS-PLD2. It was found that most PLD2 eluted during the first 3 batch fractions collected. For protein-protein interaction studies, PLD2-PtS eluate was dialyzed (5,000 MWCO standard dialysis membrane) overnight against wash buffer to remove desthiobiotin.

The ProteinA-tev-Strep protein (used a control for protein interaction experiments) was produced by transforming BL21 E. coli (Agilent) with the pET16b-PtS plasmid. Bacteria were grown at 37° C. until OD₆₀₀ reached 0.7. At that point, protein expression was induced by adding 100 mM IPTG and growing bacteria overnight at 18° C. Bacteria were lysed by incubating in lysis buffer (30 mM sodium phosphate buffer pH 7.4, 500 mM NaCl, complete protease inhibitor cocktail, and 1 mg/mL lysozyme) for 30 minutes followed by sonication. Lysates were clarified by centrifugation at 14,500×g for 30 minutes at 4° C. Lysates were loaded onto a 1 mL Hi-Trap chelating column (GE), charged with nickel sulfate and equilibrated with 5 column volumes of lysis buffer minus lysozyme and protease inhibitors. The column was washed until OD₂₈₀ returned to baseline at which point, 40 mM imidazole was run over the column to elute non-specific proteins. Once OD₂₈₀ returned to baseline, PtS was eluted in a linear imidazole gradient from 40-500 mM. Eluates were pooled and loaded onto a 120 mL Sephadex 75 gel filtration column (GE), previously equilibrated with 50 mM Tris pH 7.4, 0.5 mM EGTA, 150 mM NaCl, and 2 mM DTT. Fractions containing PtS protein were collected and pooled for use in binding assays.

N-terminal Akt PH domain 6×His-GST fusion proteins were produced by PCR amplifying the first 123 amino acids of Akt1 (Thomas, C. et al., (2002) Curr. Biol. 12, 1256-1262), ligating into pBG105 (Vanderbilt structural biology core), and transforming BL21 E. coli. Bacteria were grown at 37° C. until OD₆₀₀ reached 0.7. At that point, protein expression was induced by adding 250 mM IPTG and growing bacteria overnight at 27° C. Bacteria were lysed by incubating in lysis buffer (50 mM Tris buffer pH 7.5, 150 mM NaCl, 1 mM EGTA, 1 mM EDTA, 1 mM Na₃VO₄, 10 mM β-glycerophosphate, 50 mM NaF, 5 mM DTT, Roche complete protease inhibitor cocktail, and 1 mg/mL lysozyme) for 30 min followed by sonication. Lysates were clarified by centrifugation at 14,500×g for 30 min at 4° C. then applied to glutathione agarose (Sigma) for 2 h at 4° C. Resin was washed twice with lysis buffer and twice with wash buffer (50 mM Tris pH 7.5, 300 mM NaCl, 0.1 mM EGTA, and 5 mM DTT) and eluted by incubating in wash buffer containing 10 mM reduced glutathione.

6×His-Akt1 was purified from Sf21 cells essentially as previously described (Kumar, C. et al., (2001) Biochim. Biophys. Acta. 1526, 257-268).

p. In Vitro Protein-Protein Interaction Assays

25 nM PLD2-PtS or PtS tag were incubated with 50 nM 6×His-Akt1 for 2 h in 50 mM Tris pH 8.0, 0.01% Nonidet P-40, 150 mM NaCl, 0.5 mM EDTA, and 50 mg/mL avidin. Where indicated, 10 μM inhibitor was included in the reaction mixture. PtS-tagged proteins were captured by incubation with strep-tactin resin for 2 h. Resin was washed 3×(10 minutes per wash) and proteins eluted by boiling in 2×SDS-PAGE loading buffer.

q. Immunofluoroscence

Glass coverslips were flame sterilized and placed into 6-well plates. U87MG-tfLC3 cells were seeded at 150,000 cells/well in complete media and allowed to adhere overnight. The following day, cells were washed and treated with inhibitors in serum-free DMEM for 24 h. Cells were washed in 1×PBS then fixed for 15 minutes in 2% paraformaldehyde followed by washing in PBS. Coverslips were removed and mounted onto glass slides in Vectashield mounting media containing DAPI (Vector Labs). GPF/RFP images were acquired using Nikon A1R laser scanning confocal microscope equipped with a Plan Apo VC 60× 1.4 N.A. and 40× oil immersion lens.

r. Statistical Analysis

Statistical analyses used for figures are described herein above. Graphs of PLD activity and cell viability are representative from at least three experiments. Quantified immunoblots represent pooled data from at least three independent experiments unless otherwise noted in the text.

2. Phospholipase D2 Activity is Required for Glioma Viability Following Serum-Withdrawal.

Multiple cancer types require PLD and its product, PtdOH, for sustained survival under stress conditions (Foster, D. and Xu, L., (2003) Mol. Cancer. Res. 1, 789-800). Serum-withdrawal, a known stimulus of PLD activity in multiple cancer cell lines (Zheng, Y. et al., (2002) J. Biol. Chem. 281, 15862-15868), is frequently used to simulate the harsh growth environments encountered by neoplastic cells prior to vascularization and restoration of nutrient supply within the tumor mass. Viability is compromised when normal cells are cultured in serum-depleted conditions. Cells with elevated PI3K/Akt activity, however, continue to proliferate under these harsh culture conditions (Sun, H. et al., (1999) Proc. Natl. Acad. Sci. U.S.A. 96, 6199-6204).

To investigate the role of PLD in GBM survival, PLD activity was measured following serum-withdrawal in the PTEN-null U87MG GBM cell line. As described herein, cells were grown overnight in complete growth media (DMEM and FBS) before growth in media lacking fetal bovine serum (FBS) for times ranging from 1 to 24 h. At the completion of the indicated time-point, cells were incubated in media containing 0.3% deuterated butanol for 30 minutes followed by phospholipid extraction. In the PLD reaction, n-butanol competes with water as a nucleophile to generate a metabolically stable phosphatidylbutanol, which is measurable via mass spectrometry. Serum-withdrawal resulted in a time-dependent increase in PLD activity with the most robust activation measured after 16 hours and longer durations of serum-withdrawal did not further increase PLD activity (FIG. 1A). Referring to FIG. 1A, cells were seeded approximately 24 h prior to washing and incubation in serum-free media for the indicated length of time. n-Butanol (n-butanol-d₁₀) was added 30 min prior to glycerophospholipid extraction and subsequent phosphatidylbutanol (PtdBuOH) quantification. By contrast, an increase in PLD activity was not observed in the non-tumorigenic HEK293 line under the same conditions. Without wishing to be bound by a particular theory, the data are consistent with a cancer line specific PLD response. Serum deprivation leading to PLD activation in U87MG cells is consistent with published reports on other cancer cell lines showing similar trends (Zheng, Y. et al., (2002) J. Biol. Chem. 281, 15862-15868).

Two isoforms of PLD have been identified, PLD1 ((Hammond, S. M. et al., (1995), J. Biol. Chem. 270, 29640-29643) and PLD2 (Colley, W. C. et al., (1997) Curr. Biol. 7, 191-201), and each demonstrates distinct regulatory properties. PLD1 is quiescent under normal growth conditions and requires stimulation by proteins including small GTPases such as Arf (Brown, H. A. et al., (1993) Cell 75, 1137-1144), whereas PLD2 displays higher basal activity and is generally unresponsive to activators of PLD1 (Colley, W. C. et al., (1997) Curr. Biol. 7, 191-201). In order to understand the role of each isoform in this stress pathway, the PLD isoform preferentially activated following serum-withdrawal was determined using both genetic and pharmacological tools. In the first approach, U87MG cells were serum deprived overnight and then treated with various concentrations of PLD1 and PLD2 selective compounds described in Table 2. EVJ is a 1,700-fold PLD1-preferring compound (Lewis, J. A. et al., (2009) Bioorg. Med. Chem. Leu. 19, 1916-1920) and JWJ is a 75-fold PLD2-preferring compound (Lavieri, R. R. et al., (2010) J. Med. Chem. 53, 6706-6719), as determined with cell-based assays designed to measure activity of individual PLD isoforms. In the U87MG cells, which express both PLD 1 and PLD2, EVJ and JWJ attenuated PLD activity following serum-withdrawal with IC₅₀ values of approximately 500 nM and 100 nM, respectively (FIG. 1B). The five-fold greater potency of the PLD2-preferring compound suggests that the PLD2 isoform is responsible for the vast majority of PLD activity in these cells following serum-withdrawal, although the PLD1 isoform may partially compensate following acute inhibitor treatment. To further explore the contribution of individual isoforms to the total PLD activity, isoform-specific siRNA was used to knock down either PLD1 or PLD2 and measure PLD activity following overnight serum-withdrawal. Silencing of PLD2, but not PLD1, resulted in a significant decrease in PLD activity (FIG. 1C), further implicating PLD2 as the predominant isoform in the serum-withdrawal response.

Referring to FIG. 1B, U87MG cells were seeded as in FIG. 1A and serum was withdrawn for 24 h. Cells were pretreated with inhibitors 30 min prior to measurement of PLD activity. Data is presented as the percent activity remaining after PLD inhibitor treatment relative to control. Referring to FIG. 1C, U87MG cells were transfected with siRNA targeting either PLD 1 or PLD2 for 48 h prior to a 24-hour serum starvation before measuring PLD activity. Note the PLD2 antibody recognizes a non-specific band of similar molecular weight to PLD2 and the band of interest is directly above the non-specific band (* p<0.5, ** p<0.01, *** p<0.005, NS is not significant, unpaired Student's t-test, and error bars are standard error of the mean (SEM)).

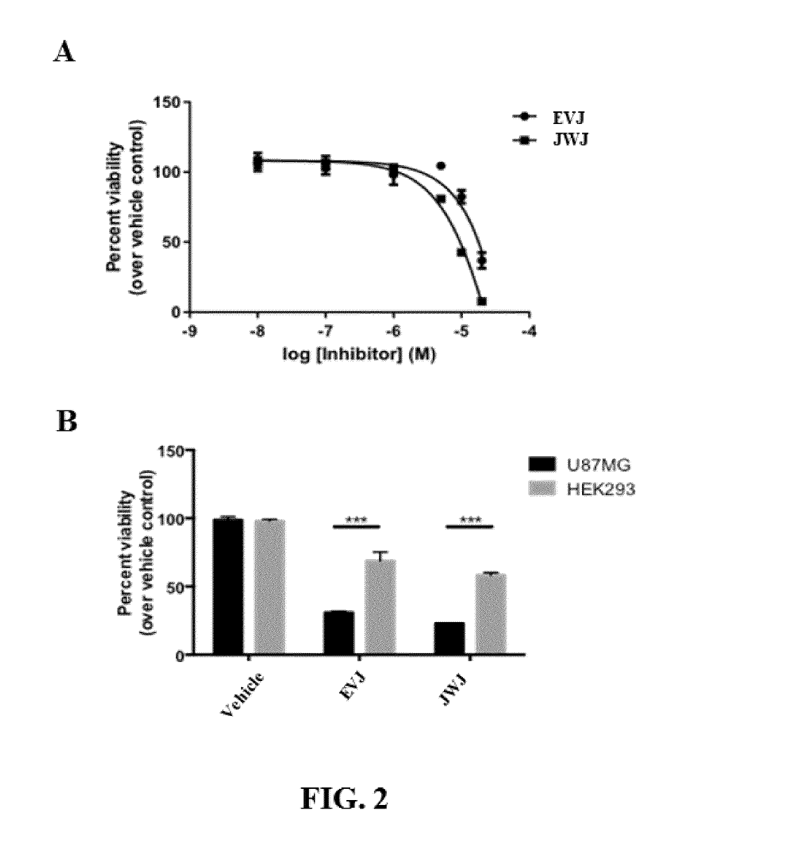

In order to determine if PLD activity was required for viability in U87MG cells following serum-withdrawal, cell viability was measured following overnight treatment with various concentrations of PLD inhibitors. U87MG cell viability decreased in a concentration-dependent manner (FIG. 2A), consistent with concentrations needed to completely ablate PLD activity (FIG. 1B), suggesting that complete suppression of PLD activity compromises viability in these cells. By contrast, treatment of HEK293 cells with PLD inhibitors resulted in significantly less cell death when compared to U87MG cells (FIG. 2B). Without wishing to be bound by a particular theory, the data are consistent with PLD as necessary for cancer cell survival.

Referring to FIG. 2A, cells were treated with the indicated concentration of PLD inhibitor for 24 h in serum-free media. Following inhibitor treatment, viability was measured using the WST-1 reagent. Referring to FIG. 2B, U87MG or HEK293 cells were grown in complete growth media for 24 h. Cells were then treated for 24 h with 10 μM EVJ or 20 μM JWJ in serum-free DMEM. Viability was measured using the WST-1 reagent. Two-way ANOVA with Sidek's post-hoc test was used to compare viability between each cell line within each inhibitor treatment group (*** p<0.001).

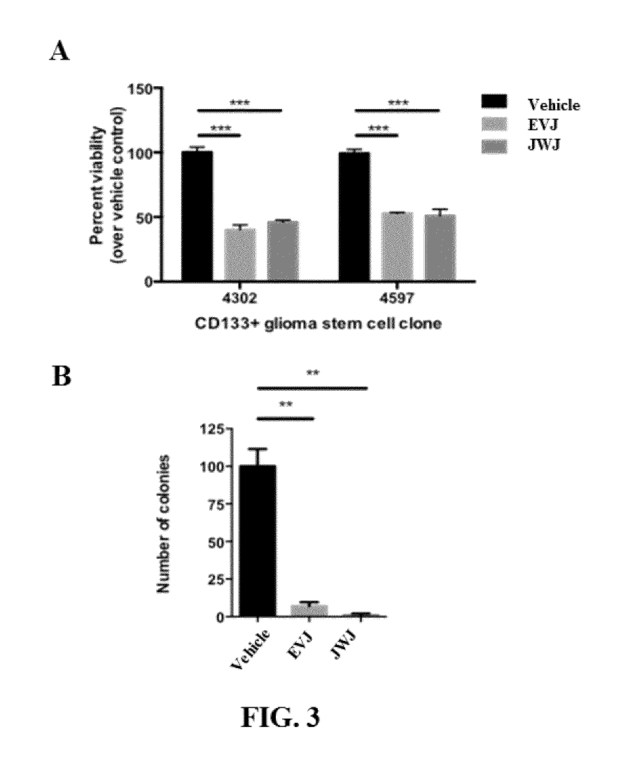

Although U87MG cells are a well-characterized glioblastoma (“GBM”) cell-line, the importance of PLD in a more disease-relevant model system was also evaluated using the various studies as described herein. Glioma stem cells can be isolated from patient tumors by sorting for surface expression of the CD133 antigen. These stem cells are tumorigenic and phenocopy the patient's original tumor when injected into immunocompromised mice (Singh et al., 2004). Two glioma stem cell clones (Wang, J. et al., (2010) Stem Cells 28, 17-28), derived from individual patients, both showed reduced viability following PLD inhibitor treatment under growth factor starvation (FIG. 3A). Anchorage-independent growth (AIG), the most important measure of tumorigenicity (Shin, S. I. et al., (1975) Proc. Natl. Acad. Sci. U.S.A. 72, 4435-4439), was then assessed in these stem cells. Following PLD inhibitor treatment, GBM stem cells formed significantly fewer colonies than vehicle control samples in soft agar, even in the presence of growth factor supplements (FIG. 3B).

Referring to FIG. 3A, CD133+ glioma stem cells were seeded into 96-well plates in media containing growth supplements and laminin to facilitate adhesion. 24 h after seeding, cells were treated with 10 μM EVJ or 20 μM JWJ in neurobasal media without supplements for an additional 24 h. Viability was measured as in FIG. 2B. Referring to FIG. 3B, anchorage independent growth of glioma stem cell clone 4302 was assessed using soft-agar colony formation. Growth media was replaced every 2-3 days with 10 μM PLD inhibitor or vehicle. Colonies were allowed to form for 8 weeks. Large colonies were scored after visualization with Crystal Violet (* p<0.5, ** p<0.01, *** p<0.005, NS is not significant, unpaired Student's t-test, and error bars are SEM).

Without wishing to be bound by a particular theory, the data are consistent with PLD activity being required for proliferation and survival in cancer cells, e.g. glioma cells.

3. PLD2 is Required for Akt Activation in Glioblastoma Cells

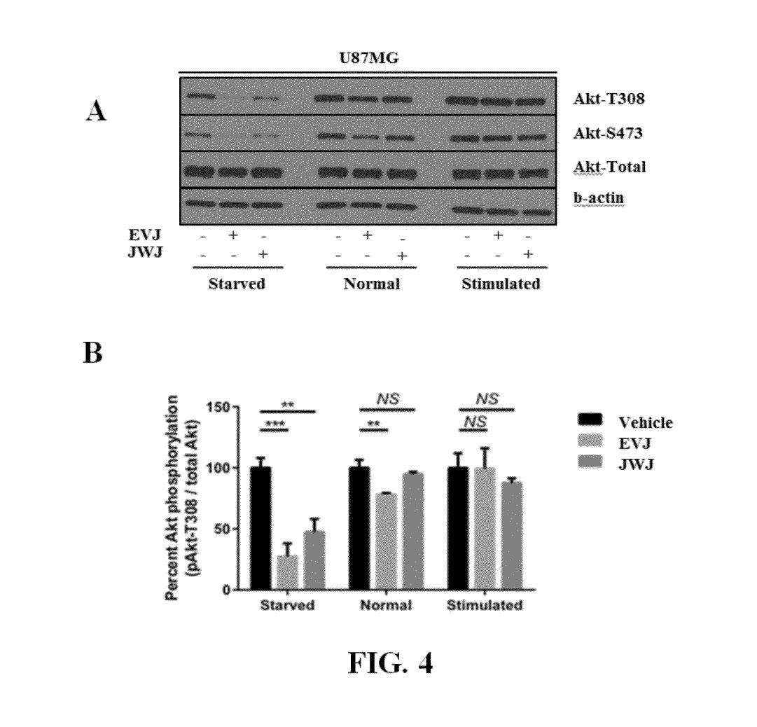

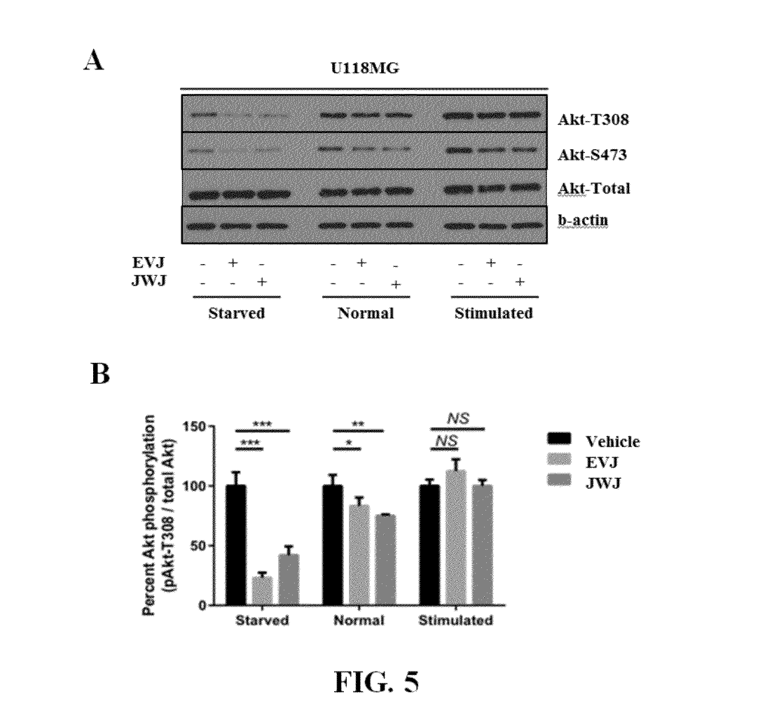

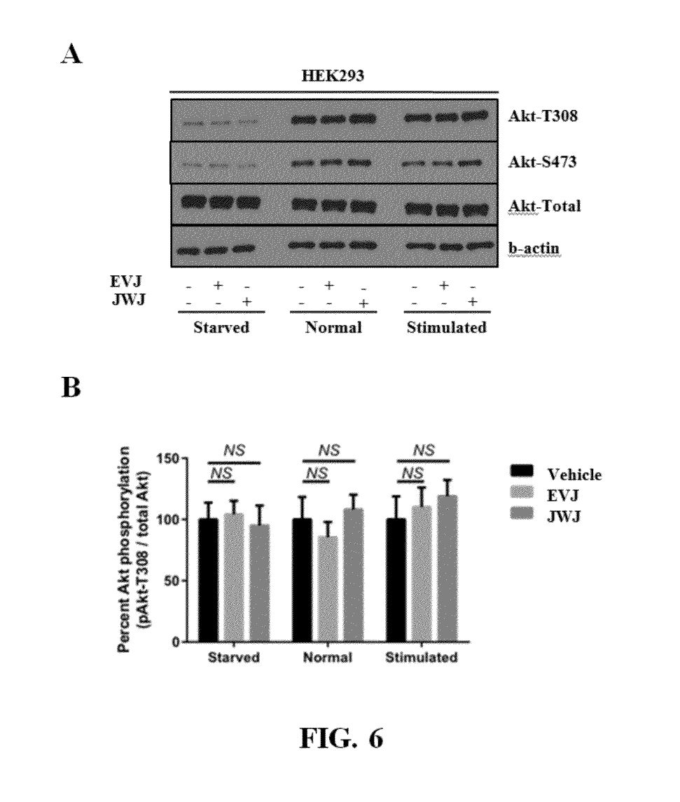

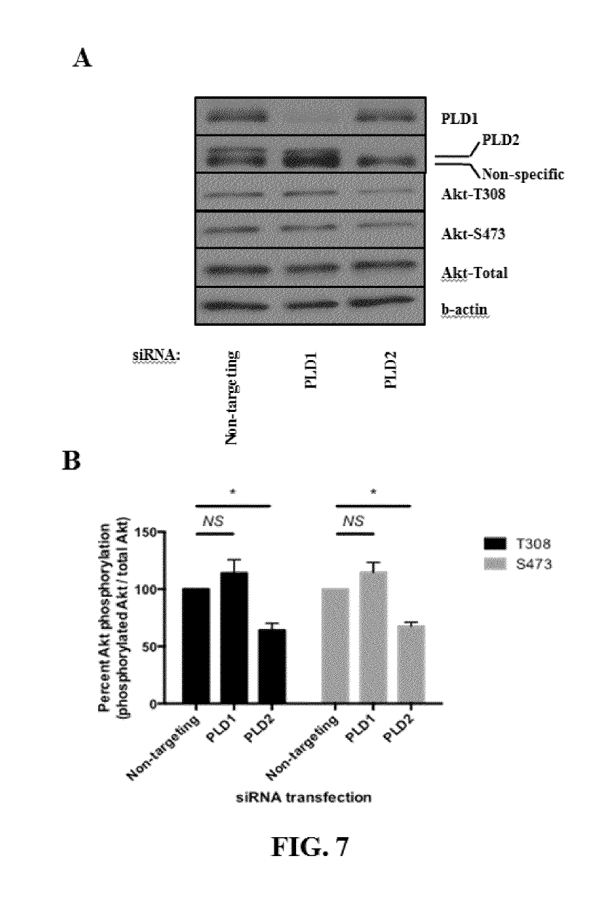

After establishing a requirement for PLD2 in glioma cell viability, the mechanism by which PLD2 regulates survival signaling was determined. The PI3-kinase/Akt pathway is frequently upregulated in cancer and promotes survival by inhibiting apoptotic processes and by regulating metabolism and nutrient utilization (Datta, S. R. et al., (1997) Cell 91, 231-241; Kennedy, S. G. et al., (1997) Genes & Development 11, 701-713; Manning and Cantley, (2007) Cell 129, 1261-1274). Additionally, extracellular pathogens are known to engage the Akt pathway upon infection, and bacterial PLD from N. gonorrhoeae was demonstrated to interact with and activate human Akt upon infection of human cervical epithelial cells (Edwards and Apicella, (2006) Cell Microbiol. 8, 1253-1271). In order to determine if human PLD2 regulated Akt activation in PTEN-null glioma lines, Akt phosphorylation following treatment with PLD inhibitors was measured under various growth conditions. Under the canonical Akt activation sequence, PI3-kinase generates PIP₃, which serves as a membrane recruitment signal for Akt (Franke, T. F. et al., (1995) Cell, 81, 727-736; James, S. R. et al., (1996) Biochem. J. 315, 709-713). Membrane-bound Akt is subsequently activated via a phosphorylation dependent mechanism whereby 3-phosphoinositide dependent kinase 1 phosphorylates Akt at threonine 308 in the activation loop and other kinases such as the mammalian target of rapamycin complex 2 (mTORC2) phosphorylate Akt in its hydrophobic motif at serine 473 (Alessi, D. R. et al., (1997) Curr. Biol. 7, 261-269; Sarbassov, D. D. et al., (2005) Science, 307, 1098-1101). As described herein, cells were treated overnight with PLD inhibitors in either DMEM alone (“starved”), in DMEM+10% FBS (“normal”), or in DMEM followed by stimulation for 10 minutes the following day with 20% FBS (“stimulated”). Since the PLD1- and PLD2-preferring inhibitors are chemically unique compounds that have few structural similarities, using either inhibitor individually at concentrations high enough to inhibit both isozymes (FIG. 1B) without causing substantial cell death (FIG. 2A) minimized possible off-target effects associated with an individual compound. Inhibition of PLD in the PTEN-null U87MG (FIGS. 4A and 4B) and U118MG (FIGS. 5A and 5B) cell lines resulted in decreased levels of activated Akt under serum-depleted conditions as assessed by phosphorylation of threaonine 308 and serine 473. Akt phosphorylation was less affected by PLD inhibition when cells were cultured normally or when stimulated with 20% FBS. These results strongly suggest that PLD regulates Akt activation predominantly under stressful, serum-depleted conditions. Without wishing to be bound by a particular theory, these results also suggest that the PLD inhibitors do not inhibit upstream kinases or Akt directly since growth factor signaling to Akt remains unperturbed. By contrast, PLD inhibitors do not reduce phosphorylated Akt in the non-tumorigenic HEK293 cell line under any condition (FIGS. 6A and 6B), suggesting a cell-type specific regulation of Akt by PLD. Next, either PLD1 or PLD2 were silenced to dismiss any off-target effects of PLD inhibitors and also to further link a specific PLD isoform to this process. Transfection of U87MG cells with PLD2, but not PLD1, siRNA resulted in a significant decrease in phosphorylated Akt at both threonine 308 and serine 473 (FIGS. 7A and 7B).

Referring to FIG. 4-6, cells were incubated overnight in the presence of 10 μM EVJ or 5 μM JWJ in either DMEM (“starved”) or DMEM+10% FBS (“normal”). Another set of cells were starved overnight then stimulated the following day with 20% FBS for 10 minutes (“stimulated”) for comparison. Akt activation was assessed by immunoblotting for phosphorylation at T308 and 5473. Blots were quantified by calculating the ratio of phosphor-Akt at T308 to total Akt using densitometry. S473 was not quantified since no qualitative differences were seen between S473 and T308. Analysis of variance (ANOVA) was used with Dunnett's post-hoc test comparing the PLD inhibitor treatment to vehicle within the growth condition (* p<0.05, ** p<0.01, *** p<0.005). Referring to FIG. 7A, U87MG cells were transfected with either PLD1 or PLD2 siRNA for 48 h prior to overnight serum starvation and immunoblotting. Referring to FIG. 7B, quantification of phosphor-Akt (T308 and S473) following PLD siRNA treatment in U87MG cells is shown. Data represent the fold change in phosphor-Akt relative to the non-targeting siRNA controls and are averages from three independent experiments. * p<0.05, paired Student's t-test, error bars are SEM, and NS is not significant.

Without wishing to be bound by a particular theory, the data are consistent with PLD regulation of Akt activation under serum-depleted conditions and that regulation is due to the PLD2 isoform.

4. PLD2 Directly Interacts with Akt

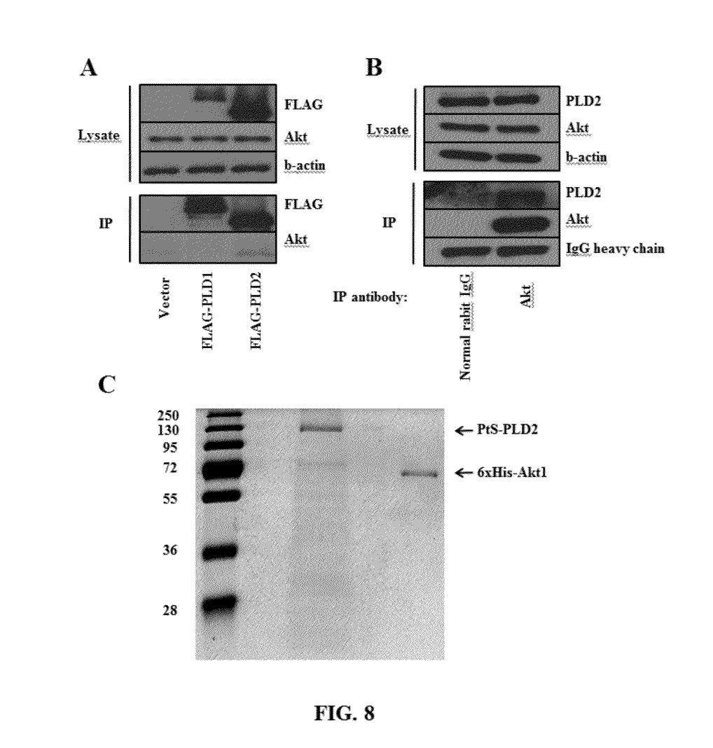

Since PLD2 is required for Akt activation following serum-withdrawal in glioblastoma cells, Akt and PLD2 were evaluated to determine whether Akt forms a protein complex with PLD2. Due to the lack of commercial antibodies suitable for immunoprecipitating (IP) endogenous PLD2, FLAG-PLD2 was transfected and immunoprecipitated from U87MG cells to probe for co-immunoprecipitation (co-IP) of endogenous Akt. Probing of PLD2 complexes with a pan-specific Akt antibody demonstrated an interaction of Akt with FLAG-PLD2 (FIG. 8A). Since overexpression of proteins is prone to artifacts, endogenous Akt was immunoprecipitated to demonstrate co-IP of endogenous PLD2 (FIG. 8B). Referring to FIG. 8A, U87MG cells were transfected with vector or FLAG-tagged PLD1 or PLD2 for 48 h. FLAG-PLD1 or PLD2 was immunoprecipitated and binding of endogenous Akt was assessed by immunoblotting FLAG-PLD complexes for Akt. Referring to FIG. 8B, U87MG cells were lysed and endogenous Akt immunoprecipitated overnight using a pan-specific Akt antibody. Non-specific proteins were immunoprecipitated using normal rabbit IgG. Note that approximately 0.5% of the material used for IP was loaded into the lysate lanes for both 8A and 8B. Together these results suggest that PLD2 and Akt form a protein complex in U87MG cells.

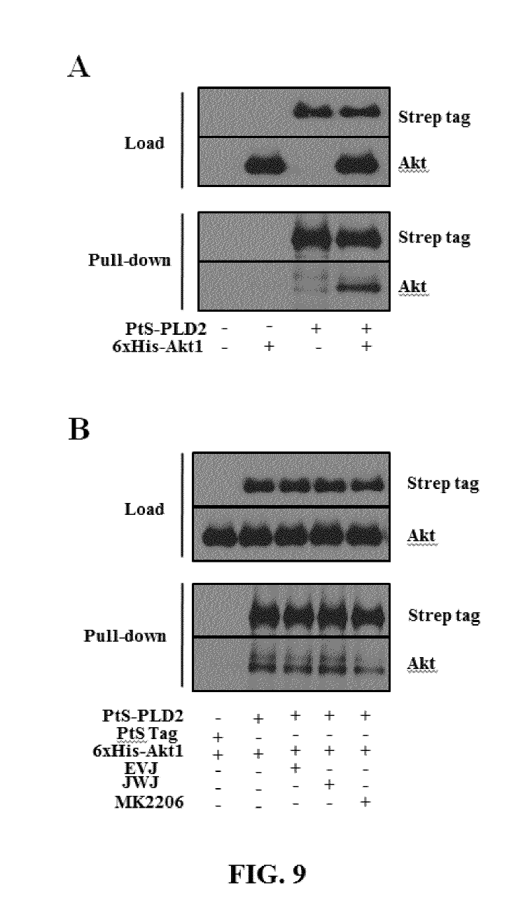

Although proteins may co-IP from cell lysate, determining whether proteins form a direct interaction requires binding experiments with purified proteins. As described herein, recombinant Akt and PLD2 was then expressed and purified from Sf21 insect cells (FIG. 8C). Referring to FIG. 8C, coomassie brilliant blue stained gel of PLD2 and Akt is shown to demonstrate protein purity. Numbers indicate molecular weights in kilodaltons. To demonstrate a direct protein-protein interaction, purified Akt (6×His-tagged) and PLD2 (ProteinA-tev-StrepII-tagged (Giannone, R. et al., (2007) Biotech. 43, 296-302)) were incubated together and PLD2 captured using strep-tactin affinity resin. Purified Akt bound PLD2 in the absence of other proteins in vitro suggesting that the PLD2 and Akt form a direct interaction (FIG. 9A). Referring to FIG. 9A, purified Akt was incubated with either PLD2 or ProteinA-tev-Strep (PtS) tag for 2 h and complexes captured using affinity resin. Bound Akt was determined by immunoblotting for Akt to demonstrate a direct protein-protein interaction between PLD2 and Akt. Since PLD2 and Akt appear to interact directly, the PLD inhibitors were evaluated to determine whether they disrupted the protein-protein interaction as a potential mechanism by which PLD2 regulates activation of Akt in cells. As described herein, purified PLD2 and Akt were incubated in the presence of EVJ, JWJ, or the allosteric pan-Akt inhibitor, MK2206 (Hirai, H. et al., (2010) Mol. Cancer Ther. 9, 1956-1967) and proteins captured using affinity resin. The PLD2-Akt interaction was detected even in the presence of PLD inhibitors. Referring to FIG. 9B, the same procedure was used as in 9A, except 10 μM EVJ, JWJ, or Akt inhibitor MK2206 was included in the reaction mixture. The Akt inhibitor, MK2206, disrupted the PLD2-Akt interaction.

Without wishing to be bound by a particular theory, the data are consistent with a previously unappreciated function of Akt inhibitors, e.g. MK2206, to disrupt Akt-protein interactions such those between PLD (e.g. PLD2) Akt.

5. Phosphatidic Acid Regulates Akt Activation

In order to determine the mechanism by which PLD2 regulates Akt activation following serum-withdrawal in GBM cells, a potential protein-protein interaction was investigated, as was demonstrated with PLD from N. gonorrhoeae (Edwards and Apicella, (2006) Cell Microbiol. 8, 1253-1271). Although a specific interaction of Akt with PLD2, but not PLD1, was detected in cell lysates (FIGS. 8A and 8B) and with recombinant, purified proteins (FIGS. 8C and 9A), PLD inhibitors did not disrupt the PLD2-Akt complex formation (FIG. 9B). This data suggests that the small molecule PLD inhibitors are not mediating Akt regulation by disrupting the PLD2-Akt protein complex.

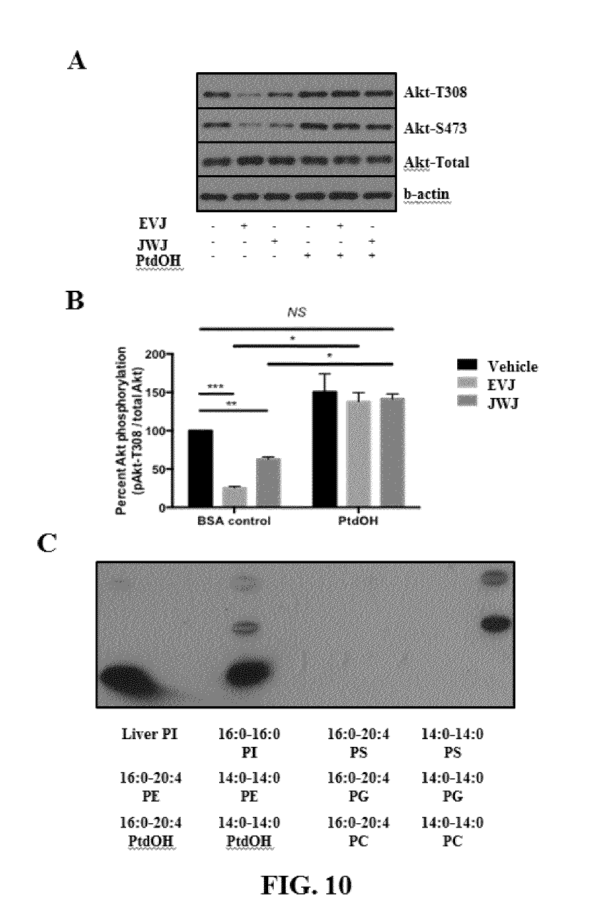

Since PLD inhibitors were not observed to disrupt the PLD2-Akt protein complex, a potential regulation of Akt by the catalytic product of PLD2, phosphatidic acid (PtdOH) was investigated. To confirm that the decrease in Akt phosphorylation following PLD inhibitor treatment or siRNA knockdown was due to the decrease in PtdOH production, the rescue of Akt phosphorylation by co-treatment of U87MG cells with PLD inhibitors and exogenously added PtdOH was attempted. A detailed lipidomic characterization of PtdOH species generated by PLD in an astrocytoma cell line was recently published (Scott, S. A. et al, (2013) J. Biol. Chem. 288, 20477-20487). Based upon this analysis and others, the rescue of Akt phosphorylation was attempted using 36:2 PtdOH, a species generated by PLD (Singh, S. K. et al., (2004) Nat. Cell Biol. 432, 396-401). Complete rescue of Akt phosphorylation with exogenously added PtdOH was observed (FIGS. 10A and 10B), suggesting that decreased Akt phosphorylation following PLD inhibitor treatment was due to decreased production of PtdOH by PLD. Referring to FIG. 10A, U87MG cells were treated overnight with 10 μM EVJ or 5 μM JWJ in DMEM containing 0.25 mg/mL fatty-acid free BSA. Where indicated, cells were co-treated with 1 mM PtdOH or BSA control and Akt phosphorylation was assessed by immunoblotting T308 and 5473. FIG. 10B shows the quantification of PtdOH rescue of Akt phosphorylation following PLD inhibitor treatment. Referring to FIG. 10B, fold changes in phosphor-Akt (T308) were determined relative to the vehicle treated, BSA control. Data were analyzed by repeated measures ANOVA across all conditions and post-hoc paired t-tests in indicated conditions (* p<0.05, ** p<0.01, *** p<0.0001).

Several studies have recently suggested that Akt binds other phospholipids in addition to phosphoinositides including phosphatidylserine (Huang, B. X. et al., (2011) J. Cell Bio. 192, 979-992) and PtdOH (Mahajan, K. et al., (2010) PLoS ONE 5, e9646). In order to compare the relative affinity of Akt for various phospholipids, Akt-lipid binding was determined using a commercially available protein-lipid overlay assay (Dowler, S. et al., (2002) Sci. STKE 2002, 6 μl-6) in which phospholipids are spotted onto nitrocellulose and binding of recombinant protein is detected immunologically. In agreement with other studies, recombinant Akt bound PtdOH and with higher affinity than other phospholipids (FIG. 10C). Referring to FIG. 10C, 5 nM recombinant Akt was incubated overnight with nitrocellulose membranes containing 2 μg of the indicated lipid. Bound Akt was measured using an Akt specific antibody. Phospholipids are denoted XX;Y, where XX refers to the number of carbon atoms in the acyl chain and Y indicates the degree of unsaturation. Error bars are SEM, PE is phosphatidylethanolamine, PC is phosphatidylcholine, PG is phosphatidylglycerol, and PS is phosphatidylserine.

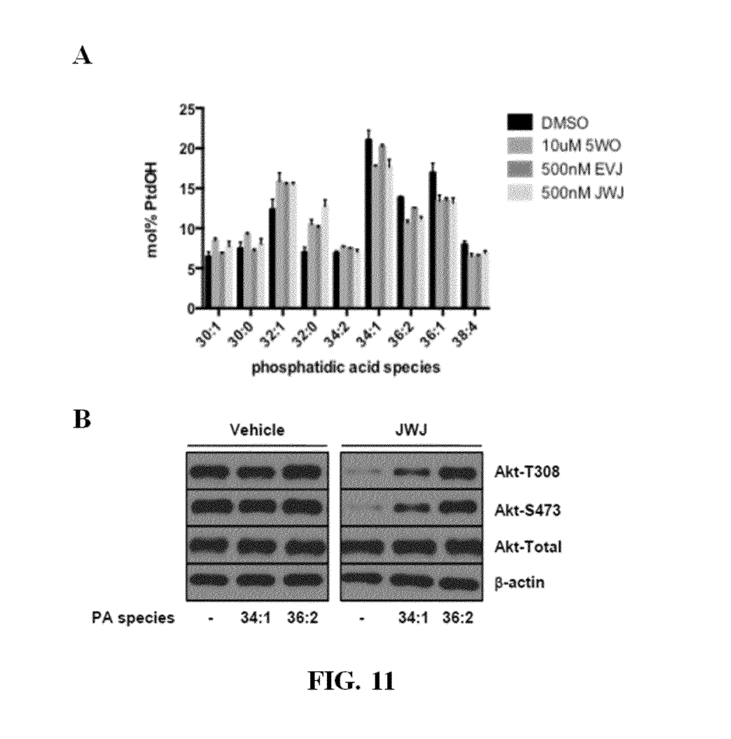

Other enzymes, besides PLD, have been reported to contribute to the overall levels of PA within the cell (Vance and Vance, (2008) J. Lipid Res. 50 Suppl, S132-7). In order determine which PA species were generated by PLD2 in the U87MG cells, a lipidomic approach was utilized to measure changes in PA species following overnight treatment with PLD inhibitors. U87MG cells were treated overnight with SWO, a dual PLD inhibitor, or isoform specific concentrations of EVJ and JWJ to distinguish between PA generated by PLD1 and PLD2. Only two PA species, 34:1 and 36:2, decreased in a PLD2 dependent manner (FIG. 11A). Referring to FIG. 11A, lipidomic analysis of PtdOH changes in U87MG cells following overnight serum withdrawal and PLD inhibitor treatment. U87MG cells were deprived of serum overnight in the presence of the indicated concentration of inhibitor. 24 hours after serum deprivation, samples were harvested for lipidomic analysis. In order to confirm that the regulation of Akt activation by PLD2 was through phosphatidic acid, Akt phosphorylation rescue following PLD inhibitor treatment was attempted by exogenously adding phosphatidic acid to U87MG cells following overnight JWJ treatment. Treatment of U87MG cells with either 34:1 or 36:2 PA partially rescued Akt phosphorylation following PLD inhibition (FIG. 11B), suggesting that phosphatidic acid is required for Akt activation following serum withdrawal. Referring to FIG. 11B, exogenous PA rescues Akt phosphorylation following PLD inhibition. U87MG cells were treated overnight with 5 μM JWJ. Lipids were dried under N2 gas then sonicated in DMEM containing 0.25 mg/ml fatty-acid free BSA to a final concentration of 1 mM lipid. Cells were treated with PA for 4 hours before cell harvest and immunoblotting.

6. Phosphatidic Acid Enhances Akt Binding to PIP₃ and Subsequent Membrane Recruitment

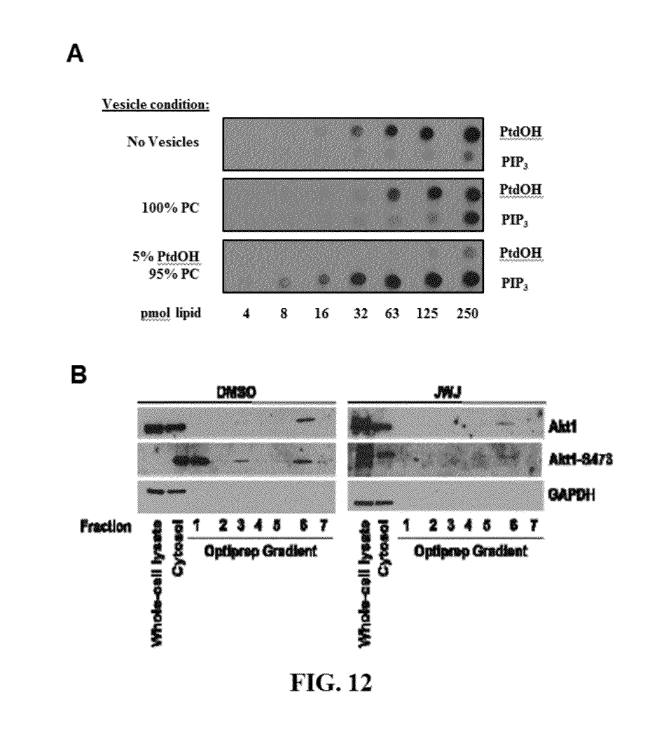

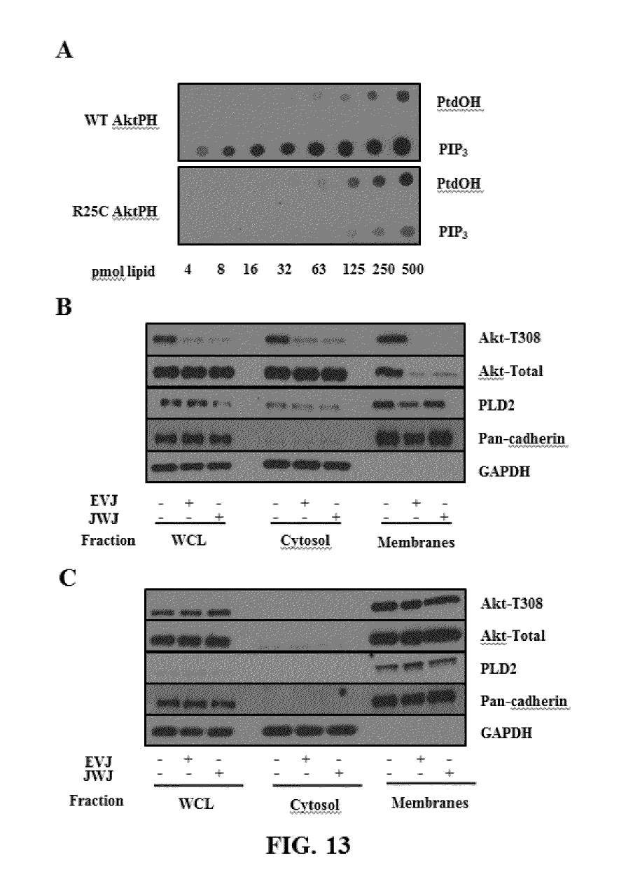

In order to understand the mechanism by which PtdOH regulates Akt activation, it was first determined whether PtdOH and PIP₃ share a binding site on Akt. Recombinant Akt was incubated with lipid vesicles composed of PC alone or PC with PtdOH. Akt binding to PtdOH and PIP₃ was then assessed using a protein-lipid overlay assay. When pre-incubated with vesicles containing PtdOH, binding of Akt to PtdOH on nitrocellulose was diminished, and this condition served as an internal control for the experiment (FIG. 12A). Referring to FIG. 12A, membrane recruitment of Akt is diminished following treatment of U87MG cells with JWJ. U87MG cells that were treated overnight with 5 μM JWJ were lysed in isosmotic buffer by nitrogen cavitation. Lysates were centrifuged at 2,000×g for 10 minutes. The crude membrane fraction was obtained by centrifuging the supernatant at 100,000 g for 1 hour. Membrane pellet was washed with lysis buffer to remove contaminating proteins and respun under the same conditions. The resulting pellet was resuspended in 30% iodixanol and layered at the bottom of a discontinuous iodixanol gradient containing approximately equal amounts of 2.5%, 10%, 17.5%, and 25% iodixanol. Membranes were floated for 3 hours at 165,00×g. 1 mL fractions were collected from the top of the gradient and immunoblotted for total and phospho-Akt1. Referring to FIG. 12B, recombinant Akt (3 nM) was incubated with 200 μM bulk lipid vesicles for 1 h then the Akt-vesicle mixtures were incubated overnight with nitrocellulose spotted with PtdOH or PIP₃. Bound Akt was determined using an Akt specific antibody. Intriguingly, binding of Akt to PIP₃ was strongly enhanced by pre-incubation with PtdOH-containing vesicles. Since PIP₃ is known to bind the Akt pleckstrin homology (PH) domain based on published crystal structures (Thomas, C. et al., (2002) Curr. Biol. 12, 1256-1262), the ability of the PH domain to mediate interactions with PtdOH was investigated. GST-Akt PH domain fusion proteins were purified from E. coli and lipid binding was again assessed using a protein-lipid overlay. The wild-type (WT) Akt PH domain and an Akt PH domain mutant deficient in PIP₃ binding, R25C (Thomas, C. et al., (2002) Curr. Biol. 12, 1256-1262), were then purified to determine if perturbing PIP₃ binding would also alter PtdOH binding. The WT PH domain of Akt was sufficient to bind PtdOH and disruption of PIP₃ binding with the R25C mutant had no effect on PtdOH binding (FIG. 13A). FIG. 13A shows a protein-lipid overlay measuring wild-type of R25C PIP₃ binding deficient mutant Akt PH domain (3 nM) binding to PtdOH or PIP₃. These results suggest that PtdOH binds a distinct site on the PH domain of Alt and that the binding of PtdOH acts cooperatively to increase the affinity of Akt for PIP₃.

Since PIP₃ recruits Akt to membranes (Bellacosa, A. et al., (1991) Science 254, 274-277; Franke, T. F. et al., (1995) Cell, 81, 727-736) and PtdOH increased Akt binding to PIP₃, the possibility that PLD-generated PtdOH regulates membrane localization of Akt was investigated. Membranes from serum-starved U87MG cells treated with vehicle or PLD inhibitors were prepared by flotation through a discontinuous iodixanol gradient. Akt and PLD2 co-fractionated in light and heavy membrane fractions, most likely corresponding to plasma membrane and ER/mitochondria, respectively based on subcellular markers (FIG. 13B). Under control conditions, Akt was present in both cytosolic and membrane fractions and the inhibition of PLD decreased the levels of both total and phosphorylated Akt in the membrane fraction. Akt membrane recruitment was less sensitive to PLD inhibition in the presence of serum and Akt was not detected in the membrane fractions of serum-starved HEK293 cells except under conditions where film was extremely overexposed, consistent with constitutive activation of Akt in PTEN-null GBM cells. To confirm that PLD inhibitors decreased Akt membrane localization in a PtdOH dependent manner, U87MG cells were co-treated with PLD inhibitors and the PLD product PtdOH before preparing membranes. Interestingly, co-treatment of cells with PtdOH not only rescued Akt membrane localization but PtdOH treatment resulted in a dramatic relocalization of cytosolic Akt to the membrane fraction (FIG. 13C). Referring to FIGS. 13B and 13C, U87MG cells were treated for 6 h with 10 μM PLD inhibitor in the absence (13B) or presence (13C) of 1 mM 36:2 PtdOH in DMEM+0.25 mg/mL BSA and separated into cytosol and membranes for immunoblotting. Glyceraldehyde 3-phosphate dehydrogenase (GADPH) and pan-cadherin were used as cytosolic and membrane markers, respectively. WCL is whole cell lysate and NS is not significant.

Without wishing to be bound by a particular theory, these data are consistent with a mechanism of PLD-generated PtdOH as a crucial mediator of Akt-membrane recruitment in cancer cells, e.g. glioblastoma cells.

7. PLD2 Inhibition Induces Autophagy-Dependent Cell Death

After establishing a requirement for PLD2-generated PtdOH in the activation of Akt in U87MG cells, the mechanism of cell death following inhibition of the PLD2-Akt pathway was determined by first measuring markers for apoptosis in U87MG cells. PLD inhibition only modestly induced caspase-3/7 cleavage relative to a well-characterized apoptotic stimulus, stauroporine (Bertrand, R. et al., (1994) Exp. Cell Res. 211, 314-321; Jacobsen, M. D. et al., (1996) J. Cell Biol. 133, 1041-1051) and treatment of U87MG cells with a pan caspase inhibitor failed to rescue PLD-inhibitor induced cell death (data not shown).

Without wishing to be bound by a particular theory, taken together, these data are consistent with PLD inhibition resulting in non-apoptotic cell death in cancer cells, e.g. glioblastoma cells.

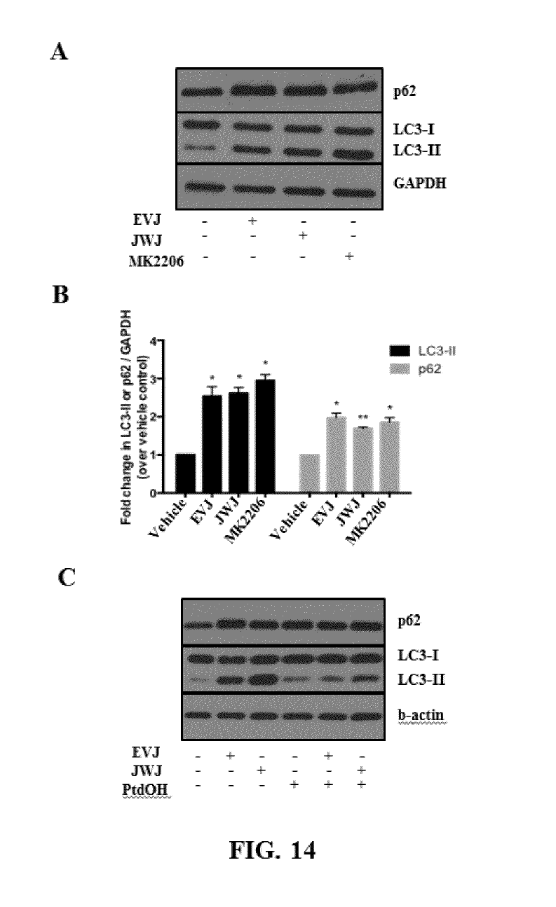

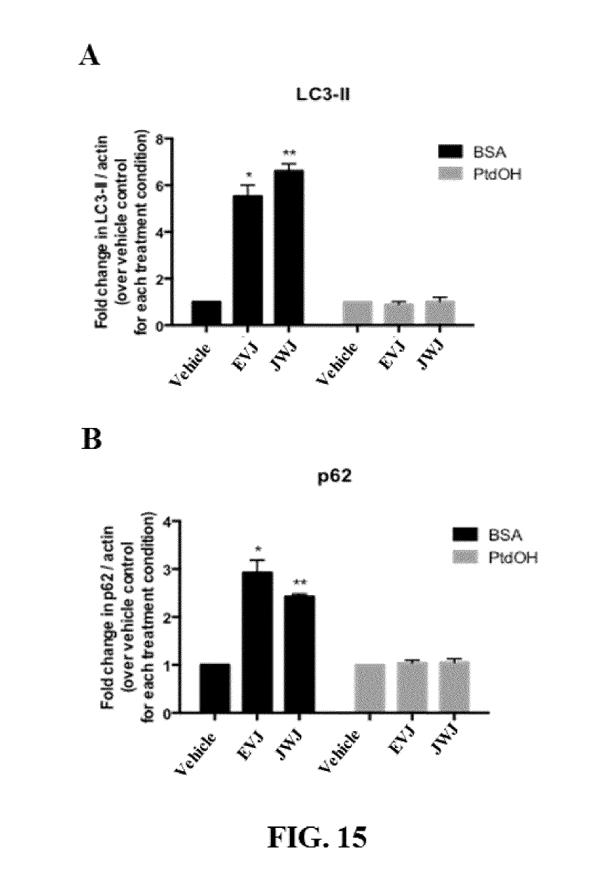

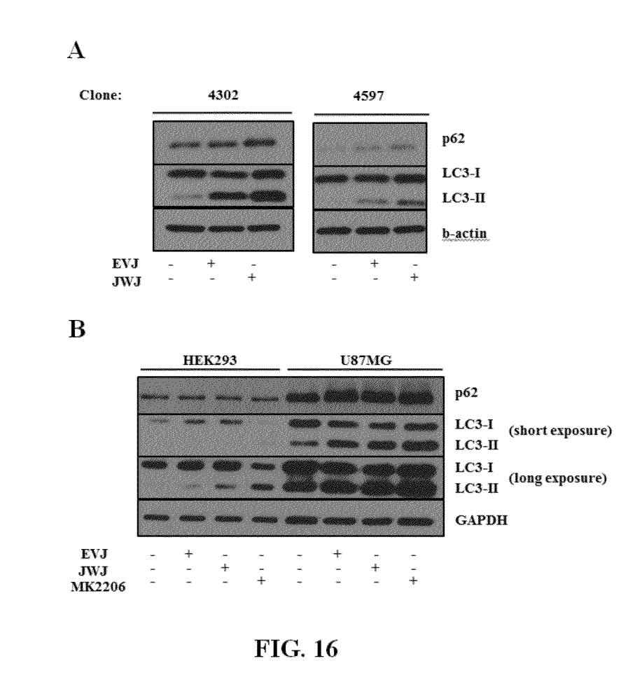

In addition to apoptosis, cells undergo another type of programmed cell death requiring autophagy (Tsujimoto and Shimizu, (2005) Cell Death Differ. 12 Suppl 2, 1528-1534), a process known to be stimulated by nutrient or growth factor deprivation (Kroemer, G. et al., (2010) Mol. Cell 40, 280-293). Autophagy is a multistep process involving formation of double-membrane autophagosomes that engulf cytosolic components and deliver cargo to lysosomes for digestion and nutrient recycling (Ravikumar, B. et al., (2009) J. Cell Sci. 122, 1707-1711). In order to determine if autophagy was perturbed following PLD or Akt inhibitor (MK2206) (Hirai, H. et al., (2010) Mol. Cancer Ther. 9, 1956-1967) treatment, the expression levels of the well-characterized autophagy makers microtubule-associated protein 1A/1B-light chain 3 (LC3) and p62 were measured. Autophagasome number is frequently assessed by measuring conversion of cytosolic LC3-I to a membrane-associated, lipidated LC3-II which is readily measurable as a faster migrating species of LC3 during SDS-PAGE (Kabeya, Y. et al., (2000) EMBO J. 19, 5720-5728). The other marker, p62, is an LC3 and ubiquitin-binding protein, involved in the regulation of protein aggregates and is degraded by autophagy (Komatsu, M. et al., (2005) J. Cell Biol. 169, 425-434). Induction of autophagy and successful degradation of autophagosomes would thus be accompanied by a decrease in p62 levels. Overnight treatment of U87MG cells with PLD or Akt inhibitors robustly induced LC3-II conversion and also increased p62 levels (FIGS. 14A and 14B). FIG. 14A shows an immunoblot of autophagy markers from U87MG cells treated overnight with 10 μM EVJ, 5 μM JWJ, or 10 μM MK2206 in serum-free DMEM. FIG. 14B shows that quantification of LC3-II and p62 increase in U87MG cells following PLD or Akt inhibition. Fold change in immunoreactivity relative to vehicle control. ANOVA with Dunnett's post-hoc test was used to compare inhibitor treatment to vehicle control (** p<0.01). These results suggest an increased number of autophagosomes resulting from a deficiency in autophagosome turnover as is often observed under conditions where autophagy is defective (Wang, Q. J. et al., (2006) J. Neurosci. 26, 8057-8068). In the presence of PtdOH, PLD inhibitors failed to increase autophagy markers, which further validates the specificity of our inhibitors (FIGS. 14C, 15A, and 15B). FIG. 14C shows an immunoblot of autophagy markers from U87MG cells treated overnight with PLD inhibitors with or without 1 mM 36:2 PtdOH in DMEM+0.25 mg/mL BSA. LC3-II and p62 levels also increased in other glioma cell lines including the CD133+ glioma stem cells (FIG. 16A) and U118MG cells (data not shown) following PLD inhibitor treatment. FIG. 16A shows an immunoblot of autophagy markers from CD133+ glioma stem cells treated overnight with 10 μM EVJ or 5 μM JWJ in neurobasal media without growth supplements.

To determine if the effects on autophagy following PLD inhibition were cell-type specific, LC3/p62 levels between U87MG cells and HEK293 cells were examined PLD and Akt inhibitors increased LC3-II conversion in both cell types (FIG. 16B). FIG. 16B shows a comparison of autophagy marker expression in U87MG or HEK293-TREx cells treated overnight with 10 μM EVJ, 5 μM JWJ, or 10 μM MK2206 in serum-free DMEM. However, the levels of LC3 and p62 under basal conditions were much higher in the U87MG cells.

Without wishing to be bound by a particular theory, these data are consistent with a mechanism wherein cancer cells, e.g. glioma cells, utilize autophagy more so than other cell types, rendering them particularly sensitive to compounds that perturb autophagy.

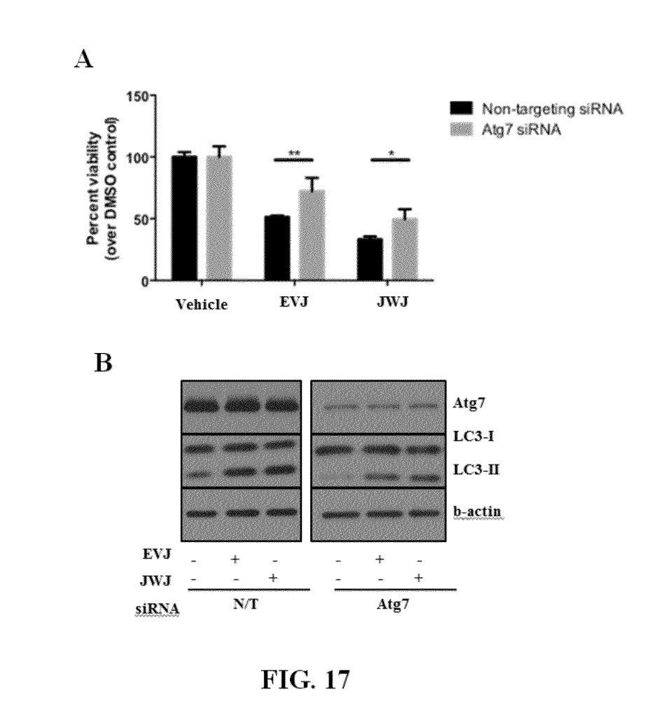

To confirm that U87MG cells were undergoing autophagy-dependent cell death, the ability of PLD inhibitors to decrease viability was examined when machinery required for autophagasome formation was perturbed by siRNA knockdown. Atg7 (Autophagy-related protein 7) is a ubiquitination E1-like enzyme required for autophagasome formation (Komatsu, M. et al., (2005) J. Cell Biol. 169, 425-434). Knockdown of Atg7 protein significantly increased viability (FIG. 17A) and decreased LC3-II conversion (FIG. 17B) following PLD inhibition in U87MG cells.

FIG. 17A shows data from a viability assay from U87MG cells treated with siRNA targeting Atg7. Cells were treated overnight with PLD or Akt inhibitors in serum-free DMEM before measuring viability with the WST-1 reagent. Two-way ANOVA with post-hoc Sidak multiple comparison test was used on Atg7 siRNA effect within inhibitor treatment groups (* p<0.05, ** p<0.01). FIG. 17B shows an immunoblot of LC3 and Atg7 for the experiment shown in 17A (error bars are SEM and N/T is non-targeting siRNA).

Without wishing to be bound by a particular theory, the data are consistent with cell death, e.g. in glioblastoma cells, resulting from PLD inhibition predominantly through an autophagy-dependent mechanism.

8. PLD and Akt Inhibition Reduces Autophagic Flux

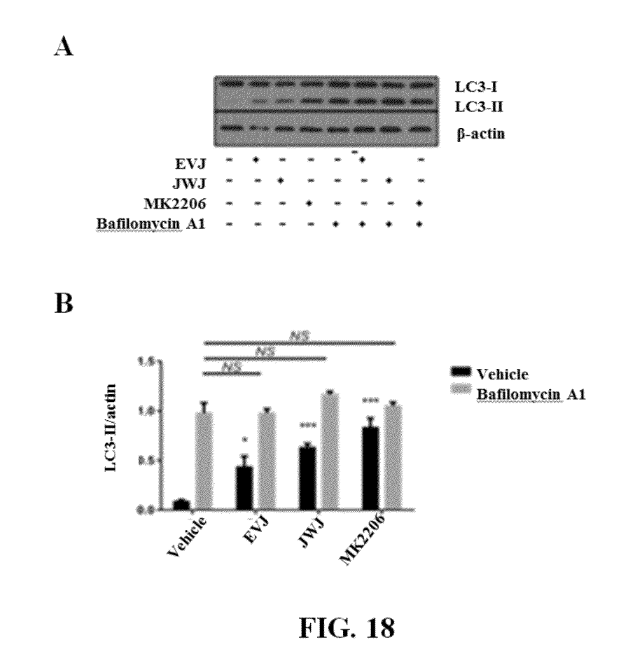

The increased conversion of LC3-II and increased expression of p62 after inhibitor treatments in glioma cells suggest that autoophagic flux requires PLD and Akt activity. To conclusively determine that flux, rather than autophagosome initiation, is regulated by PLC, LC3-II conversion in the presence of the lysosomal proton pump inhibitor bafilomycin A1, which prevents autophagosome fusion to lysosomes and inhibits degradation of autophagosomes, was measured. Thus, bafilomycin A1 is commonly used to discriminate the effects of a compound on autophagy initiation versus flux by assessing LC3-II levels in the presence of a test compound after clamping degradation of autophagosomes (Yamamoto, A. et al., (1998) Cell Struct. Funct. 23, 33-42). Bafilomycin A1, PLD, and Akt inhibitor treatment increased LC3-II levels relative to vehicle control (FIGS. 18A and 18B). FIG. 18A shows an immunoblot of LC3 from U87MG cells treated with 10 nM bafilomycin A1 for 30 minutes followed by treatment with 10 μM EVJ, 5 μM JWJ, or 10 μM MK2206 for 6 h in serum-free DMEM before cell harvest. FIG. 18B shows the quantification of LC3-II conversion in the presence of bafilomycin A1 and PLD/Akt inhibitors. Data are presented as the ratio of band intensity for LC3-II relative to Actin (* p<0.05, *** p<0.005, two-way ANOVA with Tukey's post-hoc test on PLD/Akt inhibitor effects versus vehicle control, NS—no significant impact of PLD inhibitors in conditions with bafilomycin A1).However, no additional accumulation of LC3-II was measured when PLD or Akt inhibitors were added in the presence of bafilomycin A1, confirming that PLD and Akt were controlling degradation of autophagosomes.

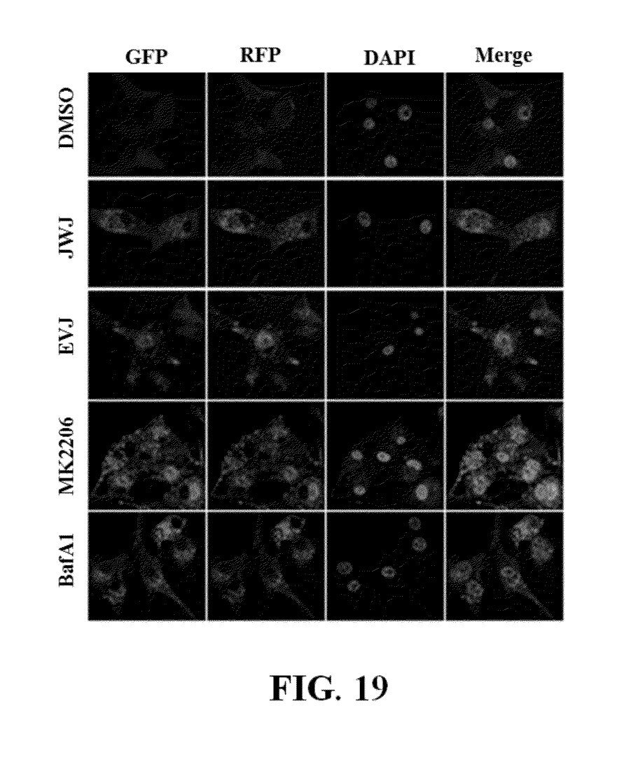

To further demonstrate decreased degradation of autophagosomes following PLD2/Akt inhibition, a stable U87GM cell line was generated to express a tandem-fluorescent LC3 reporter (tf-LC3) used to assess autophagosome maturation (Kimura, S. et al., (2007) Proc. Natl. Acad. Sci. U.S.A. 105, 19211-19216). This reporter system consists of a red fluorescent protein (RFP) and a green fluorescent protein (GFP) fused to LC3. As autophagosome numbers increase, either due to increased autophagy initiation or decreased degradation, fluorescence intensity increases as LC3 clusters on autophagosome membranes. Unlike RFP, GFP is quenched by low pH and LC3 present in lysosomes should predominantly emit an RFP signal. Under situations where autophagosome degradation is perturbed, the GFP and RFP signals co-localize since autophagosomes do not fuse to acidic lysosomes. The tF-LC3 U87MG cells were treated with PLD or Akt inhibitors overnight, fixed, and imaged for GFP and RFP signals. Under vehicle treated conditions, numbers of LC3 puncta were low and predominantly visualized with the pH-insensitive RFP tag, indicative of functional autophagy. However, PLD or Akt inhibitor treatments induced a robust relocalization of cytosolic LC3-I to large fluorescent puncta and when merged, the GFP/RFP signals highly co-localized, indicating a perturbation in the ability of the cell to effectively degrade and process autophagosomes (FIG. 19). Referring to FIG. 19, cells were treated overnight in serum-free DMEM with 10 μM EVJ, 5 μM JWJ, 10 μM MK2206, or 10 nM Bafilomycin A1 then fixed and imaged using confocal microscopy (error bars=SEM).

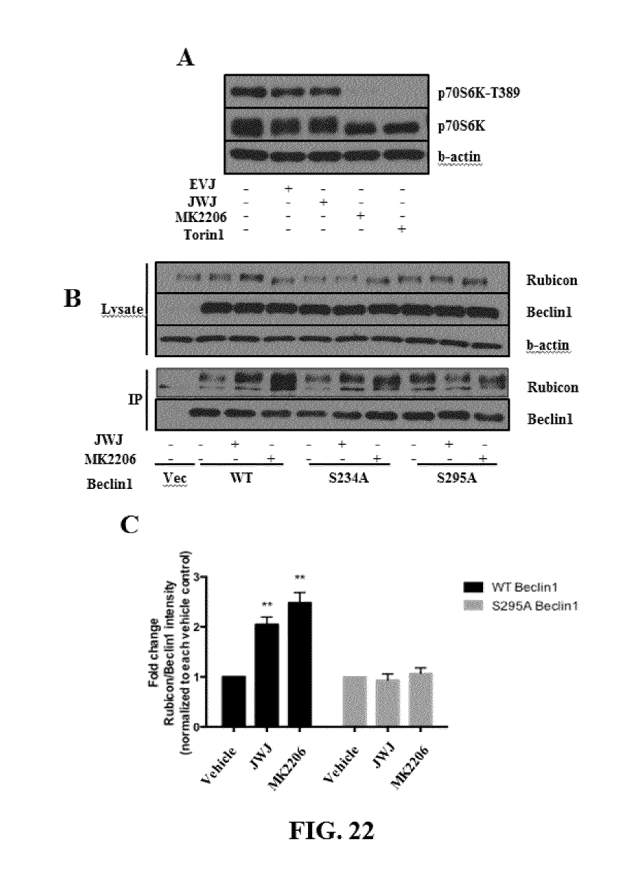

After establishing that PLD and Akt promote autophagic flux, the molecular mechanism was investigated. The mammalian target of rapamycin (mTOR) pathway suppresses autophagy under nutrient rich conditions and PLD has been implicated as an upstream positive regulator of mTOR (reviewed in Foster, D. A. (2009) Biochim. Biophys. Actas 1791, 949-955). Although the diminution of mTOR activity with Akt inhibitors was measured, little to no change in mTOR effector phosphorylation status was observed with PLD inhibition (FIG. 22A), suggesting the mTOR pathway was not mediating the effects of PLD inhibitors on autophagy and also suggesting that PLD2 and mTOR signaling are uncoupled in the U87MG cell line. Referring to FIG. 22A, U87MG cells were treated overnight with 10 μM EVJ, 5 μM JWJ, 10 μM MK2206, or 1 μM mTOR inhibitor Torin1. Cells were immunoblotted for total and phosphorylated p70S6K1. Since mTOR regulation did not explain the effects of PLD inhibition on autophagy, other Akt substrates were investigated. Recently, Akt was shown to phosphorylate Beclin1 and promote autophagy (Wang, R. C. et al., (2012) Science 338, 956-959). Beclin1 is a component of the core autophagy complex (Liang, X. H. et al., (1999) Nature 402, 672-676) and exists in multiple protein complexes during progressive stages of autophagy (Kihara, A. (2001) EMBO Reports 2, 330-335). Autophagosome maturation and subsequent degradation is, in part, regulated by the interaction of Beclin1 with RUN-domain cysteine rich domain containing, Beclin1 interacting protein (Rubicon) (Matsunaga, K. et al., (2009) Nat. Cell Biol. 11, 385-396; Zhong, Y. et al., (2009) Nat. Cell Biol. 11, 468-476), which is believed to negatively impact autophagosome maturation. The phosphorylation of Beclin1 by Akt may inhibit the interaction with Rubicon and either PLD2 or Akt inhibition would thereby enhance the interaction. As expected, the PLD2 inhibitor VU0364739 and Akt inhibitor MK2206 increased the amount of Rubicon that co-immunoprecipitated with Beclin1 from U87MG cells (FIGS. 22B and 22C). Referring to FIG. 22B, U87MG cells were transfected with HA-tagged wild type or mutant Beclin1 for 48 hours. Cells were treated with 10 μM JWJ or MK2206 for 6 h in serum-free DMEM prior to cell harvest and immunoprecipitation of HA-Beclin1 with a HA-antibody Immunoprecipitates were probed for co-IP of endogenous Rubicon. FIG. 22C shows the quantification of the increased binding of Rubicon to Beclin1 following PLD or Akt inhibition. Band intensities of Rubicon and Beclin1 were determined and the ratio of Rubicon to Beclin1 was calculated for each sample. Fold changes in this ratio were calculated by comparing inhibitor conditions to the vehicle treated conditions within the wild-type or S295A Beclin1 groups (n=4, ** p<0.01, ANOVA with Dunnett's post-hoc test, error bars=SEM). To address whether the interaction of Rubicon with Beclin1 was mediated by Akt phosphorylation, the two putative Akt phosphorylation residues on Beclin1, serine 234 and serine 295 (Wang, R. C. et al., (2012) Science 338, 956-959), were mutated and Rubicon binding assessed. Previous studies identified serine 295 as the predominant Akt phosphorylation site on Beclin1 (Wang, R. C. et al., (2012) Science 338, 956-959). Alanine mutation of serine 295, but not 234, increased Rubicon binding to Beclin1 compared to wild type controls (FIG. 22B). PLD and Akt inhibition failed to increase binding of Rubicon to the S295A mutant of Beclin1.

Without wishing to be bound by a particular theory, these data are consistent with a model wherein Akt activity enhances autophagic flux by preventing binding of Rubicon to Beclin1 (e.g. see FIG. 22B and FIG. 22C).

9. PLD Inhibition Upregulates Autophagy Through AMPK Activation

The mammalian target of rapamycin (mTOR) pathway suppresses autophagy under nutrient rich conditions (Jung, C. H. et al., (2010) FEBS Lett. 584, 1287-1295). PLD and Akt have been implicated as upstream positive regulators of mTOR in several studies (Fang, Y. et al., (2001) Science 294, 1942-1945; Foster, D. A., (2007) Cancer Res. 67, 1-4; Sun, Q. et al., (2008) Proc. Natl. Acad. Sci. U.S.A. 105, 19211-19216). In order to delineate the mechanism by which PLD inhibition induces autophagy, mTOR activity in U87MG cells following PLD inhibition was investigated. Overnight treatment with EVJ or JWJ in serum-free media failed to decrease p70S6-kinase phosphorylation (FIG. 22A), a well-established mTOR target (Chung, J. et al., (1992) Cell 69, 1227-1236).

Without wishing to be bound by a particular theory, these data are consistent with mTOR signaling being uncoupled from PLD2 signaling in cancer cells, e.g. glioblastomas.

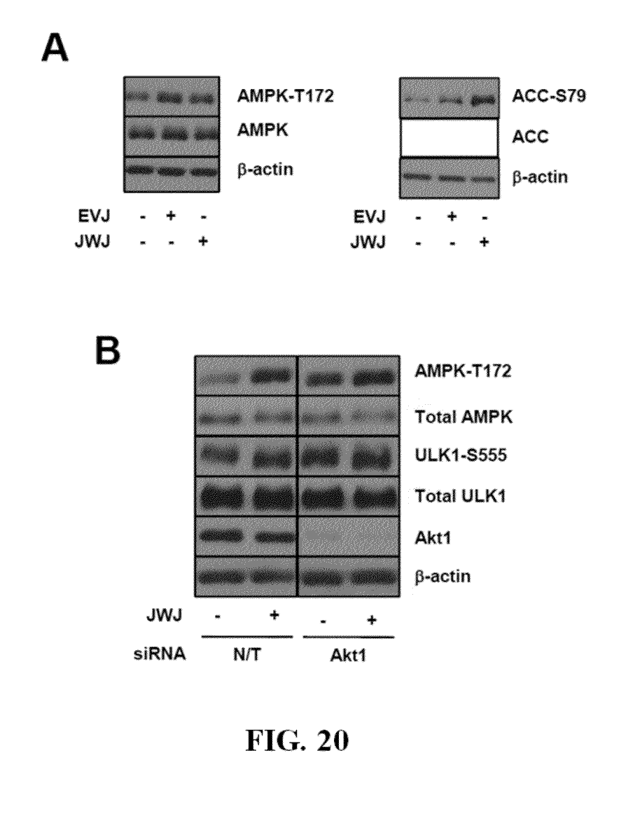

Another well-known regulator of autophagy is the adenosine monophosphate-activated protein kinase (AMPK). Changes in intracellular energy balance or metabolic stress are known to activate AMPK (Hardie, D. G. (2007) Nat. Rev. Mol. Cell Bio. 8, 774-785). AMPK serves as a metabolic checkpoint, acting to restore ATP levels through regulation of metabolic enzymes and inhibition of pro-growth anabolic pathways (Hardie, D. G. (2007) Nat. Rev. Mol. Cell Bio. 8, 774-785). Since changes in mTOR activity were not detected, AMPK was investigated tp determine whether AMPK activity was upregulated following PLD inhibition. AMPK activation was observed to increase following PLD inhibitor treatment in U87MG cells as determined by measuring phosphorylation of threonine 172 in the activation loop of the AMPK catalytic subunit (FIG. 20A) (Hawley, S. A. et al., (1996) J. Biol. Chem. 271, 27879-27887). FIG. 20A shows that PLD inhibition activates AMPK. U87MG cells were treated for 24 hours with 10 mM EVJ or 5 mM JWJ before lysis and immunoblotting. AMPK activation was measured by assessing phosphorylation at threonine 172 and also by measuring activation of acetyl coenzyme A carboxylase (ACC) at serine 79. Additionally, PLD inhibition increased phosphorylation of acetyl-coA carboxylase, a well-established AMPK substrate (Ha, J. et al., (1994) J. Biol. Chem. 269, 22162-22168).

Another AMPK substrate and component of the autophagasome initiation complex is Unc-51-like kinase 1 (ULK1). Several studies (Egan, D. F. et al., (2011) Science 331, 456-461; Kim, J. et al., (2011) Nature Cell Bio. 13, 132-141) have recently identified multiple AMPK sites on ULK1 that function to enhance initiation of autophagy. Overnight treatment with PLD inhibitors resulted in increased ULK1 phosphorylation at one of the AMPK residues, serine 555 (Egan, D. F. et al., (2011) Science 331, 456-461), providing a link between AMPK activation and initiation of autophagy following PLD inhibition. Akt is known to mediate a diverse set of functions in cellular metabolism (Manning and Cantley, (2007) Cell 129, 1261-1274) and Akt has been shown to impact AMPK activity through modulation of intracellular bioenergetics (Hahn-Windgassen, A. et al., (2005) J. Biolog. Chem. 280, 32081-32089). To determine whether the induction of autophagy through AMPK activation requires Akt, Akt1 was silenced and then evaluated for effects of PLD inhibition on AMPK and ULK1 phosphorylation. Akt1 was chosen to silence since Akt1 phosphorylation was decreased following PLD inhibition in U87MG cells. Once again, treatment with PLD inhibitors induced activation of both AMPK and ULK1. Interestingly, silencing of Akt1 also increased phosphorylation of AMPK and ULK1 and prevented further activation of AMPK following JWJ treatment (FIG. 20B). FIG. 20B shows that silencing of Akt1 activates AMPK. U87MG cells were transfected with Akt1 siRNA for 72 hours prior to cell lysis. Cells were treated with 5 mM JWJ 24 hours prior to cell lysis. JWJ does not appear to increase AMPK or ULK1 phosphorylation more than DMSO control when Akt1 is silenced.

Without wishing to be bound by a particular theory, together, these data are consistent with induction of autophagy following PLD inhibition resulting from metabolic stress induced activation of AMPK and its downstream targets through Akt.

10. Functional Rescue of Akt Restores Autophagic Flux and Viability in Glioma Cells Following PLD Inhibition

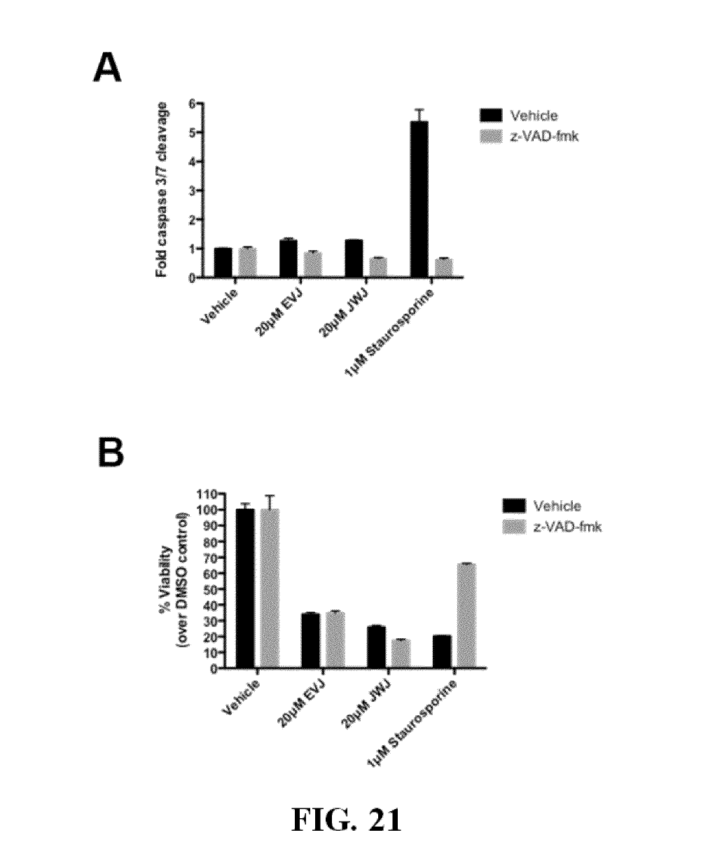

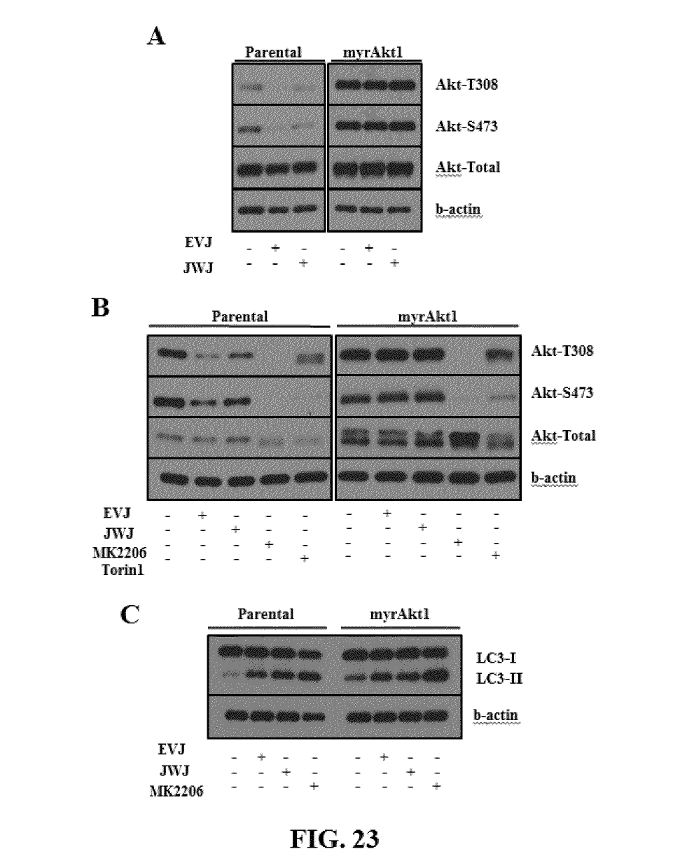

To confirm the observed effects on autophagy and cell death following PLD inhibition in glioma cells was due to the regulation of Akt by PLD2, a stable U87MG cell line was developed to expresses a constitutively active form of Akt under the transcriptional control of the tetracycline repressor protein (Yao, F. et al., (1998) Hum. Gene Ther. 9, 1939-1950). This Akt construct contains the myristoylation sequence from Src kinase (Kohn, A. D. et al., (1996) J. Biol. Chem. 271, 31372-31378) and is constitutively membrane associated and active. Without wishing to be bound by a particular theory, if PtdOH serves to enhance membrane docking of Akt then PLD inhibition should not decrease phosphorylation of myristoylated Akt (myrAkt) since this construct bypasses lipid recruitment signals for membrane association. As expected, PLD inhibitors failed to reduce levels of phosphorylated Akt in myrAkt1 U87MG cells (FIGS. 21A and 23A). FIG. 21A shows that activation of myrAkt1 is resistant to PLD inhibition. Tetracycline inducible myrAkt1-expressing U87MG cells, or parental cells, were seeded into media containing 0.1 ug/ml tetracycline and protein expression induced for approximately 24 hours. Cells were treated with 10 mM EVJ, JWJ, or MK2206 for approximately 16 hours before cell lysis and immunoblotting for Akt phosphorylation. FIG. 21B shows that myrAkt1 expression prevents PLD inhibitor induced AMPK activation. Cells were seeded and treated as in figure A and immunoblotted for phosphorylated and total AMPK. AMPK activation was quantitated by densitometric quantification of the phospho-AMPK band over the total AMPK band. Fold changes were calculated by normalizing the DMSO condition to 1 for each cell line. Unlike PLD inhibitors, Torin1, an ATP-site mTOR inhibitor (Thoreen, C. C. et al., (2009) J. Biol. Chem. 284, 8023-8032), decreased phosphorylation of myrAkt1, demonstrating that mTORC2 activity is still required for myrAkt1 phosphorylation and that inhibition of PLD activity does not decrease mTORC2 activity in this cell line (FIG. 23B). FIG. 23A shows an immunoblot of phosphorylated Akt from parental or myrAkt1-expressing U87MG cells treated overnight with 10 μM EVJ or 5 μM JWJ in serum-free DMEM. Referring to FIG. 23B, parental or myrAkt1 U87MG cells were treated with the indicated inhibitors as in 23A overnight before blotting for phosphorylated and total Akt.

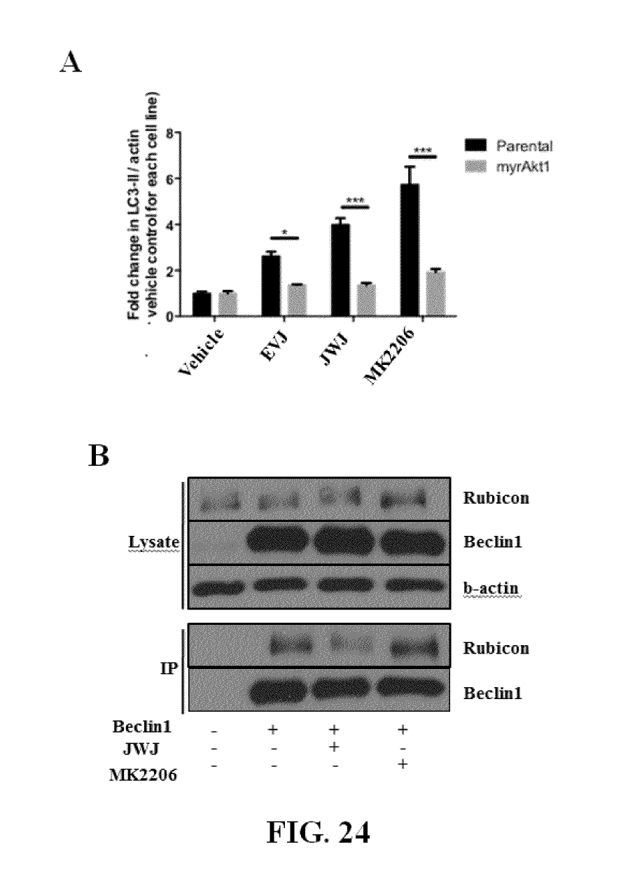

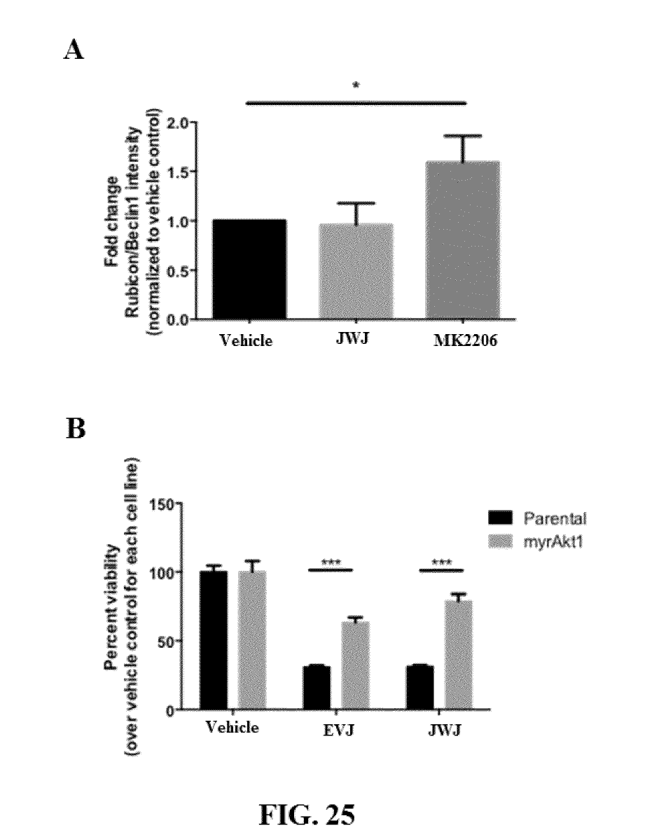

Since phosphorylation of myrAkt1 was resistant to PLD inhibition, whether autophagic flux was restored following myrAkt1 expression in PLD inhibitor treated cells was then assessed. Expression of myrAkt1 produced a modest increase in the basal level of LC3-II versus the parental U87MG line (FIG. 23C). FIG. 23C shows an immunoblot of LC3 from parental or myrAkt1 U87MG following 6 h treatment with PLD or Akt inhibitors in serum-free DMEM. This result is consistent with PLD regulating Akt by enhancing membrane recruitment rather than regulating kinases or phosphatases that modulate phosphorylation of threonine 308 and serine 473. However, the fold induction of LC3-II due to PLD inhibitor treatment versus vehicle control was significantly less than in the parental U87MG line (FIGS. 23C and 24A), suggesting that the decrease in autophagic flux was due to inactivation of Akt via a PLD dependent mechanism. Mechanistically, expression of myrAkt1 should prevent the increased binding of Rubicon to Beclin1 following treatment with PLD inhibitors. Treatment of myrAkt1 U87MG cells with Akt inhibitor MK2206, but not JWJ, increased Beclin1 binding to Rubicon even in the presence of myrAkt1 (FIGS. 24B and 25A), supporting the proposed mechanism that PLD2 inhibition results in the inactivation of Akt, which promotes the Rubicon-Beclin1 interaction and inhibits autophagic flux.

FIG. 24A shows the quantification of LC3-II conversion in parental and myrAkt1 U87MG cells following PLD and Akt inhibition. Fold changes were determined by calculating the ratio of LC3-II in inhibitor treated samples to vehicle treated samples within each cell line (* p<0.05, *** p<0.005, two-way ANOVA with Tukey's post-hoc test). Referring to FIG. 24B, MyrAkt1-U87MG cells were seeded and treated as in FIG. 22B.

Finally, to confirm that the decrease in viability following PLD inhibition was due to inhibition of Akt, U87MG cell viability in the parental and myrAkt1 lines was evaluated. Restoration of Akt function significantly increased viability and protected the GBM cells from PLD inhibitor induced cell death (FIG. 25B).

FIG. 25A shows the quantification of Rubicon binding to Beclin1 in myrAkt1 U87MG cells. Binding was quantified as in FIG. 22C and * p<0.05 using a paired student's t-test (n=4). FIG. 25B shows data from a WST-1 viability assay with parental or myrAkt1 U87MG cells treated for 24 hours with 20 μM EVJ or 10 μM JWJ in serum-free DMEM. Data is presented as the viability remaining following inhibitor treatment compared to the vehicle control within each cell type (*** p<0.005, two-way ANOVA with Sidek's post-hoc test, error bars=SEM).

Without wishing to be bound by a particular theory, the data are consistent with PLD activity being required for full Akt activation in cells, e.g. GBM cells, and that when inhibited, cells undergo autophagic death.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for treating a disorder of uncontrolled cellular proliferation in a subject, comprising the step of co-administering to the subject an Akt therapeutic agent and a phospholipase D inhibitor, thereby treating the disorder in the subject.

2. The method of claim 1, wherein the amount of the Akt therapeutic agent co-administered with the phospholipase D inhibitor is less than the amount of the Akt therapeutic agent administered in the absence of the phospholipase D inhibitor in order to achieve substantially the same therapeutic effect in the subject.

3. The method of claim 1, wherein the PLD inhibitor is a compound having a structure represented by a formula: 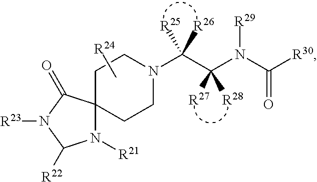
wherein each ----- independently comprises an optional covalent bond;
wherein R²¹ is an optionally substituted C3 to C9 organic residue selected from aryl, heteroaryl, cycloalkyl, heterocycloalkyl, cycloalkenyl, and heterocycloalkenyl;
wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein R²³ comprises hydrogen, an optionally substituted C1 to C6 alkyl, an optionally substituted C3 to C6 cycloalkyl, or a hydrolysable residue;
wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl;
wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl;
wherein R²⁹ comprises hydrogen, an optionally substituted C1 to C6 alkyl, an optionally substituted C3 to C6 cycloalkyl, or a hydrolysable residue; and
wherein R³⁰ comprises an optionally substituted C1 to C16 organic residue selected from alkyl, aryl, heteroaryl, cycloalkyl, heterocycloalkyl, cycloalkenyl, and heterocycloalkenyl;
or a pharmaceutically acceptable salt, hydrate, solvate, or polymorph thereof.

4. The method of claim 1, wherein the PLD inhibitor is a compound having a structure represented by a formula: 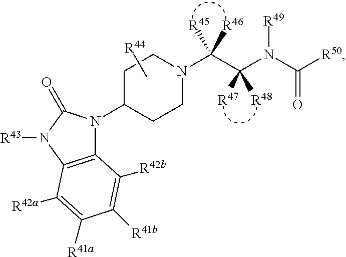
wherein each ----- independently comprises an optional covalent bond;
wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein R⁴³ comprises hydrogen, an optionally substituted C1 to C6 alkyl, an optionally substituted C3 to C6 cycloalkyl, or a hydrolysable residue;
wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl;
wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl;
wherein R⁴⁹ comprises hydrogen, an optionally substituted C1 to C6 alkyl, an optionally substituted C3 to C6 cycloalkyl, or a hydrolysable residue; and
wherein R⁵⁰ comprises an optionally substituted C1 to C16 organic residue selected from alkyl, aryl, heteroaryl, cycloalkyl, heterocycloalkyl, cycloalkenyl, and heterocycloalkenyl;
or a pharmaceutically acceptable salt, hydrate, solvate, or polymorph thereof.

5. The method of claim 1, wherein the Akt therapeutic agent is an Akt inhibitor that binds to the pleckstrin homology domain or is an ATP-competitive inhibitor.

6. The method of claim 5, wherein the Akt inhibitor is selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine.

7. The method of claim 1, wherein the Akt therapeutic agent is an antisense oligonucleotide.

8. The method of claim 7, wherein the antisense oligonucleotide is RX-0201.

9. A kit comprising an effective amount of a phospholipase D inhibitor, or a pharmaceutically acceptable prodrug, salt, solvate, or polymorph thereof; an effective amount of a mTor inhibitor, or a pharmaceutically acceptable prodrug, salt, solvate, or polymorph thereof; and one or more of:
a) an effective amount of at least one agent known to treat a disorder of uncontrolled cellular proliferation;
b) an effective amount of an Akt therapeutic agent;
c) at least one agent known to increase Akt activity; or
d) instructions for treating a disorder of uncontrolled cellular proliferation.

10. The kit of claim 9, wherein the effective amount of the PLD inhibitor is a therapeutically effective amount; and wherein the effective amount of the mTor inhibitor is a therapeutically effective amount.

11. The kit of claim 9, wherein the PLD inhibitor is a compound having a structure represented by a formula: 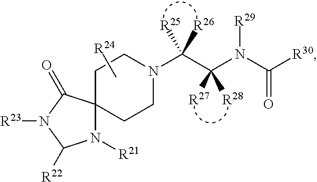
wherein each ----- independently comprises an optional covalent bond;
wherein R²¹ is an optionally substituted C3 to C9 organic residue selected from aryl, heteroaryl, cycloalkyl, heterocycloalkyl, cycloalkenyl, and heterocycloalkenyl;
wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein R²³ comprises hydrogen, an optionally substituted C1 to C6 alkyl, an optionally substituted C3 to C6 cycloalkyl, or a hydrolysable residue;
wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl;
wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl;
wherein R²⁹ comprises hydrogen, an optionally substituted C1 to C6 alkyl, an optionally substituted C3 to C6 cycloalkyl, or a hydrolysable residue; and
wherein R³⁰ comprises an optionally substituted C1 to C16 organic residue selected from alkyl, aryl, heteroaryl, cycloalkyl, heterocycloalkyl, cycloalkenyl, and heterocycloalkenyl;
or a pharmaceutically acceptable salt, hydrate, solvate, or polymorph thereof.

12. The kit of claim 9, wherein the PLD inhibitor is a compound having a structure represented by a formula: 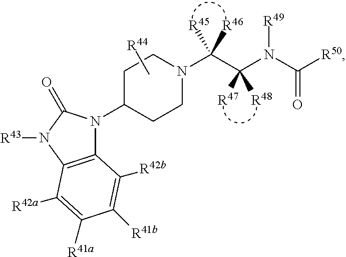
wherein each ----- independently comprises an optional covalent bond;
wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein R⁴³ comprises hydrogen, an optionally substituted C1 to C6 alkyl, an optionally substituted C3 to C6 cycloalkyl, or a hydrolysable residue;
wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl;
wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl;
wherein R⁴⁹ comprises hydrogen, an optionally substituted C1 to C6 alkyl, an optionally substituted C3 to C6 cycloalkyl, or a hydrolysable residue; and
wherein R⁵⁰ comprises an optionally substituted C1 to C16 organic residue selected from alkyl, aryl, heteroaryl, cycloalkyl, heterocycloalkyl, cycloalkenyl, and heterocycloalkenyl;
or a pharmaceutically acceptable salt, hydrate, solvate, or polymorph thereof.

13. The kit of claim 9, wherein the mTor inhibitor is selected from everolimus, rapamycin (sirolimus), temsirolimus, deforolimus, ridaforolimus, tacrolimus, zotarolimus, salirasib, curcumin, farnesylthiosalicylic acid, XL765, ABI-009, AP-23675, AP-23841, AP-23765, AZD-8055, AZD-2014, BEZ-235 (NVP-BEZ235), BGT226, GDC-0980, INK-128, KU-0063794, MK8669, MKC-1 (Ro 31-7453), NVP-BGT226, OSI-027, Palomid-529, PF-04691502, PKI-402, PKI-587, PP-242, PP-30, SB-1518, SB-2312, SF-1126, TAFA-93, TOP-216, Torin1, WAY-600, WYE-125132, WYE-354, WYE-687, and XL-765, or a pharmaceutically acceptable prodrug, salt, solvate, or polymorph thereof.

14. A pharmaceutical composition comprising:
a) a first therapeutic agent comprising an effective amount of a phospholipase D inhibitor, or a pharmaceutically acceptable prodrug, salt, solvate, or polymorph thereof; and
b) a second therapeutic agent comprising an effective amount of a mTor inhibitor, or a pharmaceutically acceptable prodrug, salt, solvate, or polymorph thereof;
and a pharmaceutically acceptable carrier.

15. The composition of claim 14, wherein the PLD inhibitor is a compound having a structure represented by a formula: 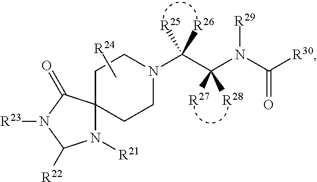
wherein each ----- independently comprises an optional covalent bond;
wherein R²¹ is an optionally substituted C3 to C9 organic residue selected from aryl, heteroaryl, cycloalkyl, heterocycloalkyl, cycloalkenyl, and heterocycloalkenyl;
wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein R²³ comprises hydrogen, an optionally substituted C1 to C6 alkyl, an optionally substituted C3 to C6 cycloalkyl, or a hydrolysable residue;
wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl;
wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl;
wherein R²⁹ comprises hydrogen, an optionally substituted C1 to C6 alkyl, an optionally substituted C3 to C6 cycloalkyl, or a hydrolysable residue; and
wherein R³⁰ comprises an optionally substituted C1 to C16 organic residue selected from alkyl, aryl, heteroaryl, cycloalkyl, heterocycloalkyl, cycloalkenyl, and heterocycloalkenyl;
or a pharmaceutically acceptable salt, hydrate, solvate, or polymorph thereof.

16. The composition of claim 14, wherein the PLD inhibitor is a compound having a structure represented by a formula: 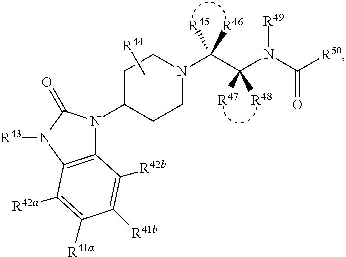
wherein each ----- independently comprises an optional covalent bond;
wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein R⁴³ comprises hydrogen, an optionally substituted C1 to C6 alkyl, an optionally substituted C3 to C6 cycloalkyl, or a hydrolysable residue;
wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue;
wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl;
wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl;
wherein R⁴⁹ comprises hydrogen, an optionally substituted C1 to C6 alkyl, an optionally substituted C3 to C6 cycloalkyl, or a hydrolysable residue; and
wherein R⁵⁰ comprises an optionally substituted C1 to C16 organic residue selected from alkyl, aryl, heteroaryl, cycloalkyl, heterocycloalkyl, cycloalkenyl, and heterocycloalkenyl;
or a pharmaceutically acceptable salt, hydrate, solvate, or polymorph thereof.

17. The composition of claim 14, wherein the mTor inhibitor is selected from everolimus, rapamycin (sirolimus), temsirolimus, deforolimus, ridaforolimus, tacrolimus, zotarolimus, salirasib, curcumin, farnesylthiosalicylic acid, XL765, ABI-009, AP-23675, AP-23841, AP-23765, AZD-8055, AZD-2014, BEZ-235 (NVP-BEZ235), BGT226, GDC-0980, INK-128, KU-0063794, MK8669, MKC-1 (Ro 31-7453), NVP-BGT226, OSI-027, Palomid-529, PF-04691502, PKI-402, PKI-587, PP-242, PP-30, SB-1518, SB-2312, SF-1126, TAFA-93, TOP-216, Torin1, WAY-600, WYE-125132, WYE-354, WYE-687, and XL-765, or a pharmaceutically acceptable prodrug, salt, solvate, or polymorph thereof.

18. The composition of claim 14, further comprising an effective amount of an Akt therapeutic agent.

19. The composition of claim 18, wherein the Akt therapeutic agent is an Akt inhibitor selected from A-443654, A-674563, Akti-1. Akti-2, Akti-1/2, AR-42, API-59CJ-OMe, ATI-13148, AZD-5363, erucylphosphocholine, GDC-0068, GSK-690693, GSK-2141795 (GSK795), KP372-1, L-418, LY294002, MK-2206, NL-71-101, PBI-05204, perifosine, PHT-427, PIA5, PX-316, SR13668, and triciribine.

20. The composition of claim 18, wherein the Akt therapeutic agent is an antisense oligonucleotide or siRNA.


SEQUENCE LISTING (6 sequences)

SEQ ID NO: 1 - DNA, 26 bp, Artificial Sequence
  synthetic construct, human p31-N-Pts forward primer
atggatccgcaacaacctggacagca

SEQ ID NO: 2 - DNA, 33 bp, Artificial Sequence
  synthetic construct, human p31-N-PtS reverse primer
aataagctttaagccatggtggacaacaaattc

SEQ ID NO: 3 - DNA, 34 bp, Artificial Sequence
  synthetic construct, human PLD2 forward primer
ataagaatgcggccgcatgacggcgacccctgag

SEQ ID NO: 4 - DNA, 29 bp, Artificial Sequence
  synthetic construct, human PLD2 reverse primer
gctctagacaactatgtccacacttctag

SEQ ID NO: 5 - DNA, 31 bp, Artificial Sequence
  synthetic construct, human PtS-PLD2 forward primer
acgcgtcgacgccatggtggacaacaaattc

SEQ ID NO: 6 - DNA, 36 bp, Artificial Sequence
  synthetic construct, human PtS-PLD2 reverse primer
ataagaatgcggccgcaactatgtccacacttctag